(19) United States
(12) Patent Application Publication
(10) Pub. No.: US 2024/0368183 A1
(43) Pub. Date: Nov. 7, 2024

(54) Macrocyclic Compounds Having ENT1 Inhibiting Activity

(71) Applicant: iTeos Belgium SA, Gosselies (BE)

(72) Inventors: Michael Deligny, Rhode Saint Genese (BE); Erica Joke Katelijne Heleen Houthuys, Halle (BE); Eric Talbot, Hoddesdon (GB); Mustafa Moroglu, Hoddesdon (GB)

(73) Assignee: iTeos Belgium SA, Gosselies (BE)

(21) Appl. No.: 18/616,553

(22) Filed: Mar. 26, 2024

Related U.S. Application Data
(63) Continuation of application No. PCT/US2022/045808, filed on Oct. 5, 2022.
(60) Provisional application No. 63/252,849, filed on Oct. 6, 2021.

(51) Int. Cl. C07D 498/08; A61K 31/395; A61K 31/439; A61K 31/4995; A61K 31/551; A61K 45/06; C07D 273/01; C07D 498/18; C07D 515/08

(52) U.S. Cl. CPC C07D 498/08; A61K 31/395; A61K 31/439; A61K 31/4995; A61K 31/551; A61K 45/06; C07D 273/01; C07D 498/18; C07D 515/08

(57) ABSTRACT

The present disclose includes, among other things, compounds of formula (I) that treat or lessen the severity of cancer, pharmaceutical compositions and methods of making and using the same.

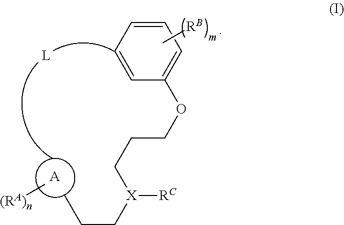

This application is a continuation of International Application No. PCT/US2022/045808, filed Oct. 5, 2022, which claims the benefit under 35 U.S.C. 119(e) of U.S. Provisional Application No. 63/252,849, filed on Oct. 6, 2021, both of which are herein incorporated by reference in their entirety.

### BACKGROUND

The equilibrative nucleoside transporter (ENT) family, also known as SLC29, is a group of plasmalemmal transport proteins which transport nucleoside substrates into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A2A, A2B, and A3. By influencing the concentration of adenosine available to these receptors, ENTs fulfil important regulatory roles in different physiological processes, such as modulation of coronary blood flow, inflammation, and neurotransmission (Griffith D A and Jarvis S M. Biochim Biophys Acta, 1996, 1286, 153-181; Shryock J C and Belardinelli L, Am J Cardiol, 1997, 79(12A), 2-10; Anderson C M et al., J Neurochem, 1999, 73, 867-873).

Adenosine is also a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358) Adenosine activates four G-protein-coupled receptor subtypes (A1, A2A, A2B, and A3). In particular, activation of the A2A receptor is believed to be the main driver of innate and adaptive immune cell suppression leading to suppression of antitumor immune responses (Ohta and Sitkovsky, Nature, 2001, 414, 916-920) (Stagg and Smyth, Oncogene, 2010, 2, 5346-5358) (Antonioli L et al., Nature Reviews Cancer, 2013, 13, 842-857) (Cekic C and Linden J. Nature Reviews, Immunology, 2016, 16, 177-192) (Allard B et al., Curr Op Pharmacol, 2016, 29, 7-16) (Vijayan D et al., Nature Reviews Cancer, 2017, 17, 709-724).

The Applicant previously evidenced in PCT/EP2019/076244 that adenosine as well as ATP profoundly suppress T cell proliferation and cytokine secretion (IL-2), and strongly reduce T cell viability. Adenosine- and ATP-mediated suppression of T cell viability and proliferation were successfully restored by using ENTs inhibitors. Moreover, the use of an ENT inhibitor in combination with an adenosine receptor antagonist enabled to restore not only adenosine- and ATP-mediated suppression of T cell viability and proliferation, but also restored T cell cytokine secretion. These results showed that ENTs inhibitors either alone or in combination with an adenosine receptor antagonist may be useful for the treatment of cancers.

A variety of drugs such as dilazep, dipyridamole, and draflazine interact with ENTs and alter adenosine levels, and were developed for their cardioprotective or vasodilatory effects.

Currently, two non-selective ENT1 inhibitors (dilazep and dipyridamole) are on the market (Vlachodimou et al., Bio-Chemical Pharmacology, 2020, 172, 113747). However, their binding kinetics are unknown; moreover, there is still a need for more potent ENTs inhibitors, and especially ENT1 inhibitors to be used for the treatment of cancers, either alone or in combination with an adenosine receptor antagonist.

Hence, this study focused on finding new and improved ENT1 inhibitors. For that purpose, the Applicant herein provides the macrocyclic diamine derivatives of formula (I) detailed below.

### SUMMARY

In some embodiments, the present disclosure includes a compound of formula (I):

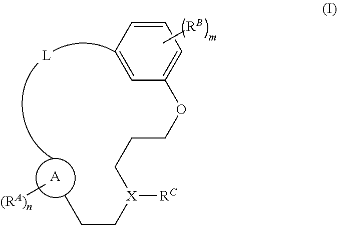

or a pharmaceutically acceptable salt thereof.

Additionally, the present disclosure includes, among other things, pharmaceutical compositions, methods of using and methods of making a compound of formula (I).

### DETAILED DESCRIPTION

In some embodiments, the present disclosure includes a compound of formula (I):

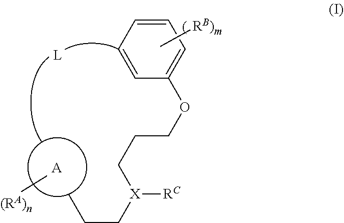

or a pharmaceutically acceptable salt thereof

wherein
L is an optionally substituted C₃-C₇ alkylene chain, wherein one, two, or three methylene units is optionally and independently replaced with —O—, —N(R¹)—, —C(O)—, —C(O)O—, —C(O)N(R¹)—, —S(O)₂—, a 5-membered heteroaryl, —CH═CH—, or —C≡C—;
A is selected from the group consisting of —N(R^{A})— and 5-7 membered heterocyclyl;
X is C(H) or N;
each R^{A} is independently selected from the group consisting of halogen and optionally substituted C₁-C₆ alkyl;
each R^{B} is independently selected from the group consisting of optionally substituted C₁-C₆ alkoxy and halogen;
R^{C} is selected from the group consisting of hydrogen, optionally substituted benzyl, —OR², —OC(O)R², —C(O)R², —OC(O)OR², —N(R²)₂, and —OC(O)N(R²)₂;
each R¹ is hydrogen or optionally substituted C₁-C₃ alkyl;
each R² is independently selected from the group consisting of optionally substituted C₁-C₆ alkyl, —(CH₂)₀₋₃phenyl, —(CH₂)₀₋₃C(O)R³, 5-10 membered heteroaryl, 3-7 membered heterocyclyl, and —N═CH-phenyl, wherein R² is optionally substituted with one, two, or three instances of R⁴;
   wherein two instances of R² may be joined together with the atom on which they are attached to form a 5-10 membered heteroaryl or 3-7 membered heterocyclyl, optionally substituted with one, two, or three instances of R⁴;
each R³ is 5-10 membered heteroaryl or 3-7 membered heterocyclyl, wherein R³ is optionally substituted with one, two, or three instances of R⁴;
each R⁴ is selected from the group consisting of halogen, —OH, —NH₂, —CN, —NHR¹, optionally substituted C₁-C₆ alkyl, optionally substituted C₁-C₆ alkoxy and —S(O)₂C₁-C₃ alkyl;
   wherein two instances of R⁴ may be joined together with the atom on which they are attached to form a 5-10 membered heteroaryl or 3-7 membered heterocyclyl, optionally substituted with one, two, or three instances of oxo, halogen or C₁-C₃ alkyl;
n is 0, 1, 2, or 3; and
m is 0, 1, 2, or 3.

The present disclosure includes a compound of Formula (I-a):

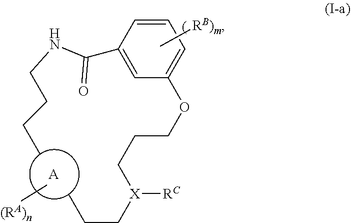

or a pharmaceutically acceptable salt thereof.

The present disclosure includes a compound of Formula (I-b):

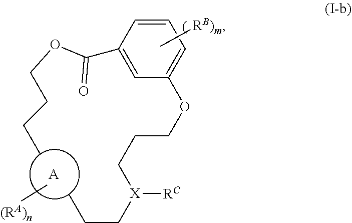

or a pharmaceutically acceptable salt thereof.

The present disclosure includes a compound of Formula (I-c):

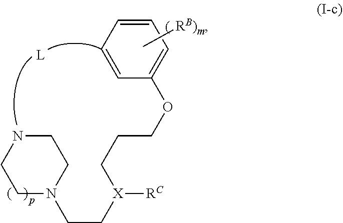

or a pharmaceutically acceptable salt thereof,
wherein p is 1 or 2.

### L

In some embodiments, L is an optionally substituted C₃-C₇ alkylene chain, wherein one, two, or three methylene units is optionally and independently replaced with —O—, —N(R¹)—, —C(O)—, —C(O)O—, —C(O)N(R¹)—, —S(O)₂—, a 5-membered heteroaryl, —CH═CH—, or —C≡C—.

In some embodiments, L is an optionally substituted C₁-C₇ alkylene chain, wherein a methylene unit is replaced with —C(O)N(R¹)—. In some embodiments, L is an optionally substituted C₃-C₆ alkylene chain, wherein a methylene unit is replaced with —C(O)N(R¹)—. In some embodiments, L is an optionally substituted C₃-C₅ alkylene chain, wherein a methylene unit is replaced with —C(O)N(R¹)—. In some embodiments, L is an optionally substituted C₄ alkylene chain, wherein a methylene unit is replaced with —C(O)N(R¹)—.

In some embodiments, L is an optionally substituted C₃-C₇ alkylene chain, wherein a methylene unit is replaced with —C(O)O—. In some embodiments, L is an optionally substituted C₃-C₆ alkylene chain, wherein a methylene unit is replaced with —C(O)O—. In some embodiments, L is an optionally substituted C₃-C₅ alkylene chain, wherein a methylene unit is replaced with —C(O)O—. In some embodiments, L is an optionally substituted C₄ alkylene chain, wherein a methylene unit is replaced with —C(O)O—.

In some embodiments, L is an optionally substituted C₃-C₇ alkylene chain, wherein a methylene unit is replaced with —O—. In some embodiments, L is an optionally substituted C₃-C₆ alkylene chain, wherein a methylene unit is replaced with —O—. In some embodiments, L is an optionally substituted C₃-C₅ alkylene chain, wherein a methylene unit is replaced with —O—. In some embodiments, L is an optionally substituted C₄ alkylene chain, wherein a methylene unit is replaced with —O—.

In some embodiments, L is an optionally substituted C₃-C₇ alkylene chain, wherein a methylene unit is replaced with —S(O)₂—. In some embodiments, L is an optionally substituted C₃-C₆ alkylene chain, wherein a methylene unit is replaced with —S(O)₂—. In some embodiments, L is an optionally substituted C₃-C₅ alkylene chain, wherein a methylene unit is replaced with —S(O)₂—. In some embodiments, L is an optionally substituted C₄ alkylene chain, wherein a methylene unit is replaced with —S(O)₂—.

In some embodiments, L is

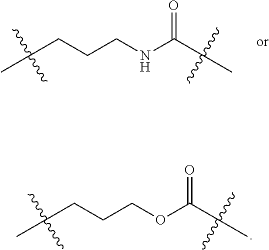 [SMILES: *CCCNC(*)=O ; *CCCOC(*)=O]

### A

In some embodiments, A is —N(R^{A})— or 5-7 membered heterocyclyl. In some embodiments, A is 5-7 membered heterocyclyl. In some embodiments, A is —N(R^{A})—. In some embodiments, A is 5-7 membered heterocyclyl containing 1-3 nitrogen atoms. In some embodiments, A is 5-7 membered heterocyclyl containing 1 nitrogen atom. In some embodiments, A is 5-7 membered heterocyclyl containing 2 nitrogen atoms.

In some embodiments, A is selected from the group consisting of

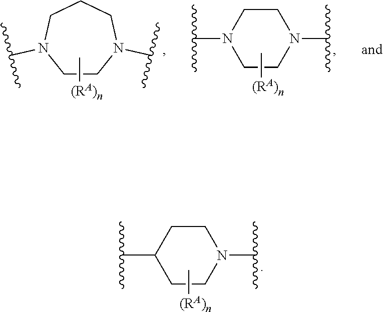

In some embodiments. A is selected from the group consisting of

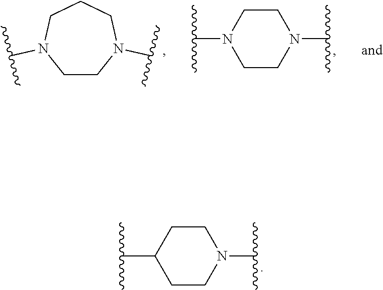

### X

In some embodiments, X is —C(H)— or —N—. In some embodiments, X is —C(H)—. In some embodiments, X is —N—.

### R^{A}

In some embodiments, each R^{A} is independently selected from the group consisting of halogen and optionally substituted C₁-C₆ alkyl. In some embodiments, each R^{A} is independently halogen. In some embodiments, each R^{A} is fluoro. In some embodiments, each R^{A} is independently optionally substituted C₁-C₆ alkyl. In some embodiments, each R^{A} is independently optionally substituted C₁-C₃ alkyl. In some embodiments, each R^{A} is optionally substituted methyl.

### R^{B}

In some embodiments, each R^{B} is independently selected from the group consisting of optionally substituted C₁-C₆ alkoxy and halogen. In some embodiments, each R^{B} is independently selected from the group consisting of optionally substituted C₁-C₃ alkoxy and halogen. In some embodiments, each R^{B} is independently C₁-C₆ alkoxy. In some embodiments, each R^{B} is independently optionally substituted C₁-C₃ alkoxy. In some embodiments, R^{B} is optionally substituted ethoxy. In some embodiments, R^{B} is optionally substituted methoxy. In some embodiments, R^{B} is halogen. In some embodiments, R^{B} is fluoro. In some embodiments, R^{B} is chloro.

### R^{C}

In some embodiments, R^{C} is selected from the group consisting of hydrogen, optionally substituted benzyl, —OR², —OC(O)R², —C(O)R², —OC(O)OR², —N(R²)₂, and —OC(O)N(R²)₂.

In some embodiments, R^{C} is —OC(O)R². In some embodiments, R^{C} is —OC(O)R², and wherein R² is 5-10-membered heteroaryl, wherein R² is optionally substituted with one, two, or three instances of R⁴. In some embodiments, R^{C} is —OC(O)R², and wherein R² is a 5-membered heteroaryl, wherein R² is optionally substituted with one, two, or three instances of R⁴. In some embodiments, R^{C} is —OC(O)R², and wherein R² is a 6-membered heteroaryl, wherein R² is optionally substituted with one, two, or three instances of R⁴. In some embodiments, R^{C} is —OC(O)R², and wherein R² is a 9-membered heteroaryl, wherein R² is optionally substituted with one, two, or three instances of R⁴. In some embodiments, R^{C} is selected from the group consisting of

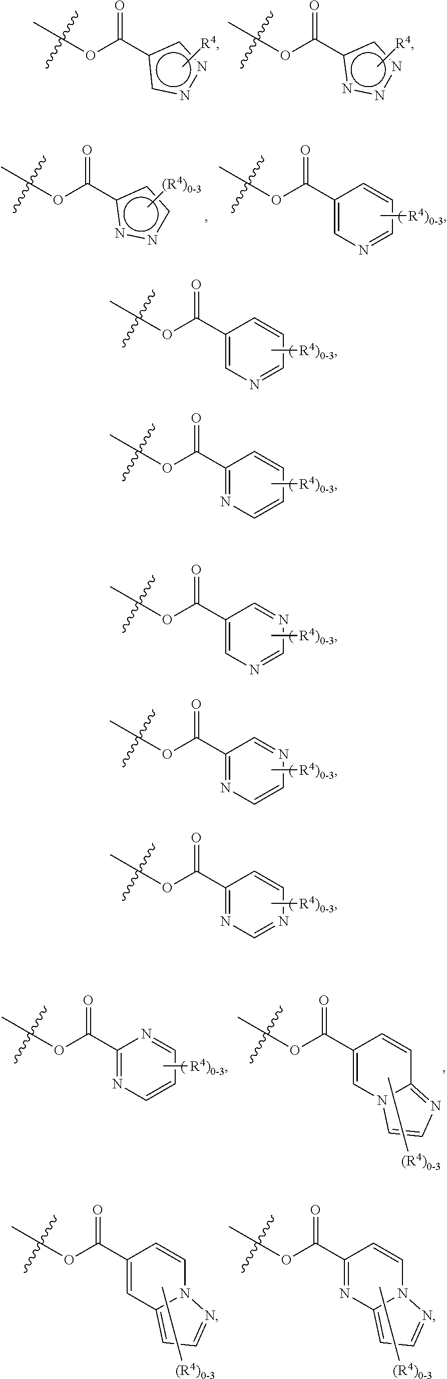

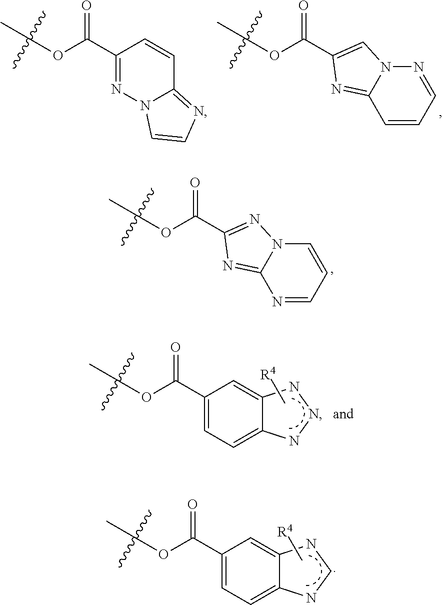

In some embodiments, R^{C} is —OC(O)R², and wherein R² is 5-10-membered heterocyclyl, wherein R² is optionally substituted with one, two, or three instances of R⁴. In some embodiments, R^{C} is —OC(O)R², and wherein R² is 5-membered heterocyclyl, wherein R² is optionally substituted with one, two, or three instances of R⁴. In some embodiments, R^{C} is —OC(O)R², and wherein R² is 6-membered heterocyclyl, wherein R² is optionally substituted with one, two, or three instances of R⁴. In some embodiments, R^{C} is —OC(O)R², and wherein R² is 7-membered heterocyclyl, wherein R² is optionally substituted with one, two, or three instances of R⁴. In some embodiments, R^{C} is —OC(O)R², and wherein R² is 9-membered heterocyclyl, wherein R² is optionally substituted with one, two, or three instances of R⁴. In some embodiments R^{C} is selected from the group consisting of

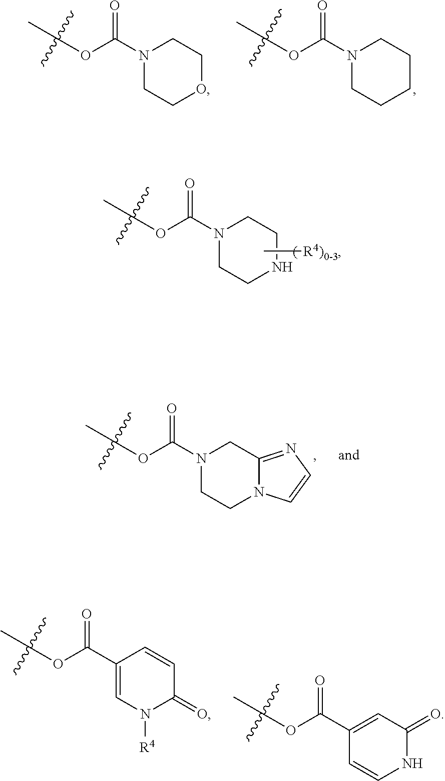

In some embodiments, R^{C} is —OC(O)R², wherein R² is —(CH₂)₀₋₃C(O)R³. In some embodiments, R^{C} is —OC(O)R², wherein R² is —(CH₂)₀₋₃C(O)R³, and R³ is 5-10 membered heteroaryl or 3-7 membered heterocyclyl, wherein R³ is optionally substituted with one, two, or three instances of R⁴. In some embodiments, R^{C} is —OC(O)R², wherein R² is —(CH₂)₀₋₃C(O)R³, and R³ is 3-7 membered heterocyclyl, wherein R³ is optionally substituted with one, two, or three instances of R⁴. In some embodiments, R^{C} is —OC(O)R², wherein R² is —(CH₂)₀₋₃C(O)R³, and R³ is 6-membered heterocyclyl, wherein R³ is optionally substituted with one, two, or three instances of R⁴. In some embodiments, R^{C} is —OC(O)R², wherein R² is —(CH₂)₀₋₃C(O)R³, and R³ is 7-membered heterocyclyl, wherein R³ is optionally substituted with one, two, or three instances of R⁴. In some embodiments, R^{C} is —OC(O)R², wherein R² is —(CH₂)₀₋₃C(O)R³, and R³ is 9-membered heterocyclyl, wherein R³ is optionally substituted with one, two, or three instances of R⁴.

In some embodiments, R^{C} is selected from the group consisting of

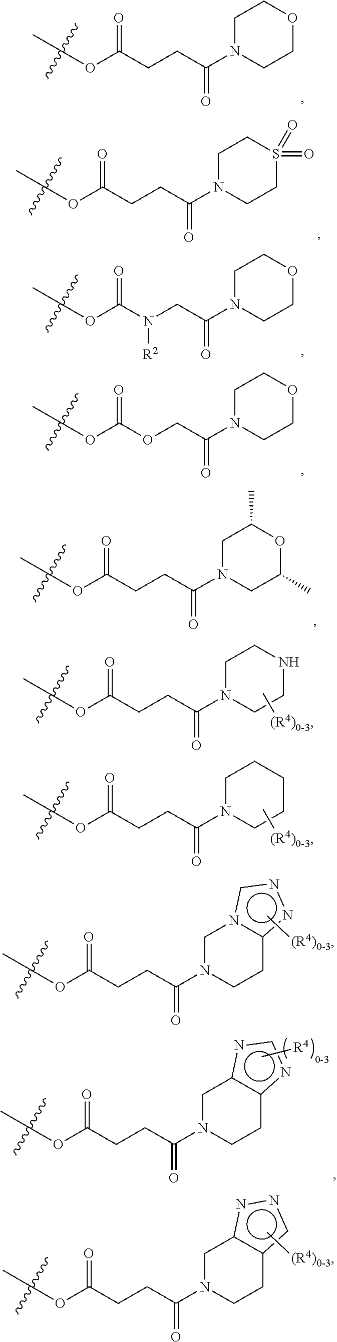

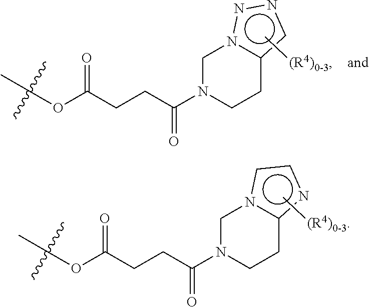

In some embodiments, R^{C} is selected from the group consisting of optionally substituted benzyl. In some embodiments, R^{C} is selected from the group consisting of —OR². In some embodiments, R^{C} is selected from the group consisting of —C(O)R². In some embodiments, R^{C} is selected from the group consisting of —OC(O)OR². In some embodiments, R^{C} is selected from the group consisting of —N(R²)₂. In some embodiments, R^{C} is selected from the group consisting of —OC(O)N(R²)₂.

In some embodiments, R^{C} is selected from the group consisting of

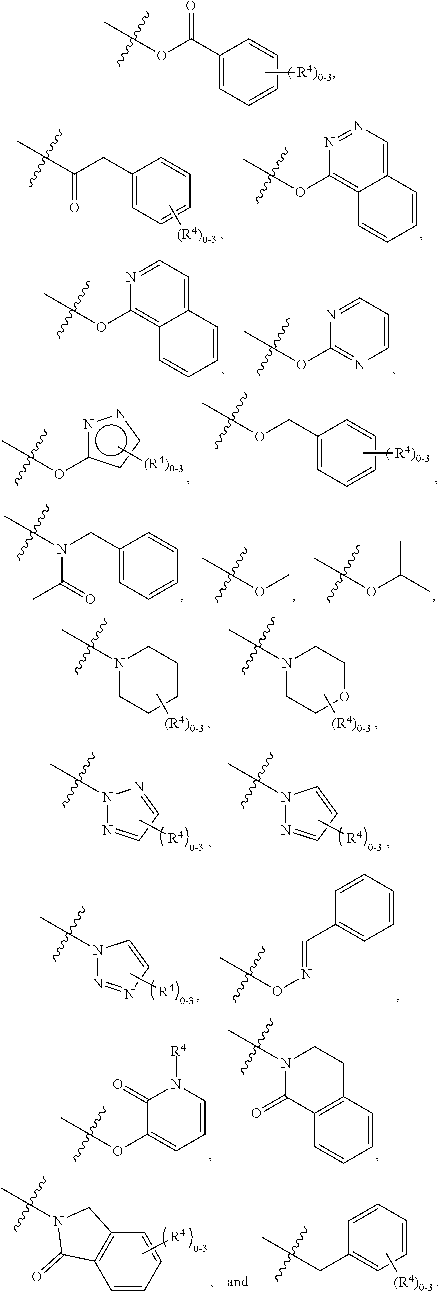

### R¹

In some embodiments, each R¹ is independently hydrogen or optionally substituted C₁-C₃ alkyl. In some embodiments, R¹ is hydrogen. In some embodiments each R¹ is independently optionally substituted C₁-C₃ alkyl. In some embodiments each R¹ is independently optionally substituted methyl. In some embodiments each R¹ is independently optionally substituted ethyl. In some embodiments each R¹ is independently optionally substituted n-propyl. In some embodiments each R¹ is independently optionally substituted i-propyl.

### R²

In some embodiments, each R² is independently selected from the group consisting of optionally substituted C₁-C₆ alkyl, —(CH₂)₀₋₃phenyl, —(CH₂)₀₋₃C(O)R³, 5-10 membered heteroaryl, 3-7 membered heterocyclyl, and —N═CH-phenyl, wherein R² is optionally substituted with one, two, or three instances of R⁴;
wherein two instances of R² may be joined together with the atom on which they are attached to form a 5-10 membered heteroaryl or 3-7 membered heterocyclyl, optionally substituted with one, two, or three instances of R⁴.

In some embodiments, each R² is independently selected from the group consisting of optionally substituted C₁-C₆ alkyl, —(CH₂)₀₋₃phenyl, —(CH₂)₀₋₃C(O)R³, 5-10 membered heteroaryl, 3-7 membered heterocyclyl, and —N═CH-phenyl, wherein R² is optionally substituted with one, two, or three instances of R⁴. In some embodiments, R² is 5-10-membered heteroaryl, wherein R² is optionally substituted with one, two, or three instances of R⁴. In some embodiments, R² is 5-membered heteroaryl, wherein R² is optionally substituted with one, two, or three instances of R⁴. In some embodiments, R² is 6-membered heteroaryl, wherein R² is optionally substituted with one, two, or three instances of R⁴. In some embodiments. R² is 9-membered heteroaryl, wherein R² is optionally substituted with one, two, or three instances of R⁴.

In some embodiments, R² is 5-10-membered heterocyclyl, wherein R² is optionally substituted with one, two, or three instances of R⁴. In some embodiments, R² is 5-membered heterocyclyl, wherein R² is optionally substituted with one, two, or three instances of R⁴. In some embodiments. R² is 6-membered heterocyclyl, wherein R² is optionally substituted with one, two, or three instances of R⁴. In some embodiments, R² is 7-membered heterocyclyl, wherein R² is optionally substituted with one, two, or three instances of R⁴. In some embodiments, R² is 9-membered heterocyclyl, wherein R² is optionally substituted with one, two, or three instances of R⁴.

### R³

In some embodiments, each R³ is 5-10 membered heteroaryl or 3-7 membered heterocyclyl, wherein R³ is optionally substituted with one, two, or three instances of R⁴. In some embodiments, each R³ is 3-7 membered heterocyclyl, wherein R³ is optionally substituted with one, two, or three instances of R⁴. In some embodiments, each R³ is 6-membered heterocyclyl, wherein R³ is optionally substituted with one, two, or three instances of R⁴. In some embodiments, each R³ is 9-membered heterocyclyl, wherein R³ is optionally substituted with one, two, or three instances of R⁴.

### R⁴

In some embodiments, each R⁴ is selected from the group consisting of halogen, —OH, —NH₂, —CN, —NHR¹, optionally substituted C₁-C₆ alkyl, optionally substituted C₁-C₆ alkoxy and —S(O)₂C₁-C₃ alkyl;
wherein two instances of R⁴ may be joined together with the atom on which they are attached to form a 5-10 membered heteroaryl or 3-7 membered heterocyclyl, optionally substituted with one, two, or three instances of oxo, halogen or C₁-C₃ alkyl.

In some embodiments, the present disclosure includes compounds described in Table 1.

TABLE 1

| Com | |
|---|---|
| pound | |
| No. | Structure |
| 1 | 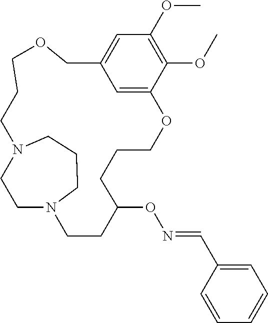 [SMILES: COc1cc2cc(c1OC)OCCCC(O/N=C/c1ccccc1)CCN1CCCN(CCCOC2)CC1] |
| 2 | 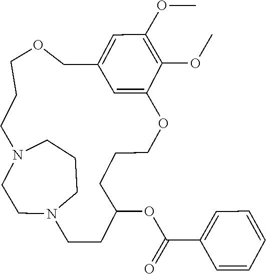 [SMILES: COc1cc2cc(c1OC)OCCCC(OC(=O)c1ccccc1)CCN1CCCN(CCCOC2)CC1] |
| 3 | 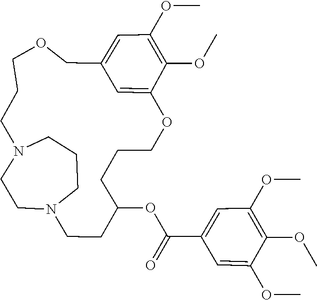 [SMILES: COc1cc(C(=O)OC2CCCOc3cc(cc(OC)c3OC)COCCCN3CCCN(CC2)CC3)cc(OC)c1OC] |
| 4 | 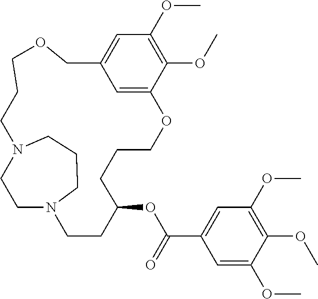 [SMILES: COc1cc(C(=O)O[C@@H]2CCCOc3cc(cc(OC)c3OC)COCCCN3CCCN(CC2)CC3)cc(OC)c1OC] |
| 5 | 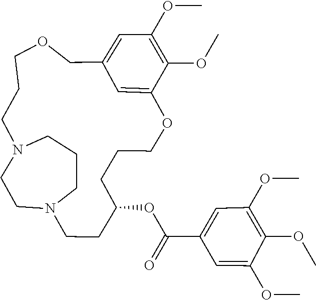 [SMILES: COc1cc(C(=O)O[C@H]2CCCOc3cc(cc(OC)c3OC)COCCCN3CCCN(CC2)CC3)cc(OC)c1OC] |
| 6 | 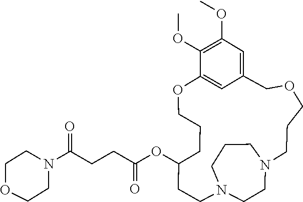 [SMILES: COc1cc2cc(c1OC)OCCCC(OC(=O)CCC(=O)N1CCOCC1)CCN1CCCN(CCCOC2)CC1] |
| 7 | 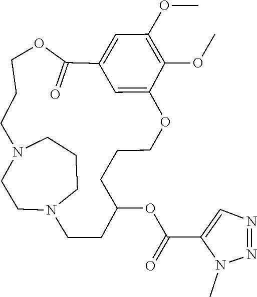 [SMILES: COc1cc2cc(c1OC)OCCCC(OC(=O)c1cnnn1C)CCN1CCCN(CCCOC2=O)CC1] |
| 8 | 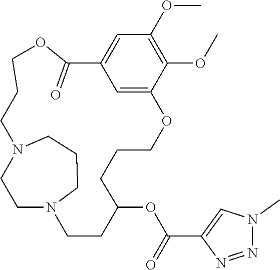 [SMILES: COc1cc2cc(c1OC)OCCCC(OC(=O)c1cn(C)nn1)CCN1CCCN(CCCOC2=O)CC1] |
| 9 | 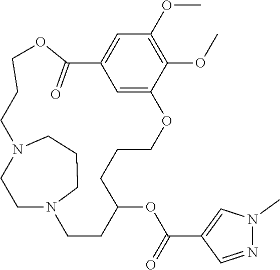 [SMILES: COc1cc2cc(c1OC)OCCCC(OC(=O)c1cnn(C)c1)CCN1CCCN(CCCOC2=O)CC1] |
| 10 | 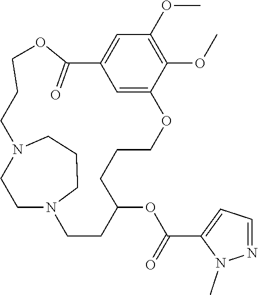 [SMILES: COc1cc2cc(c1OC)OCCCC(OC(=O)c1ccnn1C)CCN1CCCN(CCCOC2=O)CC1] |
| 11 | 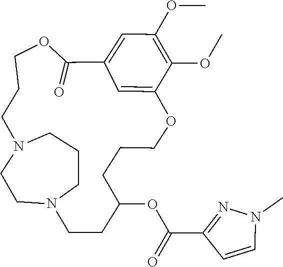 [SMILES: COc1cc2cc(c1OC)OCCCC(OC(=O)c1ccn(C)n1)CCN1CCCN(CCCOC2=O)CC1] |
| 12 | 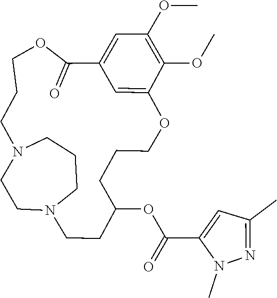 [SMILES: COc1cc2cc(c1OC)OCCCC(OC(=O)c1cc(C)nn1C)CCN1CCCN(CCCOC2=O)CC1] |
| 13 | 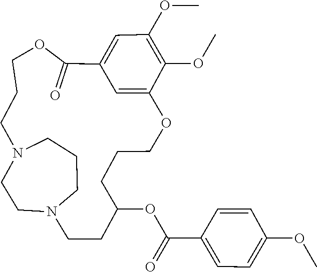 [SMILES: COc1ccc(C(=O)OC2CCCOc3cc(cc(OC)c3OC)C(=O)OCCCN3CCCN(CC2)CC3)cc1] |
| 14 | 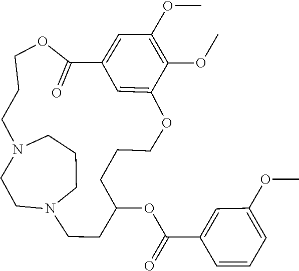 [SMILES: COc1cccc(C(=O)OC2CCCOc3cc(cc(OC)c3OC)C(=O)OCCCN3CCCN(CC2)CC3)c1] |
| 15 | 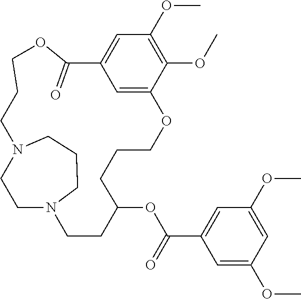 [SMILES: COc1cc(OC)cc(C(=O)OC2CCCOc3cc(cc(OC)c3OC)C(=O)OCCCN3CCCN(CC2)CC3)c1] |
| 16 | 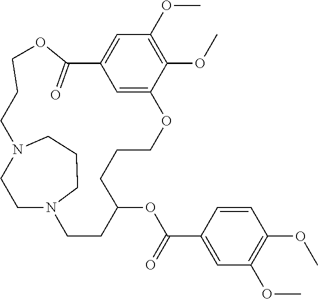 [SMILES: COc1ccc(C(=O)OC2CCCOc3cc(cc(OC)c3OC)C(=O)OCCCN3CCCN(CC2)CC3)cc1OC] |
| 17 | 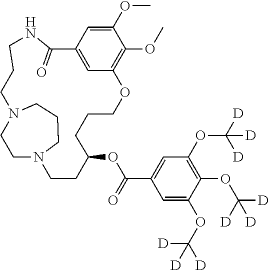 [SMILES: [2H]C([2H])([2H])Oc1cc(C(=O)O[C@@H]2CCCOc3cc(cc(OC)c3OC)C(=O)NCCCN3CCCN(CC2)CC3)cc(OC([2H])([2H])[2H])c1OC([2H])([2H])[2H]] |
| | (absolute stereochemistry) |
| 18 | 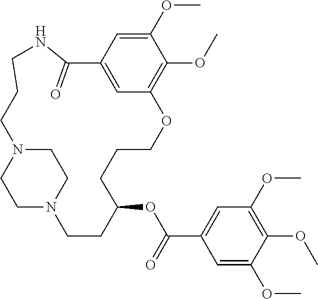 [SMILES: COc1cc(C(=O)O[C@@H]2CCCOc3cc(cc(OC)c3OC)C(=O)NCCCN3CCN(CC2)CC3)cc(OC)c1OC] |
| 19 | 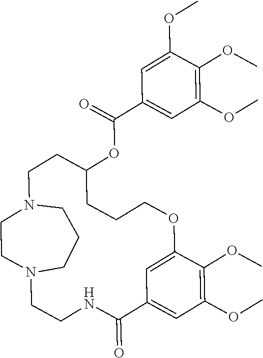 [SMILES: COc1cc(C(=O)OC2CCCOc3cc(cc(OC)c3OC)C(=O)NCCN3CCCN(CC2)CC3)cc(OC)c1OC] |
| 20 | 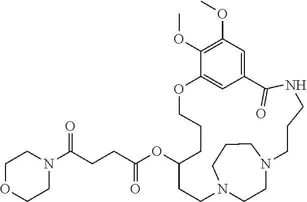 [SMILES: COc1cc2cc(c1OC)OCCCC(OC(=O)CCC(=O)N1CCOCC1)CCN1CCCN(CCCNC2=O)CC1] |
| 21 | 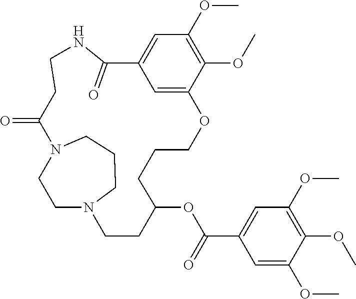 [SMILES: COc1cc(C(=O)OC2CCCOc3cc(cc(OC)c3OC)C(=O)NCCC(=O)N3CCCN(CC2)CC3)cc(OC)c1OC] |
| 22 | 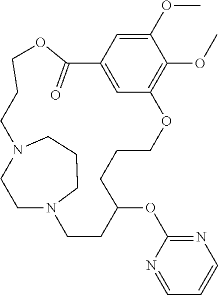 [SMILES: COc1cc2cc(c1OC)OCCCC(Oc1ncccn1)CCN1CCCN(CCCOC2=O)CC1] |
| 23 | 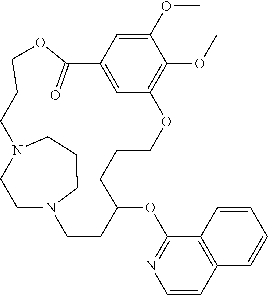 [SMILES: COc1cc2cc(c1OC)OCCCC(Oc1nccc3ccccc13)CCN1CCCN(CCCOC2=O)CC1] |
| 24 | 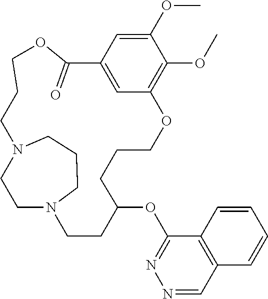 [SMILES: COc1cc2cc(c1OC)OCCCC(Oc1nncc3ccccc13)CCN1CCCN(CCCOC2=O)CC1] |
| 25 | 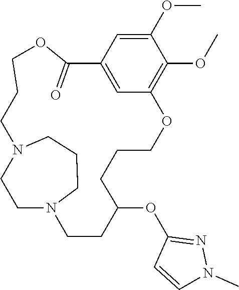 [SMILES: COc1cc2cc(c1OC)OCCCC(Oc1ccn(C)n1)CCN1CCCN(CCCOC2=O)CC1] |
| 26 | 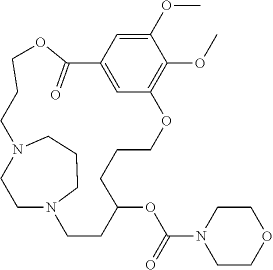 [SMILES: COc1cc2cc(c1OC)OCCCC(OC(=O)N1CCOCC1)CCN1CCCN(CCCOC2=O)CC1] |
| 27 | 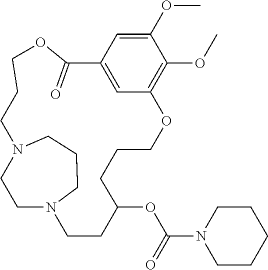 [SMILES: COc1cc2cc(c1OC)OCCCC(OC(=O)N1CCCCC1)CCN1CCCN(CCCOC2=O)CC1] |
| 28 | 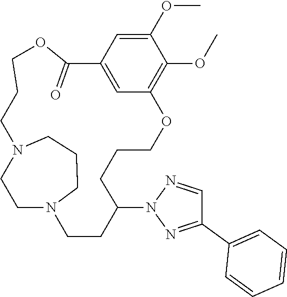 [SMILES: COc1cc2cc(c1OC)OCCCC(n1ncc(-c3ccccc3)n1)CCN1CCCN(CCCOC2=O)CC1] |
| 29 | 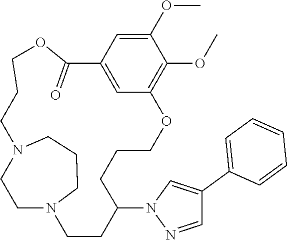 |
| 30 | 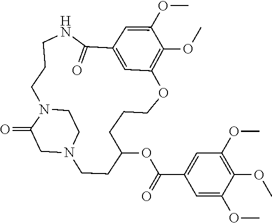 [SMILES: COc1cc(C(=O)OC2CCCOc3cc(cc(OC)c3OC)C(=O)NCCCN3CCN(CC2)CC3=O)cc(OC)c1OC] |
| 31 | 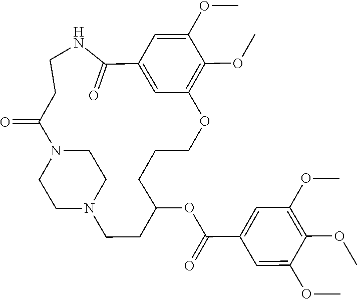 [SMILES: COc1cc(C(=O)OC2CCCOc3cc(cc(OC)c3OC)C(=O)NCCC(=O)N3CCN(CC2)CC3)cc(OC)c1OC] |
| 32 | 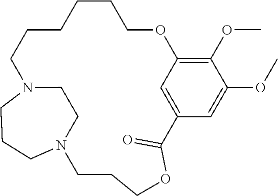 [SMILES: COc1cc2cc(c1OC)OCCCCCCN1CCCN(CCCOC2=O)CC1] |
| 33 | 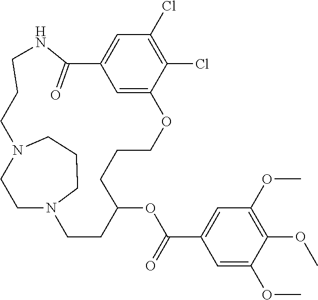 [SMILES: COc1cc(C(=O)OC2CCCOc3cc(cc(Cl)c3Cl)C(=O)NCCCN3CCCN(CC2)CC3)cc(OC)c1OC] |
| 34 | 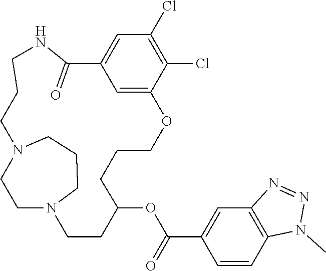 [SMILES: Cn1nnc2cc(C(=O)OC3CCCOc4cc(cc(Cl)c4Cl)C(=O)NCCCN4CCCN(CC3)CC4)ccc21] |
| 35 | 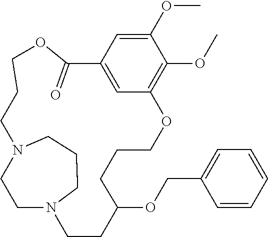 [SMILES: COc1cc2cc(c1OC)OCCCC(OCc1ccccc1)CCN1CCCN(CCCOC2=O)CC1] |
| 36 | 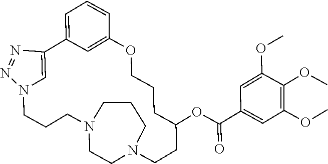 [SMILES: COc1cc(C(=O)OC2CCCOc3cccc(c3)-c3cn(nn3)CCCN3CCCN(CC2)CC3)cc(OC)c1OC] |
| 37 | 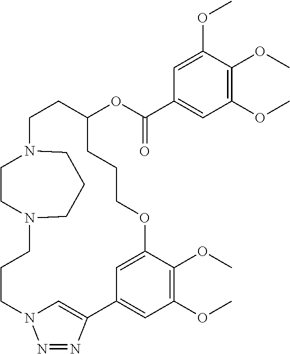 [SMILES: COc1cc(C(=O)OC2CCCOc3cc(cc(OC)c3OC)-c3cn(nn3)CCCN3CCCN(CC2)CC3)cc(OC)c1OC] |
| 38 | 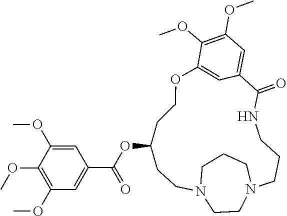 [SMILES: COc1cc(C(=O)O[C@H]2CCOc3cc(cc(OC)c3OC)C(=O)NCCCN3CCCN(CC2)CC3)cc(OC)c1OC] |
| 39 | 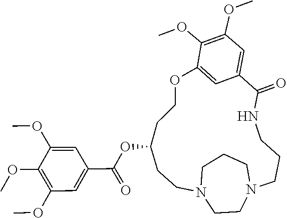 [SMILES: COc1cc(C(=O)O[C@@H]2CCOc3cc(cc(OC)c3OC)C(=O)NCCCN3CCCN(CC2)CC3)cc(OC)c1OC] |
| 40 | 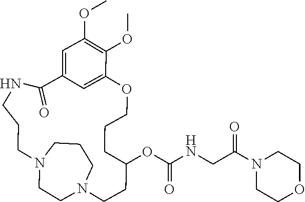 [SMILES: COc1cc2cc(c1OC)OCCCC(OC(=O)NCC(=O)N1CCOCC1)CCN1CCCN(CCCNC2=O)CC1] |
| 41 | 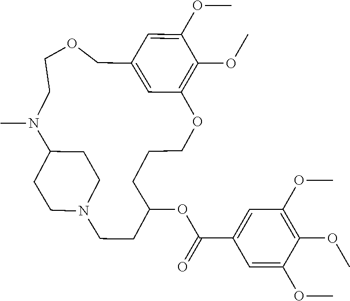 |
| 42 | 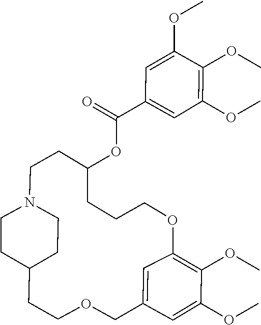 [SMILES: COc1cc(C(=O)OC2CCCOc3cc(cc(OC)c3OC)COCCC3CCN(CC3)CC2)cc(OC)c1OC] |
| 43 | 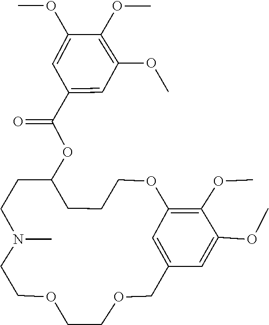 [SMILES: COc1cc(C(=O)OC2CCCOc3cc(cc(OC)c3OC)COCCOCCN(C)CC2)cc(OC)c1OC] |
| 44 | 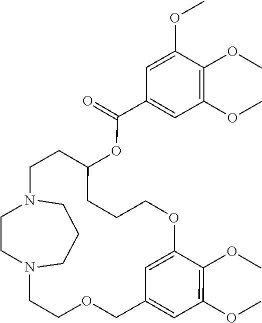 [SMILES: COc1cc(C(=O)OC2CCCOc3cc(cc(OC)c3OC)COCCN3CCCN(CC2)CC3)cc(OC)c1OC] |
| 45 | 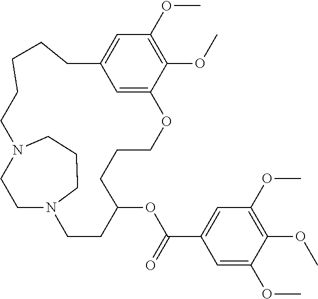 [SMILES: COc1cc(C(=O)OC2CCCOc3cc(cc(OC)c3OC)CCCCCN3CCCN(CC2)CC3)cc(OC)c1OC] |
| 46 | 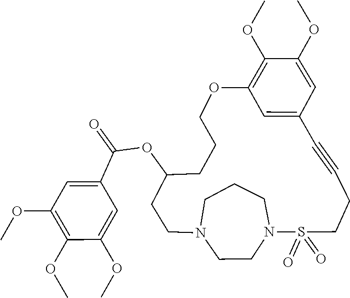 [SMILES: COc1cc(C(=O)OC2CCCOc3cc(cc(OC)c3OC)C#CCCS(=O)(=O)N3CCCN(CC2)CC3)cc(OC)c1OC] |
| 47 | 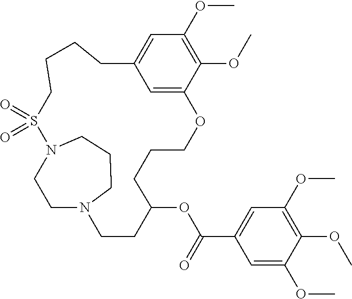 [SMILES: COc1cc(C(=O)OC2CCCOc3cc(cc(OC)c3OC)CCCCS(=O)(=O)N3CCCN(CC2)CC3)cc(OC)c1OC] |

or a pharmaceutically acceptable salt thereof

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The present disclosure also includes the salt form of any of the compounds disclosed herein.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein.

In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

In one embodiment, the present disclosures relates to enantiomers and isomers (including optical, geometric and tautomeric isomers) of compounds of formula I and subformula thereof. Indeed, the compounds of formula I and subformula thereof may contain an asymmetric center and thus may exist as different stereoisomeric forms. Accordingly, the present disclosure includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient Intermediate compound, or by chiral chromatographic methods as each are known in the art. Resolution of the final product, an Intermediate compound, or a starting material may be performed by any suitable method known in the art. Unless otherwise indicated, the stereochemistry of a compound has been arbitrarily assigned. Accordingly, the absolute stereochemistry of a molecule is unknown, unless otherwise indicated.

In one embodiment, the present invention also relates to salts of compounds of formula I subformula thereof. Especially, the compounds of the invention may be in the form of pharmaceutically acceptable salts. Pharmaceutically acceptable salts of the compounds of formula I ate, ammonium salt, aspartate, benzoate, besylate, benzenesulfonate, bicarbonate/carbonate, bisulphate/sulphate, bitartrate, borate, calcium edetate, camsylate, citrate, clavulanate, cyclamate, dihydrochloride, edetate, edisylate, estolate, esylate, formate, fumarate, gluceptate, gluconate, glucuronate, glutamate, glycollylarsanilate, hexafluorophosphate, hexylresorcinate, hibenzate, hydrabamine, hydrochloride/chloride, hydrobromide/bromide, hydroiodide/iodide, hydroxynaphthoate, isethionate, isothionate, lactate, lactobionate, laurate, malate, maleate, malonate, mandelate, mesylate, methylbromide, N-methylglucamine, methylnitrate, methylsulphate, mucate, panoate, naphthylate, 2-napsylate, nicotinate, nitrate, oleate, orotate, oxalate, palmitate, pamoate, pantothenate, phosphate/hydrogen phosphate/dihydrogen phosphate, polygalacturonate, pyroglutamate, saccharate, salicylate, stearate, subacetate, succinate, tannate, tartrate, teoclate, tosylate, triethiodide, trifluoroacetate, valerate and xinofoate salts. Preferred pharmaceutically acceptable acid addition salts include hydrochloride/chloride, hydrobromide/bromide, bisulphate/sulphate, nitrate, citrate, tosylate, esylate and acetate. Suitable base salts are formed from bases which form non-toxic salts. Examples include the aluminum, ammonia, arginine, benzathine, N-benzylphenethyl-amine, calcium, chloroprocaine, choline, N,N′-dibenzylethylene-diamine, diethanolamine, diethylamine, 2-(diethylamino)ethanol, diolamine, ethanolamine, ethylenediamine, glycine, lithium, lysine, magnesium, meglumine, N-methyl-glutamine, morpholine, 4-(2-hydroxyethyl)morpholine, olamine, ornithine, piperazine, potassium, procaine, sodium, tetramethylammonium hydroxide, tris(hydroxymethyl)aminomethane, tromethamine and zinc salts. Hemisalts of acids and bases may also be formed, for example, hemisulphate and hemicalcium salts. When the compounds of the invention contain a hydrogen-donating heteroatom (e.g. NH), the invention also covers salts and/or isomers formed by transfer of said hydrogen atom to a basic group or atom within the molecule.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

The present disclosure is further directed to the use of the compounds of the present disclosure, or pharmaceutically acceptable salts and solvates thereof, as inhibitors of ENT family transporters. Accordingly, in a particularly preferred embodiment, the present disclosure relates to the use of compounds of formula I and subformula in particular those of Table 1 above, or pharmaceutically acceptable salts and solvates thereof, as inhibitors of ENT family transporters.

In one embodiment, the compounds of the present disclosure are inhibitors of ENT1, ENT2, ENT3 and/or ENT4 In one embodiment, the compounds of the present disclosure are inhibitors of ENT1 and ENT2. In one embodiment, the compounds of the present disclosure are inhibitors of ENT1, preferably selective inhibitors of ENT1. In one embodiment, the compounds of the present disclosure are inhibitors selective of ENT1, with respect to other ENT family transporters, especially with respect to ENT2 and ENT4.

The present disclosure also provides a method for inhibiting ENT family transporters, especially ENT1, in a patient, preferably a warm-blooded animal, and even more preferably a human, in need thereof, which comprises administering to said patient an effective amount of a compound of the present disclosure, or a pharmaceutically acceptable salt and solvate thereof.

The present disclosure is further directed to the use of the compounds of the present disclosure as a medicament, i.e. for medical use. Thus, in one embodiment, the present disclosure provides the use of the compounds of the present disclosure for the manufacturing of a medicament. Especially, the present disclosure provides the use of the compounds of the present disclosure for the manufacturing of a medicament.

Especially, the present disclosure provides the compounds of the present disclosure, for use in the treatment and/or prevention of proliferative disorders, including cancers. Thus, in one embodiment, the present disclosure provides the use of the compounds of the present disclosure for the manufacture of a medicament for treating and/or preventing cancer. The present disclosure also provides a method of treatment of cancer, which comprises administering to a mammal species in need thereof a therapeutically effective amount of a compound of the present disclosure.

The present disclosure also provides for a method for delaying in patient the onset of cancer comprising the administration of a pharmaceutically effective amount of a compound of the present disclosure to a patient in need thereof.

Various cancers are known in the art. Cancers that can be treated using the methods of the present disclosure include solid cancers and non-solid cancers, especially benign and malignant solid tumors and benign and malignant non-solid tumors. The cancer may be metastatic or non-metastatic. The cancer may be may be familial or sporadic.

In one embodiment, the cancer to be treated according to the present disclosure is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present disclosure is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, the cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

In another specific embodiment, the cancer is selected from the group consisting of: leukemia and multiple myeloma.

Preferably, the patient is a warm-blooded animal, more preferably a human.

In one embodiment, the subject receiving the ENT inhibitor of the present disclosure is treated with an additional therapeutic agent in combination with the ENT inhibitor of the present disclosure, or has received the additional therapeutic agent within about fourteen days of administration of the ENT inhibitor of the present disclosure. In one embodiment, the additional therapeutic agent comprises an adenosine receptor antagonist.

In one embodiment, the subject has previously received at least one prior therapeutic treatment, and has progressed subsequent to the administration of the at least one prior therapeutic treatment and prior to administration of the ENT inhibitor of the present disclosure.

In one embodiment, the prior therapeutic treatment is selected from the group consisting of chemotherapy, immunotherapy, radiation therapy, stem cell transplant, hormone therapy, and surgery.

In one embodiment, ENT inhibitor of the present disclosure is administered prior to, concomitant with, or subsequent to administration of the additional therapeutic agent, such as an adenosine receptor antagonist.

The present disclosure also provides pharmaceutical compositions comprising a compound of formula i or subformula thereof, or a pharmaceutically acceptable salt and solvate thereof, and at least one pharmaceutically acceptable carrier, diluent, excipient and/or adjuvant.

Another object of this present disclosure is a medicament comprising at least one compound of the present disclosure, or a pharmaceutically acceptable salt and solvate thereof, as active ingredient.

Generally, for pharmaceutical use, the compounds of the present disclosure may be formulated as a pharmaceutical preparation comprising at least one compound of the present disclosure and at least one pharmaceutically acceptable carrier, diluent, excipient and/or adjuvant, and optionally one or more further pharmaceutically active compounds. Details regarding the presence of further pharmaceutically active compounds are provided hereafter.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person, reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the present disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, the active compound of the present disclosure may be administered as a single daily dose, divided over one or more daily doses, or essentially continuously, e.g. using a drip infusion.

Combined Use with Adenosine Receptor Antagonist

The present disclosure further relates to the combined use of an ENT inhibitor of the present disclosure, of formula I and subformula thereof, as defined above, with an adenosine receptor antagonist.

The present disclosure thus relates to a combination comprising:

(a) an effective amount of an ENT inhibitor of the present disclosure, of formula I and subformula thereof, as defined above; and (b) an effective amount of an adenosine receptor antagonist.

In the context of the present disclosure the term “combination” preferably means a combined occurrence of the ENT inhibitor and of an A2AR antagonist. Therefore, the combination of the present disclosure may occur either as one composition, comprising all the components in one and the same mixture (e.g. a pharmaceutical composition), or may occur as a kit of parts, wherein the different components form different parts of such a kit of parts. The administration of the ENT inhibitor and of the A2AR antagonist may occur either simultaneously or timely staggered, with similar or different timing of administration (i.e. similar or different numbers of administration of each component), either at the same site of administration or at different sites of administration, under similar of different dosage form.

The present disclosure further relates to a method of treating cancer, comprising: administering, to a patient in need thereof, a combination of an adenosine receptor antagonist and the ENT inhibitor of the present disclosure.

Above embodiments relative to the ENT inhibitors of the present disclosure also apply to the combination of the present disclosure. Especially, in one embodiment, in the combination of the present disclosure, the ENT inhibitor may be of formula I or the subformula defined above.

As a second component, the combination of the present disclosure includes at least one adenosine receptor antagonist.

As defined above, “adenosine receptor antagonist” refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

Adenosine receptors (or P1 receptors) are a class of purinergic G protein-coupled receptors with adenosine as endogenous ligand. There are four known types of adenosine receptors in humans: A1, A2A, A2B and A3; each is encoded by a different gene (ADOARA1, ADORA2A, ADORA2B, and ADORA3 respectively).

In one embodiment, the adenosine receptor antagonist is an antagonist of A1 receptor, A2A receptor, A2B receptor, A3 receptor or of a combination thereof.

In one embodiment, the adenosine receptor antagonist is an antagonist of A2A receptor, A2B receptor or of a combination thereof. In one embodiment, the adenosine receptor antagonist is an A2A or A2B receptor antagonist.

In one embodiment, the adenosine receptor antagonist is an antagonist of A2A receptor (A2AR antagonist). In one embodiment, the adenosine receptor antagonist is an antagonist of A2B receptor (A2BR antagonist).

In one embodiment, the adenosine receptor antagonist is an antagonist which is selective of A2A receptor with respect to other adenosine receptors. In one embodiment, the adenosine receptor antagonist is an antagonist which is selective of A2A receptor with respect to A2B receptor.

In one embodiment, the adenosine receptor antagonist is an antagonist which is selective of A2B receptor with respect to other adenosine receptors. In one embodiment, the adenosine receptor antagonist is an antagonist which is selective of A2B receptor with respect to A2A receptor.

In a specific embodiment, the combination of the present disclosure comprises at least one A2A receptor antagonist as herein defined and at least one ENT inhibitor of formula I as defined above.

### A2A Receptor Antagonist

In one embodiment, the combination of the present disclosure includes at least one A2AR antagonist.

An “A2AR antagonist” refers to a compound that, upon administration to a patient, results in inhibition or down-regulation of a biological activity associated with activation of A2A receptor in the patient, including any of the downstream biological effects otherwise resulting from the binding to A2A receptor of its natural ligand. Such A2AR antagonists include any agent that can block activation of A2A receptor or any of the downstream biological effects of A2A receptor activation.

Examples of A2AR antagonists include: Preladenant (SCH-420,814), Vipadenant (BIIB-014), Tozadenant (SYK-115), ATL-444, Istradefylline (KW-6002), MSX-3, SCH-58261, SCH-412,348, SCH-442,416, ST-1535, Caffeine, VER-6623, VER-6947, VER-7835, ZM-241,385, theophylline. It also includes A2AR antagonists disclosed in WO2018/178338, WO2011/121418, WO2009/156737, WO2011/095626 or WO2018/136700, the content of which is herein incorporated by reference.

In one embodiment, the A2AR antagonist is a thiocarbamate derivative, especially a thiocarbamate derivative as those disclosed in WO2018/178338. More preferably the A2AR antagonist is a thiocarbamate derivative of formula (III) as described below.

Thus, in a specific embodiment, the present disclosure provides a combination comprising:
an ENT inhibitor according to the present disclosure, of formula I or a subformula thereof, as defined above; and (b) an A2AR antagonist being a thiocarbamate derivative of Formula (III) according to WO2018/178338:

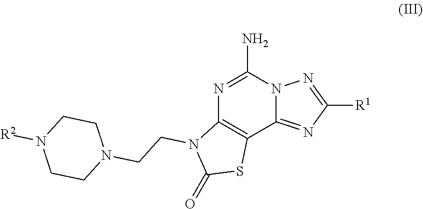

or a pharmaceutically acceptable salt or solvate thereof, wherein R¹ and R² are as defined below.

In a preferred embodiment, the A2AR antagonist is thus a compound of Formula (III), or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C₁-C₆ alkyl (preferably methyl) and halo (preferably fluoro or chloro); preferably R¹ represents 5-membered heteroaryl; more preferably R¹ represents furyl;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred A2AR antagonists of Formula (III) are of Formula (IIIa)

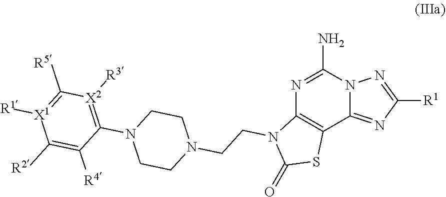

or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C₁-C₆ alkyl (preferably methyl) and halo (preferably fluoro or chloro); preferably R¹ represents 5-membered heteroaryl; more preferably R¹ represents furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl,
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl,
R^{3′} is absent when X² is N; or when X² is C, R^{3′} represents H or halo, preferably H or F;
R^{4′} represents H or halo, preferably H or F; and
R^{5′} represents H or halo, preferably H or F.

In one embodiment, preferred A2AR antagonists of Formula (IIIa) are those of Formula (IIIa-1).

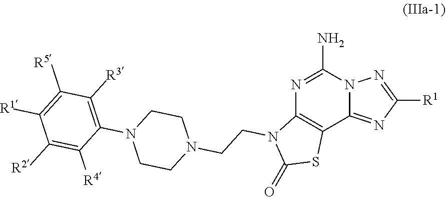

or a pharmaceutically acceptable salt or solvate thereof, wherein R¹, R^{1′}, R^{2′}, R^{3′}, R^{4′} and R^{5′} are as defined in Formula (IIIa).

In one embodiment, preferred A2AR antagonists of Formula (IIIa-1) are those of Formula (IIIa-1a).

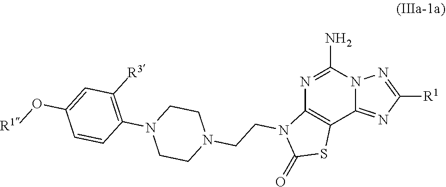

or a pharmaceutically acceptable salt or solvate thereof, wherein:
R¹ and R^{3′} are as defined in Formula (IIIa), and
R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred A2AR antagonists of Formula (IIIa-1) are those of Formula (IIIa-1b):

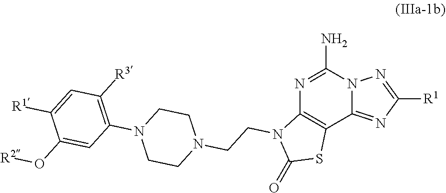

or a pharmaceutically acceptable salt or solvate thereof, wherein:
R¹ and R^{3′} are as defined in Formula (IIIa);
R^{1′} represents H or halo, preferably H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred A2AR antagonists of Formula (IIIa-1) are those of Formula (IIIa-1c) or (IIIa-1d):

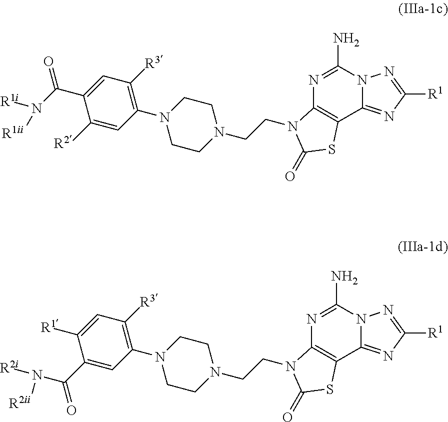

or a pharmaceutically acceptable salt or solvate thereof, wherein:
R¹ and R^{3′} are as defined in Formula (IIIa);
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In one embodiment, preferred A2AR antagonists of Formula (IIIa) are those of Formulae (IIIa-2) or (IIIa-3):

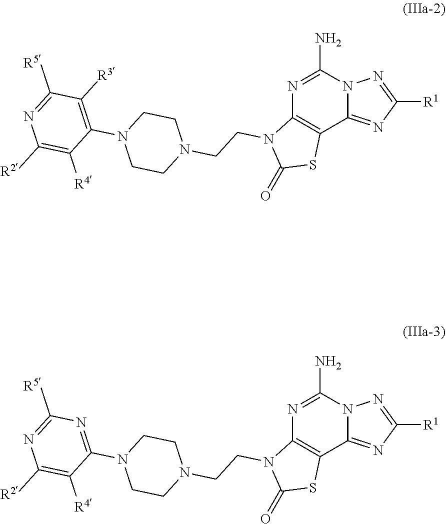

or a pharmaceutically acceptable salt or solvate thereof, wherein R¹, R^{2′}, R^{3′}, R^{4′} and R^{5′} are as defined in Formula (IIIa).

Particularly preferred A2AR antagonists of Formula (III) are those listed hereafter:
3-(2-(4-(4-((1H-1,2,3-triazolo-4yl)methoxy-2fluorophenyl)piperazine-1-yl)ethyl)-5-amino-(8-(furan-2-yl)thiazolo[5,4-e][1,2,4]triazolo[1,5-c]pyrimidine-2(3H)-one
5-((4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetamide
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-8-(furan-2-yl)-3-(2-(4-(4-(2-hydroxyethoxy)phenyl)piperazin-1-yl)ethyl)thiazolo[5,4-e][1,2,4]triazolo[1,5-c]pyrimidin-2(3H)-one
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)phenoxy)acetic acid
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)phenoxy)acetamide
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide
5-amino-8-(furan-2-yl)-3-(2-(4-(4-(methylsulfonyl)phenyl)piperazin-1-yl)ethyl)thiazolo[5,4-e][1,2,4]triazolo[1,5-c]pyrimidin-2(3H)-one
5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide
5-amino-8-(furan-2-yl)-3-(2-(4-(3-(2-hydroxyethoxy)phenyl)piperazin-1-yl)ethyl)thiazolo[5,4-e][1,2,4]triazolo[1,5-c]pyrimidin-2(3H)-one
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperidin-4-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperazine-1-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid
2-(4-(4-(2-(5-amino-8-(furan-2-yl)-2-oxothiazolo [5,4-e][1,2,4]triazolo[1,5-c]pyrimidin-3(2H)-yl)ethyl) piperazin-1-yl)-3,5-difluorophenoxy) acetic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid
(S)-2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)propanoic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid
4-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)butanoic acid
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy) acetic acid
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetic acid
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid
2-((2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)ethyl) amino)acetamide
2-((2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)ethyl)(methyl)amino)acetamide
5-amino-3-(2-(4-(2-fluoro-4-(piperidin-4-yloxy)phenyl)piperazin-1-yl)ethyl)-8-(furan-2-yl) thiazolo[5,4-e][1,2,4]triazolo[1,5-c]pyrimidin-2(3H)-one
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one
3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-(methylamino)ethyl)acetamide
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-(dimethylamino)ethyl)acetamide
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-aminoethyl)acetamide
(R)-2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)propanoic acid
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)acetamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide
(R)-4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-N-(1-(dimethylamino) propan-2-yl)-3-fluorobenzamide
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-methyl-N-(2-(methylamino)ethyl) acetamide
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-2-methylpropanoic acid
(S)-2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) propanoic acid
(R)-2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) propanoic acid
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl)acetamide
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-N-(2-(dimethylamino)ethyl)acetamide
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide
4-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) butanoic acid
3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one
5-amino-3-(2-(4-(2-fluoro-4-((1-methyl-1H-1,2,4-triazol-3-yl)methoxy) phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-((1-methyl-1H-1,2,4-triazol-3-yl) methoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methyl (oxetan-3-yl)amino)ethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide
2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)acetamide
(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide
ethyl 2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetate
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetonitrile
5-amino-8-(furan-2-yl)-3-(2-(4-(pyridin-4-yl) piperazin-1-yl)ethyl)thiazolo[5,4-e][1,2,4]triazolo[1,5-c]pyrimidin-2(3H)-one
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(morpholin-3-ylmethyl)benzamide
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide
5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one

In one embodiment, the A2AR antagonist of Formula (III) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7);
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a) and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8b).

In a specific embodiment, the A2AR antagonist of Formula (III) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7);
and
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In preferred embodiment, the A2AR antagonist of Formula (III) is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In another preferred embodiment, the A2AR antagonist of Formula (III) is (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8b).

The embodiments relative to salts, solvates, enantiomers, isomers (including optical, geometric and tautomeric isomers), polymorphs, multi-component complexes, liquid crystals, prodrugs and isotopically-labeled ENT inhibitors of the present disclosure also apply to the A2AR antagonists Formula (III) and subformula thereof detailed above

In another embodiment, the A2AR antagonist is an A2AR antagonist disclosed in WO2011/121418. Especially, the A2AR antagonist is the compound of example 1 of WO2011/121418, namely 5-bromo-2,6-di-(1H-pyrazol-1-yl)pyrimidin-4-amine, also known as NIR178:

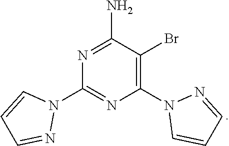 [SMILES: Nc1nc(-n2cccn2)nc(-n2cccn2)c1Br]

In another embodiment, the A2AR antagonist is an A2AR antagonist disclosed in WO2009/156737. Especially, the A2AR antagonist is the compound of example 1S of WO2009/156737, namely (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, also known as CPI-444:

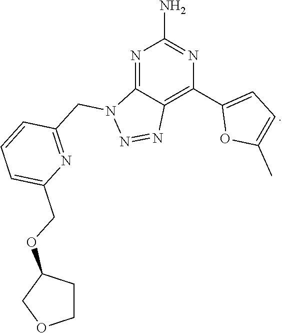 [SMILES: Cc1ccc(-c2nc(N)nc3c2nnn3Cc2cccc(CO[C@H]3CCOC3)n2)o1]

In another embodiment, the A2AR antagonist is an A2AR antagonist disclosed in WO2011/095626. Especially, the A2AR antagonist is the compound (cxiv) of WO2011/095626, namely 6-(2-chloro-6-methylpyridin-4-yl)-5-(4-fluorophenyl)-1,2,4-triazin-3-amine, also known as AZD4635:

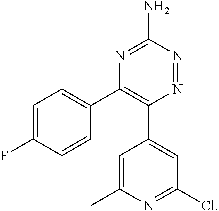 [SMILES: Cc1cc(-c2nnc(N)nc2-c2ccc(F)cc2)cc(Cl)n1]

In another embodiment, the A2AR antagonist is an A2AR antagonist disclosed in WO2018/136700. Especially, the A2AR antagonist is the compound of example 1 of WO2018/136700, namely 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, also known as AB928:

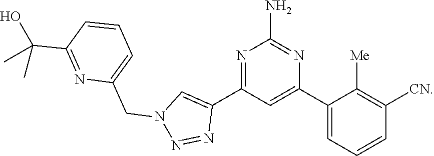 [SMILES: Cc1c(C#N)cccc1-c1cc(-c2cn(Cc3cccc(C(C)(C)O)n3)nn2)nc(N)n1]

In another embodiment, the A2AR antagonist is Preladenant (SCH-420,814), namely 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine.

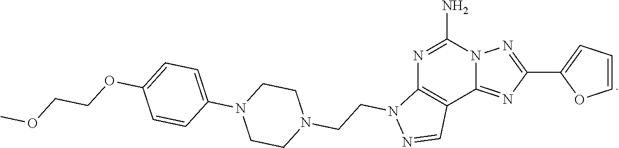 [SMILES: COCCOc1ccc(N2CCN(CCn3ncc4c3nc(N)n3nc(-c5ccco5)nc43)CC2)cc1]

In another embodiment, the A2AR antagonist is Vipadenant (BIIB-014), namely 3-(4-amino-3-methylbenzyl)-7-(2-furyl)-3H-(1,2,3)triazolo(4,5-d)pyrimidine-5-amine:

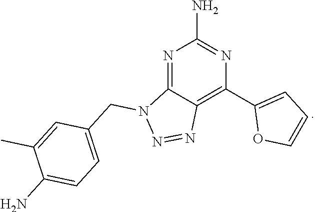 [SMILES: Cc1cc(Cn2nnc3c(-c4ccco4)nc(N)nc32)ccc1N]

In another embodiment, the A2AR antagonist is Tozadenant (SYK-115), namely 4-hydroxy-N-(4-methoxy-7-morpholinobenzo[d]thiazol-2-yl)-4-methylpiperidine-1-carboxamide:

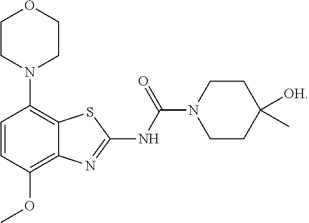 [SMILES: COc1ccc(N2CCOCC2)c2sc(NC(=O)N3CCC(C)(O)CC3)nc12]

Thus, in one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In one embodiment, the adenosine receptor antagonist is 5-bromo-2,6-di-(1H-pyrazol-1-yl)pyrimidin-4-amine. In one embodiment, the adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In one embodiment, the adenosine receptor antagonist is 6-(2-chloro-6-methylpyridin-4-yl)-5-(4-fluorophenyl)-1,2,4-triazin-3-amine. In one embodiment, the adenosine receptor antagonist is 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile.

### A2B Receptor Antagonist

In one embodiment, the combination of the present disclosure includes at least one A2BR antagonist.

An “A2BR antagonist” refers to a compound that, upon administration to a patient, results in inhibition or down-regulation of a biological activity associated with activation of A2B receptor in the patient, including any of the downstream biological effects otherwise resulting from the binding to A2B receptor of its natural ligand. Such A2BR antagonists include any agent that can block activation of A2B receptor or any of the downstream biological effects of A2B receptor activation.

Examples of A2BR antagonists include. Vipadenant (BIIB-014), CVT-6883, MRS-1706, MRS-1754, PSB-603, PSB-0788, PSB-1115, OSIP-339,391, ATL-801, theophylline, Caffeine,

### Specific Combinations

In one embodiment, the combination of the present disclosure comprises:
(a) an effective amount of an ENT inhibitor of the present disclosure, of formula I or a subformula thereof, and
(b) an effective amount of an adenosine receptor antagonist, preferably an A2AR antagonist, preferably selected from.
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
5-bromo-2,6-di-(1H-pyrazol-1-yl)pyrimidin-4-amine,
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-anine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;

and pharmaceutically acceptable salts thereof.

In one embodiment, the combination of the present disclosure comprises:
(a) an effective amount of an ENT inhibitor of the present disclosure, of formula I or a subformula thereof, and
(b) an effective amount of (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one as A2AR antagonist.

In one embodiment, the combination of the present disclosure comprises:
(a) an effective amount of an ENT inhibitor of the present disclosure, of formula I or a subformula thereof, and
(b) an effective amount of (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one as A2AR antagonist

### Combined Formulation and Kit of Parts

The present disclosure further provides a combined formulation, comprising the combination of the present disclosure. Especially, the present disclosure provides a combined formulation, comprising: an effective amount of an adenosine receptor antagonist in combination with an effective amount of an ENT inhibitor of the present disclosure, as defined above, along with a pharmaceutically acceptable excipient.

The present disclosure further relates to a combined pharmaceutical composition comprising the combination of the present disclosure. In one embodiment, the pharmaceutical composition comprises:

(a) an effective amount of an ENT inhibitor of the present disclosure, of formula I or a subformula thereof, as defined above; (b) an effective amount of an adenosine receptor antagonist; and (c) at least one pharmaceutically acceptable carrier, diluent, excipient and/or adjuvant.

The specific embodiments relative to the adenosine receptor antagonists and to the ENT inhibitor of the present disclosure recited above also apply in the context of the combined formulation and pharmaceutical composition of the present disclosure.

In a preferred embodiment, the present disclosure provides a combined pharmaceutical composition comprising: (a) an effective amount of an ENT inhibitor of the present disclosure, of formula I or a subformula thereof, as defined above; (b) an effective amount an A2AR antagonist being a thiocarbamate derivative, more preferably a thiocarbamate derivative of Formula (III)

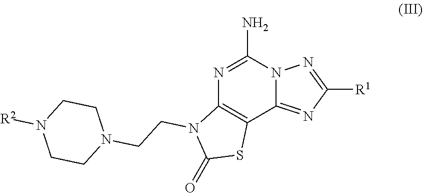

or a pharmaceutically acceptable salt or solvate thereof, as defined above; and
(c) at least one pharmaceutically acceptable carrier, diluent, excipient and/or adjuvant.

In one embodiment, the combined formulation or the pharmaceutical composition of the present disclosure further comprises an additional therapeutic agent.

The at least one pharmaceutically acceptable carrier, diluent, excipient and/or adjuvant for use in the preparation of the administration forms will be clear to the skilled person; reference is made to the latest edition of Remington's Pharmaceutical Sciences. The specific embodiments relative to formulations comprising an ENT inhibitor of the present disclosure also apply in the context of the combined formulation and pharmaceutical composition of the present disclosure.

The present disclosure further relates to a kit of parts comprising the combination of the present disclosure. In one embodiment, the kit of parts of the present disclosure comprises
(a) a first part comprising an effective amount of an ENT inhibitor of the present disclosure, of formula I or a subformula thereof, as defined above; and
(b) a second part comprising an effective amount of an adenosine receptor antagonist.

Above embodiments relative to the ENT inhibitor of the present disclosure and adenosine receptor antagonists also apply to the kit of parts of the present disclosure.

In a preferred embodiment, the present disclosure provides a kit of parts comprising:
(a) a first part comprising an effective amount of an ENT inhibitor of the present disclosure, of formula I or a subformula thereof, as defined above; and
(b) a second part comprising an effective amount an A2AR antagonist being a thiocarbamate derivative, more preferably a thiocarbamate derivative of Formula (III)

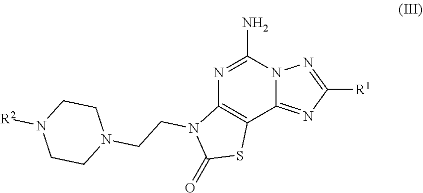

or a pharmaceutically acceptable salt or solvate thereof, as defined above.

Depending on the ENT inhibitor and adenosine receptor antagonist, the first and second parts of the kit may be under the form of pharmaceutical compositions. Excipients, dosage form and administration route of such pharmaceutical compositions will be clear to the skilled person (reference is made to the latest edition of Remington's Pharmaceutical Sciences), and especially may be those listed above with regards to the pharmaceutical compositions of the present disclosure.

In one embodiment, the kit of parts of the present disclosure further comprises an additional therapeutic agent.

In the context of the present disclosure, the administration of the ENT inhibitor and the adenosine receptor antagonist may occur either simultaneously or timely staggered, either at the same site of administration or at different sites of administration, under similar or different dosage forms as further outlined below.

In one embodiment, the ENT inhibitor is administered prior to, concomitant with, or subsequent to administration of an adenosine receptor antagonist. To ensure that the separate mechanisms elicited by the ENT inhibitor and the adenosine receptor antagonist are not negatively influenced by each other, the adenosine receptor antagonist and the ENT inhibitor may be administered separated in time (in a time-staggered manner), i.e. sequentially, and/or are administered at different administration sites. This means that the adenosine receptor antagonist may be administrated e.g. prior, concurrent or subsequent to the ENT inhibitor, or vice versa. Alternatively, or additionally, the adenosine receptor antagonist and the ENT inhibitor may be administered at different administration sites, or at the same administration site, preferably, when administered in a time staggered manner.

In one embodiment, the adenosine receptor antagonist is to be administered prior to and/or concomitantly with an ENT inhibitor. In one embodiment, the adenosine receptor antagonist is to be administered prior to the day or on the same day that the ENT inhibitor is administered. In another embodiment, the ENT inhibitor is to be administered prior to and/or concomitantly with an adenosine receptor antagonist. In one embodiment, the ENT inhibitor is to be administered prior to the day or on the same day that the adenosine receptor antagonist is administered. In one embodiment, the adenosine receptor antagonist is to be administered prior to and/or concomitantly with an ENT inhibitor and continuously thereafter. In another embodiment, the ENT inhibitor is to be administered prior to and/or concomitantly with an adenosine receptor antagonist and continuously thereafter.

Depending on the condition to be prevented or treated and the form of administration, the ENT inhibitor and the adenosine receptor antagonist may be administered as a single daily dose, divided over one or more daily doses.

It will be understood that the total daily usage of adenosine receptor antagonist and ENT inhibitor will be decided by the attending physician within the scope of sound medical judgment. The specific dose for any particular subject will depend upon a variety of factors such as the cancer to be treated; the age, body weight, general health, sex and diet of the patient; and like factors well-known in the medical arts.

Another object of this present disclosure is the use of the combination as a medicament, i.e. for medical use. Thus, in one embodiment, the present disclosure provides the use of the combination of the present disclosure for the manufacturing of a medicament. Especially, the present disclosure provides the use of the combined pharmaceutical composition of the present disclosure or the kit of the present disclosure for the manufacturing of a medicament.

Especially, the present disclosure provides the combination, the combined pharmaceutical composition or the kit of parts of the present disclosure, for use in the treatment and/or prevention of cancer. The present disclosure further provides the use of the combination, combined pharmaceutical composition or kit of parts of the present disclosure for the manufacture of a medicament for treating and/or preventing cancer. The present disclosure further provides a method of treating of cancer, which comprises administering to a mammal species in need thereof a therapeutically effective amount of the combination, combined pharmaceutical composition or kit of parts of the present disclosure.

Especially, the present disclosure provides a method of treating cancer, comprising: administering, to a patient in need thereof, a combination of an adenosine receptor antagonist and an ENT inhibitor. The specific embodiments relative to the adenosine receptor antagonists and ENT inhibitors recited above also applies in the context of the methods of treatment of the present disclosure.

The present disclosure also provides for a method for delaying in patient the onset of cancer comprising the administration of a pharmaceutically effective amount of the combination, combined pharmaceutical composition or kit of parts of the present disclosure to a patient in need thereof.

### Enumerated Embodiments

The present disclosure includes embodiments 1-34 described below.

1. A compound represented by Formula (I):

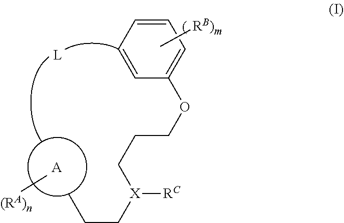

or a pharmaceutically acceptable salt thereof,
   wherein
L is an optionally substituted C₃-C₇ alkylene chain, wherein one, two, or three methylene units is optionally replaced with —O—, —N(R¹)—, —C(O)—, —C(O)O—, —C(O)N(R¹)—, —S(O)₂—, a 5-membered heteroaryl, —CH═CH—, or ≡C≡C—;
A is selected from the group consisting of —N(R^{A})— and 5-7 membered heterocyclyl;
X is —C(H)— or —N—;
each R^{A} is independently selected from the group consisting of halogen and optionally substituted C₁-C₆ alkyl;
each R^{B} is independently selected from the group consisting of optionally substituted C₁-C₆ alkoxy and halogen;
R^{C} is selected from the group consisting of hydrogen, optionally substituted benzyl, —OR², —OC(O)R², —C(O)R², —OC(O)OR², —N(R²)₂, and —OC(O)N(R²)₂;
each R¹ is hydrogen or optionally substituted C₁-C₃ alkyl;
each R² is independently selected from the group consisting of optionally substituted C₁-C₆ alkyl, —(CH₂)₀₋₃phenyl, —(CH₂)₀₋₃C(O)R³, 5-10 membered heteroaryl, 3-7 membered heterocyclyl, and —N═CH-phenyl, wherein R² is optionally substituted with one, two, or three instances of R⁴;
   wherein two instances of R² may be joined together with the atom on which they are attached to form a 5-10 membered heteroaryl or 3-7 membered heterocyclyl, optionally substituted with one, two, or three instances of R⁴;
each R³ is 5-10 membered heteroaryl or 3-7 membered heterocyclyl, wherein R³ is optionally substituted with one, two, or three instances of R⁴;
each R⁴ is selected from the group consisting of halogen, —OH, —NH₂, —CN, —NHR¹, optionally substituted C₁-C₆ alkyl, optionally substituted C₁-C₆ alkoxy and —S(O)₂C₁-C₃ alkyl;
   wherein two instances of R⁴ may be joined together with the atom on which they are attached to form a 5-10 membered heteroaryl or 3-7 membered heterocyclyl, optionally substituted with one, two, or three instances of oxo, halogen or C₁-C₃ alkyl;
n is 0, 1, 2, or 3; and
m is 0, 1, 2, or 3.
   2. The compound of claim 1, wherein L is an optionally substituted C₃-C₇ alkylene chain, wherein a methylene unit is replaced with —C(O)N(R¹)—.
   3. The compound of claim 1, wherein L is an optionally substituted C₃-C₇ alkylene chain, wherein a methylene unit is replaced with —C(O)O—.
   4. The compound of embodiment 1, wherein L is an optionally substituted C₃-C₇ alkylene chain, wherein a methylene unit is replaced with —O—.
   5. The compound of embodiment 1, wherein L is an optionally substituted C₃-C₇ alkylene chain, wherein a methylene unit is replaced with —S(O)₂—.
   6. The compound of embodiment 1 represented by Formula (I-a):

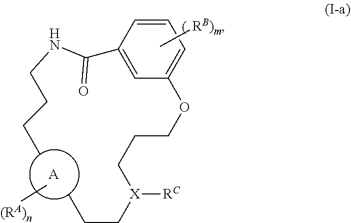

or a pharmaceutically acceptable salt thereof.
7. The compound of embodiment 1 represented by Formula (I-b):

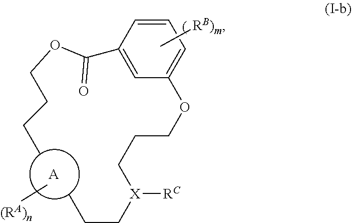

or a pharmaceutically acceptable salt thereof.
8. The compound of any of embodiments 1-7, wherein
R^{C} is selected from the group consisting of hydrogen, optionally substituted benzyl, —OR², —OC(O)R², —C(O)R², —OC(O)OR², —N(R²)₂, and —OC(O)N(R²)₂; and
R² is 5-6-membered heteroaryl, wherein R² is optionally substituted with one, two, or three instances of R⁴.
   9. The compound of any of embodiments 1-8, wherein A is selected from the group consisting of

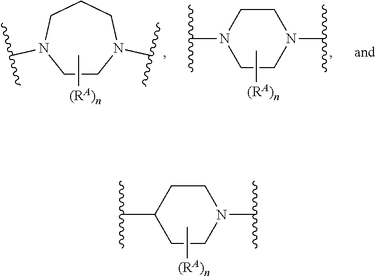

10. The compound of embodiment 9, wherein A is selected from the group consisting of

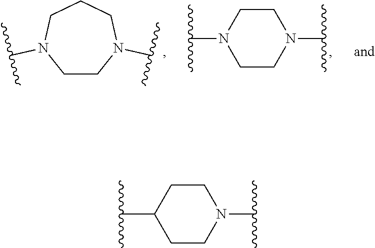

11. The compound of embodiment 1 represented by Formula (I-c):

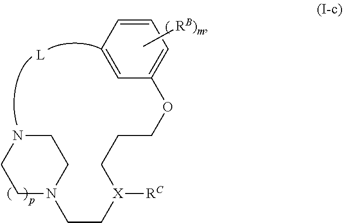

or a pharmaceutically acceptable salt thereof
   wherein,
p is 1 or 2.
   12. The compound of any of embodiments 1-11, wherein R^{C} is —OC(O)R².
   13. The compound of embodiment 12, wherein R² is 5-10-membered heteroaryl, wherein R² is optionally substituted with one, two, or three instances of R⁴.
   14. The compound of embodiment 13, wherein R^{C} is selected from the group consisting of

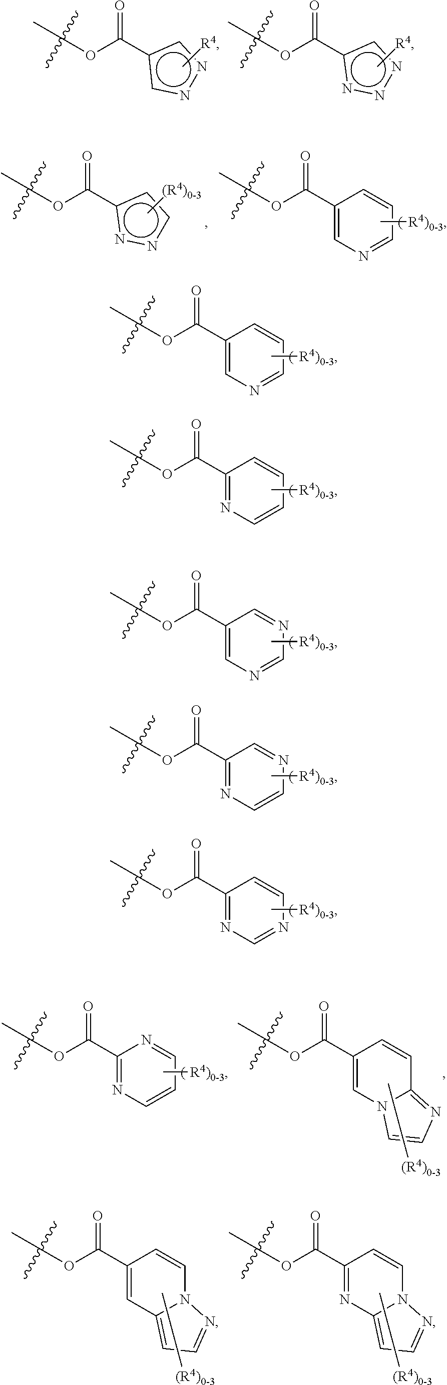

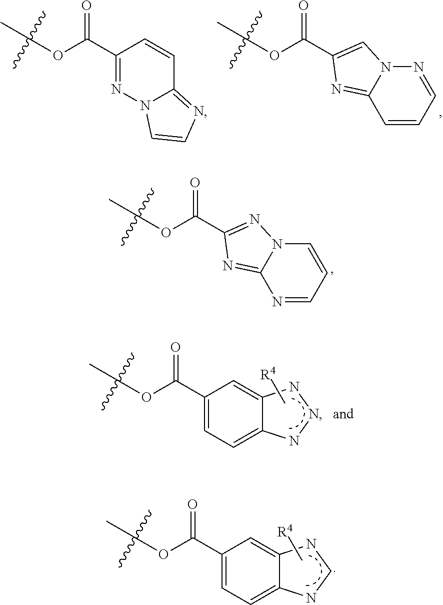

15. The compound of embodiment 12, wherein R² is 5-10-membered heterocyclyl, wherein R² is optionally substituted with one, two, or three instances of R⁴.
16. The compound of embodiment 15, wherein R^{C} is selected from the group consisting of

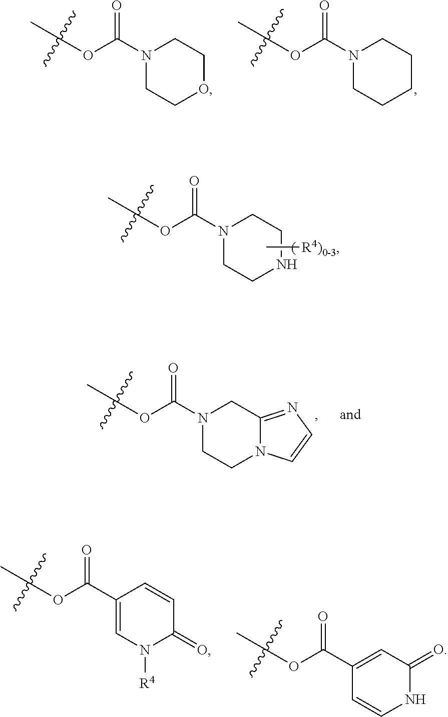

17. The compound of embodiment 12, wherein R² is —(CH₂)₀₋₃C(O)R³.
18. The compound of embodiment 17, wherein R^{C} is selected from the group consisting of

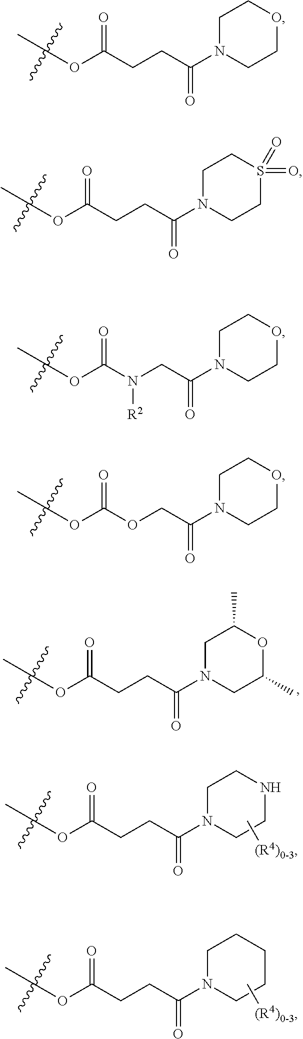

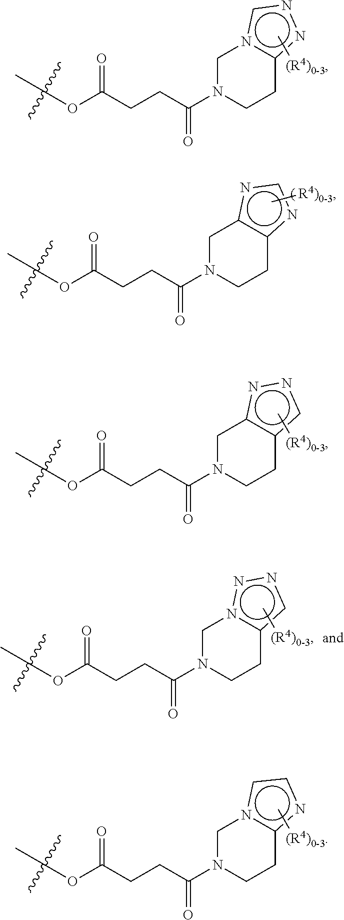

19. The compound of any of embodiments 1-11, wherein R^{C} is selected from the group consisting of

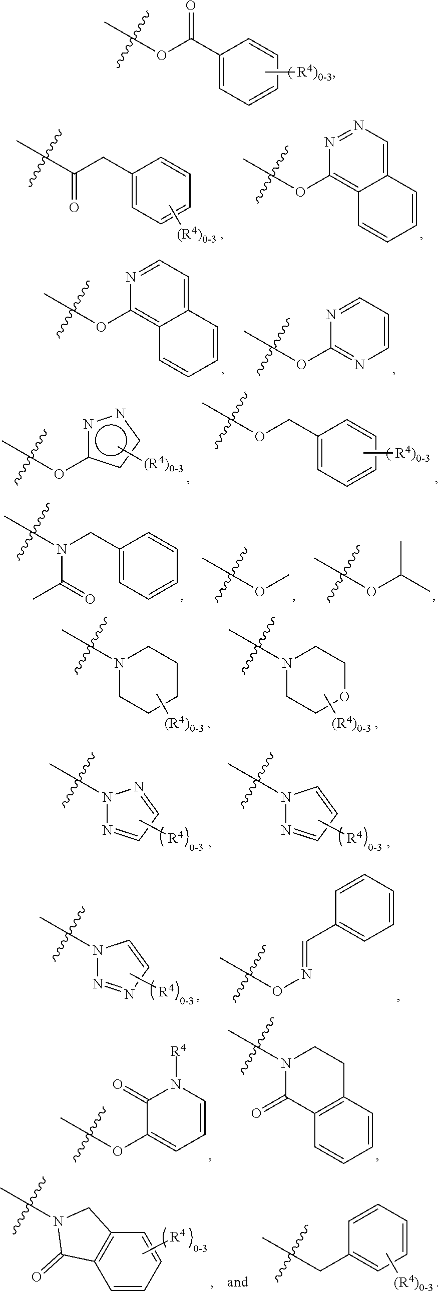

20. The compound of any of embodiments 1-19, wherein X is C(H).
21. The compound of any of embodiments 1-20, wherein n is 0.
22. The compound of any of embodiments 1-21, wherein m is 0.
23. The compound of any of embodiments 1-21, wherein m is 1.
24. The compound of any of embodiments 1-21, wherein m is 2.
25. The compound of any of embodiments 1-21, wherein m is 3.
26. The compound of any of embodiments 1-25, wherein each R⁴ is selected from the group consisting of halogen, optionally substituted C₁-C₆ alkyl, optionally substituted C₁-C₆ alkoxy, and —S(O)₂C₁-C₃ alkyl.
27. The compound of any of embodiments 1-26, wherein each R⁴ is selected from the group consisting of halogen, optionally substituted C₁-C₃ alkyl, optionally substituted C₁-C₃ alkoxy, and —S(O)₂C₁-C₃ alkyl.
28. A compound selected from the group consisting of

| Compound No. | Structure |
|---|---|
| 1 | 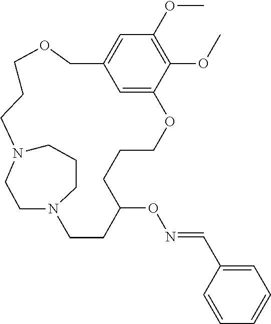 [SMILES: COc1cc2cc(c1OC)OCCCC(O/N=C/c1ccccc1)CCN1CCCN(CCCOC2)CC1] |
| 2 | 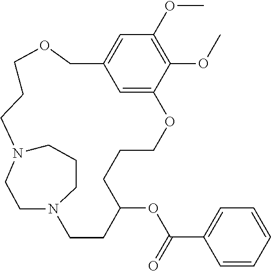 |
| 3 | 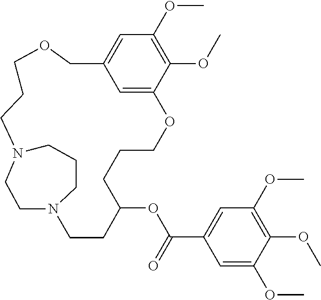 [SMILES: COc1cc(C(=O)OC2CCCOc3cc(cc(OC)c3OC)COCCCN3CCCN(CC2)CC3)cc(OC)c1OC] |
| 4 | 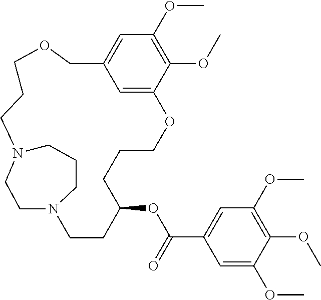 [SMILES: COc1cc(C(=O)O[C@@H]2CCCOc3cc(cc(OC)c3OC)COCCCN3CCCN(CC3)C(C)C2)cc(OC)c1OC] |
| 5 | 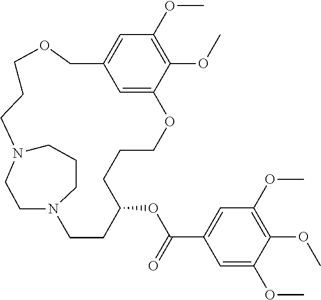 [SMILES: COc1cc(C(=O)O[C@H]2CCCOc3cc(cc(OC)c3OC)COCCCN3CCCN(CC3)CC2C)cc(OC)c1OC] |
| 6 | 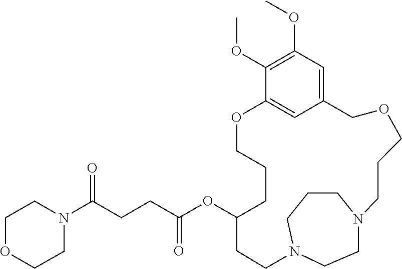 [SMILES: COc1cc2cc(c1OC)OCCCC(OC(=O)CCC(=O)N1CCOCC1)CCN1CCCN(CCCOC2)CC1] |
| 7 | 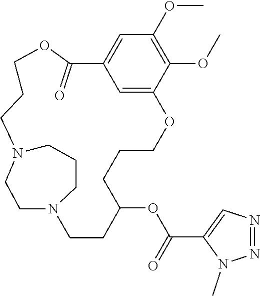 [SMILES: CCC(CCCOc1cc(C(=O)OCCCN2CCCN(C)CC2)cc(OC)c1OC)OC(=O)c1cnnn1C] |
| 8 | 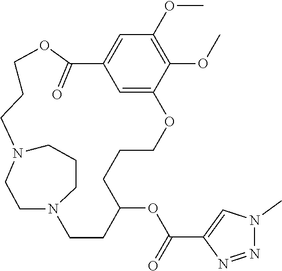 [SMILES: COc1cc2cc(c1OC)OCCCC(OC(=O)c1cn(C)nn1)CCN1CCCN(CCCOC2=O)CC1] |
| 9 | 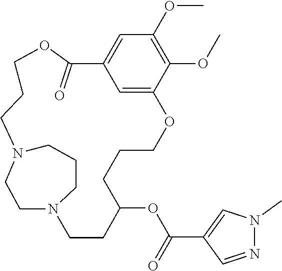 [SMILES: COc1cc2cc(c1OC)OCCCC(OC(=O)c1cnn(C)c1)CCN1CCCN(CCCOC2=O)CC1] |
| 10 | 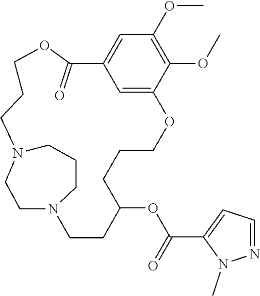 [SMILES: COc1cc2cc(c1OC)OCCCC(OC(=O)c1ccnn1C)CCN1CCCN(CCCOC2=O)CC1] |
| 11 | 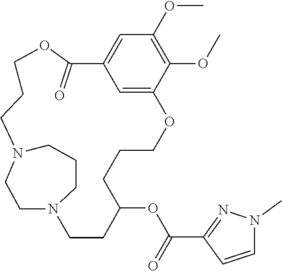 [SMILES: COc1cc2cc(c1OC)OCCCC(OC(=O)c1ccn(C)n1)CCN1CCCN(CCCOC2=O)CC1] |
| 12 | 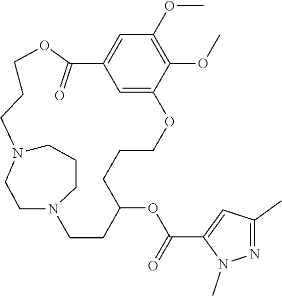 [SMILES: COc1cc2cc(c1OC)OCCCC(OC(=O)c1cc(C)nn1C)CCN1CCCN(CCCOC2=O)CC1] |
| 13 | 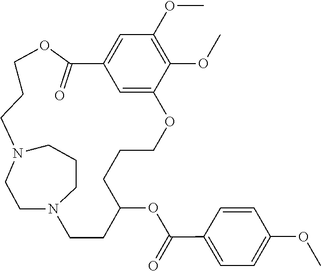 [SMILES: COc1ccc(C(=O)OC2CCCOc3cc(cc(OC)c3OC)C(=O)OCCCN3CCCN(CC2)CC3)cc1] |
| 14 | 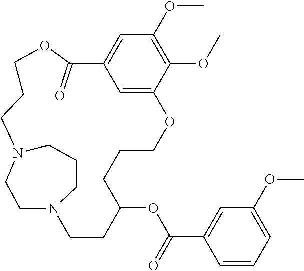 [SMILES: COc1cccc(C(=O)OC2CCCOc3cc(cc(OC)c3OC)C(=O)OCCCN3CCCN(CC2)CC3)c1] |
| 15 | 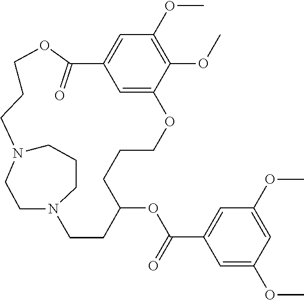 [SMILES: COc1cc(OC)cc(C(=O)OC2CCCOc3cc(cc(OC)c3OC)C(=O)OCCCN3CCCN(CC2)CC3)c1] |
| 16 | 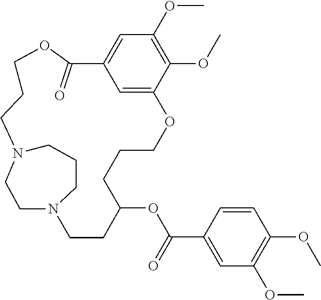 [SMILES: COc1ccc(C(=O)OC2CCCOc3cc(cc(OC)c3OC)C(=O)OCCCN3CCCN(CC2)CC3)cc1OC] |
| 17 | 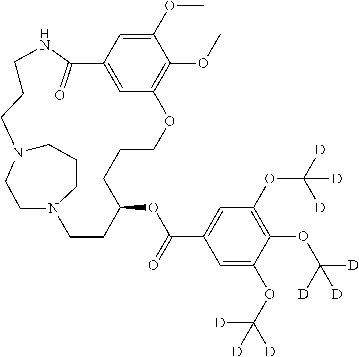 [SMILES: [2H]C([2H])([2H])Oc1cc(C(=O)O[C@@H]2CCCOc3cc(cc(OC)c3OC)C(=O)NCCCN3CCCN(CC2)CC3)cc(OC([2H])([2H])[2H])c1OC([2H])([2H])[2H]] |
| | (absolute stereochemistry) |
| 18 | 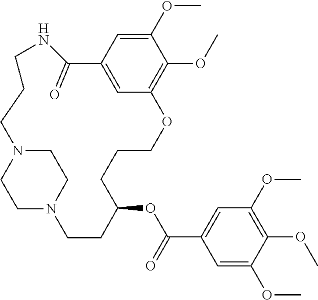 [SMILES: COc1cc(C(=O)O[C@@H]2CCCOc3cc(cc(OC)c3OC)C(=O)NCCCN3CCN(CC2)CC3)cc(OC)c1OC] |
| 19 | 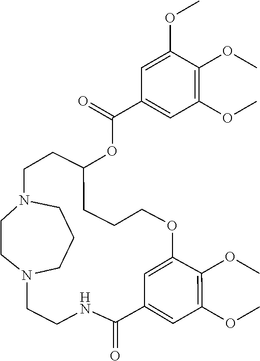 [SMILES: COc1cc(C(=O)OC2CCCOc3cc(cc(OC)c3OC)C(=O)NCCN3CCCN(CC2)CC3)cc(OC)c1OC] |
| 20 | 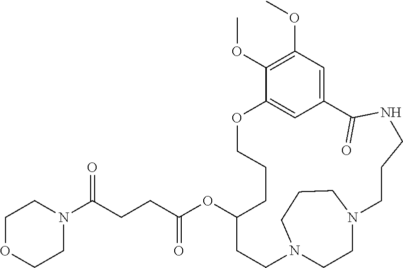 [SMILES: COc1cc2cc(c1OC)OCCCC(OC(=O)CCC(=O)N1CCOCC1)CCN1CCCN(CCCNC2=O)CC1] |
| 21 | 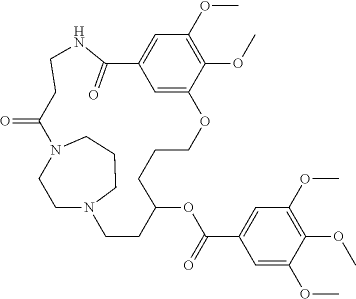 [SMILES: COc1cc(C(=O)OC2CCCOc3cc(cc(OC)c3OC)C(=O)NCCC(=O)N3CCCN(CC2)CC3)cc(OC)c1OC] |
| 22 | 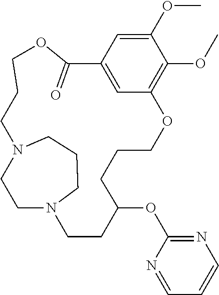 [SMILES: COc1cc2cc(c1OC)OCCCC(Oc1ncccn1)CCN1CCCN(CCCOC2=O)CC1] |
| 23 | 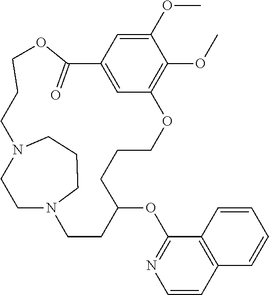 [SMILES: COc1cc2cc(c1OC)OCCCC(Oc1nccc3ccccc13)CCN1CCCN(CCCOC2=O)CC1] |
| 24 | 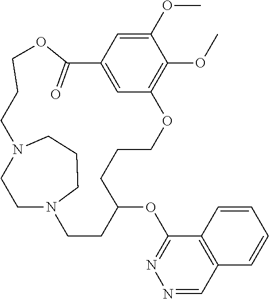 [SMILES: COc1cc2cc(c1OC)OCCCC(Oc1nncc3ccccc13)CCN1CCCN(CCCOC2=O)CC1] |
| 25 | 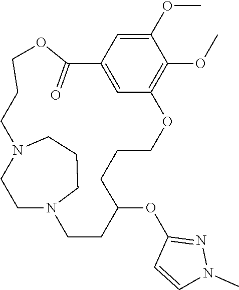 [SMILES: COc1cc2cc(c1OC)OCCCC(Oc1ccn(C)n1)CCN1CCCN(CCCOC2=O)CC1] |
| 26 | 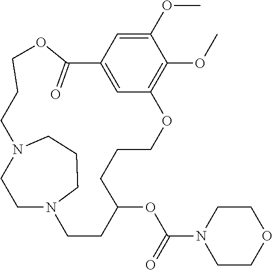 [SMILES: COc1cc2cc(c1OC)OCCCC(OC(=O)N1CCOCC1)CCN1CCCN(CCCOC2=O)CC1] |
| 27 | 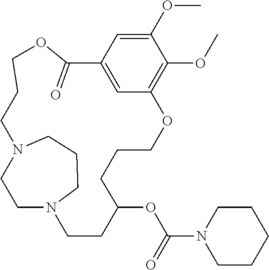 [SMILES: COc1cc2cc(c1OC)OCCCC(OC(=O)N1CCCCC1)CCN1CCCN(CCCOC2=O)CC1] |
| 28 | 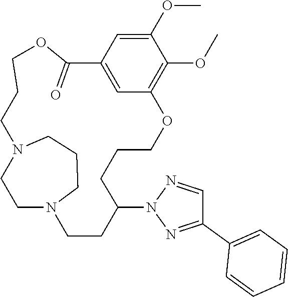 [SMILES: COc1cc2cc(c1OC)OCCCC(n1ncc(-c3ccccc3)n1)CCN1CCCN(CCCOC2=O)CC1] |
| 29 | 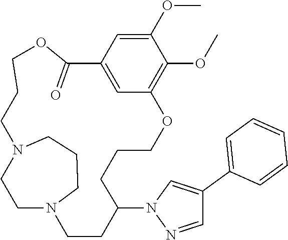 |
| 30 | 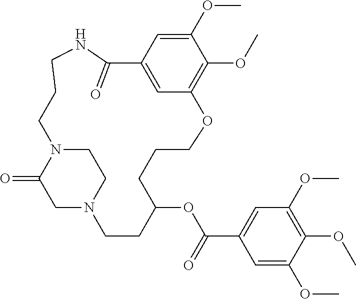 [SMILES: COc1cc(C(=O)OC2CCCOc3cc(cc(OC)c3OC)C(=O)NCCCN3CCN(CC2)CC3=O)cc(OC)c1OC] |
| 31 | 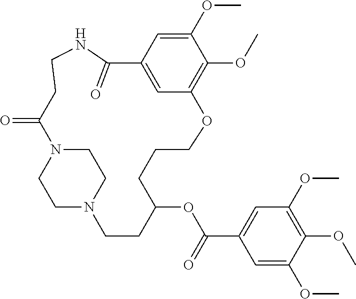 [SMILES: COc1cc(C(=O)OC2CCCOc3cc(cc(OC)c3OC)C(=O)NCCC(=O)N3CCN(CC2)CC3)cc(OC)c1OC] |
| 32 | 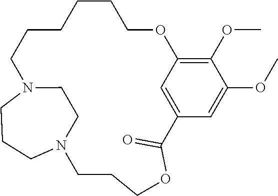 [SMILES: COc1cc2cc(c1OC)OCCCCCCN1CCCN(CCCOC2=O)CC1] |
| 33 | 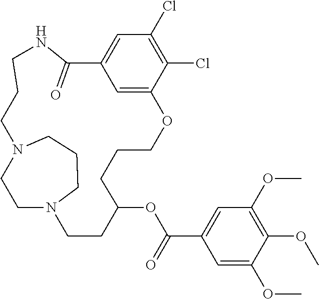 [SMILES: COc1cc(C(=O)OC2CCCOc3cc(cc(Cl)c3Cl)C(=O)NCCCN3CCCN(CC2)CC3)cc(OC)c1OC] |
| 34 | 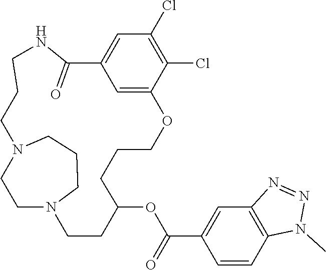 [SMILES: Cn1nnc2cc(C(=O)OC3CCCOc4cc(cc(Cl)c4Cl)C(=O)NCCCN4CCCN(CC3)CC4)ccc21] |
| 35 | 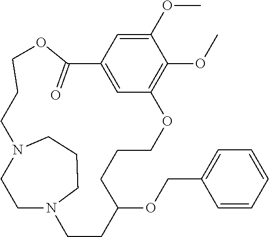 [SMILES: COc1cc2cc(c1OC)OCCCC(OCc1ccccc1)CCN1CCCN(CCCOC2=O)CC1] |
| 36 | 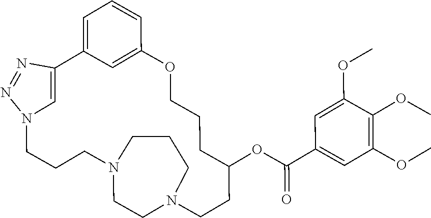 [SMILES: COc1cc(C(=O)OC2CCCOc3cccc(c3)-c3cn(nn3)CCCN3CCCN(CC2)CC3)cc(OC)c1OC] |
| 37 | 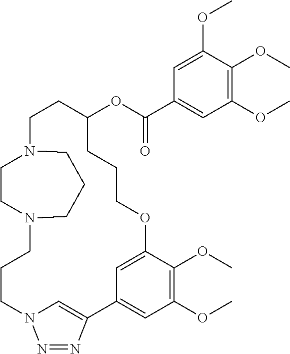 [SMILES: COc1cc(C(=O)OC2CCCOc3cc(cc(OC)c3OC)-c3cn(nn3)CCCN3CCCN(CC2)CC3)cc(OC)c1OC] |
| 38 | 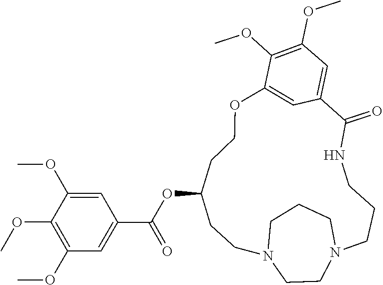 [SMILES: COc1cc(C(=O)O[C@H]2CCOc3cc(cc(OC)c3OC)C(=O)NCCCN3CCCN(CC2)CC3)cc(OC)c1OC] |
| 39 | 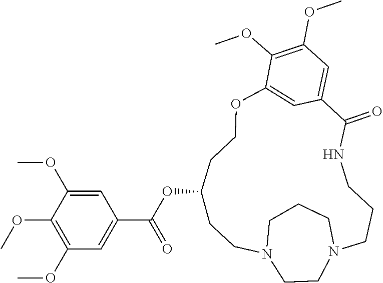 [SMILES: COc1cc(C(=O)O[C@@H]2CCOc3cc(cc(OC)c3OC)C(=O)NCCCN3CCCN(CC2)CC3)cc(OC)c1OC] |
| 40 | 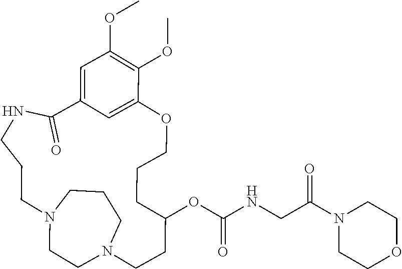 [SMILES: COc1cc2cc(c1OC)OCCCC(OC(=O)NCC(=O)N1CCOCC1)CCN1CCCN(CCCNC2=O)CC1] |
| 41 | 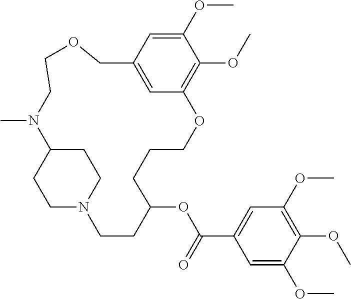 |
| 42 | 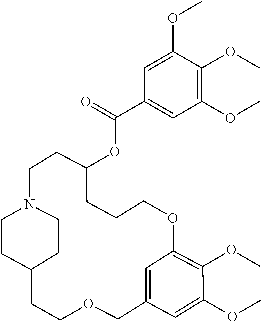 [SMILES: COc1cc(C(=O)OC2CCCOc3cc(cc(OC)c3OC)COCCC3CCN(CC3)CC2)cc(OC)c1OC] |
| 43 | 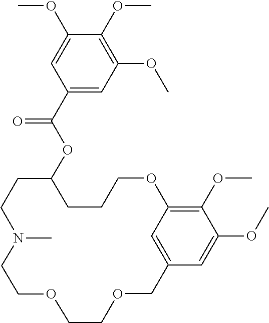 [SMILES: COc1cc(C(=O)OC2CCCOc3cc(cc(OC)c3OC)COCCOCCN(C)CC2)cc(OC)c1OC] |
| 44 | 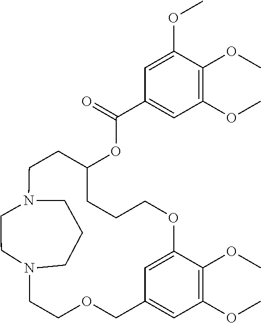 [SMILES: COc1cc(C(=O)OC2CCCOc3cc(cc(OC)c3OC)COCCN3CCCN(CC2)CC3)cc(OC)c1OC] |
| 45 | 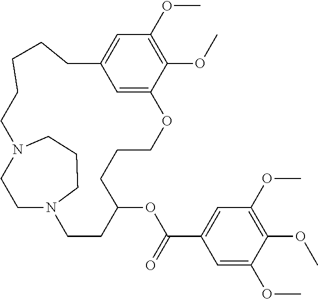 [SMILES: COc1cc(C(=O)OC2CCCOc3cc(cc(OC)c3OC)CCCCCN3CCCN(CC2)CC3)cc(OC)c1OC] |
| 46 | 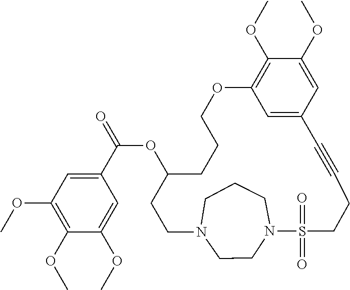 [SMILES: COc1cc(C(=O)OC2CCCOc3cc(cc(OC)c3OC)C#CCCS(=O)(=O)N3CCCN(CC2)CC3)cc(OC)c1OC] |
| 47 | 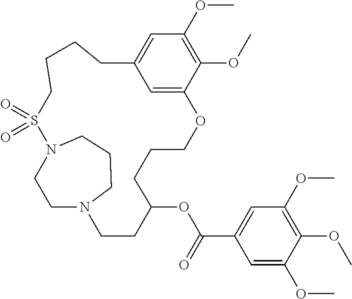 [SMILES: COc1cc(C(=O)OC2CCCOc3cc(cc(OC)c3OC)CCCCS(=O)(=O)N3CCCN(CC2)CC3)cc(OC)c1OC] |

or a pharmaceutically acceptable salt thereof.

29. A pharmaceutical composition comprising a compound according to any of embodiments 1-28 and a pharmaceutically acceptable excipient.
30. A method of inhibiting ENT1 in a patient need thereof, comprising: administering to said patient an effective amount of a compound according to any of embodiments 1-28 or a pharmaceutical composition of embodiment 29.
31. A method of treating cancer in a patient need thereof, comprising: administering to said patient an effective amount of a compound according to any of embodiments 1-28 or a pharmaceutical composition of embodiment 29.
32. A method of treating cancer in a patient need thereof, comprising: administering to said patient a combination of a compound according to any of embodiments 1-28 or a pharmaceutical composition of embodiment 29 and an adenosine receptor antagonist.
33. The method according to embodiment 32, wherein the adenosine receptor antagonist is an A2A or A2B receptor antagonist.
34. The method according to embodiment 32, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.
   35. A compound according to any of embodiments 1-28, for use to inhibit ENT1 in a patient need thereof.
   36. A compound according to any of embodiments 1-28, for use in the treatment of cancer.
   37. A combination comprising a compound according to any of embodiments 1-28 and an adenosine receptor antagonist.
   38. The combination according to embodiment 37, wherein the adenosine receptor antagonist is an A2A or A2B receptor antagonist.
   39. The combination according to embodiments 37 or 38, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.
   40. A combination according to any of embodiments 37-39, for use in the treatment of cancer.

### EXEMPLIFICATION

### Examples

The present disclosure will be better understood with reference to the following examples. These examples are intended to representative of specific embodiments of the present disclosure, and are not intended as limiting the scope of the present disclosure.

The following abbreviations are used:
THF: tetrahydrofuran;
DCM: dichloromethane;
EtOAC: ethyl acetate;
ACN: acetonitrile;
TEA: triethylamine;
DIPEA: N,N-Diisopropylethylamine;
EDCI: 1-Ethyl-3-(3-diméthylaminopropyl)carbodiimide;
HAUT: (1-[Bis(dimethylamino)methylene]-1H-1,2,3-triazolo[4,5-b]pyridinium 3-oxide hexafluorophosphate;
DPPF: 1,1′-Bis(diphenylphosphino)ferrocene
HOBt: 1-Hydroxybenzotriazole;
DTAD: Di-tert-butyl azodicarboxylate;
EDC·HCl: N′-ethylcarbodiimide hydrochloride
N2: nitrogen gas;
min: minute;
hr: hour;
Na₂SO₄: sodium sulfate;
MgSO₄: magnesium sulfate
TLC: Thin layer chromatography;
prep-HPLC: preparative High-Pressure Liquid Chromatography;
HPLC: High Pressure Liquid Chromatography;
SiO₂: silica gel;
K₂CO₃: potassium carbonate;
LiOH: lithium hydroxide.
DCC: N,N′-Dicyclohexylcarbodiimide
DMAP: 4-Dimethylaminopyridine
DEAD: Diethyl azodicarboxylate
PPh₃: triphenylphosphine
TBAF: tetra-n-butylammonium fluoride
TFA: trifluoroacetic acid
PE/EA: Petrol ether/Ethyl acetate
LiAlH4: Lithium aluminium hydride
TBAF: Tetra-n-butylammonium fluoride
CHCl3: chloroform
DBU: 1,8-Diazabicyclo[5.4.0]undec-7-ene
ADDP: 1,1′-(Azodicarbonyl)dipiperidine
T3P: Propanephosphonic acid anhydride

### I. Chemistry Examples

The MS data provided in the examples described below were obtained as follows: LCMS were recorded using Agilent 6130 or 6130B multimode (ESI+APCI).

### LCMS Methods

### Method A

This method was used for the LCMS analysis of intermediates. The column used for chromatography was a ZORBAX Eclipse XDB-C18 2.1*30 mm, (3.5 um particles. Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 5-95% B in 2.20 min, 5% B in 0.01 min, 5-95% B (0.01-1.00 min), 95-100% B (1.00-1.80 min), 5% B in 1.81 min with a hold at 5% B for 0.39 min. The flow rate was 1.0 mL/min.

### Method B

This method was used for the LCMS analysis of compounds. The column used for chromatography was a Kinetex C18 50*2.1 mm column (5 um particles). Detection methods are diode array (DAD) and evaporative light scattering (ELSD) detection as well as positive electrospray ionization. MS range was 100-1000. The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile.

### Chiral HPLC:

### Condition 1

(Column: YMC Cellulose-SB, 100*4.6 mm, 3 um 121AB00077, Mobile Phase, A: n-Hexane; B: Ethanol (0.1% DEA); Flow Rate: 1 mL/min; Conc. of Pump B: 30%; Detection: 254 nm;

### Condition 2

Column: YMC Cellulose-SC, 100*4.6 mm, 3 um 119IA70110, Mobile Phase, A: n-Hexane (0.1% DEA); B: Ethanol; Flow Rate: 1 mL/min; Conc. of Pump B: 50.0%; Detection: 254 nm;

### NMR Analysis

The NMR data provided in the examples described below were obtained as followed: 1H-NMR: Bruker DPX 400 MHz. Abbreviations for multiplicities observed in NMR spectra are as follows: s (singlet), d (doublet), t (triplet), q (quadruplet), m (multiplet), br (broad) Solvents, reagents and starting materials were purchased and used as received from commercial vendors unless otherwise specified.

### Synthesis of Intermediate Compounds

### Intermediate Compound 1

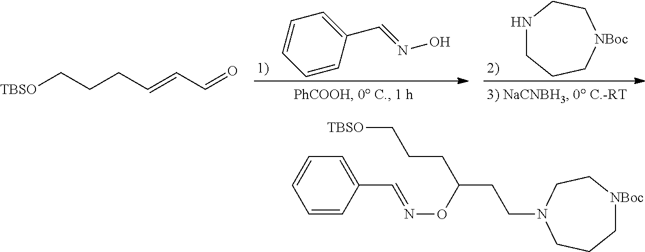 [SMILES: CC(C)(C)[Si](C)(C)OCCC/C=C/C=O ; O/N=C/c1ccccc1 ; CC(C)(C)OC(=O)N1CCCNCC1 ; CC(C)(C)OC(=O)N1CCCN(CCC(CCCO[Si](C)(C)C(C)(C)C)O/N=C/c2ccccc2)CC1]

To a stirred solution of (2S)-2-[bis[3,5-bis(trifluoromethyl)phenyl][(trimethylsilyl)oxy]methyl]pyrrolidine (261 mg, 0.43 mmol, 0.1 equiv) and benzoic acid (54 mg, 0.43 mmol, 0.1 equiv) in toluene (2.2 mL) at 0° C. was added intermediate compound 107 (1.0 g, 4.4 mmol, 1.0 equiv), followed by E-benzaldoxime (1.6 g, 13.1 mmol, 3.0 equiv) and the solution was stirred for 4 h at 0° C. The reaction mixture was diluted with DCM (15 mL), followed by the addition of tert-butyl 1,4-diazepane-1-carboxylate (1.4 g, 7.0 mmol, 1.6 equiv) and the reaction mixture was stirred at room temperature for a further 1 h. Sodium borohydride (324 mg, 8.8 mmol, 2.0 equiv) was added and the reaction mixture was stirred at room temperature for a further 1 h. The reaction mixture was diluted with saturated aqeuous solution of NH4Cl and extracted with DCM (3×20 mL). The combined organic layers were washed with brine, dried over MgSO4 and evaporated under reduced pressure. The crude oil was purified by preparative HPLC (Column (C18-I, 20-40 μm); mobile phase (MeOH/H2O=40% to 100%:6 min; 100%:5 min); Detector (254 and 220 nm)), to give the intermediate compound 108 (650 mg, 28% yield) as a colourless oil Note: the compound has been determined as racemic.

LC-MS (ES+) m/z: 534 (M+H)+ (calculated: 533.4)

### Intermediate Compound 2

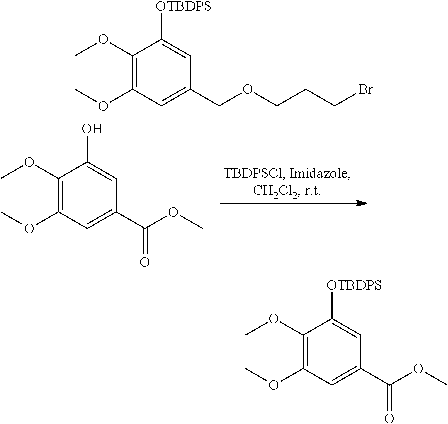

To a solution of methyl 3-hydroxy-4,5-dimethoxybenzoate (5.00 g, 23.6 mmol, 1.0 equiv) and imidazole (2.41 g, 35.3 mmol, 1.15 equiv) in DCM (160 mL) was added tert-butyl(chloro)diphenylsilane (7.05 mL, 27.1 mmol, 1.15 equiv.) dropwise at 0° C. under a nitrogen atmosphere. The reaction mixture was stirred overnight at room temperature under a nitrogen atmosphere. The reaction was quenched by the addition of water (200 mL) at room temperature. The resulting mixture was extracted with EtOAc (3×100 mL). The combined organic layers were washed with brine (1×100 mL), dried over anhydrous Na₂SO₄ and concentrated under reduced pressure. The residue was purified by silica gel column chromatography, eluting with PE/EA (0-50% gradient) to afford methyl 3-((tert-butyldiphenylsilyl)oxy)-4,5-dimethoxybenzoate (10 g, 95% yield) as a colourless solid.

1H NMR (400 MHz, CDCl3-d) δ 7.76-7.66 (m, 4H), 7.47-7.32 (m, 6H), 7.17 (s, 1H), 6.97 (s, 1H), 3.85 (s, 3H), 3.75 (s, 3H), 3.72 (s, 3H), 1.13 (s, 9H).

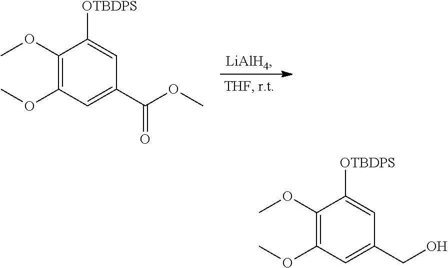

To a solution of methyl 3-((tert-butyldiphenylsilyl)oxy)-4,5-dimethoxybenzoate (5.30 g, 11.7 mmol, 1.0 equiv) in THF (51 mL) was added 2 M LiAlH₄ in THF (11.8 mL, 23.52 mmol, 2.0 equiv.) dropwise at 0° C. under a nitrogen atmosphere. The reaction mixture was stirred overnight at room temperature under a nitrogen atmosphere. The reaction mixture was cooled to 0° C. (ice bath) and diluted with THF (50 mL) The reaction was quenched by addition of water (1 mL) dropwise, followed by 15% aq. NaOH (1 mL) and additional water (3 mL). The reaction mixture was warmed to room temperature and stirred for 15 mins. The quenched reaction mixture was dried over anhydrous MgSO₄ and concentrated under reduced pressure. The residue was purified by silica gel column chromatography, eluting with PE/EA (20-100% gradient) to afford (3-((tert-butyldiphenylsilyl)oxy)-4,5-dimethoxyphenyl)methanol (3.45 g, 69% yield) as a colourless oil.

1H NMR (400 MHz, CDCl3-d) δ 7.77-7.68 (m, 4H), 7.46-7.32 (m, 6H), 6.51 (s, 1H), 6.14 (s, 1H), 4.29 (s, 2H), 3.83 (s, 3H), 3.79 (s, 3H), 1.12 (s, 9H).

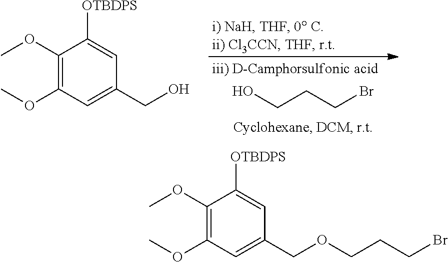

To a solution of (3-((tert-butyldiphenylsilyl)oxy)-4,5-dimethoxyphenyl)methanol (3.42 g, 8.09 mmol, 1.5 equiv) in anhydrous THF (5.25 mL) under a nitrogen atmosphere was slowly added sodium hydride (43.1 mg, 1.08 mmol, 0.2 equiv.) in anhydrous THF and the reaction mixture was stirred at room temperature for 30 minutes. The reaction mixture was cooled to 0° C. and trichloroacetonitrile (0.81 mL, 8.09 mmol, 1.5 equiv.) was added and the reaction mixture was slowly warmed to room temperature and stirred for 4 h. The reaction mixture was concentrated under reduced pressure. The residue was suspended in heptane (50 mL) and methanol (0.2 mL) and filtered through celite. The filtrate was concentrated under reduced pressure to afford a yellow oil. The crude oil was dissolved in cyclohexane (9.00 mL) and a solution of 3-bromopropanol (750 mg, 5.39 mmol, 1.0 equiv.) in DCM (4.50 mL) was added. The resulting mixture was cooled to 0° C. (ice bath) and (±)-10-camphorsulfonic acid (125 mg, 0.540 mmol, 0.1 equiv.) was added. The reaction mixture was warmed to room temperature and stirred overnight. The resulting colourless precipitate formed was filtered though celite and washed with cyclohexane/DCM (1.2, 40 mL) The filtrate was concentrated under reduced pressure and the crude residue was purified by silica gel column chromatography, eluting with PE/EA (0-50% gradient) to afford Intermediate compound 2 (2.5 g, 85% yield) as a colourless solid.

1H NMR (400 MHz, CDCl3-d) δ 7.78-7.72 (m, 4H), 7.46-7.33 (m, 6H), 6.45 (s, 1H), 6.14 (s, 1H), 4.13 (s, 2H), 3.83 (m, 6H), 3.34 (t, J=6.9 Hz, 2H), 3.24 (d, J=6.2 Hz, 2H), 1.96-1.87 (m, 2H), 1.13 (s, 9H).

### Intermediate Compound 3

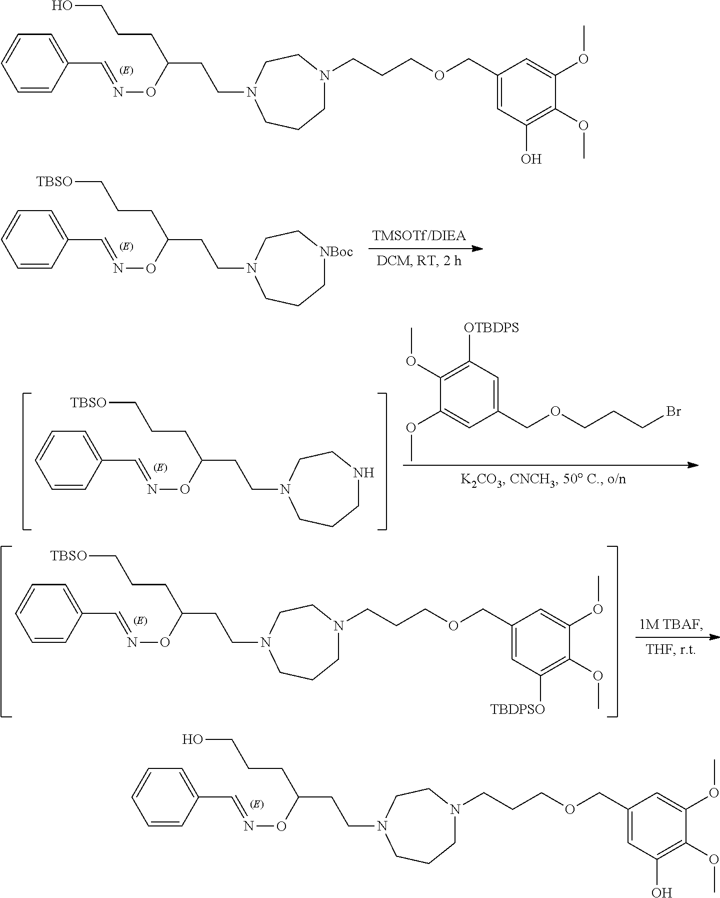

To a solution of intermediate compound 1 (797 mg, 1.49 mmol, 1.0 equiv) and DIPEA (1.12 mL, 6.41 mmol, 4.0 equiv) in DCM (14 mL), was added trimethylsilyl trifluoromethanesulfonate (0.81 mL, 4.48 mmol, 3.0 equiv) at 0° C. The resulting solution was stirred for 2 h at room temperature, and then quenched by the addition of water (10 mL). The organic phase was separated and washed with water (10 mL), followed by brine (10 mL). The organic layer was dried over anhydrous sodium sulfate and concentrated. The residue was dissolved in MeCN (10 mL) and K₂CO₃ (308 mg, 2.23 mmol, 1.5 equiv) was added, followed by intermediate compound 2 (890 mg, 1.64 mmol, 1.1 equiv) at room temperature under a nitrogen atmosphere. The reaction mixture was stirred overnight at 50° C., and was allowed to cool down to room temperature. The reaction was diluted with water (200 mL) and the resulting mixture was extracted with EtOAc (3×50 mL). The combined organic layers were washed with brine (1×50 mL), dried over anhydrous MgSO₄ and concentrated under reduced pressure. The residue was dissolved in anhydrous THF (13.5 mL) and 1 M TBAF (6.02 mLm 6.02 mmol, 4 equiv.) was added dropwise at 0° C. under a nitrogen atmosphere. The reaction mixture was stirred for 2 h at room temperature. The reaction was quenched by the addition of water (100 mL) at room temperature. The resulting mixture was extracted with EtOAc (3×70 mL) The combined organic layers were washed with brine (1×100 mL), dried over anhydrous MgSO4 and concentrated under reduced pressure. The residue was purified by reverse flash chromatography (column, C18 silica gel; mobile phase, MeCN (0.1% Formic Acid) in water (0.1% Formic Acid), (5-60% gradient), UV 254 nm and 220 nm) to afford a yellow oil. To the residue was added sat. aq. NaHCO₃ (50 mL) and extracted with CHCl3 (3×20 mL). The combined organic layers were dried over anhydrous MgSO₄ and concentrated under reduced pressure to afford the intermediate compound 3 (529 mg, 65% yield over 3 steps) as a light yellow oil.

LCMS (ESI position ion) m/z: 544.6 (M+H)+ (calculated: 544.7)

1H NMR (400 MHz, CDCl3-d) δ 8.06 (s, 1H), 7.60-7.53 (m, 2H), 7.42-7.32 (m, 3H), 6.60 (s, 1H), 6.44 (s, 1H), 4.38 (s, 2H), 4.33-4.20 (m, 1H), 3.88 (s, 3H), 3.86 (s, 3H), 3.72-6.65 (m, 2H), 3.50 (t, J=6.2 Hz, 2H), 3.00-2.62 (m, 12H), 2.09-1.59 (m, 10H).

### Intermediate Compound 4

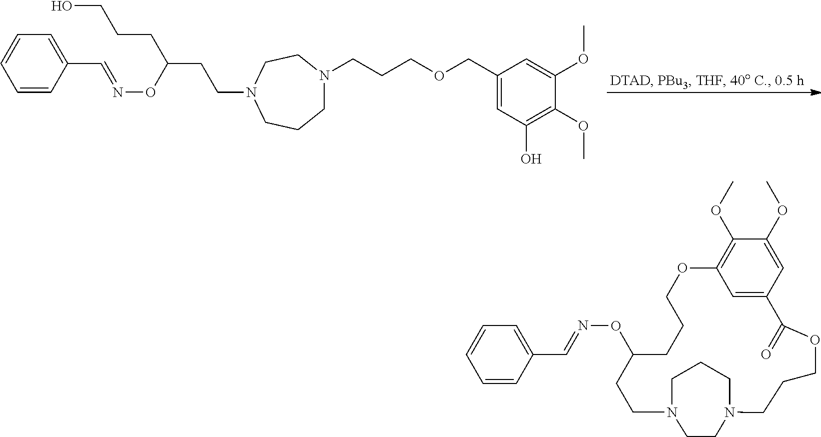 [SMILES: COc1cc(COCCCN2CCCN(CCC(CCCO)O/N=C/c3ccccc3)CC2)cc(O)c1OC ; COc1cc2cc(c1OC)OCCCC(O/N=C/c1ccccc1)CCN1CCCN(CCCOC2=O)CC1]

A solution of DTAD (0.74 g, 3.0 mmol, 1.5 equiv) and n-butylphosphine (0.60 g, 3.0 mmol, 1.5 equiv) in dry THF (20 mL) was stirred under nitrogen for 15 min, after which a solution of intermediate compound 111 (1.1 g, 2.0 mmol, 1.0 equiv) in THF (13 mL) was added. The mixture was stirred for 30 min at 40° C., and then quenched by the addition of H2O (50 mL). The resulting solution was extracted with EtOAc (2×15 mL). The combined organic layers were dried over Na2SO4 and concentrated. The residue was purified by preparative HPLC (Column (C18-I, 20-40 m); mobile phase (MeOH/H2O=30% to 100%:7 min; 100%:3 min); Detector (254 and 220 nm)) to give the compound 37 (0.48 g, 45% yield) as an off-white solid.

LC-MS (ES+) m/z: 540 (M+H)+ (calculated: 539.3).

1H NMR (300 MHz, DMSO-d6) δ 8.31 (s, 1H), 7.57-7.55 (m, 2H), 7.46-7.35 (m, 4H), 7.32 (s, 1H), 4.51-4.48 (m, 1H), 4.32-4.20 (m, 3H), 4.11-4.05 (m, 1H), 3.84 (s, 3H), 3.74 (s, 3H), 2.89-2.84 (m, 1H), 2.72-2.54 (m, 11H), 1.97-1.71 (m, 10H).

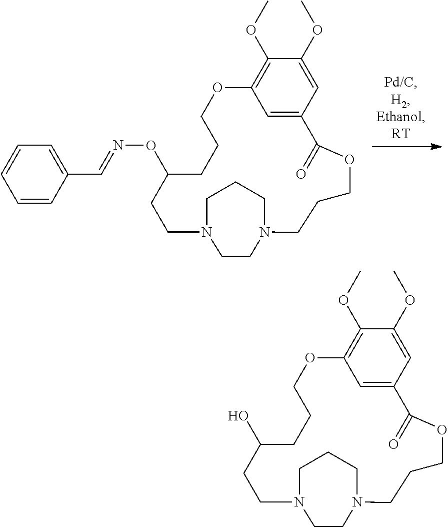 [SMILES: COc1cc2cc(c1OC)OCCCC(O/N=C/c1ccccc1)CCN1CCCN(CCCOC2=O)CC1 ; COc1cc2cc(c1OC)OCCCC(O)CCN1CCCN(CCCOC2=O)CC1]

A suspension of compound 37 (300 mg, 0.56 mmol, 1.0 equiv) and Pd/C (30 mg) in MeOH (5 mL) was stirred for 2 h at room temperature under H2 (1 atm). The resulting mixture was then filtered; and the solid residue was washed with MeOH (15 mL). The filtrate was concentrated under reduced pressure, and the residue was purified by preparative HPLC (Column: Atlantis Prep T3 OBD Column, 19*150 mm 5 um; Mobile Phase A: Water (0.1% FA), Mobile Phase B: ACN; Flow rate: 20 mL/min; Gradient: 15% B to 35% B in 7 min, 35% B; Wave Length 220 nm)) to give the compound 38 (HCO(OH salt, 166 mg, 62% yield) as an off-white solid.

LC-MS (ES+) m/z: 437 (M+H)+ (calculated: 436.2)

1H NMR (300 MHz, DMSO-d6) δ 8.22 (s, 1H), 7.32 (s, 1H), 7.22 (s, 1H), 4.27-3.99 (m, 4H), 3.83 (s, 3H), 3.80-3.76 (m, 4H), 2.92-2.87 (m, 1H), 2.75-2.46 (m, 11H), 1.97-1.88 (m, 10H).

### Intermediate Compound 5

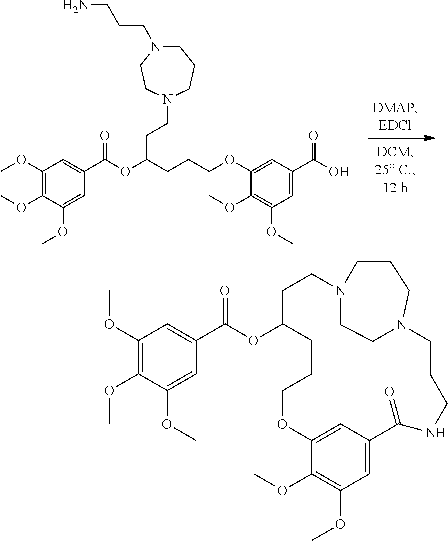

To a solution of intermediate compound 26 (380 mg, 586.64 umol, 1 eq) in DCM (120 mL) was added EDCI (337.38 mg, 1.76 mmol, 3 eq) and DMAP (286.68 mg, 2.35 mmol, 4 eq). The reaction was stirred at 25° C. for 12 hr. The reaction mixture was concentrated under vacuum. The residue was diluted with H2O (60 mL) and then extracted with ethyl acetate (3×20 mL). The combined organic layers were washed with brine (20 mL), dried over Na2SO4, filtered and concentrated under vacuum. The crude product was dissolved in DMF (5 mL) and then purified by Prep-HPLC (column. Phenomenex Synergi C18 150*25 mm*10 um; mobile phase. [water (0.225% FA)-ACN]; B %: 8%-38%, 10 min) to give the compound 8 (150 mg, 41% yield) as a white solid.

LCMS (ESI position ion) m/z: 630.3 (M+H)+ (calculated: 629.3)

1H NMR (400 MHz, CD3OD-d4) δ 7.34 (s, 2H), 7.22 (d, J=2.0 Hz, 1H), 7.13 (d, J=2.0 Hz, 1H), 5.52 (br d, J=6.2 Hz, 1H), 4.38-4.28 (m, 1H), 4.25-4.16 (m, 1H), 3.92-3.87 (m, 9H), 3.85 (s, 3H), 3.83 (s, 3H), 3.68-3.58 (m, 1H), 3.51-3.41 (m, 1H), 3.04-2.95 (m, 1H), 2.89-2.54 (m, 11H), 2.01-1.73 (m, 10H)

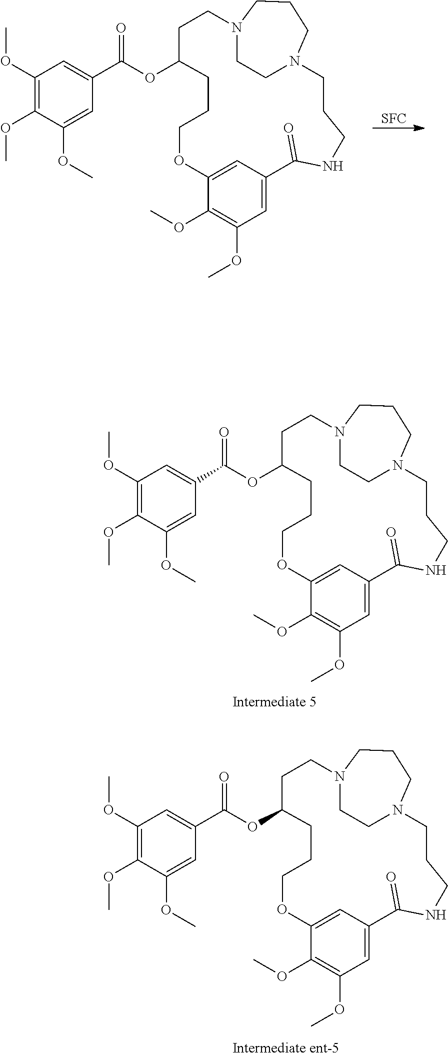

Compound 5 was separated from the racemic mixture by preparative SFC. The column used for chromatography was a Kinetex C18 50*2.1 mm column (5 um particles). Detection methods are diode array (DAD) and evaporative light scattering (ELSD) detection as well as positive electrospray ionization. MS range was 100-1000. The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile.

LCMS (ESI position ion) m/z: 630.6 (M+H)+ (calculated: 629.3)

SFC: retention time=4.099 min, ee=95.48%

1H NMR (400 MHz, MeOD) δ 7.36-7.30 (m, 3H), 7.21 (d, J=1.8 Hz, 1H), 5.43 (br d, J=3.9 Hz, 1H), 4.42-4.31 (m, 1H), 4.27-4.16 (m, 1H), 3.89 (s, 3H), 3.85 (s, 6H), 3.81 (d, J=6.1 Hz, 6H), 3.72-3.61 (m, 1H), 3.53-3.44 (m, 1H), 3.14-2.97 (m, 4H), 2.94 (br t, J=6.4 Hz, 2H), 2.90-2.78 (m, 4H), 2.74 (br t, J=6.7 Hz, 2H), 2.16-2.04 (m, 1H), 2.00-1.89 (m, 9H)

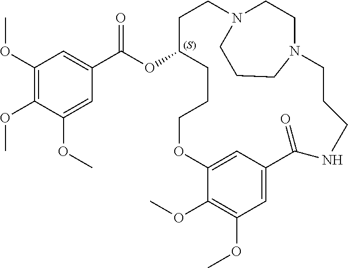

### Intermediate Compound 6

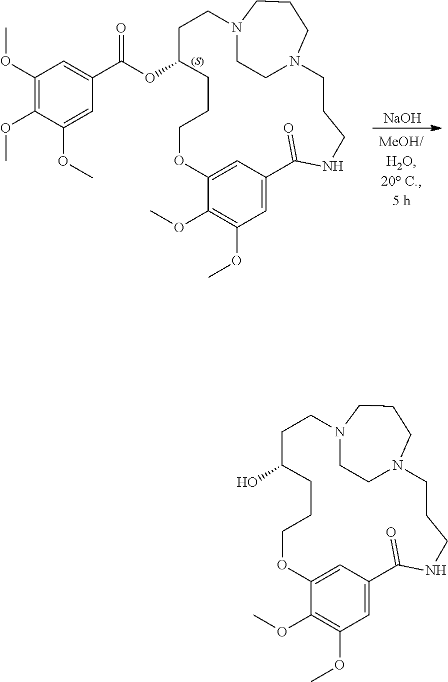

To a solution of intermediate compound 5 (31.1 g, 49.39 mmol, 1 eq) in MeOH (310 mL) and H₂O (155 mL) was added NaOH (5.93 g, 148.16 mmol, 3 eq). The mixture was stirred at 20° C. for 5 hr. The solvent MeOH was removed under reduced pressure at 25° C. The mixture was diluted with H₂O (100 mL) and extracted with DCM (2×200 mL). The organic layer was washed with brine, dried over anhydrous Na₂SO₄. The solution was concentrated to afford the intermediate compound 6 (21.76 g, crude) as yellow solid.

LCMS (ESI position ion) m/z: 436.3 (M+H)+ (calculated: 436.3)

1H NMR (400 MHz, CD3OD-d4) δ 7.17-7.12 (m, 2H), 4.30-4.14 (m, 2H), 3.98-3.90 (m, 1H), 3.88 (s, 3H), 3.85-3.80 (m, 3H), 3.57-3.40 (m, 2H), 2.87-2.61 (m, 11H), 2.60-2.53 (m, 1H), 2.01-1.93 (m, 1H), 1.90-1.71 (m, 5H), 1.71-1.53 (m, 4H)

### Intermediate Compound 7

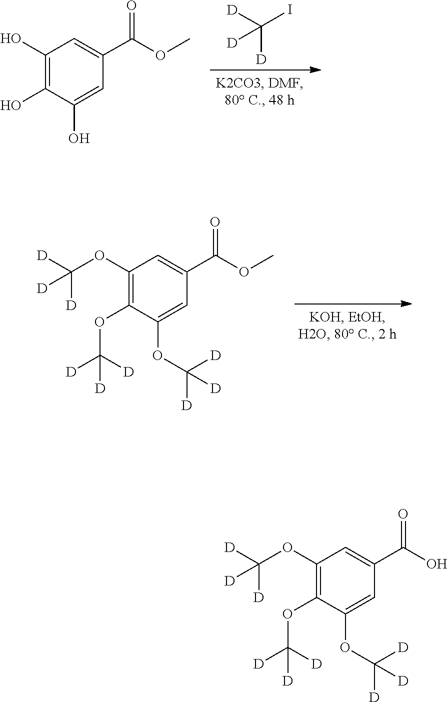

To a solution of methyl 3,4,5-trihydroxybenzoate (2 g, 10.86 mmol, 1 eq) in DMF (60 mL) were added K₂CO₃ (4.50 g, 32.58. mmol, 3 eq) and iodomethane-d3 (7.87 g, 54.30 mmol, 3.38 mL, 5 eq). The mixture was stirred at 80° C. for 48 hr in a sealed tube. The reaction mixture was added H₂O (100 mL) and extracted with EtOAc (2×100 mL). The organic layer was washed with brine, dried over anhydrous Na₂SO₄ and concentrated under reduced pressure. The residue was purified by column chromatography (SiO2, Petroleum ether/Ethyl acetate=25/1 to 20/1, (Rf=0.45)) to afford the methyl 3,4,5-tris(methoxy-d3)benzoate (2.3 g, 90% yield) as white solid

LCMS (ESI position ion) m/z: 236.1 (M+H)+ (calculated: 236.1)

To a solution of methyl 3,4,5-tris(methoxy-d3)benzoate (2.29 g, 9.73 mmol, 1 eq) in EtOH (24 mL) and H₂O (12 mL) was added KOH (1.64 g, 29.20 mmol, 3 eq) The mixture was stirred at 80° C. for 2 hr. The reaction mixture was diluted with 21-10 (100 mL) and extracted with EtOAc (100 mL). The aqueous layer was adjusted pH=2-3 with an aqueous solution of HCl (3 M) at 0° C. The mixture was extracted with DCM (2×100 mL). The organic layer was washed with brine, dried over anhydrous Na₂SO₄. The solution was concentrated to afford the intermediate compound 7 (1.9 g, 88% yield) as white solid

LCMS (ESI position ion) m/z: 222.1 (M+H)+ (calculated: 222.1)

1H NMR (400 MHz, DMSO-d6) δ 7.22 (s, 2H)

### Intermediate Compound 8

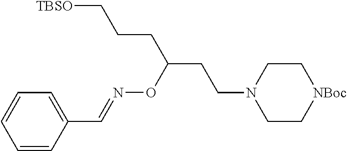 [SMILES: CC(C)(C)OC(=O)N1CCN(CCC(CCCO[Si](C)(C)C(C)(C)C)O/N=C/c2ccccc2)CC1]

The intermediate 8 has been prepared using the protocol described for the intermediate compound 1 from the tert-butyl tert-butyl piperazine-1-carboxylate (1.83 g, 9.85 mmol, 1.50 equiv). The intermediate compound 8 (1.33 g, 39% yield) has been isolated as a yellow oil.

LCMS (ESI position ion) m/z: 520.4 (M+H)+ (calculated: 520.4)

### Intermediate Compound 9

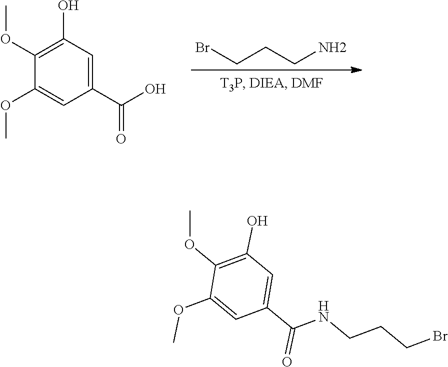 [SMILES: COc1cc(C(=O)O)cc(O)c1OC ; NCCCBr ; COc1cc(C(=O)NCCCBr)cc(O)c1OC]

To a mixture of 3-hydroxy-4,5-dimethoxybenzoic acid (20.0 g, 101 mmol, 1.0 equiv) and 3-bromopropan-1-amine (16.7 g, 121 mmol, 1.2 equiv) in DMF (400 mL) were added DIEA (39.1 g, 303 mmol, 3.0 equiv) and propylphosphonic anhydride solution (77.0 g, 121 mmol, 1.2 equiv) dropwise at 0° C. under a nitrogen atmosphere. The resulting mixture was stirred overnight at room temperature. The reaction was quenched by the addition of water (1.2 L) at room temperature. The resulting mixture was extracted with EtOAc (3×500 mL). The combined organic layers were washed with brine (1×500 mL), dried over Na2SO4 and concentrated under reduced pressure. The residue was purified by silica gel column chromatography (petroleum ether/EtOAc=5/1 to 1/1) to give the intermediate compound 112 (15 g, 47% yield) as an off-white solid.

LC-MS (ES+) m/z: 318 (M+H)+ (calculated: 317.0)

### Intermediate Compound 10

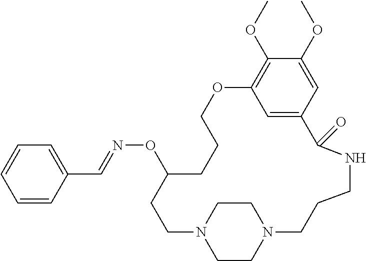 [SMILES: COc1cc2cc(c1OC)OCCCC(O/N=C/c1ccccc1)CCN1CCN(CCCNC2=O)CC1]

The intermediate compound 10 has been prepared using the protocol described for the compound 13 from intermediate 8 and intermediate 9 and purified by reverse flash chromatography with the following conditions: column, C18 Mobile Phase A: Water (0.05% TFA), Mobile Phase B: ACN; Flow rate: 90 mL/min; Gradient: 10% B to 33% B in 10 min, 33% B to 33% B in 18 min, 33% B; Wave Length: 220 nm.

The intermediate compound 10 (400 mg, TFA salt) has been isolated as a yellow solid.

LCMS (ESI position ion) m/z: 525.3 (M+H)+ (calculated: 525.3)

1H NMR (400 MHz, CD3OD-d4) δ 8.13 (s, 1H), 7.60-7.57 (m, 2H), 7.40-7.37 (m. 3H), 7.29 (s, 1H), 7.24 (s, 1H), 4.41-4.30 (m, 3H), 3.87 (s, 3H), 3.84 (s, 3H), 3.57-3.53 (m, 2H), 3.29-2.95 (m, 12H), 2.06-1.80 (m, 8H)

### Intermediate Compound 11

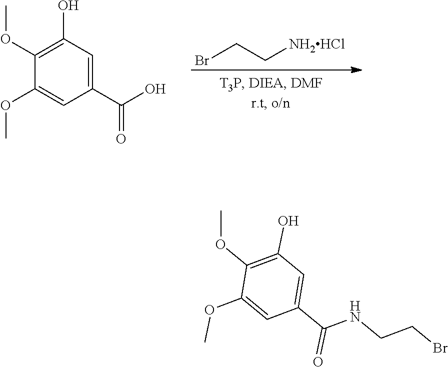

To a mixture of 3-hydroxy-4,5-dimethoxybenzoic acid (3.0 g, 15.14 mmol, 1.00 equiv) and 2-bromoethanamine hydrochloride (3.64 g, 22.70 mmol, 1.50 equiv) in DMF (60 mL) were added DIEA (5.87 g, 45.41 mmol, 3.00 equiv) and T3P (5.30 g, 16.65 mmol, 1.10 equiv) dropwise at 0 degrees C. under nitrogen atmosphere. The resulting mixture was stirred for additional overnight at room temperature. The reaction was quenched by the addition of Water (200 mL) at room temperature. The resulting mixture was extracted with EtOAc (3×200 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (5:1-1:1) to afford the intermediate compound 11 (2.2 g, 48% yield) as an off-white solid

LCMS (ESI position ion) m/z: 304.0 (M+H)+ (calculated: 304.0)

### Intermediate Compound 12

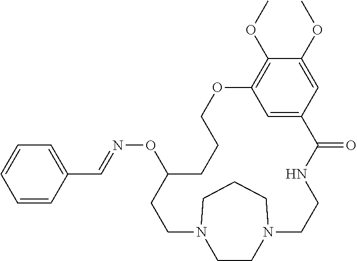 [SMILES: COc1cc2cc(c1OC)OCCCC(O/N=C/c1ccccc1)CCN1CCCN(CCNC2=O)CC1]

Intermediate compound 12B was prepared using a protocol similar to intermediate compound 13 using intermediate compound 11 instead of intermediate compound 9.

To a stirred mixture of intermediate compound 12B (1.0 equiv) and ADDP (2.0 equiv) in THF was added n-butylphosphine (2.0 equiv) in portions at room temperature under nitrogen atmosphere. The resulting mixture was stirred for additional 2 h at 40° C., and then allowed to cool down to room temperature. The reaction was quenched with saturated aqueous solution of NH₄Cl (100 mL) at room temperature. The resulting mixture was extracted with EtOAc (3×100 mL). The combined organic layers were dried over Na₂SO₄ and concentrated under reduced pressure, and purified by reverse flash chromatography with the following conditions: column, C18; mobile phase, MeCN in water (0.01% HCl), 17% to 43% gradient in 18 min; detector, UV 220 nm.

The intermediate compound 12 (400 mg, TFA salt) has been isolated as a yellow solid.

LCMS (ESI position ion) m/z: 525.4 (M+H)+ (calculated: 525.3)

1H NMR (400 MHz, CD3OD-d4) δ 8.17 (s, 1H), 7.61-7.58 (m, 3H), 7.40-7.37 (m, 3H), 7.27 (s, 1H), 4.70-4.13 (m, 4H), 3.89-3.53 (m, 19H), 2.40-1.80 (m, 8H)

### Intermediate Compound 13

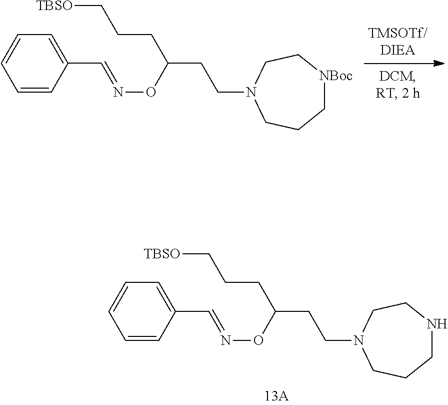 [SMILES: CC(C)(C)OC(=O)N1CCCN(CCC(CCCO[Si](C)(C)C(C)(C)C)O/N=C/c2ccccc2)CC1 ; CC(C)(C)[Si](C)(C)OCCCC(CCN1CCCNCC1)O/N=C/c1ccccc1]

To a solution of intermediate compound 1 (5.6 g, 10.5 mmol, 1.0 equiv) and DIEA (5.4 g, 42 mmol, 4.0 equiv) in DCM (100 mL), was added trimethylsilyl trifluoromethanesulfonate (7.0 g, 31.5 mmol, 3.0 equiv) at 0° C. The resulting solution was stirred for 2 h at room temperature, and then quenched by the addition of 20 mL of water. The organic phase was washed with 20 mL of water and brine (2×30 mL) The organic layer was dried over Na2SO4 and concentrated. The residue was purified by preparative HPLC (Column (C18-I, 20-40 μm); mobile phase (MeOH/H2O=20% to 100%:7 min; 100%:3 min); Detector (254 and 220 nm)) to give the intermediate compound 13A (4 g, 75% yield) as a light brown oil.

LC-MS (ES+) m/z: 434 (M+H)+ (calculated: 433.3)

1H NMR (300 MHz, DMSO-d6) δ 7.21 (s, 1H), 7.10 (s, 1H), 4.30-4.26 (m, 2H), 3.84-3.74 (m, 6H), 2.80-2.71 (m, 4H), 2.65-2.57 (m, 6H), 1.84-1.60 (m, 4H), 0.99 (s, 9H), 0.16 (s, 6H).

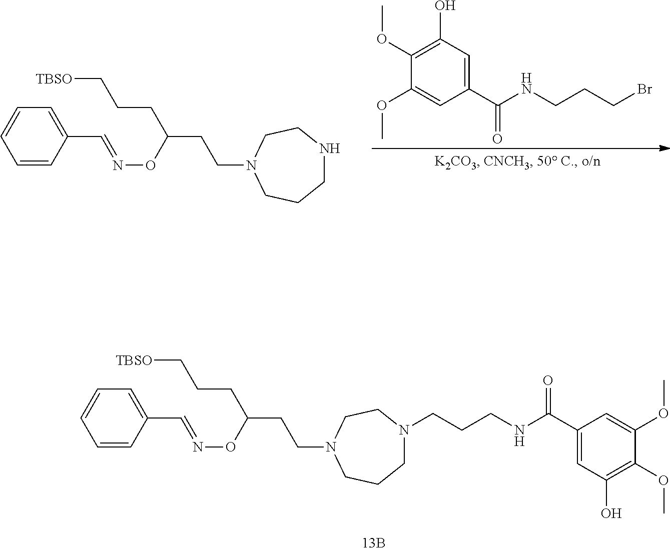 [SMILES: CC(C)(C)[Si](C)(C)OCCCC(CCN1CCCNCC1)O/N=C/c1ccccc1 ; COc1cc(C(=O)NCCCBr)cc(O)c1OC ; COc1cc(C(=O)NCCCN2CCCN(CCC(CCCO[Si](C)(C)C(C)(C)C)O/N=C/c3ccccc3)CC2)cc(O)c1OC]

To a stirred solution of intermediate compound 13A (10.0 g, 23.1 mmol, 1.0 equiv) and K2CO3 (7.97 g, 57.7 mmol, 2.5 equiv) in CH₃CN (250 mL) was added intermediate compound 112 (11.0 g, 34.6 mmol, 1.5 equiv) in portions at room temperature under a nitrogen atmosphere. The reaction mixture was stirred overnight at 50° C., and was allowed to cool down to room temperature. The resulting suspension was filtered, the precipitate was washed with acetonitrile (1×100 mL), and the filtrate was concentrated under reduced pressure. The residue was purified by reverse flash chromatography (column, C18 silica gel; mobile phase, MeOH in water, 80% to 95% gradient in 8 min and 95% to 100% gradient in 9 min; detector. UV 254 nm and 220 nm) to give the intermediate compound 13B (5 g, 32% yield) as a light yellow oil.

LC-MS (ES+) m/z: 671 (M+H)+ (calculated: 670.4)

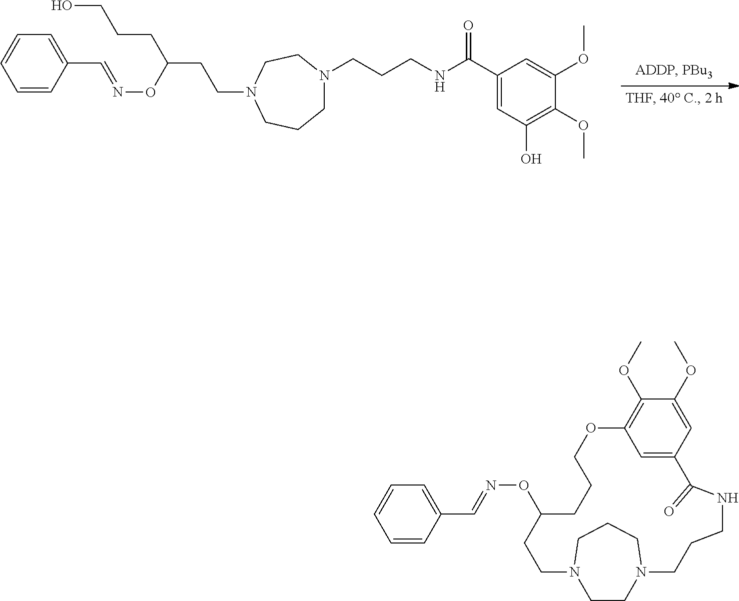 [SMILES: COc1cc(C(=O)NCCCN2CCCN(CCC(CCCO)O/N=C/c3ccccc3)CC2)cc(O)c1OC ; COc1cc2cc(c1OC)OCCCC(O/N=C/c1ccccc1)CCN1CCCN(CCCNC2=O)CC1]

To a stirred mixture of intermediate compound 13B (3.6 g, 6.5 mmol, 1.0 equiv) and ADDP (3.24 g, 12.9 mmol, 2.0 equiv) in THF (100 mL) was added n-butylphosphine (2.62 g, 12.9 mmol, 2.0 equiv) in portions at room temperature under nitrogen atmosphere. The resulting mixture was stirred for additional 2 h at 40° C., and then allowed to cool down to room temperature. The reaction was quenched with saturated aqueous solution of NH4Cl (100 mL) at room temperature. The resulting mixture was extracted with EtOAc (3×100 mL). The combined organic layers were dried over Na2SO4 and concentrated under reduced pressure. The residue was purified by preparative HPLC (Column: C18-I, 20-40 μm; mobile phase: MeOH/H2O=30% to 100%, 7 min; 100%, 3 min; detector 254 and 220 nm) to give the compound 114 (1.5 g, 43% yield) as a yellow oil.

LC-MS (ES+) m/z: 539 (M+H)+ (calculated: 538.3).

1H-NMR (300 MHz, MeOD-d4) δ ppm 8.09 (s, 1H), 7.57-7.54 (m, 2H), 7.37-7.33 (m, 3H), 7.18-7.14 (m, 2H), 4.50-4.49 (m, 1H), 4.28-4.24 (m, 2H), 3.89 (s, 3H), 3.84 (s, 3H), 3.54-3.48 (m, 2H), 2.84-2.54 (m, 12H), 1.96-1.73 (m, 10H).

### Intermediate Compound 14

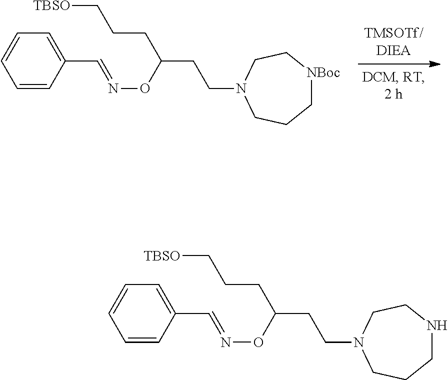 [SMILES: CC(C)(C)OC(=O)N1CCCN(CCC(CCCO[Si](C)(C)C(C)(C)C)O/N=C/c2ccccc2)CC1 ; CC(C)(C)[Si](C)(C)OCCCC(CCN1CCCNCC1)O/N=C/c1ccccc1]

To a solution of intermediate compound 1 (5.6 g, 10.5 mmol, 1.0 equiv) and DIEA (5.4 g, 42 mmol, 4.0 equiv) in DCM (100 mL), was added trimethylsilyl trifluoromethanesulfonate (7.0 g, 31.5 mmol, 3.0 equiv) at 0° C. The resulting solution was stirred for 2 h at room temperature, and then quenched by the addition of 20 mL of water. The organic phase was washed with 20 mL of water and brine (2×30 mL). The organic layer was dried over Na2SO4 and concentrated. The residue was purified by preparative HPLC (Column (C18-I, 20-40 μm); mobile phase (MeOH/H2O=20% to 100% 7 min; 100% 3 min); Detector (254 and 220 nm)) to give the intermediate compound 14 (4 g, 75% yield) as a light brown oil

LC-MS (ES+) m/z: 434 (M+H)+ (calculated: 433.3)

1H NMR (300 MHz, DMSO-d6) δ 7.21 (s, 1H), 7.10 (s, 1H), 4.30-4.26 (m, 2H), 3.84-3.74 (m, 6H), 2.80-2.71 (m, 4H), 2.65-2.57 (m, 6H), 1.84-1.60 (m, 4H), 0.99 (s, 9H), 0.16 (s, 6H)

### Intermediate Compound 15

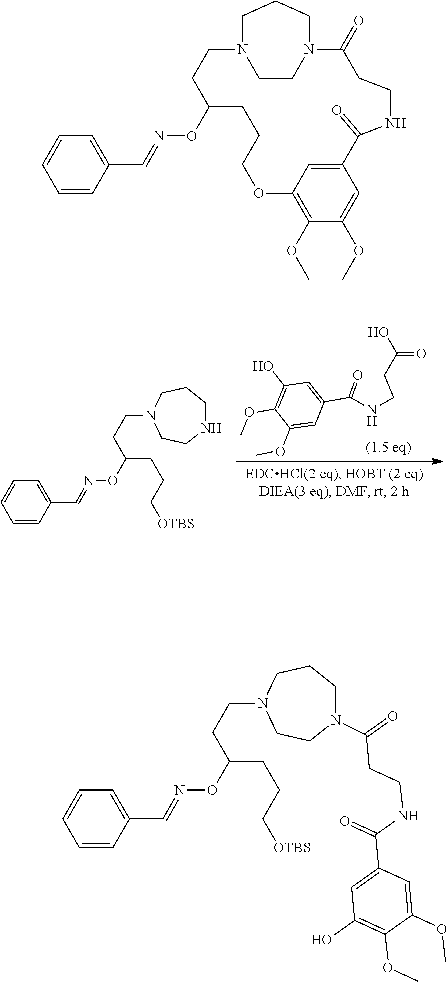

To a stirred solution of intermediate compound 14 (1 g, 2.31 mmol, 1.00 equiv) and 3-[(3-hydroxy-4,5-dimethoxyphenyl)formamido]propanoic acid (0.93 g, 3.46 mmol, 1.50 equiv) in DMF (20 mL) were added EDC·HCl (0.88 g, 4.61 mmol, 2.00 equiv), HOBT (0.62 g, 4.61 mmol, 2.00 equiv) and DIEA (0.89 g, 6.92 mmol, 3.00 equiv) at rt. The resulting mixture was stirred for 4 h at rt. The reaction was quenched with 50 mL sat. NH₄Cl. The resulting mixture was extracted with ethyl acetate (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with THF/PE (1:4-4:1) to the corresponding amide (700 mg, 44% yield) as a light-yellow oil

LC-MS (ES+) m/z: 685.5 (M+H)+ (calculated: 685.4)

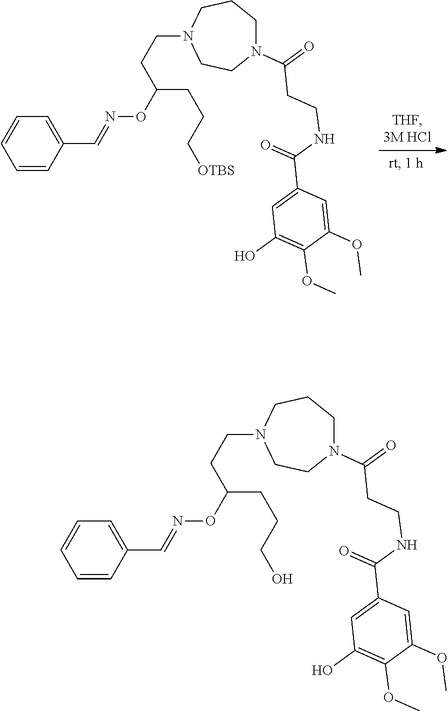 [SMILES: COc1cc(C(=O)NCCC(=O)N2CCCN(CCC(CCCO[Si](C)(C)C(C)(C)C)O/N=C/c3ccccc3)CC2)cc(O)c1OC ; COc1cc(C(=O)NCCC(=O)N2CCCN(CCC(CCCO)O/N=C/c3ccccc3)CC2)cc(O)c1OC]

A solution of the above amide (700 mg, 1.02 mmol, 1.00 equiv) in THF (20 mL) and 3M HCl (10 mL) was stirred for 3 h at rt. The resulting mixture was extracted with ethyl acetate (1×20 mL) The aqueous layer was basified to pH=8 with sat NaHCO₃. The resulting mixture was extracted with ethyl acetate (3×100 mL). The combined organic layers were washed with brine (1×100 mL), dried over anhydrous Na₂SO₄. After filtration, the filtrate was concentrated under reduced pressure. The isolated off white solid (400 mg, 69% yield) was used without further purification.

LC-MS (ES+) m/z: 571.3 (M+H)+ (calculated: 571.3)

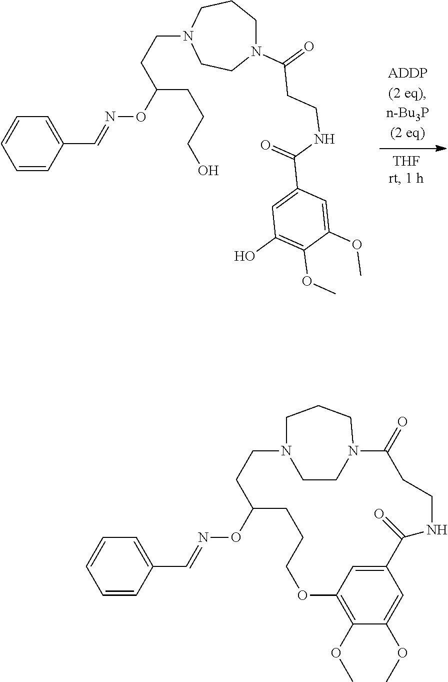 [SMILES: COc1cc(C(=O)NCCC(=O)N2CCCN(CCC(CCCO)O/N=C/c3ccccc3)CC2)cc(O)c1OC ; COc1cc2cc(c1OC)OCCCC(O/N=C/c1ccccc1)CCN1CCCN(CC1)C(=O)CCNC2=O]

To a stirred solution of the above isolated white solid (400 mg, 0.70 mmol, 1.00 equiv) in THF (20 mL) were added ADDP (350.93 mg, 1.40 mmol, 2.00 equiv) and tri(n-Buthyl)phosphine (283.61 mg, 1.40 mmol, 2.00 equiv) at room temperature under N2 atmosphere. The resulting mixture was stirred for 1h at room temperature under N2 atmosphere. The reaction was quenched with saturated solution of NH₄Cl (40 mL). The resulting mixture was extracted with ethyl acetate (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with THF/PE (1:5-4:1) then MeOH/DCM (1:20-1:10) to afford the intermediate compound 15 (300 mg, 70% yield) as a yellow oil.

LC-MS (ES+) m/z: 553.3 (M+H)+ (calculated: 553.3)

### Intermediate Compound 16

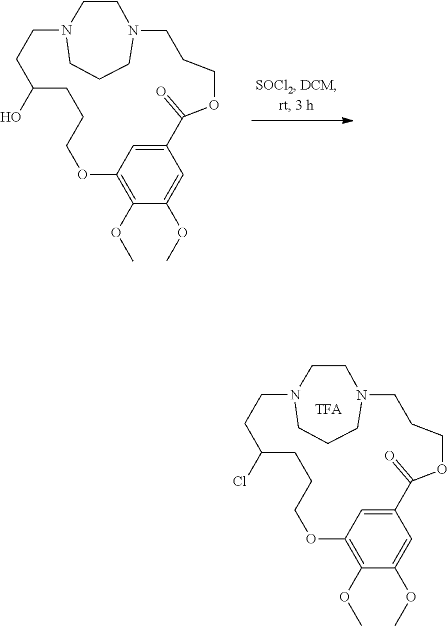 [SMILES: COc1cc2cc(c1OC)OCCCC(O)CCN1CCCN(CCCOC2=O)CC1 ; COc1cc2cc(c1OC)OCCCC(Cl)CCN1CCCN(CCCOC2=O)CC1]

A solution of intermediate compound 4 (300 mg, 0.69 mmol, 1.0 equiv) and SOCl2 (409 mg, 3.4 mmol, 5.0 equiv) in DCM (10 mL) was stirred for 3 h at room temperature. The resulting mixture was then concentrated under reduced pressure, and the residue was purified by Prep-HPLC with the following conditions (Column: C18-I, 20-40 μm; Mobile Phase A: Water: 0.05% TFA, Mobile Phase B: ACN; Flow rate: 80 mL/min; Gradient: 10% B to 60% B in 7 min, 55% B; Detector 254 and 220 nm) to afford intermediate compound 16 (230 mg, 74% yield) as a colorless oil.

LC-MS (ES+) m/z: 455.2 (M+H)+ (calculated: 455.2)

### Intermediate Compound 17

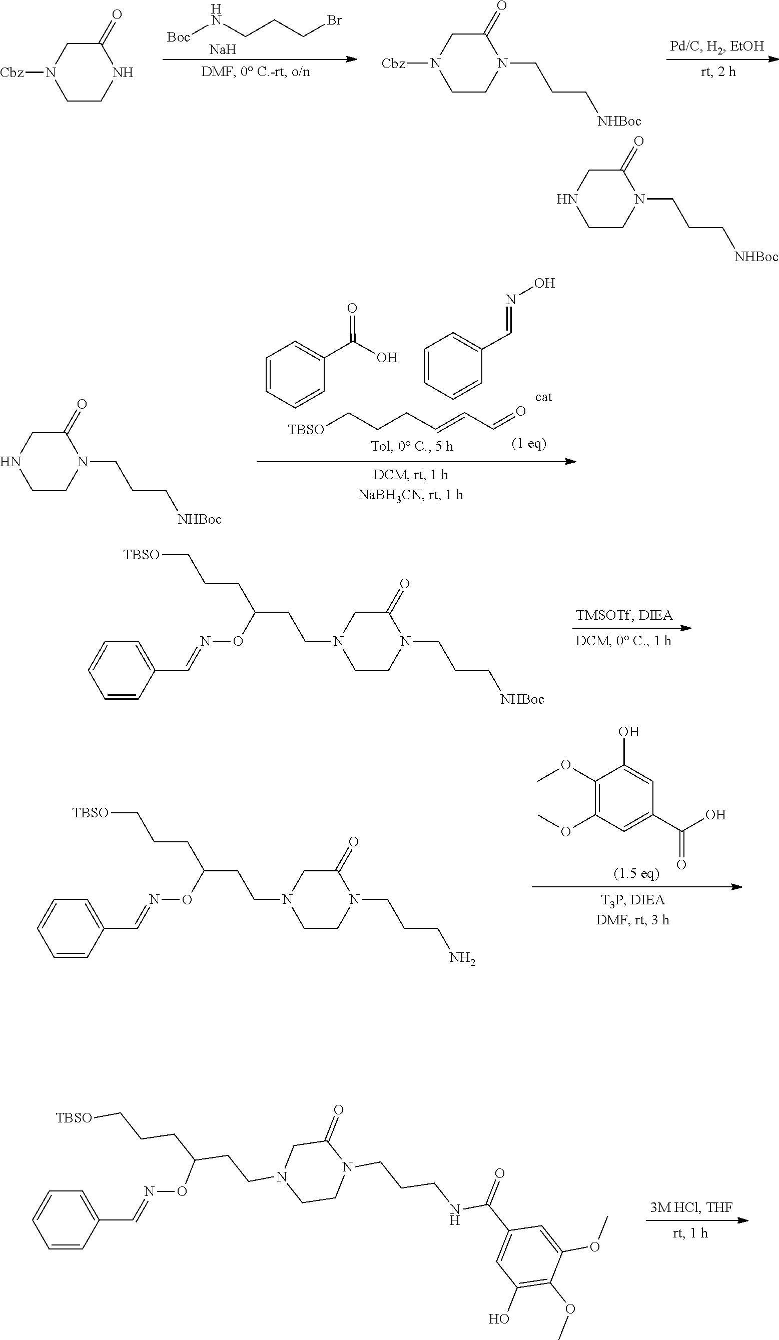

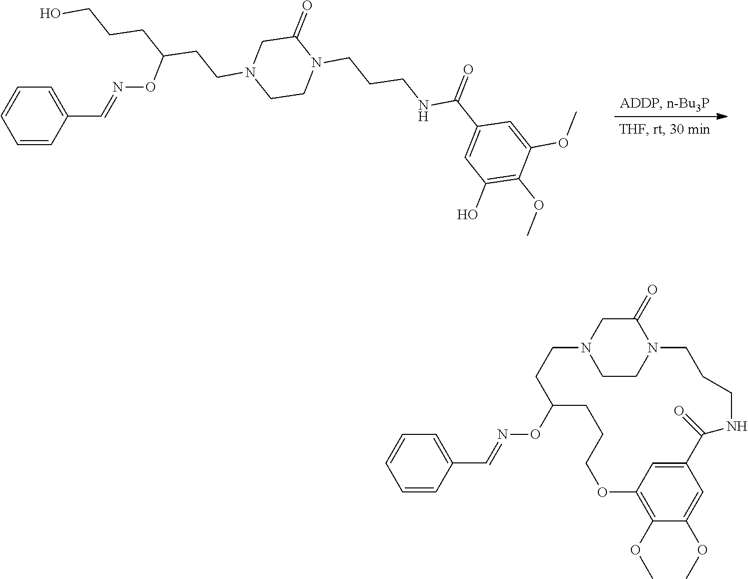

To a stirred solution of benzyl 3-oxopiperazine-1-carboxylate (10 g, 42.69 mmol, 1.00 equiv) in DMF (200 mL) was added NaH (2.05 g, 51.23 mmol, 1.20 equiv) in portions at 0° C. under N2 atmosphere. The resulting mixture was stirred for 0.5h at 0° C. under N2 atmosphere. To the above mixture was added tert-butyl N-(3-bromopropyl)carbamate (11.18 g, 46.958 mmol, 1.10 equiv) in portions at 0° C. The resulting mixture was stirred overnight at rt. The reaction was quenched with a saturated solution of NH₄Cl (300 mL). The resulting mixture was extracted with ethyl acetate (3×300 mL). The combined organic layers were washed with brine (1×300 mL), dried over anhydrous Na₂SO₄. After filtration, the filtrate was concentrated under reduced pressure. The residue was purified by silica gel column chromatography, eluted with THF/PE (1:5-1:3) to afford benzyl 4-{3-[(tert-butoxycarbonyl)amino]propyl}-3-oxopiperazine-1-carboxylate (8 g, 48% yield) as a colorless oil.

LC-MS (ES+) m/z: 392.2 (M+H)+ (calculated: 392.2)

To a solution of benzyl 4-{3-[(tert-butoxycarbonyl)amino]propyl}-3-oxopiperazine-1-carboxylate (8 g, 20.44 mmol, 1.00 equiv) in EtOH (100 mL) was added Pd/C (1.09 g) under nitrogen atmosphere in a 250 mL round bottom flask. The mixture was hydrogenated at room temperature for 2 h under hydrogen atmosphere using a hydrogen balloon, filtered through a Celite pad and concentrated under reduced pressure. This resulted in tert-butyl N-[3-(2-oxopiperazin-1-yl)propyl]carbamate (5 g, 95% yield) as a colorless oil.

LC-MS (ES+) m/z: 258.2 (M+H)+ (calculated: 258.2)

To a stirred solution of benzoic acid (0.11 g, 0.88 mmol, 0.10 equiv) and (2S)-2-{bis[3,5-bis(trifluoromethyl)phenyl][(trimethylsilyl)oxy]methyl}pyrrolidine (0.52 g, 0.88 mmol, 0.10 equiv) in Toluene (5 mL) were added (2E)-6-[(tert-butyldimethylsilyl)oxy]hex-2-enal (2 g, 8.76 mmol, 1.00 equiv) and benzaldoxime,syn (3.18 g, 26.27 mmol, 3.00 equiv) at 0° C. under N₂ atmosphere. The resulting mixture was stirred for 5 h at 0° C. under N₂ atmosphere. To the above mixture was added DCM (20 mL) and tert-butyl N-[3-(2-oxopiperazin-1-yl)propyl]carbamate (3.61 g, 14.01 mmol, 1.60 equiv) at rt. The resulting mixture was stirred for additional 1 h at rt. To the above mixture was added NaBH₃CN (0.88 g, 14.01 mmol, 1.60 equiv) in portions at rt. The resulting mixture was stirred for additional 1 h at rt. The reaction was quenched with a saturated solution of NH₄C (40 mL). The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with THF/PE (1:10-1:2) to afford tert-butyl N-[3-(4-{6-[(tert-butyldimethylsilyl)oxy]-3-{[(E)-(phenylmethylidene)amino]oxy}hexyl}-2-oxopiperazin-1-yl)propyl]carbamate (2.5 g, 41% yield) as a light-yellow oil

LC-MS (ES+) m/z: 591.4 (M+H)+ (calculated: 591.4)

To a stirred solution of tert-butyl N-[3-(4-{6-[(tert-butyldimethylsilyl)oxy]-3-{[(E)-(phenylmethylidene)amino]oxy}hexyl}-2-oxopiperazin-1-yl)propyl]carbamate (2.5 g, 3.60 mmol, 1.00 equiv, 85%) and DIEA (2.32 g, 17.98 mmol, 5.00 equiv) in DCM (30 mL) was added TMSOTf (2.40 g, 10.79 mmol, 3.00 equiv) dropwise at 0° C. under N₂ atmosphere. The resulting mixture was stirred for 1 h at 0° C. under N₂ atmosphere. The reaction was quenched by the addition of a saturated solution of NH₄Cl (30 mL) at 0° C. The resulting mixture was extracted with ethyl acetate (3×50 mL). The combined organic layers were washed with brine (1×50 mL), dried over anhydrous Na₂SO₄. After filtration, the filtrate was concentrated under reduced pressure. This resulted in 1-(3-aminopropyl)-4-{6-[(tert-butyldimethylsilyl)oxy]-3-{[(E)-(phenylmethylidene)amino]oxy}hexyl}piperazin-2-one (1 g, 57% yield) as a brown yellow oil

LC-MS (ES+) m/z: 491.4 (M+H)+ (calculated: 491.3)

To a stirred solution of 1-(3-aminopropyl)-4-{6-[(tert-butyldimethylsilyl)oxy]-3-{[(E)-(phenylmethylidene)amino]oxy}hexyl}piperazin-2-one (1 g, 2.04 mmol, 1.00 equiv) and 3-hydroxy-4,5-dimethoxybenzoic acid (0.61 g, 3.06 mmol, 1.50 equiv) in DMF (20 mL) were added DIEA (0.79 g, 6.11 mmol, 3.00 equiv) at rt. To the above mixture was added T3P (0.97 g, 3.06 mmol, 1.50 equiv) dropwise at rt. The resulting mixture was stirred for additional 3h at rt. The reaction was quenched with a saturated solution of NH₄Cl (50 mL) at rt. The resulting mixture was extracted with ethyl acetate (3×100 mL). The combined organic layers were washed with 5% NaCl (1×100 mL), dried over anhydrous NaSO₄. After filtration, the filtrate was concentrated under reduced pressure. The residue was purified by silica gel column chromatography, eluted with THF/PE (1:10-1:2) to afford N-[3-(4-{6-[(tert-butyldimethylsilyl)oxy]-3-{[(E)-(phenylmethylidene)amino]oxy}hexyl}-2-oxopiperazin-1-yl)propyl]-3-hydroxy-4,5-dimethoxybenzamide (600 mg, 44% yield) as a brown yellow oil.

LC-MS (ES+) m/z: 671.4 (M+H)+ (calculated: 671.4)

To a stirred solution of N-[3-(4-{6-[(tert-butyldimethylsilyl)oxy]-3-{[(E)-(phenylmethylidene)amino]oxy}hexyl}-2-oxopiperazin-1-yl)propyl]-3-hydroxy-4,5-dimethoxybenzamide (600 mg, 0.89 mmol, 1.00 equiv) in THF (10 mL) was added HCl (3M) (10 mL) at rt. The resulting mixture was stirred for 1 h at rt. The resulting mixture was concentrated under reduced pressure. The resulting mixture was diluted with H₂O (20 mL). The aqueous layer was extracted with ethyl acetate (1×30 mL). Then was basified to pH 8-9 with NaHCO₃. The resulting mixture was extracted with ethyl acetate (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with THA/PE (1:5-5:1) to afford 3-hydroxy-N-{3-[4-(6-hydroxy-3-{[(E)-(phenylmethylidene)amino]oxy}hexyl)-2-oxopiperazin-1-yl]propyl}-4,5-dimethoxybenzamide (350 mg, 70% yield) as a light-brown solid.

LC-MS (ES+) m/z: 557.4 (M+H)+ (calculated: 557.3)

To a stirred solution of 3-hydroxy-N-{3-[4-(6-hydroxy-3-{[(E)-(phenylmethylidene)amino]oxy}hexyl)-2-oxopiperazin-1-yl]propyl}-4,5-dimethoxybenzamide (130 mg, 0.23 mmol, 1.00 equiv) in THF (10 mL) were added ADDP (467.71 mg, 1.87 mmol, 8.00 equiv) and n-Bu₃P (377.99 mg, 1.87 mmol, 8.00 equiv) at rit. The resulting mixture was stirred for 30 min at rt. The reaction was quenched with 10 mL sat. NH₄Cl. The resulting mixture was extracted with ethyl acetate (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with THF/PE (1:5-5:1) to afford the intermediate compound 17 (80 mg, 40% yield) as a light-yellow oil.

LC-MS (ES+) m/z: 539.3 (M+H)+ (calculated: 539.3)

### Intermediate Compound 18

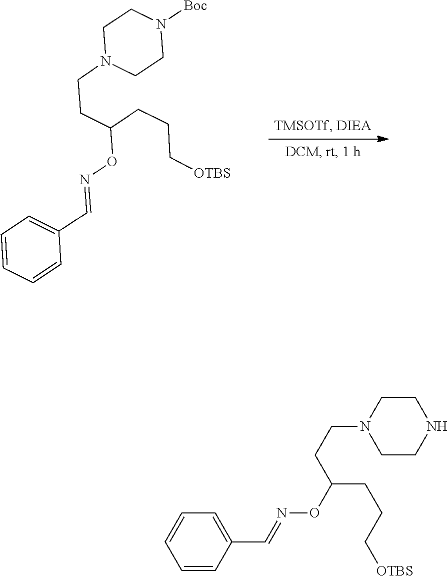 [SMILES: CC(C)(C)OC(=O)N1CCN(CCC(CCCO[Si](C)(C)C(C)(C)C)O/N=C/c2ccccc2)CC1 ; CC(C)(C)[Si](C)(C)OCCCC(CCN1CCNCC1)O/N=C/c1ccccc1]

To a stirred solution of intermediate compound 8 (1.8 g, 3.46 mmol, 1.00 equiv) and DIEA (2.24 g, 17.32 mmol, 5.00 equiv) in DCM (40 mL) was added TMSOTf (2.31 g, 10.39 mmol, 3.00 equiv) dropwise at room temperature under N2 atmosphere. The resulting mixture was stirred for 1 h at room temperature under N2 atmosphere. The reaction was quenched with a saturated solution of NH₄Cl (50 mL). The aqueous layer was extracted with DCM (3×50 mL) The combined organic layers were washed with brine (1×50 mL), dried over anhydrous Na₂SO₄. After filtration, the filtrate was concentrated under reduced pressure. The residue was purified by silica gel column chromatography, eluted with THF/PE (1:5-1:0) to afford the intermediate compound 18 (1.2 g, 83% yield) as a yellow oil.

LC-MS (ES+) m/z: 420.4 (M+H)+ (calculated: 420.4)

### Intermediate Compound 19

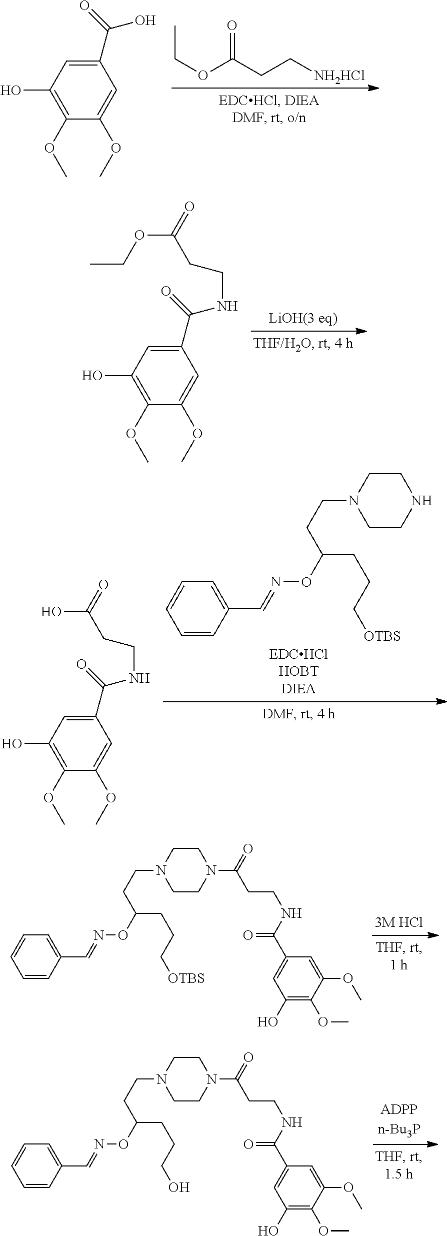

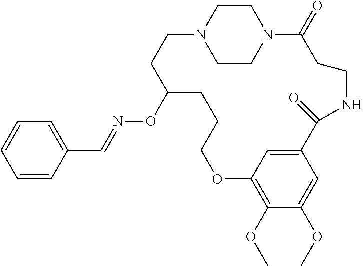 [SMILES: COc1cc2cc(c1OC)OCCCC(O/N=C/c1ccccc1)CCN1CCN(CC1)C(=O)CCNC2=O]

To a stirred solution of 3-hydroxy-4,5-dimethoxybenzoic acid (10 g, 50.46 mmol, 1.00 equiv) and β-alanine ethyl ester hydrochloride (9.30 g, 60.55 mmol, 1.20 equiv) in DMF (150 mL) were added EDC HCl (14.51 g, 75.69 mmol, 1.50 equiv) and DIEA (19.57 g, 151.38 mmol, 3.00 equiv) at room temperature under N2 atmosphere. The resulting mixture was stirred overnight at room temperature under N2 atmosphere. The reaction was quenched with a saturated solution of NH₄Cl (300 mL). The resulting mixture was extracted with ethyl acetate (5×400 mL) The combined organic layers were washed with brine (1×400 mL), dried over anhydrous Na₂SO₄. After filtration, the filtrate was concentrated under reduced pressure. The residue was purified by silica gel column chromatography, eluted with THF/PE (1:5-1:3) to afford ethyl 3-[(3-hydroxy-4,5-dimethoxyphenyl)formamido]propanoate (10 g, 67% yield) as a off-white solid.

LC-MS (ES+) m/z: 298.1 (M+H)+ (calculated: 298.1)

To a stirred solution of ethyl 3-[(3-hydroxy-4,5-dimethoxyphenyl)formamido]propanoate (3 g, 10.09 mmol, 1.00 equiv) in THF (30 mL) and H2O (5 mL) was added LiOH (0.72 g, 30.27 mmol, 3.00 equiv) at rt. The resulting mixture was stirred for 4 h at rt. The mixture was acidified to pH 3-4 with a solution of HCl (3 M). The resulting mixture was concentrated under reduced pressure. The residue was purified by reverse flash chromatography with the following conditions: column, C18 silica gel; mobile phase, MeCN in 0.05% TFA, 10% to 40% gradient in 10 min, detector, UV 254 nm. This resulted in 3-[(3-hydroxy-4,5-dimethoxyphenyl)formamido]propanoic acid (2.4 g, 88% yield) as a brown yellow oil.

LC-MS (ES+) m/z: 270.1 (M+H)+ (calculated: 270.1)

To a stirred solution of intermediate compound 18 (1.2 g, 2.86 mmol, 1.00 equiv) and 3-[(3-hydroxy-4,5-dimethoxyphenyl)formamido]propanoic acid (1.15 g, 4.29 mmol, 1.50 equiv) in DMF (20 mL) were added EDC HCl (1.10 g, 5.78 mmol, 2.00 equiv), HOBT (0.77 g, 5.72 mmol, 2.00 equiv) and DIEA (1.11 g, 8.58 mmol, 3.00 equiv) at room temperature under N2 atmosphere. The resulting mixture was stirred for 4h at room temperature under N2 atmosphere. The reaction was quenched with a saturated solution of NH₄Cl (50 mL). The resulting mixture was extracted with ethyl acetate (3×100 mL) The combined organic layers were washed with brine (1×100 mL), dried over anhydrous Na₂SO₄. After filtration, the filtrate was concentrated under reduced pressure. The residue was purified by silica gel column chromatography, eluted with THF/PE (1:5-4:1) to afford N-[3-(4-{6-[(tert-butyldimethylsilyl)oxy]-3-{[(E)-(phenylmethylidene)amino]oxy}hexyl}piperazin-1-yl)-3-oxopropyl]-3-hydroxy-4,5-dimethoxybenzamide (800 mg, 42% yield) as a light-yellow oil

LC-MS (ES+) m/z: 671.5 (M+H)+ (calculated: 671.5)

To a stirred solution of N-[3-(4-{6-[(tert-butyldimethylsilyl)oxy]-3-{[(E)-(phenylmethylidene)amino]oxy}hexyl}piperazin-1-yl)-3-oxopropyl]-3-hydroxy-4,5-dimethoxybenzamide (800 mg, 1.19 mmol, 1.00 equiv) in THF (20 mL) was added 3 M HCl (10 mL) at rt. The resulting mixture was stirred for 1 h at rt. The resulting mixture was diluted with H₂O (30 mL). The aqueous layer was extracted with ethyl acetate (1×50 mL). The aqueous layer was basified to pH 7-8 with a saturated solution of NaHCO₃. The resulting mixture was extracted with ethyl acetate (3×100 mL). The combined organic layers were washed with brine (1×100 mL), dried over anhydrous Na₂SO₄. After filtration, the filtrate was concentrated under reduced pressure. This resulted in 3-hydroxy-N-{3-[4-(6-hydroxy-3-{[(E)-(phenylmethylidene)amino]oxy}hexyl)piperazin-1-yl]-3-oxopropyl}-4,5-dimethoxybenzamide (400 mg, 60% yield) as a off-white semi-solid.

LC-MS (ES+) m/z: 557.3 (M+H)+ (calculated: 557.3)

To a stirred solution of 3-hydroxy-N-{3-[4-(6-hydroxy-3-{[(E)-(phenylmethylidene)amino]oxy}hexyl)piperazin-1-yl]-3-oxopropyl}-4,5-dimethoxybenzamide (50 mg, 0.09 mmol, 1.00 equiv) in THF (5 mL) were added ADDP (44.97 mg, 0.18 mmol, 2.00 equiv) and n-Bu3P (36.34 mg, 0.18 mmol, 2.00 equiv) at rt. The resulting mixture was stirred for 1.5h at rt. The reaction was quenched with a saturated solution of NH₄Cl (10 mL). The resulting mixture was extracted with ethyl acetate (3×10 mL). The combined organic layers were washed with brine (1×10 mL), dried over anhydrous Na₂SO₄. After filtration, the filtrate was concentrated under reduced pressure. The residue was purified by reverse flash chromatography with the following conditions. Column: SunFire Prep C18 OB) Column, 19*150 mm, 5 μm; Mobile Phase A: Water (0.05% TFA), Mobile Phase B: ACN; Flow rate: 20 mL/min; Gradient: 23% B to 37% B in 7 min; Wave Length 220 nm; RT1(min): 6.9) to afford the intermediate compound 19 (11 mg, TFA salt) as a off-white solid.

LC-MS (ES+) m/z: 539.3 (M+H)+ (calculated: 539.3)

1H NMR (400 MHz, CDCl3-d) δ 8.15 (s, 1H), 7.63-7.61 (m, 2H), 7.41-7.39 (m, 3H), 7.33 (s, 1H), 7.26 (s, 1H), 4.80-4.18 (m, 5H), 3.94 (s, 3H), 3.89 (s, 3H), 3.85-3.34 (m, 6H), 3.24-2.30 (m, 6H), 2.20-1.70 (m, 6H)

### Intermediate Compound 20

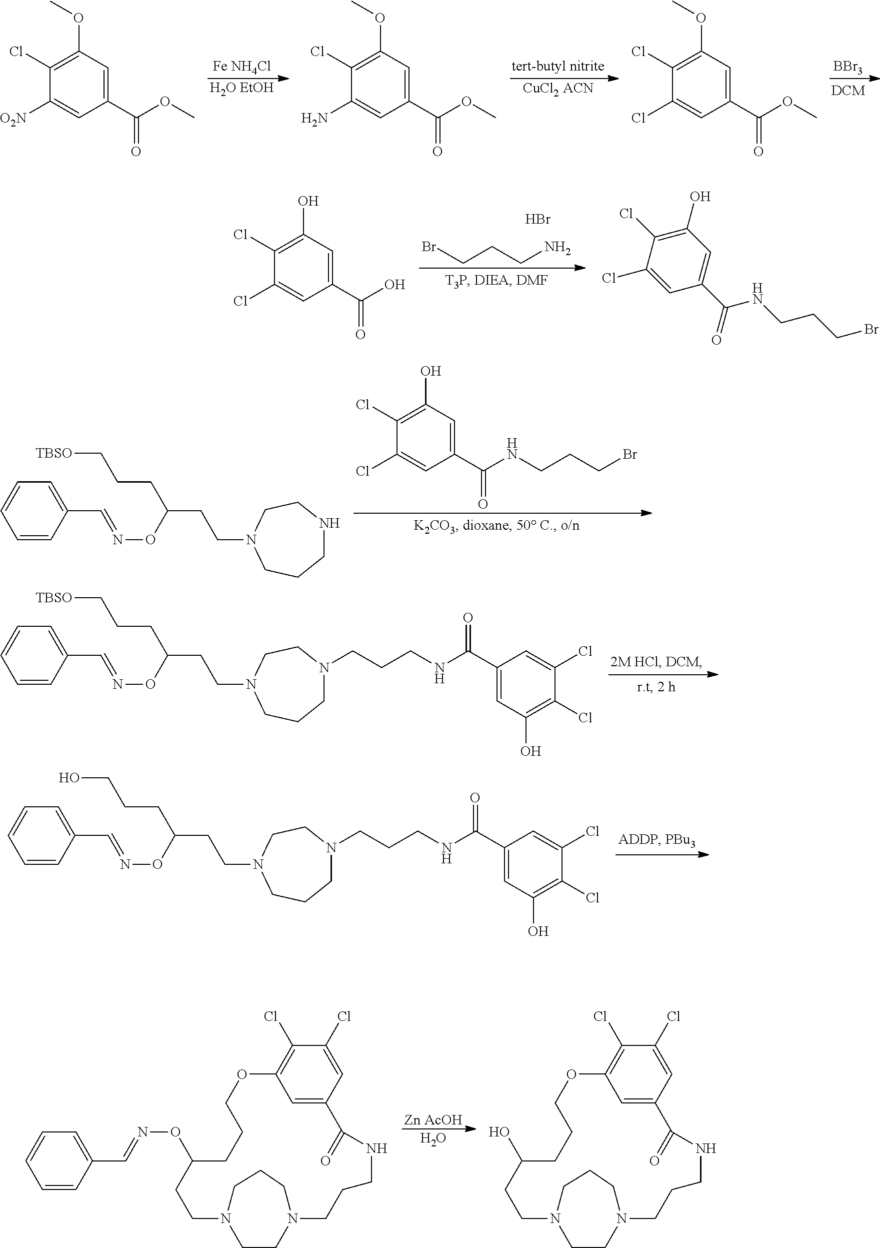

A suspension of methyl 4-chloro-3-methoxy-5-nitrobenzoate (450 g, 1.8 mol, 1.0 equiv), NH₄Cl (490 g, 9.2 mol, 5.0 equiv), iron (511.6 g, 9.2 mol, 5.0 equiv) in H2O (7.65 L)/EtOH (7.65 L) was stirred for 3 h at 70° C. under nitrogen. The mixture was then allowed to cool to room temperature and filtered. The solids were washed with EtOH (3×0.9 L), and the filtrate was concentrated to remove EtOH. The resulting aqueous mixture was extracted with EtOAc (2×3 L). The combined organic layers were dried over anhydrous Na₂SO₄ and concentrated under reduced pressure, affording methyl 3-amino-4-chloro-5-methoxybenzoate (380 g, 96% yield) as a light yellow solid.

LC-MS (ES+) m/z: 216.0 (M+H)+ (calculated: 216.0)

A solution of methyl 3-amino-4-chloro-5-methoxybenzoate (380 g, 1.8 mol, 1.0 equiv) in acetonitrile (874 mL) was added dropwised to a solution of CuCl₂ (284.3 g, 2.1 mol, 1.2 equiv), tert-butyl nitrite (272.6 g, 2.6 mol, 1.5 equiv) in acetonitrile (1.5 L) at 65° C. under nitrogen. Once the addition was complete the resulting mixture was stirred for 2 h at 65° C., and then allowed to cool to room temperature. The mixture was poured into a solution of 1 M HCl (3.8 L) and adjusted to pH=7 with a saturated solution of NaHCO₃. The resulting mixture was extracted with DCM (3×1.5 L). The combined organic layers were dried over anhydrous Na₂SO₄ and concentrated under reduced pressure, affording methyl 3,4-dichloro-5-methoxybenzoate (263.6 g, 63% yield) as a brown solid.

LC-MS (ES+) m/z: 235.0 (M+H)+ (calculated: 235.0)

A solution of methyl 3,4-dichloro-5-methoxybenzoate (263.6 g, 1.1 mol, 1.0 equiv) and DCM (6.6 L), boron tribromide (702 g, 2.8 mol, 2.5 equiv) was stirred for 15 h at room temperature under nitrogen. The mixture was poured into iced water (10 L) and the mixture stirred for 10 min. The mixture was then extracted with EtOAc (5 L), the organic solution was washed with brine (2×2.5 L) and dried over MgSO₄, filtered and evaporated under reduced pressure. The resulting residue was purified by silica gel chromatography (EA:PE 0:1-1:0) to afford 3,4-dichloro-5-hydroxybenzoic acid (188 g, 81% yield) as a white solid.

LC-MS (ES+) m/z: 207.0 (M+H)+ (calculated: 207.0)

T3P (55.3 g, 174 mmol, 1.2 equiv) was added at room temperature to a solution of 3,4-dichloro-5-hydroxybenzoic acid (30 g, 145 mmol, 1.0 equiv), DIEA (56.2 g, 435 mmol, 3.0 equiv), 3-bromopropan-1-amine hydrobromide (38.1 g, 174 mmol, 1.2 equiv) in DMF (600 mL). The resulting mixture was stirred for 2 h at room temperature under nitrogen, and then was poured into H₂O (3 L) The aqueous mixture was extracted with EtOAc (3×500 mL), and the combined organic layers were dried over anhydrous Na₂SO₄ and concentrated under reduced pressure. The residue was purified by silica gel column chromatography, eluted with PE/EA (1:1) to afford N-(3-bromopropyl)-3,4-dichloro-5-hydroxybenzamide (30 g, 70% purity) as an off-white solid, which was used directly in the next step.

LC-MS (ES+) m/z: 326.0 (M+H)+ (calculated: 326.0)

(E)-benzaldehyde O-(6-((tert-butyldimethylsilyl)oxy)-1-(1,4-diazepan-1-yl)hexan-3-yl) oxime was prepared from intermediate 1 as described for intermediate compound 3.

A mixture of (E)-benzaldehyde O-(6-((tert-butyldimethylsilyl)oxy)-1-(1,4-diazepan-1-yl)hexan-3-yl) oxime (30 g, 69.2 mmol, 1.0 equiv), K2CO3 (11.5 g, 83.0 mmol, 1.2 equiv) and N-(3-bromopropyl)-3,4-dichloro-5-hydroxybenzamide (30 g, crude) in dioxane (600 mL) was stirred for 16 h at 55° C. under nitrogen. The mixture was allowed to cool to room temperature, and then was filtered. The solid was washed with EtOAc (3×50 mL) and the filtrate was concentrated under reduced pressure. The residue was purified by reverse flash chromatography with the following conditions: column, C18 silica gel; mobile phase, MeOH in water, 30% to 100% gradient in 16 min; detector, UV 220 nm. This resulted in N-[3-(4-{6-[(tert-butyldimethylsilyl)oxy]-3-{[(E)-(phenylmethylidene)amino]oxy}hexyl}-1,4-diazepan-1-yl)propyl]-3,4-dichloro-5-hydroxybenzamide (17 g) as a light yellow oil.

LC-MS (ES+) m/z: 679.3 (M+H)+ (calculated: 679.3)

A solution of N-[3-(4-{6-[(tert-butyldimethylsilyl)oxy]-3-{[(E)-(phenylmethylidene)amino]oxy}hexyl}-1,4-diazepan-1-yl)propyl]-3,4-dichloro-5-hydroxybenzamide (17 g, 25.0 mmol, 1.0 equiv), in MTBE (170 mL)/2 M HCl (170 mL) was stirred for 1 h at room temperature under nitrogen. The aqueous layer was extracted with MTBE (2×100 mL) and the aqueous layer was basified to pH=8 with a saturated solution of NaHCO₃. The aqueous layer was extracted with DCM (3×100 mL), and the combined organic layers dried over anhydrous Na₂SO₄ and concentrated under reduced pressure, affording 3,4-dichloro-5-hydroxy-N-{3-[4-(6-hydroxy-3-{[(E)-(phenylmethylidene)amino]oxy}hexyl)-1,4-diazepan-1-yl]propyl}benzamide (8.0 g, 56% yield) as a yellow oil.

LC-MS (ES+) m/z: 565.3 (M+H)+ (calculated: 565.2)

A mixture of 3-hydroxy-N-{3-[4-(6-hydroxy-3-{[(E)-(phenylmethylidene)amino]oxy}hexyl)-1,4-diazepan-1-yl]propyl}-4,5-dimethoxybenzamide (8.0 g, 14.4 mmol, 1.0 equiv), ADDP (5.4 g, 21.6 mmol, 1.5 equiv), and tributylphosphane (4.4 g, 21.6 mmol, 1.5 equiv) was stirred for 15 h at 55° C. under nitrogen. The mixture was then allowed to cool to room temperature and poured into water (500 mL). The resulting mixture was extracted with EtOAc (3×100 mL), and the combined organic layers were dried over anhydrous NaSO₄ and concentrated under reduced pressure. The residue was purified by Prep-HPLC with the following conditions (Column: Xselect CSH F-Phenyl OBD Column 19×150 mm 5 μm, Mobile Phase A: Water (0.05% HCl), Mobile Phase B: ACN; Flow rate: 20 mL/min; Gradient: 23% B to 33% B in 9 min, 33% B; Wave Length: 220 nm; RT1 (min): 7.9), affording the (E)-benzaldehyde O-(7⁴,7⁵-dichloro-6-oxo-8-oxa-5-aza-1(1,4)-diazepana-7(1,3)-benzenacyclotetradecaphane-12-yl) oxime (2.0 g, HCl. salt) as a off-white solid.

LC-MS (ES+) m/z: 547.2 (M+H)+ (calculated: 547.2)

1H NMR (400 MHz, CDCl3-d) δ 8.06 (s, 1H), 7.80-7.50 (m, 4H), 7.34-7.29 (m, 3H), 4.55-4.37 (m, 3H), 3.79-3.38 (m, 14H), 2.60-1.75 (m, 10H)

A suspension of the (E)-benzaldehyde O-(7′⁴,7⁵-dichloro-6-oxo-8-oxa-5-aza-1(1,4)-diazepana-7(1,3)-benzenacyclotetradecaphane-12-yl) oxime (190 mg, 0.35 mmol, 1.0 equiv), AcOH (2.9 mL), H₂O (0.97 mL) and zinc (160 mg, 2.4 mmol, 7.0 equiv) was stirred for 2 h at room temperature under nitrogen. The mixture was then basified to pH=8 with a saturated solution of NaHCO3 and extracted with EtOAc (4×4 mL). The combined organic layers were dried over anhydrous Na₂SO₄ and concentrated under reduced pressure. The crude product (200 mg) was purified by Prep-HPLC with the following conditions (Column: Sunfire Prep C18 OBD Column, 50×250 mm, 5 μm; Mobile Phase A Water (0.05% TFA), Mobile Phase B: ACN; Flow rate: 90 mL/min; Gradient: 5% B to 40% B in 12 min, 40% B; Wave Length: 220 nm; RT1 (min): 12) to afford the intermediate compound 20 (130 mg, TFA salt) as a white solid

LC-MS (ES+) m/z: 444.2 (M+H)+ (calculated: 444.2)

1H NMR (400 MHz, CDCl3-d) δ 7.71 (s, 2H), 4.47-4.39 (m, 2H), 3.94-3.90 (m, 1H), 3.60-3.20 (m, 14H), 2.42-2.30 (m, 2H), 2.15-1.70 (m, 8H)

### Intermediate Compound 21

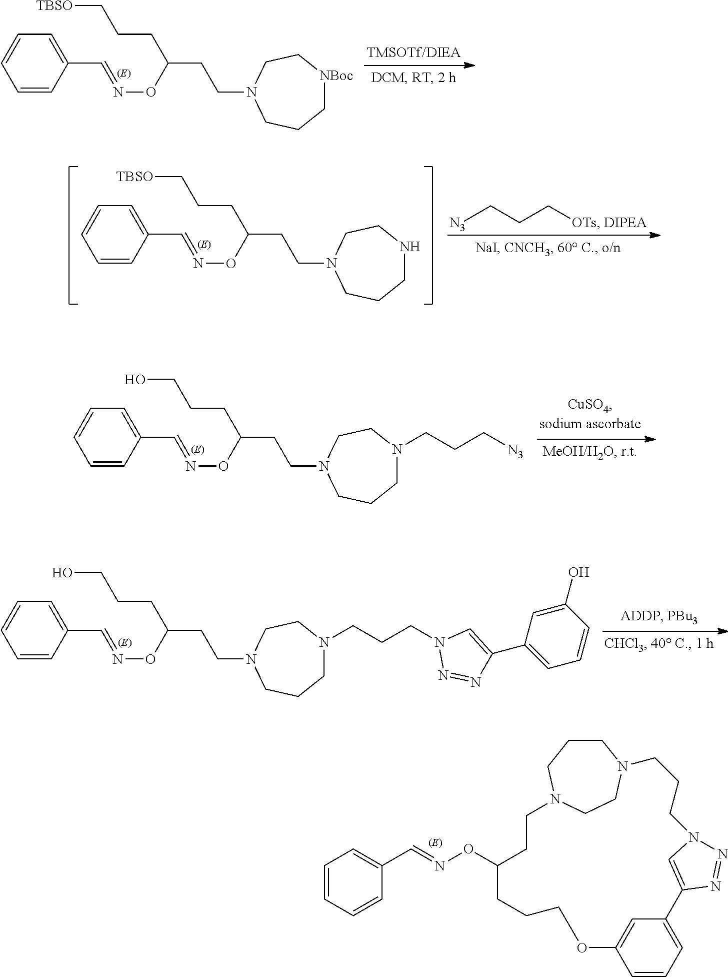

To a solution of intermediate compound 1 (500 mg, 0.937 mmol, 1.0 equiv) and DIPEA (0.65 mL, 3.75 mmol, 4.0 equiv) in DCM (8.9 mL), was added TMSOTf (0.51 mL, 2.81 mmol, 3.0 equiv) at 0° C. The resulting solution was stirred for 2 h at room temperature, and then quenched by the addition of water (10 mL). The organic phase was separated and washed with water (10 mL), followed by brine (10 mL). The organic layer was dried over anhydrous sodium sulfate and concentrated. The residue was dissolved in MeCN (4.8 mL) and DIPEA (0.32 mL, 1.84 mmol, 2.0 equiv) and sodium iodide (69 mg, 0.461 mmol, 0.5 equiv) was added, followed by 1-(3-azidopropoxysulfonyl)-4-methylbenzene (353 mg, 1.38 mmol, 1.5 equiv) at room temperature under a nitrogen atmosphere. The reaction mixture was stirred overnight at 60° C., and was allowed to cool down to room temperature. The reaction was diluted with water (50 mL) and the resulting mixture was extracted with EtOAc (3×50 mL). The combined organic layers were washed with brine (1×50 mL), dried over anhydrous MgSO₄ and concentrated under reduced pressure. The residue was purified by reverse flash chromatography (column, C18 silica gel; mobile phase, MeCN (0.1% Formic Acid) in water (0.1% Formic Acid), (0-100% gradient), L 254 nm and 220 nm) to afford (E)-benzaldehyde O-(1-(4-(3-azidopropyl)-1,4-diazepan-1-yl)-6-hydroxyhexan-3-yl) oxime (220 mg, 53% yield over 2 steps) as a light yellow oil (Note: Evaporation of the fractions with formic acid modifier led to the deprotection of the TBS group)

LC-MS (ES+) m/z: 403.6 (M+H)+ (calculated: 403.3)

1H NMR (400 MHz, CDCl3-d) δ 8.06 (s, 1H), 7.60-7.52 (m, 2H), 7.44-7.35 (m, 3H), 4.31 (dd, J=7.7, 4.0 Hz, 1H), 3.74-3.63 (m, 2H), 3.41-3.29 (m, 6H), 3.26-3.07 (m, 4H), 3.03 (t, J=5.9 Hz, 2H), 2.81 (t, J=7.1 Hz, 2H), 2.30-2.09 (m, 4H), 1.90-1.61 (m, 6H)

To a stirred solution of (E)-benzaldehyde O-(1-(4-(3-azidopropyl)-1,4-diazepan-1-yl)-6-hydroxyhexan-3-yl) oxime (400 mg, 0.892 mmol, 1.0 equiv) in MeOH (3.2 mL) and water (7.6 mL) was added copper(II) sulfate pentahydrate (44 mg, 0.178 mmol, 0.2 equiv), sodium ascorbate (177 mg, 0.892 mmol, 1.0 equiv) and 3-ethynylphenol (107 μL, 0.981 mmol, 1.1 equiv) and the reaction mixture was stirred overnight at room temperature under a nitrogen atmosphere. The reaction mixture was diluted with water (50 mL) and extracted with DCM/MeOH (9:1, 3×50 mL) The combined organic components were washed with brine (1×50 mL), dried over anhydrous MgSO4 and concentrated under reduced pressure affording (E)-benzaldehyde O-(6-hydroxy-1-(4-(3-(4-(3-hydroxyphenyl)-1H-1,2,3-triazol-1-yl)propyl)-1,4-diazepan-1-yl)hexan-3-yl) oxime (375 mg, 81% yield) as a yellow oil. Material was used without further purification.

LC-MS (ES+) m/z: 521.5 (M+H)+ (calculated: 521.3)

1H NMR (400 MHz, CDCl3-d) δ 8.27 (s, 1H), 8.11 (s, 1H), 7.63-7.56 (m, 2H), 7.42-7.35 (m, 3H), 7.29-7.24 (m, 3H), 6.83-6.75 (m, 1H), 4.51 (t, J=6.8 Hz, 2H), 4.25-4.21 (m, 1H), 3.64-3.56 (m, 3H), 2.85-2.66 (m, 9H), 2.58-2.50 (m, 2H), 2.14-1.99 (m, 2H), 2.00-1.58 (m, 8H)

To a stirred suspension of (E)-benzaldehyde O-(6-hydroxy-1-(4-(3-(4-(3-hydroxyphenyl)-1H-1,2,3-triazol-1-yl)propyl)-1,4-diazepan-1-yl)hexan-3-yl) oxime (60 mg, 0.345 mmol, 1.0 equiv), and ADDP (87 mg, 0.345 mmol, 3.0 equiv) in CHCl3 (2.2 mL) under nitrogen atmosphere was added n-Bu₃P (87 μL, 0.345 mmol, 3.0 equiv). The resulting mixture was stirred for 1 h at 40° C., and then allowed to cool down to room temperature. The reaction was quenched with 50% brine (30 mL) at room temperature. The resulting mixture was extracted with EtOAc (3×50 mL). The combined organic layers were dried over anhydrous Na₂SO₄ and concentrated under reduced pressure. The residue was purified by reverse flash chromatography (column, C18 silica gel; mobile phase, MeCN (0.1% Formic Acid) in water (0.1% Formic Acid), (10-70% gradient), UV 254 nm and 220 nm), affording the intermediate compound 21 (20 mg, 35% yield) as a colourless solid.

LC-MS (ES+) m/z: 503.6 (M+H)+ (calculated: 503.3)

1H NMR (400 MHz, CDCl3-d) δ 8.03 (s, 1H), 7.66 (s, 1H), 7.59-7.49 (m, 3H), 7.46-7.28 (m, 5H), 6.94 (d, J=2.5 Hz, 1H), 4.62-4.48 (m, 2H), 4.35-4.28 (m, 3H), 3.00-2.77 (m, 5H), 2.76-2.43 (m, 5H), 2.29-1.66 (m, 12H).

### Intermediate Compound 22

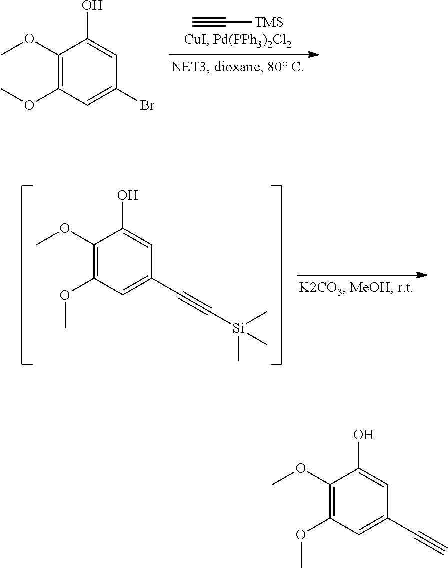

To a sealed microwave vial charged with 5-bromo-2,3-dimethoxyphenol (269 mg, 1.15 mmol, 1.0 equiv), tetrakis(triphenylphosphine)palladium(0) (67 g, 0.058 mmol, 0.05 equiv) and copper(I) iodide (22 mg, 0.115 mmol, 0.1 equiv) under a nitrogen atmosphere was added trimethylamine (3.15 mL), followed by ethynyltrimethylsilane (0.24 mL, 1.73 mmol, 1.5 equiv) and the reaction mixture was stirred overnight at 90° C. The reaction mixture was cooled to room temperature and was diluted with water (50 mL) and extracted with EtOAc (3×50 mL). The combined organic components were washed with brine (1×50 mL), dried over anhydrous MgSO₄ and concentrated under reduced pressure to afford a brown oil. The crude oil was dissolved in methanol (6 mL) and K₂CO₃ (70 mg) was added to the solution. The reaction mixture stirred for 30 mins at room temperature. The reaction mixture was concentrated under reduced pressure to afford a brown solid. The crude was diluted with 1 M aq. K₂CO₃ (50 mL) and extracted with EtOAc (3×50 mL). The combined organic components were washed with brine (1×50 mL), dried over anhydrous MgSO₄ and concentrated under reduced pressure to afford a brown oil. The residue was purified by silica gel column chromatography, eluting with PE/EA (0-100% gradient) to afford the intermediate compound 22 (195 mg, 95% yield) as a yellow solid.

LC-MS (ES+) m/z: 179.0 (M+H)+ (calculated: 179.0)

### Intermediate Compound 23

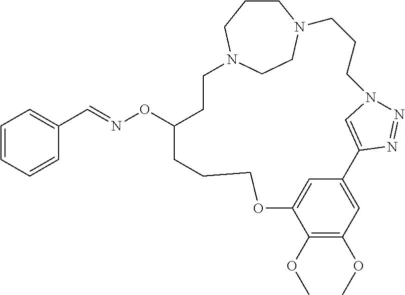 [SMILES: COc1cc2cc(c1OC)OCCCC(O/N=C/c1ccccc1)CCN1CCCN(CCCn3cc-2nn3)CC1]

The intermediate compound 23 was prepared using the intermediate 22 (79 mg, 0.441 mmol, 1.1 equiv) following the protocol described for the intermediate compound 21 The intermediate compound 23 (5 mg, 21% yield) was isolated as a colorless solid.

LC-MS (ES+) m/z: 563.3 (M+H)+ (calculated: 563.3)

1H NMR (400 MHz, CDCl3-d) δ 8.02 (s, 1H), 7.57-7.48 (m, 2H), 7.39-7.33 (m, 3H), 7.23 (s, 1H), 7.19 (s, 1H), 4.75-4.47 (m, 2H), 4.34 (s, 2H), 3.95 (s, 2H), 3.87 (s, 2H), 3.13-2.90 (m, 4H), 2.72-2.60 (m, 2H), 2.59-2.42 (m, 3H), 2.23-2.09 (m, 3H), 1.94-1.80 (m, 3H), 1.67-1.46 (m, 8H).

### Intermediate Compound 24

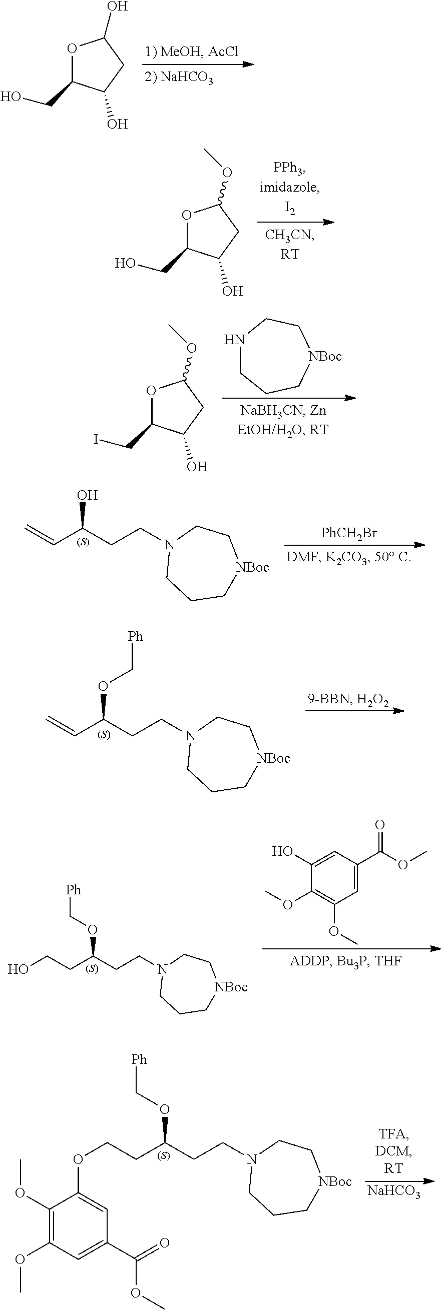

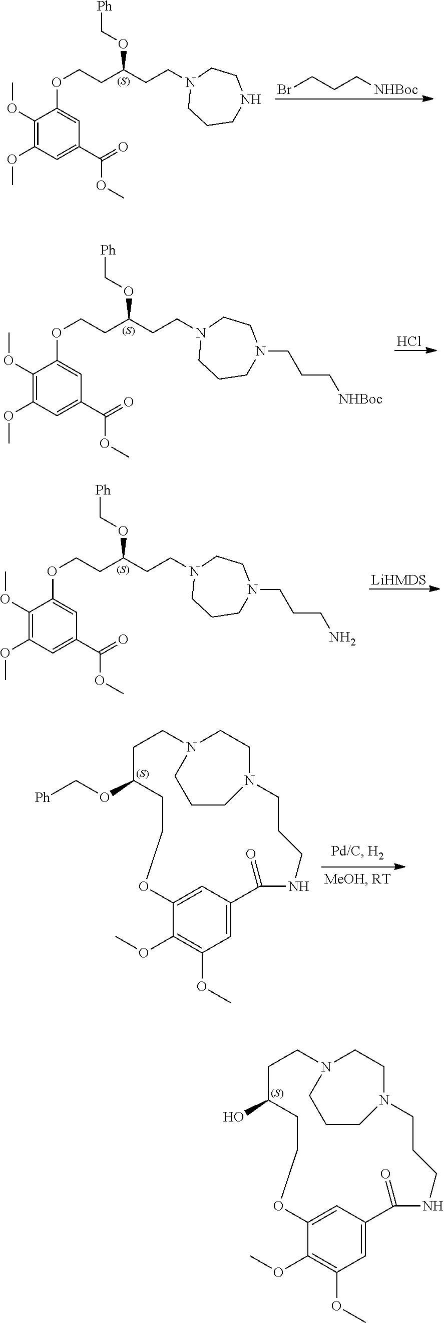

Methanol (1.2 L) is charged into a reactor, stirred for 10-15 minutes, and cooled to 0-5° C., then acetyl chloride (2.34 g, 29.8 mmol, 0.05 eq) is added and the mixture is stirred for 10-15 minutes at 0-5° C. The obtained methanolic hydrogen chloride is transferred into another container. Methanol (400 mL) is charged into a clean reactor and stirred for 10-15 minutes at 25-35° C. 2-deoxy-D-ribose (80.0 g, 596.43 mmol, 1.00 eq) is charged into the reactor and the mixture is stirred at 25-35° C. for 10-15 minutes. The mass is cooled to 0-5° C. and the methanolic hydrogen chloride solution prepared above is charged into the reactor at same temperature. The obtained mass is maintained at 0-5° C. for 2-3 hours. Sodium bicarbonate (3.0 g, 35.78 mmol, 0.06 eq) is charged into the mass at 0-5° C. and the mass is filtered. The filtrate is collected in another container and the filter bed is washed with methanol (100 mL) The combined filtrate was concentrated. The residue was purified by silica gel column chromatography, eluted with PE/THF (5:1) to afford (2R,3S)-2-(hydroxymethyl)-5-methoxyoxolan-3-ol (83 g, 94% yield) as a white solid

To a stirred solution of (2R,3S)-2-(hydroxymethyl)-5-methoxyoxolan-3-ol (80 g, 539.96 mmol, 1.00 equiv) and PPh3 (212.44 g, 0.81 mol, 1.50 equiv) in THF (1.6 L) were added imidazole (73.52 g, 1.08 mol, 2.00 equiv) and 12 (205.57 g, 0.81 mol, 1.50 equiv) at room temperature under nitrogen atmosphere. The resulting mixture was stirred for 16 h at room temperature under nitrogen atmosphere. The reaction was quenched with a saturated solution of NaHSO3 at room temperature. The organic phase was washed with brine. The organic layers were dried over anhydrous Na₂SO₄ and concentrated under reduced pressure. The residue was purified by silica gel column chromatography, eluted with PE/THF (5:1) to afford (2S,3S)-2-(iodomethyl)-5-methoxyoxolan-3-ol (98 g, 70% yield) as light oil.

To a stirred solution of (2S,3S)-2-(iodomethyl)-5-methoxyoxolan-3-ol (6.1 g, 23.63 mmol, 1.00 equiv) and zinc (15.46 g, 236.38 mmol, 10.00 equiv) in EtOH (120 mL) and AcOH (1.7 g, 28.36 mmol, 1.20 equiv) were added tert-butyl 1,4-diazepane-1-carboxylate (4.73 g, 23.63 mmol, 1.00 equiv) and NaBH₃CN (4.46 g, 70.91 mmol, 3.00 equiv) dropwise at room temperature. The resulting mixture was stirred for 2 h at room temperature. The resulting mixture was diluted with DCM (20 mL). The resulting mixture was filtered; the filter cake was washed with DCM (10 mL). The filtrate was concentrated under reduced pressure. The residue was purified by silica gel column chromatography, eluted with PE:THF (1:2) to afford tert-butyl 4-[(3S)-3-hydroxypent-4-en-1-yl]-1,4-diazepane-1-carboxylate (2.8 g, 42% yield) as a colorless oil.

LC-MS (ES+) m/z: 285.2 (M+H)+ (calculated: 285.2)

A solution of tert-butyl 4-[(3S)-3-hydroxypent-4-en-1-yl]-1,4-diazepane-1-carboxylate (2.0 g, 7 mmol, 1.00 equiv) in DMF (40 mL) was treated with NaH (2.7 g, 11.2 mmol, 1.60 equiv) at 0° C. followed by the addition of benzyl bromide (18.04 g, 10.5 mmol, 1.50 equiv). The resulting mixture was stirred for 2 h at 0° C. The reaction was quenched with Water/Ice at 0° C. The aqueous layer was extracted with EtOAc (2×20 mL). The residue was purified by silica gel column chromatography, eluted with PE/THF (4:1) to afford tert-butyl 4-[(3S)-3-(benzyloxy) pent-4-en-1-yl]-1,4-diazepane-1-carboxylate (1.8 g, 68% yield) as a light red oil.

LC-MS (ES+) m/z: 375.3 (M+H)+ (calculated: 375.3)

A solution of tert-butyl 4-[(3S)-3-(benzyloxy)pent-4-en-1-yl]-1,4-diazepane-1-carboxylate (1.6 g, 4.27 mmol, 1.00 equiv) and 9-borabicyclo[3.3.1]nonane (1.56 g, 12.81 mmol, 3.00 equiv) in THF (5 mL) was stirred for 1.5 h at room temperature under nitrogen atmosphere. After the reaction was cooled to 0° C. The NaOH (0.51 g, 12.81 mmol, 3.00 equiv) and H2O2 (30%) (0.44 g, 12.81 mmol, 3.00 equiv) was added. The resulting mixture was stirred for 2 h at 0° C. under nitrogen atmosphere. The resulting mixture was extracted with EtOAc (2×10 mL). The combined organic layers were washed with brine (20 mL), dried over anhydrous Na₂SO₄. After filtration, the filtrate was concentrated under reduced pressure. The residue was purified by silica gel column chromatography, eluted with THF/PE (1:4) to afford tert-butyl 4-[(3S)-3-(benzyloxy)-5-hydroxypentyl]-1,4-diazepane-1-carboxylate (1.2 g, 72% yield) as a colorless oil

LC-MS (ES+) m/z: 393.3 (M+H)+ (calculated: 393.3)

To a stirred mixture of tert-butyl 4-[(3S)-3-(benzyloxy)-5-hydroxypentyl]-1,4-diazepane-1-carboxylate (1.5 g, 3.82 mmol, 1.00 equiv) and methyl 3-hydroxy-4,5-dimethoxybenzoate (0.81 g, 3.82 mmol, 1.00 equiv) in THE (30 mL) were added ADDP (1.43 g, 5.73 mmol, 1.50 equiv) and n-Bu₃P (1.16 g, 5.73 mmol, 1.50 equiv) at room temperature. The resulting mixture was stirred for 1 h at room temperature. The reaction was quenched with a saturated solution of NH₄Cl. The resulting mixture was extracted with EtOAc (2×20 mL). The combined organic layers were washed with brine (20 mL), dried over anhydrous Na₂SO₄. After filtration, the filtrate was concentrated under reduced pressure. The crude product was purified by Prep-HPLC to afford tert-butyl 4-[(3S)-3-(benzyloxy)-5-[2,3-dimethoxy-5-(methoxycarbonyl)phenoxy]pentyl]-1,4-diazepane-1-carboxylate (1.1 g, 49% yield) as an off-white solid.

LC-MS (ES+) m/z: 587.3 (M+H)+ (calculated: 587.3)

Into a 20 mL vial were added tert-butyl 4-[(3S)-3-(benzyloxy)-5-[2,3-dimethoxy-5-(methoxycarbonyl)phenoxy] pentyl]-1,4-diazepane-1-carboxylate (1.1 g, 1.87 mmol, 1.00 equiv) and TFA (2.14 g, 18.75 mmol, 10.00 equiv) in DCM (10 mL) at room temperature. The resulting mixture was stirred for 1 hi at room temperature. The resulting mixture was concentrated under low temperature. The crude product was dissolved into ethyl acetate (20 mL) and washed with NaHCO3 (aq.). The organic layers were washed with brine (10 mL), dried over anhydrous Na₂SO₄. After filtration, the filtrate was concentrated under reduced pressure. The product methyl (S)-3-((3-(benzyloxy)-5-(1,4-diazepan-1-yl)pentyl)oxy)-4,5-dimethoxybenzoate (0.7 g, 78% yield) as an off-white solid.

LC-MS (ES+) m/z: 487.3 (M+H)+ (calculated: 487.3)

To a stirred solution of methyl 3-{[(3S)-3-(benzyloxy)-5-(1,4-diazepan-1-yl)pentyl]oxy}-4,5-dimethoxybenzoate (300 mg, 0.617 mmol, 1.00 equiv) and tert-butyl N-(3-bromopropyl)carbamate (176.17 mg, 0.74 mmol, 1.20 equiv) in CH₃CN (8 mL) was added K₂CO₃ (127.81 mg, 0.925 mmol, 1.50 equiv) at room temperature. The resulting mixture was stirred for 16 h at 50° C. The resulting mixture was diluted with DCM (10 mL) The resulting mixture was filtered; the filter cake was washed with DCM (5 mL). The filtrate was concentrated under reduced pressure. The residue was purified by silica gel column chromatography, eluted with PH:THF=6:1 to afford methyl-3-{[(3S)-3-(benzyloxy)-5-(4-{3-[(tert-butoxy-carbonyl)amino]propyl}-1,4-diazepan-1-yl)pentyl]oxy}-4,5-dimethoxybenzoate (240 mg, 60% yield) as a colorless oil.

LC-MS (ES+) m/z: 644.4 (M+H)+ (calculated: 644.4)

Into a 8 ml, sealed tube were added methyl 3-{[(3S)-3-(benzyloxy)-5-(4-{3-[(tert-butoxycarbonyl)amino]propyl}-1,4-diazepan-1-yl)pentyl]oxy}-4,5-dimethoxybenzoate (60 mg, 0.093 mmol, 1.00 equiv) and 1M HCl(gas) in MeOH (l mL) at room temperature. The resulting mixture was stirred for 1 h at room temperature. The resulting mixture was concentrated under reduced pressure. The residue was purified by trituration with diethyl ether (10 mL) to afford methyl 3-{[(3S)-5-[4-(3-aminopropyl)-1,4-diazepan-1-yl]-3-(benzyloxy)pentyl] oxy}-4,5-dimethoxybenzoate dihydrochloride (50 mg, 87% yield, HCl salt) as a white solid.

LC-MS (ES+) m/z: 544.3 (M+H)+ (calculated: 544.3)

Into a 20 mL vial were added methyl 3-{[(3S)-5-[4-(3-aminopropyl)-1,4-diazepan-1-yl]-3-(benzyloxy)pentyl] oxy}-4,5-dimethoxybenzoate dihydrochloride (250 mg, 0.405 mmol, 1.00 equiv) and THF (10 mL). Then 1 M LiHMDS (4 mL, 10.00 equiv) was added at 0° C. The resulting mixture was stirred for 2 h at 0° C. under N2 atmosphere. The reaction was quenched with a saturated solution of NH₄Cl (aq.) at 0° C. The resulting mixture was extracted with EtOAc (2×5 mL). The combined organic layers were dried over anhydrous Na₂SO₄. After filtration, the filtrate was concentrated under reduced pressure. The residue was purified by silica gel column chromatography, eluted with PE/THF (2.1) to afford (15S)-15-(benzyloxy)-9,10-dimethoxy-12-oxa-1,5,18-triazatricyclo[16.3.2.1\hat{ }{7,11}] tetracosa-7,9,11(24)-trien-6-one (131 mg, 63% yield) as an off-white solid.

LC-MS (ES+) m/z: 512.3 (M+H)+ (calculated: 512.3)

To a stirred solution of (15S)-15-(benzyloxy)-9,10-dimethoxy-12-oxa-1,5,18-triazatricyclo[16.32.1\hat{ }{7,11}]tetracosa-7,9,11(24)-trien-6-one (121 mg, 0.236 mmol, 1.00 equiv) in MeOH (10 mL) was added Pd/C (75 mg) at room temperature under hydrogen atmosphere. The resulting mixture was stirred for 1 h at room temperature under hydrogen atmosphere. The resulting mixture was filtered; the filter cake was washed with MeOH (3×5 mL). The filtrate was concentrated to afford the intermediate compound 24 (63 mg, 63% yield) as an off-white solid.

LC-MS (ES+) m/z: 422.3 (M+H)+ (calculated: 422.3)

### Intermediate Compound 25

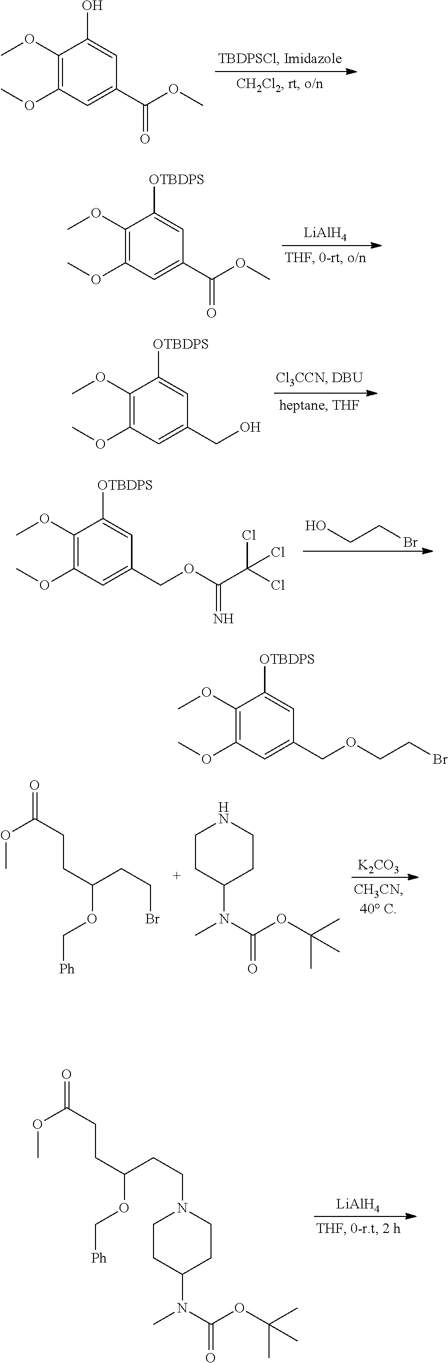

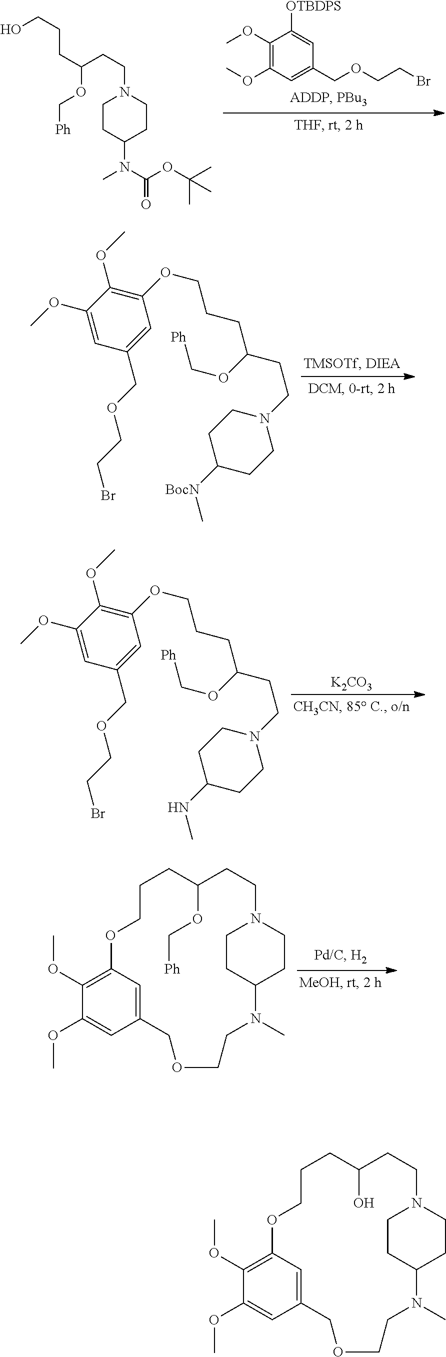

To a stirred solution of methyl 3-hydroxy-4,5-dimethoxybenzoate (2.5 g, 11.78 mmol, 1.00 equiv) and imidazole (1.20 g, 17.67 mmol, 1.50 equiv) in DCM (100 mL) were added tert-Butyl(chloro)diphenylsilane (3.72 g, 13.55 mmol, 1.15 equiv) at 0° C. and the mixture was stirred overnight at room temperature under nitrogen atmosphere. The reaction was quenched with water (200 mL) at room temperature and the resulting mixture was extracted with DCM (2×100 mL). The combined organic layers were washed with brine (1×200 mL), dried over anhydrous Na₂SO₄. After filtration, the filtrate was concentrated under reduced pressure. The crude product was re-crystallized from PE/ethyl acetate=20:1 to afford methyl 3-((tert-butyldiphenylsilyl)oxy)-4,5-dimethoxybenzoate (4.5 g, 85% yield) as an off-white solid.

LC-MS (ES+) m/z: 451.2 (M+H)+ (calculated: 451.2)

To a stirred solution of methyl 3-((tert-butyldiphenylsilyl)oxy)-4,5-dimethoxybenzoate (4.5 g, 9.98 mmol, 1.00 equiv) in THF (90 mL) was added LiAlH₄ (0.76 g, 20.08 mmol, 2.00 equiv) at 0° C. under nitrogen atmosphere. The resulting mixture was stirred overnight at room temperature under nitrogen atmosphere. The mixture was allowed to cool down to 0° C. Water (1 mL) was added dropwise to the cooled reaction mixture, followed by 15% aq. NaOH (1 mL) and additional water (3 mL). The reaction mixture was warmed to room temperature and stirred for 15 mins. The solids were filtered out and the organic layer was dried over anhydrous MgSO₄ and concentrated under reduced pressure. The crude product was re-crystallized from PE/EA (10:1, 15 mL) to afford (3-((tert-butyldiphenylsilyl)oxy)-4,5-dimethoxyphenyl)methanol (3.0 g, 71% yield) as an off-white solid.

LC-MS (ES+) m/z: 423.2 (M+H)+ (calculated: 423.2)

A mixture of (3-((tert-butyldiphenylsilyl)oxy)-4,5-dimethoxyphenyl)methanol (1.0 g, 2.37 mmol, 1.00 equiv) and trichloroacetonitrile (1.02 g, 7.10 mmol, 3.00 equiv) in THF (5 mL) and heptane (15 mL) was stirred at 0° C. under nitrogen atmosphere. To the above mixture was added DBU (0.04 g, 0.24 mmol, 0.10 equiv) in portions at 0° C. The resulting mixture was stirred overnight at room temperature. The reaction was quenched with a saturated solution of NH₄Cl (100 mL) at room temperature. The resulting mixture was extracted with EtOAc (2×50 mL). The combined organic layers were washed with brine (1×100 mL), dried over anhydrous Na₂SO₄. After filtration, the filtrate was concentrated under reduced pressure. The crude product of 3-((tert-butyldiphenylsilyl)oxy)-4,5-dimethoxybenzyl 2,2,2-trichloroacetimidate (1.2 g) was used in the next step directly without further purification.

To a mixture of 3-((tert-butyldiphenylsilyl)oxy)-4,5-dimethoxybenzyl 2,2,2-trichloroacetimidate (1.2 g, 2.10 mmol, 1.00 equiv) and 2-bromoethan-1-ol (0.29, 2.33 mmol, 1.10 equiv) in cyclohexane (12 mL) and DCM (6 mL) was added DL-Camphor sulfonic acid (0.05 g, 0.21 mmol, 0.10 equiv) in portions at 0° C. under nitrogen atmosphere. The resulting mixture was stirred overnight at room temperature. The mixture was concentrated and the residue was purified by silica gel column chromatography, eluted with PE/THF (20:1-8-1) to afford (5-((2-bromoethoxy)methyl)-2,3-dimethoxyphenoxy)(tert-butyl)diphenylsilane (0.88 g, 78% yield) as a light yellow oil.

LC-MS (ES+) m/z: 529.1 (M+H)+ (calculated: 529.1)

To a stirred solution of methyl 4-(benzyloxy)-6-bromohexanoate (1.5 g, 4.76 mmol, 1.00 equiv) and tert-butyl N-methyl-N-(piperidin-4-yl)carbamate (1.22 g, 5.71 mmol, 1.20 equiv) in CH₃CN (30 mL) was added K₂CO₃ (1.64 g, 11.89 mmol, 2.50 equiv) in portions at 40° C. under nitrogen atmosphere. The resulting mixture was stirred overnight at 40° C. The reaction was quenched by the addition of a saturated solution of NH₄Cl (100 mL) at room temperature. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by silica gel column chromatography, eluted with PE/THF (10:1-3:1) to afford methyl 4-(benzyloxy)-6-(4-((tert-butoxycarbonyl)(methyl)amino)piperidin-1-yl)hexanoate (1.8 g, 84% yield) as a light yellow oil.

LC-MS (ES+) m/z: 449.3 (M+H)+ (calculated: 449.3)

To a stirred solution of methyl methyl 4-(benzyloxy)-6-(4-((tert-butoxycarbonyl)(methyl)amino)piperidin-1-yl)hexanoate (1.80 g, 4.01 mmol, 1.00 equiv) in THF (50 mL) was added LiAlH₄ (0.23 g, 6.02 mmol, 1.50 equiv) in portions at 0° C. under nitrogen atmosphere. The resulting mixture was stirred for 2 h at room temperature under nitrogen atmosphere. The reaction mixture was cooled to 0° C. (ice bath) and diluted with THF (50 mL).

Water (0.5 mL) as added dropwise to the cooled reaction mixture, followed by 15% aq. NaOH (0.5 mL) and additional water (1.5 mL). The resulting mixture was stirred for 15 min at room temperature. The resulting mixture was filtered and the filtrate was concentrated. The residue was purified by silica gel column chromatography, eluted with PE/THF (10:1-1:2) to afford tert-butyl (1-(3-(benzyloxy)-6-hydroxyhexyl)piperidin-4-yl)(methyl)carbamate (1.50 g, 89% yield) as a light yellow oil.

LC-MS (ES+) m/z: 421.3 (M+H)+ (calculated: 421.3)

To a stirred mixture of tert-butyl (1-(3-(benzyloxy)-6-hydroxyhexyl)piperidin-4-yl)(methyl)carbamate (1.10 g, 2.62 mmol, 1.00 equiv) and (5-((2-bromoethoxy)methyl)-2,3-dimethoxyphenoxy)(tert-butyl)diphenylsilane (0.84 g, 2.89 mmol, 1.10 equiv) in THF (40 mL) were added ADDP (1.64 g, 6.54 mmol, 2.50 equiv) and n-tributylphosphane (1.32 g, 6.52 mmol, 2.50 equiv) in portions at room temperature under nitrogen atmosphere. The resulting mixture was stirred for 2 h at room temperature under nitrogen atmosphere. The reaction was quenched by the addition of a saturated solution of NH₄Cl (100 mL) at room temperature. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/THF (1:5-1:1) to afford tert-butyl (1-(3-(benzyloxy)-6-(5-((2-bromoethoxy)methyl)-2,3-dimethoxyphenoxy)hexyl)piperidin-4-yl)(methyl)carbamate (0.80 g, 44% yield) as a light yellow oil.

LC-MS (ES+) m/z: 693.3 (M+H)+ (calculated: 693.3)

To a stirred mixture of tert-butyl (1-(3-(benzyloxy)-6-(5-((2-bromoethoxy)methyl)-2,3-dimethoxyphenoxy)hexyl)piperidin-4-yl)(methyl)carbamate (0.8 g, 1.15 mmol, 1.00 equiv) and DIEA (0.75 g, 5.76 mmol, 5.00 equiv) in DCM (20 mL) was added TMSOTf (0.77 g, 3.46 mmol, 3.00 equiv) dropwise at 0° C. The resulting mixture was stirred for 2 h at room temperature under nitrogen atmosphere. The reaction was quenched by the addition of a saturated solution of NaHCO3 (30 mL) at room temperature. The resulting mixture was extracted with DCM (2×20 mL). The combined organic layers were washed with brine (1×30 mL), dried over anhydrous Na₂SO₄. After filtration, the filtrate was concentrated under reduced pressure. The crude product 1-(3-(benzyloxy)-6-(5-((2-bromoethoxy)methyl)-2,3-dimethoxyphenoxy)hexyl)-N-methylpiperidin-4-amine (0.6 g) was used in the next step directly without further purification.

LC-MS (ES+) m/z: 593.3 (M+H)+ (calculated: 593.3)

To a stirred solution of 1-(3-(benzyloxy)-6-(5-((2-bromoethoxy)methyl)-2,3-dimethoxyphenoxy)hexyl)-N-methylpiperidin-4-amine (0.6 g, 1.01 mmol, 1.00 equiv) in CH3CN (12 mL) was added K₂CO₃ (0.35 g, 2.53 mmol, 2.50 equiv) in portions at room temperature under nitrogen atmosphere. The resulting mixture was stirred for overnight at 85° C. under nitrogen atmosphere. The mixture was allowed to cool down to room temperature. The resulting mixture was diluted with acetonitrile (20 mL). The resulting mixture was filtered, the filter cake was washed with acetonitrile (2×10 mL) The filtrate was concentrated under reduced pressure. This resulted in 12-(benzyloxy)-7⁴,7⁵-dimethoxy-2-methyl-5,8-dioxa-2-aza-1(4,1)-piperidine-7(1,3)-benzenacyclotetradecaphane (0.3 g, 57% yield) as a light yellow solid.

LC-MS (ES+) m/z: 513.3 (M+H)+ (calculated: 513.3)

A mixture of 12-(benzyloxy)-74,75-dimethoxy-2-methyl-5,8-dioxa-2-aza-1(4,1)-piperidina-7(1,3)-benzenacyclotetradecaphane (0.3 g, 0.59 mmol, 1.00 equiv) and Pd/C (0.30 g) in MeOH (20 mL) was stirred for 2 h at room temperature under hydrogen atmosphere. The resulting mixture was filtered and concentrated under reduced pressure. The crude intermediate compound 25 (0.15 g, 60% yield) was used in the next step directly without further purification.

LC-MS (ES+) m/z: 423.3 (M+H)+ (calculated: 423.3)

### Intermediate Compound 26

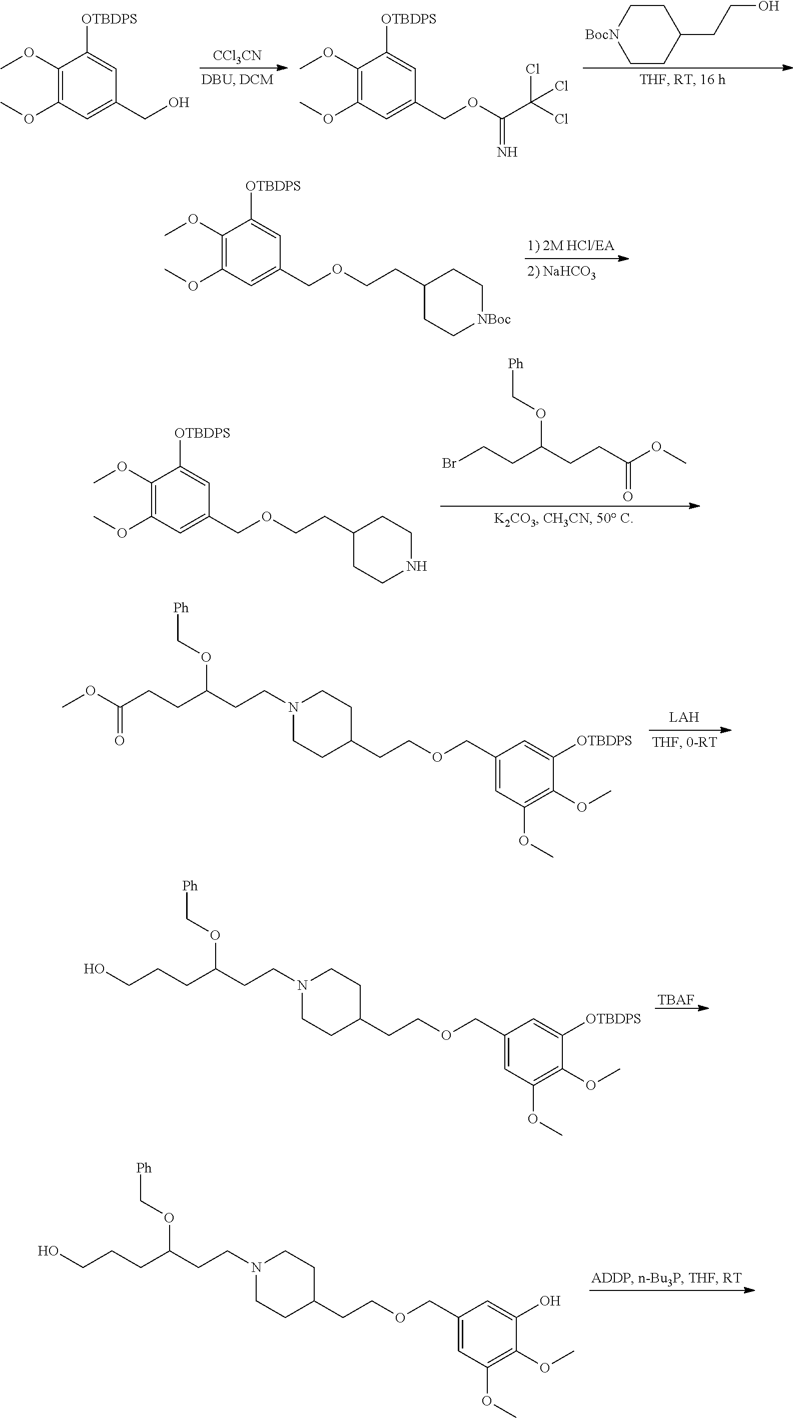

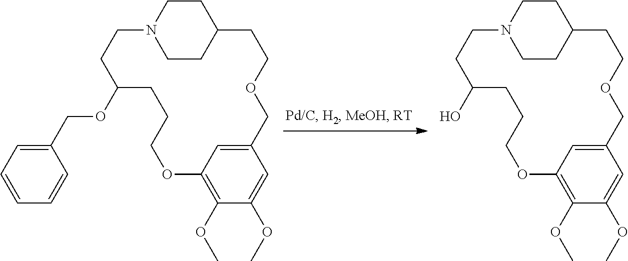 [SMILES: COc1cc2cc(c1OC)OCCCC(OCc1ccccc1)CCN1CCC(CCOC2)CC1 ; COc1cc2cc(c1OC)OCCCC(O)CCN1CCC(CCOC2)CC1]

To a stirred solution of (3-((tert-butyldiphenylsilyl)oxy)-4,5-dimethoxyphenyl)methanol (10 g, 23.66 mmol, 1.00 equiv) and trichloroacetonitrile (5.13 g, 35.49 mmol, 1.50 equiv) in n-heptane (30 mL) and THF (150 mL). The mixture was allowed to cool down to 0° C. Then DBU (0.36 g, 2.36 mmol, 0.1 equiv) was added. The resulting mixture was stirred for additional 16 h at room temperature. The reaction was quenched with a saturated solution of NH₄Cl and extracted with EtOAc. The organic layers were dried over anhydrous Na₂SO₄, and concentrated under reduced pressure. The crude product (13 g) as an off-white solid was used without purification in next step.

LC-MS (ES+) m/z: 566.1 (M+H)+ (calculated: 566.1)

To a stirred solution of 3-(tert-butyldiphenylsilyloxy)-4,5-dimethoxybenzyl 2,2,2-trichloroacetimidate (14.2 g, 25.05 mmol, 1.00 equiv) and tert-butyl 4-(2-hydroxyethyl)piperidine-1-carboxylate (14.36 g, 62.61 mmol, 2.50 equiv) in cyclohexane (220 mL) and DCM (110 mL) The mixture was allowed to cool down to 0° C. Then DL-Camphor sulfonic acid (1.16 g, 5.01 mmol, 0.20 equiv) was added. The resulting mixture was stirred for 16 h at room temperature. The resulting mixture was diluted with DCM (300 mL) and washed with water (2×100 mL). The combined organic layers were dried over anhydrous Na₂SO₄. After filtration, the filtrate was concentrated under reduced pressure. The residue was purified by silica gel column chromatography, eluted with PE/THF (2.1) to afford tert-butyl 4-(2-((3-((tert-butyldiphenylsilyl)oxy)-4,5-dimethoxybenzyl)oxy)ethyl)piperidine-1-carboxylate (12 g, 75% yield) as a light yellow oil.

LC-MS (ES+) m/z: 634.4 (M+H)+ (calculated: 634.4)

To a solution of tert-butyl 4-(2-((3-((tert-butyldiphenylsilyl)oxy)-4,5-dimethoxybenzyl)oxy)ethyl)piperidine-1-carboxylate (200 mg, 0.32 mmol, 1.00 equiv) in ethyl acetate (3 mL) was added a solution of HCl (2 M) in ethyl acetate (1 mL) at 0° C. The resulting mixture was stirred for 1 h at room temperature. The resulting mixture was concentrated under low temperature. The crude product was dissolved into ethyl acetate (10 mL) and washed with a saturated solution of NaHCO₃. The organic layers were washed with brine (10 mL), dried over anhydrous Na₂SO₄. After filtration, the filtrate was concentrated under reduced pressure. The 4-[2-({3-[(tert-butyldiphenylsilyl)oxy]-4,5-dimethoxyphenyl}meth-oxy)ethyl]piperidine (160 mg, 95% yield) as a light yellow oil was used without further purification.

LC-MS (ES+) m/z: 534.4 (M+H)+ (calculated: 534.4)

To a mixture of 4-[2-({3-[(tert-butyldiphenylsilyl)oxy]4,5-dimethoxyphenyl}methoxy)ethyl]piperidine (3.6 g, 6.74 mmol, 1.00 equiv) and K₂CO₃ (1.86 g, 13.48 mmol, 2.0 equiv) in acetonitrile (80 mL) was added methyl 4-(benzyloxy)-6-bromohexanoate (3.19 g, 10.12 mmol, 1.50 equiv) at room temperature. The resulting mixture was stirred for 16 h at 50° C. The resulting mixture was diluted with DCM (100 mL). The resulting mixture was filtered; the filter cake was washed with DCM (20 mL). The filtrate was concentrated under reduced pressure. The residue was purified by silica gel column chromatography, eluted with PE/THF (1:1) to afford 4-(benzyloxy)-6-{4-[2-({3-[(tert-butyldiphenylsilyl)oxy]-4,5-dimethoxyphenyl}methoxy)ethyl]piperidin-1-yl}hexan-1-ol (3.6 g, 72% yield) as a colorless oil.

LC-MS (ES+) m/z: 768.4 (M+H)+ (calculated: 768.4).

To a stirred solution of 4-(benzyloxy)-6-{4-[2-({3-[(tert-butyldiphenylsilyl)oxy]-4,5-dimethoxyphenyl})methoxy)ethyl] piperidin-1-yl}hexan-1-ol (3.6 g, 4.86 mmol, 1.00 equiv) in THF (72 mL) was added LiAlH4 (0.36 g, 9.37 mmol, 2.00 equiv) in portions at 0° C. under nitrogen atmosphere. The resulting mixture was stirred for 1 h at 0′C under nitrogen atmosphere. The reaction was quenched by the addition of water (2 mL) at 0° C. The resulting mixture was filtered, the filter cake was washed with THF (20 mL). The filtrate were dried over Na₂SO₄ and concentrated to give 5-[(2-{1-[3-(benzyloxy)-6-hydroxyhexyl]piperidin-4-yl}ethoxy)methyl]-2,3-dimethoxyphenol (2.2 g, 63% yield) as colorless oil.

LC-MS (ES+) m/z: 740.4 (M+H)+ (calculated: 740.4).

To a stirred solution of 4-(benzyloxy)-6-{4-[2-({3-[(tert-butyldiphenylsilyl)oxy]-4,5-dimethoxyphenyl}methoxy)ethyl] piperidin-1-yl}hexan-1-ol (2.2 g, 2.97 mmol, 1.00 equiv) in THF (20 mL) was added TBAF (2.33 g, 8.92 mmol, 3.00 equiv) at room temperature. The resulting mixture was stirred for 2 h at room temperature. The resulting mixture was concentrated under reduced pressure. The residue was purified by reverse flash chromatography with the following conditions: column, C18 silica gel; mobile phase, MeOH in water, 40% to 95% gradient in 7 min; detector, UV 220 nm to afford 5-[(2-{1-[3-(benzyloxy)-6-hydroxyhexyl]piperidin-4-yl}ethoxy)methyl]-2,3-dimethoxyphenol (630 mg, 42% yield) as colorless oil.

LC-MS (ES+) m/z: 502.3 (M+H)+ (calculated: 502.3).

To a stirred solution of 5-[(2-{1-[3-(benzyloxy)-6-hydroxyhexyl]piperidin-4-yl}ethoxy)methyl]-2,3-dimethoxyphenol (2.5 g, 4.983 mmol, 1.00 equiv) and in THF (100 mL) was added ADDP (1.25 g, 4.98 mmol, 2.00 equiv) and n-Bu3P (2.02 g, 4.98 mmol, 2.00 equiv) at room temperature under nitrogen atmosphere. The resulting mixture was stirred for 2 h at room temperature under nitrogen atmosphere. The reaction was quenched with a saturated solution of NH₄Cl. The resulting mixture was extracted with EtOAc (2×50 mL). The combined organic layers were dried over anhydrous Na₂SO₄. After filtration, the filtrate was concentrated under reduced pressure. The residue was purified by reverse flash chromatography with the following conditions (column, C18 silica gel; mobile phase, MeOH in water, 45% to 100% gradient in 10 min; detector, UV 220 nm) to afford 15-(benzyloxy)-8,9-dimethoxy-4,11-dioxa-18-azatricyclo [16.2.2.1\hat{ }{6,10}]tricosa-6(23),7,9-triene (0.43 g, 36% yield) as a colorless oil

LC-MS (ES+) m/z: 484.3 (M+H)+ (calculated: 484.3).

To a stirred mixture of 15-(benzyloxy)-8,9-dimethoxy-4,11-dioxa-18-azatricyclo[16.2.2.1\hat{ }{6,10}]tricosa-6(23), 7,9-triene (200 mg, 0.414 mmol, 1.00 equiv) in MeOH (8 mL) was added Pd/C (20 mg) at room temperature under hydrogen atmosphere. The resulting mixture was stirred for 1 h under hydrogen atmosphere. The resulting mixture was filtered; the filter cake was washed with MeOH (10 mL). The filtrate was concentrated under reduced pressure to give the intermediate compound 26 (120 mg, 74% yield) as a colorless oil, used without further purification.

LC-MS (ES+) m/z: 394.3 (M+H)+ (calculated: 394.3).

### Intermediate Compound 27

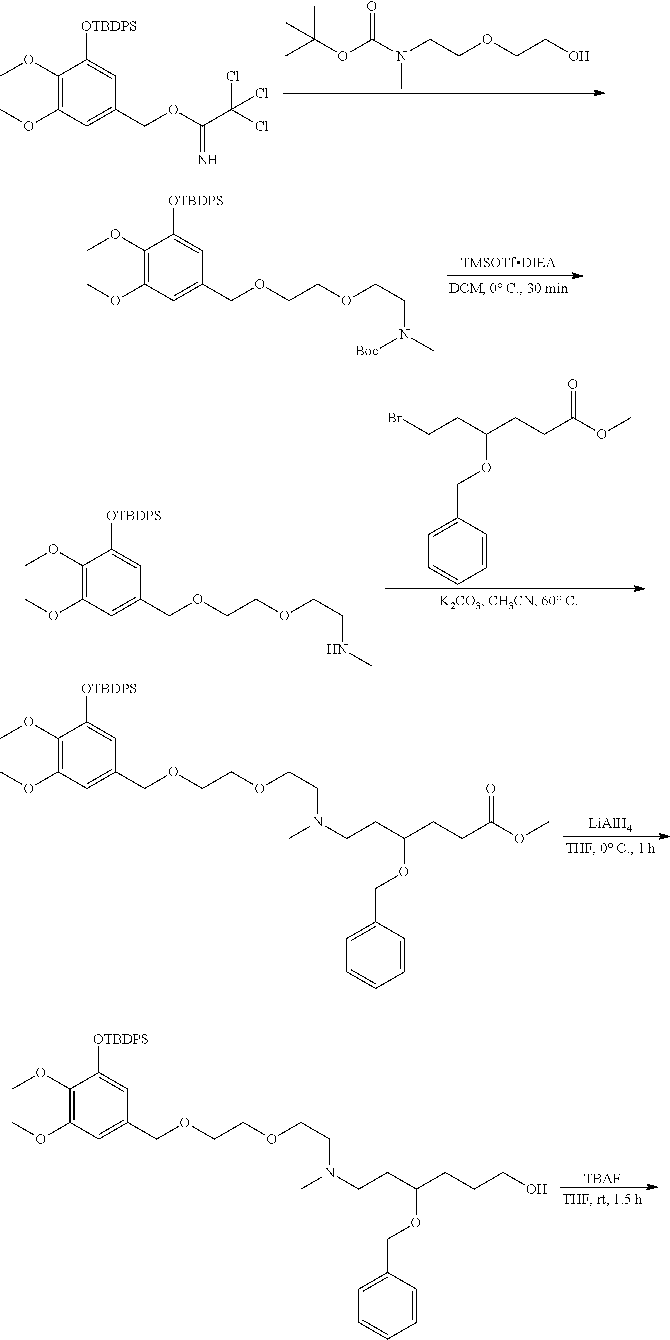

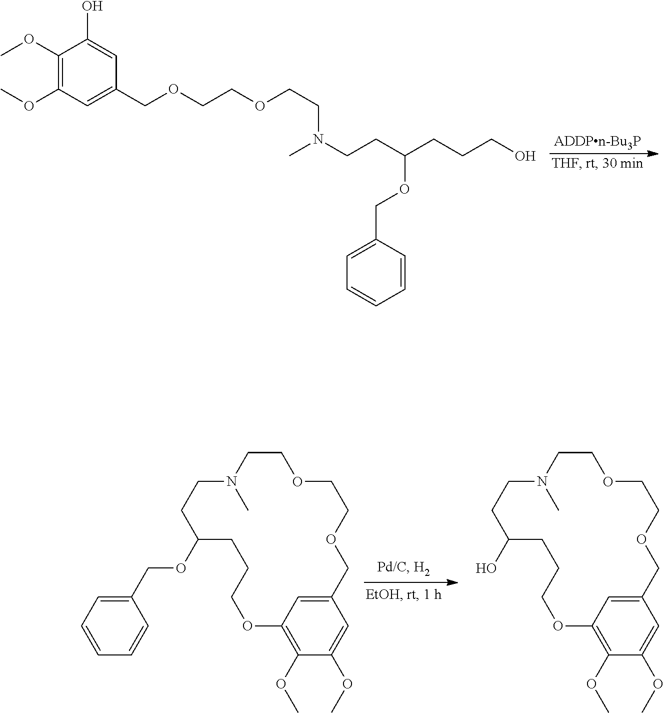 [SMILES: COc1cc(COCCOCCN(C)CCC(CCCO)OCc2ccccc2)cc(O)c1OC ; "THF, rt, 30 min" ; COc1cc2cc(c1OC)OCCCC(OCc1ccccc1)CCN(C)CCOCCOC2 ; COc1cc2cc(c1OC)OCCCC(O)CCN(C)CCOCCOC2 ; "ADDP·n-Bu3P"]

To a stirred solution of {3-[(ten-butyldiphenylsilyl)oxy]-4,5-dimethoxyphenyl}methyl 2,2,2-trichloroethanimidate (5.6 g, 9.88 mmol, 1.00 equiv) and tert-butyl N-[2-(2-hydroxyethoxy)ethyl]-N-methylcarbamate (3.25 g, 14.82 mmol, 1.50 equiv) in cyclohexane (120 mL) and DCM (60 mL) were added camphorsulfonic acid (0.46 g, 1.98 mmol, 0.20 equiv) at room temperature under N2 atmosphere. The resulting mixture was stirred for 24h at room temperature under N2 atmosphere. The reaction was quenched with a saturated solution of NaHCO3 (50 mL) at rt. The resulting mixture was extracted with ethyl acetate (3×100 mL) The combined organic layers were washed with brine (1×100 mL), dried over anhydrous Na₂SO₄. After filtration, the filtrate was concentrated under reduced pressure. The residue was purified by silica gel column chromatography, eluted with THF/PE (1:10-5:1) to afford tert-butyl N-{2-[2-({3-[(tert-butyldiphenylsilyl)oxy]-4,5-dimethoxyphenyl}methoxy)ethoxy]ethyl}-N-methylcarbamate (5.5 g, 76% yield) as a brown yellow oil.

LC-MS (ES+) m/z: 624.3 (M+H)+ (calculated: 624.3).

To a stirred solution of tert-butyl N-{2-[2-({3-[(tert-butyldiphenylsilyl)oxy]-4,5-dimethoxyphenyl}methoxy)ethoxy]ethyl}-N-methylcarbamate (5.5 g, 8.82 mmol, 1.00 equiv) and DIEA (6.84 g, 52.90 mmol, 6.00 equiv) in DCM (110 mL) was added TMSOTf (7.84 g, 35.26 mmol, 4.00 equiv) dropwise at 0° C. under N2 atmosphere. The resulting mixture was stirred for 30 min at 0° C. under N2 atmosphere. The reaction was quenched with a saturated solution of NH₄Cl (100 mL) at 0° C. The resulting mixture was extracted with ethyl acetate (3×100 mL) The combined organic layers were washed with brine (1×100 mL), dried over anhydrous Na₂SO₄. After filtration, the filtrate was concentrated under reduced pressure. This resulted in (2-[2-({3-[(tert-butyldiphenylsilyl)oxy]-4,5-dimethoxyphenyl)methoxy)ethoxy]ethyl}(methyl)amine (5 g, 86% yield) as a brown oil.

LC-MS (ES+) m/z: 524.3 (M+H)+ (calculated: 524.3).

To a stirred solution of {2-[2-({3-[(tert-butyldiphenylsilyl)oxy]-4,5-dimethoxyphenyl}methoxy)ethoxy]ethyl}(methyl)amine (5 g, 9.55 mmol, 1.00 equiv) and methyl 4-(benzyloxy)-6-bromohexanoate (4.51 g, 14.32 mmol, 1.50 equiv) in CH₃CN (100 mL) were added K₂CO₃ (3.96 g, 28.64 mmol, 3.00 equiv) at room temperature under N2 atmosphere. The resulting mixture was stirred for 14 h at 60° C. under N2 atmosphere. The mixture was allowed to cool down to rt. The resulting mixture was filtered, the filter cake was washed with CH3CN (2×20 mL). The filtrate was concentrated under reduced pressure. The residue was purified by silica gel column chromatography, eluted with THF/PE (1:3-5:1) to afford methyl 4-(benzyloxy)-6-({2-[2-({3-[(tert-butyldiphenylsilyl)oxy]-4,5-dimethoxyphenyl}methoxy)ethoxy]ethyl}(methyl)amino)hexanoate (3.5 g, 41% yield) as a brown yellow oil.

LC-MS (ES+) m/z: 758.4 (M+H)+ (calculated: 758.4).

To a stirred solution of methyl 4-(benzyloxy)-6-({2-[2-({3-[(tert-butyldiphenylsilyl)oxy]-4,5-dimethoxyphenyl}methoxy)ethoxy]ethyl}(methyl)amino)hexanoate (3.5 g, 4.62 mmol, 1.00 equiv) in THF (50 mL) was added LiAlH₄ (0.53 g, 13.85 mmol, 3.00 equiv) in portions at 0° C. under N2 atmosphere. The resulting mixture was stirred for 1 h at 0° C. under N2 atmosphere. The reaction was quenched by the addition of H₂O (1 mL) at 0° C., then 3 mL 15% NaOH and 1 mL H₂O. To the mixture was added Na₂SO₄ (20 g) at rt. The resulting mixture was stirred for additional 2h at rt. The resulting mixture was filtered, the filter cake was washed with ethyl acetate (3×20 mL). The filtrate was concentrated under reduced pressure. This resulted in 4-(benzyloxy)-6-({2-[2-({3-[(tert-butyldiphenylsilyl)oxy]-4,5-dimethoxyphenyl}methoxy)ethoxy]ethyl}(methyl)amino)hexan-1-ol (3.3 g, 83% yield) as a light-yellow oil

LC-MS (ES+) m/z: 730.4 (M+H)+ (calculated: 730.4).

To a stirred solution of 4-(benzyloxy)-6-({2-[2-({3-[(tert-butyldiphenylsilyl)oxy]-4,5-dimethoxyphenyl}methoxy)ethoxy]ethyl}(methyl)amino)hexan-1-ol (3.3 g, 4.52 mmol, 1.00 equiv) in THF (50 mL) was added TBAF (6.78 mL, 6.78 mmol, 1.50 equiv) at rt. The resulting mixture was stirred for 1.5h at rt. The reaction was quenched with a saturated solution of NH₄Cl (100 mL) at rt. The resulting mixture was extracted with ethyl acetate (5×150 mL). The combined organic layers were washed with brine (1×100 mL), dried over anhydrous Na₂SO₄. After filtration, the filtrate was concentrated under reduced pressure. The residue was purified by silica gel column chromatography, eluted with MeOH/DCM (1:10-1:4), then purified by reverse flash chromatography with the following conditions: column, C18 silica gel; mobile phase, MeCN in 0.1% NH3·H2O, 30% to 80% gradient in 10 min, detector, UV 254 nm. to afford 5-[11-(3-hydroxypropyl)-8-methyl-13-phenyl-2,5,12-trioxa-8-azatridecan-1-yl]-2,3-dimethoxyphenol (900 mg, 40% yield) as a light-yellow oil.

LC-MS (ES+) m/z: 492.3 (M+H)+ (calculated: 492.3).

To a stirred solution of 5-[11-(3-hydroxypropyl)-8-methyl-13-phenyl-2,5,12-trioxa-8-azatridecan-1-yl]-2,3-dimethoxyphenol (900 mg, 1.83 mmol, 1.00 equiv) in THF (40 mL) were added ADDP (1.37 g, 5.49 mmol, 3.00 equiv) and n-Bu3P (1.11 g, 5.49 mmol, 3.00 equiv) at room temperature under N2 atmosphere. The resulting mixture was stirred for 30 min at room temperature under N2 atmosphere. The reaction was quenched with a saturated solution of NH4Cl (100 mL) at rt. The resulting mixture was extracted with ethyl acetate (3×100 mL) The combined organic layers were washed with brine (1×100 mL), dried over anhydrous Na₂SO₄. After filtration, the filtrate was concentrated under reduced pressure. The residue was purified by silica gel column chromatography, eluted with THF/PE (1:3-1:0), then MeOH/DCM (1:10-1:4) to afford 6-(benzyloxy)-19,20-dimethoxy-9-methyl-2,12,15-trioxa-9-azabicyclo[15.3.1]henicosa-1(20),17(21),18-triene (350 mg, 40% yield) as a light-yellow oil.

LC-MS (ES+) m/z: 474.3 (M+H)+ (calculated: 474.3).

To a solution of 6-(benzyloxy)-19,20-dimethoxy-9-methyl-2,12,15-trioxa-9-azabicyclo[15.3.1]henicosa-1(20),17(2l),18-triene (350 mg, 0.74 mmol, 1.00 equiv) in EtOH (20 mL) was added Pd/C (50 mg) under nitrogen atmosphere in a 100 mL round bottom flask. The mixture was hydrogenated at room temperature for 1 h under hydrogen atmosphere using a hydrogen balloon, filtered through a Celite pad and concentrated under reduced pressure to give the intermediate compound 27 (200 mg, 63% yield) as a colorless oil.

LC-MS (ES+) m/z: 384.3 (M+H)+ (calculated: 384.3).

### Intermediate Compound 28

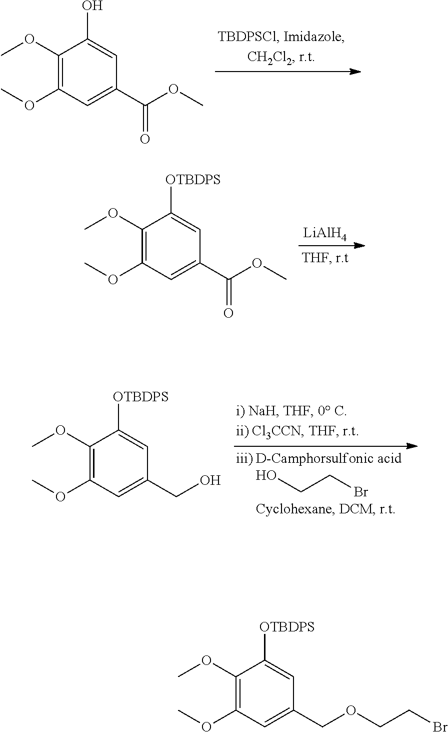

To a solution of methyl 3-hydroxy-4,5-dimethoxybenzoate (5.00 g, 23.6 mmol, 1.0 equiv) and imidazole (2.41 g, 35.3 mmol, 1.5 equiv) in DCM (160 mL) was added tert-butyl(chloro)diphenylsilane (7.05 mL, 27.1 mmol, 1.15 equiv.) dropwise at 0° C. under a nitrogen atmosphere. The reaction mixture was stirred overnight at room temperature under a nitrogen atmosphere. The reaction was quenched by the addition of water (200 mL) at room temperature. The resulting mixture was extracted with EtOAc (3×100 mL). The combined organic layers were washed with brine (1×100 mL), dried over anhydrous Na₂SO₄ and concentrated under reduced pressure. The residue was purified by silica gel column chromatography, eluting with PE/EA (0-50% gradient) to afford methyl 3-((tert-butyldiphenylsilyl)oxy)-4,5-dimethoxybenzoate (10 g, 95% yield) as a colourless solid.

1H NMR (400 MHz, CDCl3-d) δ 7.76-7.66 (m, 4H), 7.47-7.32 (m, 6H), 7.17 (s, 1H), 6.97 (s, 1H), 3.85 (s, 3H), 3.75 (s, 3H), 3.72 (s, 3H), 1.13 (s, 9H)

To a solution of methyl 3-((tert-butyldiphenylsilyl)oxy)-4,5-dimethoxybenzoate (5.30 g, 11.7 mmol, 1.0 equiv) in THF (51 mL) was added 2 M LiAlH₄ in THF (11.8 mL, 23.52 mmol, 2.0 equiv.) dropwise at 0° C. under a nitrogen atmosphere. The reaction mixture was stirred overnight at room temperature under a nitrogen atmosphere. The reaction mixture was cooled to 0° C. (ice bath) and diluted with THF (50 mL). Water (1 mL) as added dropwise to the cooled reaction mixture, followed by 15% aq. NaOH (1 mL) and additional water (3 mL) The reaction mixture was warmed to room temperature and stirred for 15 mins. The quenched reaction mixture was dried over anhydrous MgSO₄ and concentrated under reduced pressure. The residue was purified by silica gel column chromatography, eluting with PE/EA (20-100% gradient) to afford (3-((tert-butyldiphenylsilyl)oxy)-4,5-dimethoxyphenyl)methanol (3.45 g, 69% yield) as a colourless oil.

1H NMR (400 MHz, CDCl3-d) δ 7.77-7.68 (m, 4H), 7.46-7.32 (m, 6H), 6.51 (s, 1H), 6.14 (s, 1H), 4.29 (s, 2H), 3.83 (s, 3H), 3.79 (s, 3H), 1.12 (s, 9H).

To a solution of (3-((tert-butyldiphenylsilyl)oxy)-4,5-dimethoxyphenyl)methanol (1.27 g, 3.00 mmol, 1.5 equiv) in anhydrous THF (1.95 mL) under a nitrogen atmosphere was slowly added sodium hydride (10 mg, 0.400 mmol, 0.2 equiv.) in anhydrous THE (2.22 mL) and the reaction mixture was stirred at room temperature for 30 mins. The reaction mixture was cooled to 0° C. and trichloroacetonitrile (0.30 mL, 3.00 mmol, 1.5 equiv.) was added and the reaction mixture was slowly warmed to room temperature and stirred for 4 h. The reaction mixture was concentrated under reduced pressure. The residue was suspended in heptane (50 mL) and methanol (0.2 mL) and filtered through celite. The filtrate was concentrated under reduced pressure to afford a yellow oil. The crude oil was dissolved in cyclohexane (9.00 mL) and a solution of 2-bromoethanol (250 mg, 2.00 mmol, 1.0 equiv.) in DCM (1.70 mL) was added. The resulting mixture was cooled to 0° C. (ice bath) and (±)-10-camphorsulfonic acid (46 mg, 0.20 mmol, 0.1 equiv.) was added. The reaction mixture was warmed to room temperature and stirred overnight. The resulting colourless precipitate formed was filtered though celite and washed with cyclohexane/DCM (1.2, 40 mL) The filtrate was concentrated under reduced pressure and the crude residue was purified by silica gel column chromatography, eluting with PE/EA (0-50% gradient) to afford the intermediate compound 28 (575 mg, 54% yield) as a colourless solid.

1H NMR (400 MHz, CDCl3-d) δ 7.78-7.67 (m, 4H), 7.45-7.32 (m, 6H), 6.48 (d, J=1.9 Hz, 1H), 6.12 (d, J=1.8 Hz, 1H), 4.19 (s, 2H), 3.88-3.80 (m, 6H), 3.43-3.35 (m, 2H), 3.27-3.20 (m, 2H), 1.12 (d, J=3.3 Hz, 9H)

### Intermediate Compound 29

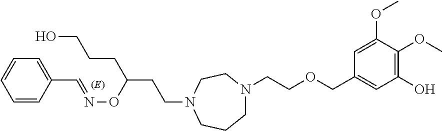 [SMILES: COc1cc(COCCN2CCCN(CCC(CCCO)O/N=C/c3ccccc3)CC2)cc(O)c1OC]

The intermediate compound 29 was prepared from the intermediate compound 1 (161 mg, 0.302 mmol, 1.0 equiv) and the intermediate compound 28 (174 mg, 0.330 mmol, 1.1 equiv) using the protocol described for the intermediate compound 3 The intermediate compound 29 (90 mg, 57% yield over 3 steps) was isolated as a light yellow oil.

LCMS (ESI position ion) m/z: 530.5 (M+H)+ (calculated: 530.3)

### Intermediate Compound 30

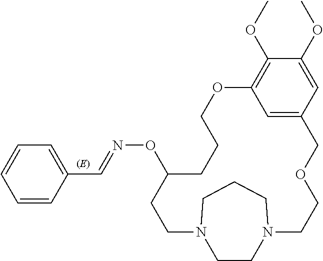 [SMILES: COc1cc2cc(c1OC)OCCCC(O/N=C/c1ccccc1)CCN1CCCN(CCOC2)CC1]

The intermediate compound 30 was prepared from the intermediate compound 29 (80 mg, 0.151 mmol, 1.0 equiv) using the protocol described for the compound 1. After purification by reverse flash chromatography (column: C18 silica gel; mobile phase, MeCN (0.1% Formic Acid) in water (0.1% Formic Acid), (10-60% gradient), UV 254 nm and 220 nm), the intermediate compound 30 (39 mg, 39% yield) was isolated as a white solid LCMS (ESI position ion) m/z: 512.5 (M+H)+ (calculated: 512.3)

### Intermediate Compound 31

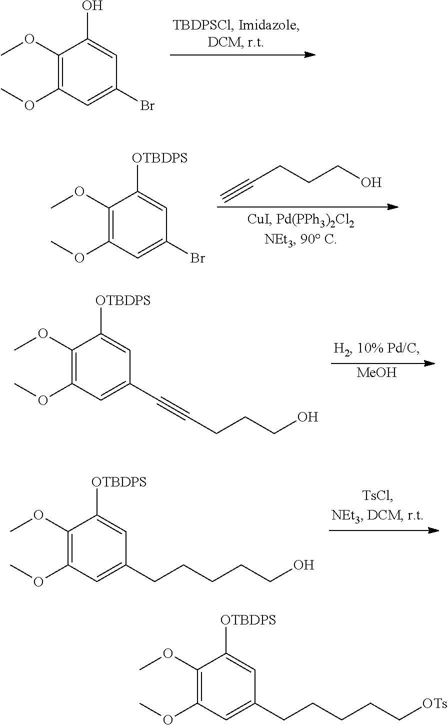

To a solution of 5-bromo-2,3-dimethoxyphenol (850 mg, 3.674 mmol, 1.0 equiv) and imidazole (372 mg, 5.47 mmol, 1.5 equiv) in DCM (25 mL) was added tert-butyl(chloro)diphenylsilane (1.04 mL, 4.01 mmol, 1.1 equiv.) dropwise at 0° C. under a nitrogen atmosphere. The reaction mixture was stirred overnight at room temperature under a nitrogen atmosphere. The reaction was quenched by the addition of water (100 mL) at room temperature. The resulting mixture was extracted with DCM (3×50 mL). The combined organic layers were washed with brine (1×50 mL), dried over anhydrous Na₂SO₄ and concentrated under reduced pressure. The residue was purified by silica gel column chromatography, eluting with PE/EA (0-50% gradient) to afford (5-bromo-2,3-dimethoxyphenoxy)(tert-butyl)diphenylsilane (1.38 g, 80% yield) as a colourless solid.

1H NMR (400 MHz, CDCl3-d) δ 7.72 (dt, J=7.9, 1.4 Hz, 4H), 7.47-7.33 (m, 6H), 6.60 (d, J=2.2 Hz, 1H), 6.35 (d, J=2.2 Hz, 1H), 3.80 (s, 3H), 3.73 (s, 3H), 1.11 (s, 9H)

To a sealed microwave vial charged with (5-bromo-2,3-dimethoxyphenoxy)(tert-butyl)diphenylsilane (600 mg, 1.27 mmol, 1.0 equiv), pent¬4 yn 1 of (117 mg, 1.40 mmol, 1.1 equiv), tetrakis(triphenylphosphine)palladium(0) (74 g, 0.064 mmol, 0.05 equiv) and copper(I) iodide (24 mg, 0.127 mmol, 0.1 equiv) under a nitrogen atmosphere was added triethylamine (4.8 mL) and the reaction mixture was stirred overnight at 90° C. The reaction mixture was cooled to room temperature and was diluted with water (50 mL) and extracted with EtOAc (3×50 mL). The combined organic components were washed with brine (1×50 mL), dried over anhydrous MgSO₄ and concentrated under reduced pressure to afford a brown oil. The residue was purified by silica gel column chromatography, eluting with PE/EA (0-100% gradient) to afford 5-(3-((tert-butyldiphenylsilyl)oxy)-4,5-dimethoxyphenyl)pent-4-yn-1-ol (500 mg, 83% yield) as a colourless oil.

LCMS (ESI position ion) m/z: 475.2 (M+H)+ (calculated: 475.3)

1H NMR (400 MHz, CDCl3-d) δ 7.77-7.68 (m, 5H), 7.43-7.33 (m, 7H), 6.51 (d, J=1.9 Hz, 1H), 6.31 (d, J=1.9 Hz, 1H), 3.79 (s, 3H), 3.75-3.68 (m, 5H), 2.40 (t, J=6.9 Hz, 2H), 1.81-1.70 (m, 2H), 1.10 (s, 9H)

A solution of 5-(3-((tert-butyldiphenylsilyl)oxy)-4,5-dimethoxyphenyl)pent-4-yn-1-ol (500 mg, 1.05 mmol, 1.0 equiv) and 10% palladium on carbon (112 mg) in methanol (10.5 mL) under H2 atmosphere (Endeavor, 15 psi) was stirred at room temperature for 12 h. The reaction mixture was filtered through celite and washed with methanol. The filtrate was evaporated in vacuo to afford a colourless oil. The oil was purified by column chromatography (0-100% EtOAc/Heptane) to afford 5-(3-((tert-butyldiphenylsilyl)oxy)-4,5-dimethoxyphenyl)pentan-1-ol (452 mg, 90% yield) as a colourless oil.

1H NMR (400 MHz, CDCl3-d) δ 7.79-7.68 (m, 4H), 7.48-7.30 (m, 6H), 6.26 (d, J=1.9 Hz, 1H), 5.96 (d, J=1.9 Hz, 1H), 3.81 (s, 6H), 3.53 (t, J=6.6 Hz, 2H), 2.23 (t, J=7.5 Hz, 2H), 1.48-1.35 (m, 6H), 1.12 (s, 9H)

To a stirred solution of 5-(3-((tert-butyldiphenylsilyl)oxy)-4,5-dimethoxyphenyl)pentan-1-ol (450 mg, 0.940 mmol, 1.0 equiv) and trimethylamine (0.26 mL, 1.88 mmol, 1.0 equiv) in DCM (2.75 mL) was added portionwise para-toluenesulfonyl chloride (269 mg, 1.41 mmol, 1.5 equiv) and the reaction mixture stirred overnight at room temperature under a nitrogen atmosphere. The reaction was quenched by the addition of water (50 mL) at room temperature. The resulting mixture was extracted with DCM (3×50 mL). The combined organic layers were washed with brine (1×50 mL), dried over anhydrous Na₂SO₄ and concentrated under reduced pressure. The residue was purified by silica gel column chromatography, eluting with PE/EA (50-100% gradient) to afford the intermediate compound 31 (375 mg, 63% yield) as a yellow oil.

1H NMR (400 MHz, CDCl3-d) δ 7.87-7.68 (m, 6H), 7.48-7.30 (m, 8H), 6.22 (d, J=1.9 Hz, 1H), 5.90 (d, J=1.9 Hz, 1H), 3.92-3.88 (m, 2H), 3.81 (d, J=5.0 Hz, 6H), 2.43 (d, J=2.8 Hz, 3H), 2.16 (t, J=7.4 Hz, 2H), 1.54-1.42 (m, 4H), 1.11 (s, 7H), 0.92-0.84 (m, 2H).

### Intermediate Compound 32

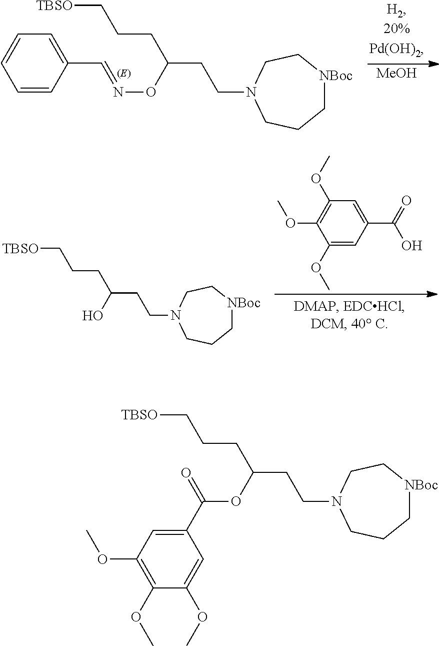

A solution of intermediate compound 1 (931 mg, 1.74 mmol, 1.0 equiv) and 20% palladium hydroxide on carbon (195 mg) in methanol (15 mL) under 12 atmosphere (Endeavor, 15 psi) was stirred at room temperature for 3 h. The reaction mixture was filtered through celite and washed with methanol. The filtrate was evaporated in vacuo to afford a colourless oil. The oil was purified by column chromatography (0-100% EtOAc/Heptane) to afford tert-butyl 4-(6-((tert-butyldimethylsilyl)oxy)-3-hydroxyhexyl)-1,4-diazepane-1-carboxylate as a colourless oil (730 mg, 97% yield).

1H NMR (400 MHz, CDCl3-d) δ 3.82-3.74 (m, 1H), 3.70-3.59 (m, 2H), 3.58-3.33 (m, 4H), 2.88-2.52 (m, 6H), 1.89 (br. s, 2H), 1.76-1.48 (m, 6H), 1.45 (s, 9H), 0.89 (s, 9H), 0.04 (s, 6H)

To a solution of tert-butyl 4-[(3R)-6-[(tert-butyldimethylsilyl)oxy]-3-hydroxyhexyl]-1,4-diazepane-1-carboxylate (730 mg, 1.695 mmol, 1.0 equiv), EDC·HCl (650 mg, 3.390 mmol, 2.0 equiv.) and 3,4,5-trimethoxybenzoic acid (648 mg, 3.051 mmol, 1.5 equiv.) in DCM (8.8 mL) was added DMAP (414 mg, 3.390 mmol, 2.0 equiv.) and the reaction mixture was stirred overnight at 40° C. under a nitrogen atmosphere. The reaction was quenched by the addition of water (50 mL) at room temperature. The resulting mixture was extracted with EtOAc (3×50 mL). The combined organic layers were washed with brine (1×50m L), dried over anhydrous Na₂SO₄ and concentrated under reduced pressure. The residue was purified by silica gel column chromatography, eluting with PE/EA (0-100% gradient) to afford the intermediate compound 32 (375 mg, 35% yield) as a colourless solid.

LCMS (ESI position ion) m/z: 626.0 (M+H)+ (calculated: 626.4)

### Intermediate Compound 33

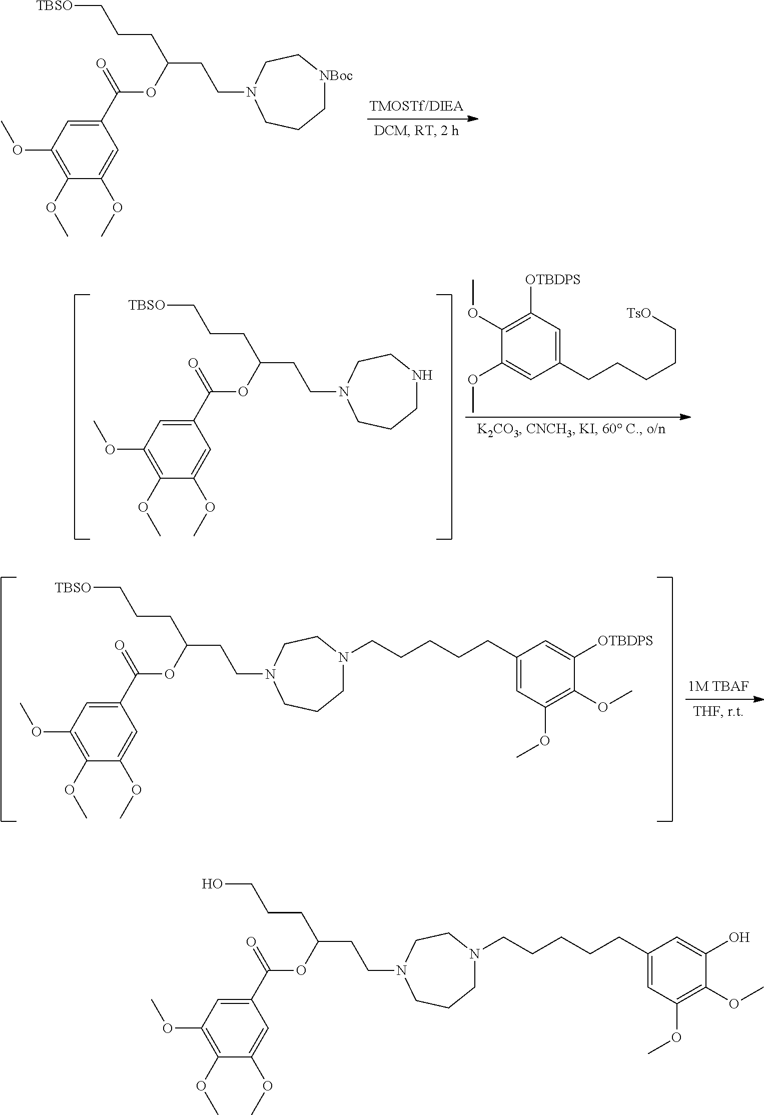

To a solution of intermediate compound 32 (235 mg, 0.376 mmol, 1.0 equiv) and DIPEA (0.26 mL, 1.29 mmol, 4.0 equiv) in DCM (3.6 mL), was added TMSOTf (0.20 mL, 1.28 mmol, 3.0 equiv) at 0° C. The resulting solution was stirred for 2 h at room temperature, and then quenched by the addition of water (10 mL). The organic phase was separated and washed with water (10 mL), followed by brine (10 mL). The organic layer was dried over anhydrous sodium sulfate and concentrated. The residue was dissolved in MeCN (2.0 mL) and DIPEA (0.13 mL, 0.7.62 mmol, 2.0 equiv) was added, followed by intermediate compound 31 (362 mg, 0.572 mmol, 1.5 equiv) and sodium iodide (29 mg, 0.191 mmol, 0.5 equiv) at room temperature under a nitrogen atmosphere. The reaction mixture was stirred overnight at 60° C., and was allowed to cool down to room temperature. The reaction was diluted with water (50 mL) and the resulting mixture was extracted with EtOAc (3×50 mL). The combined organic layers were washed with brine (1×50 mL), dried over anhydrous MgSO₄ and concentrated under reduced pressure. The residue was dissolved in anhydrous THF (3.4 mL) and 1 M TBAF (1.52 mL 1.52 mmol, 4 equiv.) was added dropwise at 0° C. under a nitrogen atmosphere. The reaction mixture was stirred for 2 h at room temperature. The reaction was quenched by the addition of water (50 mL) at room temperature. The resulting mixture was extracted with CHCl₃/MeOH (9.1, 3×50 mL). The combined organic layers were washed with brine (1×50 mL), dried over anhydrous MgSO₄ and concentrated under reduced pressure. The residue was purified by reverse flash chromatography (column, C18 silica gel; mobile phase, MeCN (0.1% Formic Acid) in water (0.1% Formic Acid), (10-60% gradient), UV 254 nm and 220 nm) to afford a yellow oil. To the residue was added a saturated solution of NaHCO₃ (50 mL) and extracted with CHCl3/MeOH (9:1, 3×20 mL). The combined organic layers were dried over anhydrous MgSO₄ and concentrated under reduced pressure to afford the intermediate compound 33 (77 mg, 32% yield over 3 steps) as a light yellow oil and was used without further purification.

LCMS (ESI position ion) m/z: 633.5 (M+H)+ (calculated: 633.4)

### Intermediate Compound 34

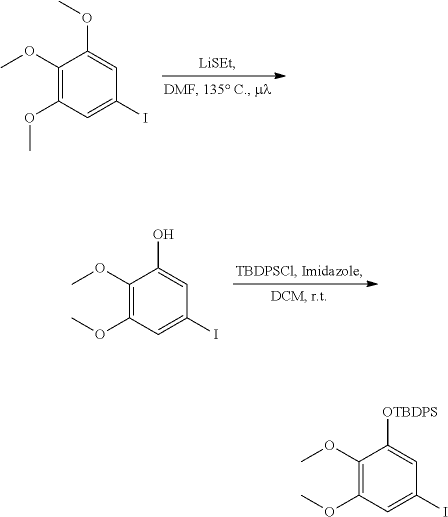

To a sealed microwave vial with 5-iodo-1,2,3-trimethoxy benzene (500 mg, 1.70 mmol, 1.0 equiv) and (ethylsulfanyl)lithium (289 mg, 3.4 mmol, 2.0 equiv), evacuated with nitrogen, was added DMF (13 mL) and the reaction mixture was irradiated in a microwave at 135° C. for 15 mins. The cooled mixture was diluted with TMBE (50 mL) and 1 M aq. HCl (50 mL) was added. The organic component was separated and the aqueous layer was extracted with TBME (2×50 mL). The combined organic components were washed with brine, dried (MgSO4) and evaporated in vacuo to afford a crude oil. The crude oil was purified by silica gel column chromatography, eluting with PE/EA (0-50% gradient) to afford 5-iodo-2,3-dimethoxyphenol (173 mg, 36% yield) as a colourless solid.

1H NMR (400 MHz, CDCl3-d) δ 6.97 (d, J=2.0 Hz, 1H), 6.78 (d, J=2.0 Hz, 1H), 5.73 (s, 1H), 3.87 (s, 3H), 3.84 (s, 3H).

To a solution of 5-iodo-2,3-dimethoxyphenol (173 mg, 0.618 mmol, 1.0 equiv) and imidazole (63 mg, 0.927 mmol, 1.5 equiv) in DCM (4.2 mL) was added tert-butyl(chloro)diphenylsilane (0.176 mL, 0.680 mmol, 1.1 equiv.) dropwise at 0° C. under a nitrogen atmosphere. The reaction mixture was stirred overnight at room temperature under a nitrogen atmosphere. The reaction was quenched by the addition of water (50 mL) at room temperature. The resulting mixture was extracted with DCM (3×50 mL). The combined organic layers were washed with brine (1×50 mL), dried over anhydrous Na₂SO₄ and concentrated under reduced pressure. The residue was purified by silica gel column chromatography, eluting with PE/EA (0-50% gradient) to afford tert-butyl(5-iodo-2,3-dimethoxyphenoxy)diphenylsilane (160 mg, 50% yield) as a colourless solid.

1H NMR (400 MHz, CDCl3-d) δ 7.75-7.67 (m, 4H), 7.48-7.34 (m, 5H), 6.76 (d, J=2.0 Hz, 1H), 6.53 (d, J=2.0 Hz, 1H), 3.79 (s, 3H), 3.72 (s, 3H), 1.11 (s, 9H).

### Intermediate Compound 35

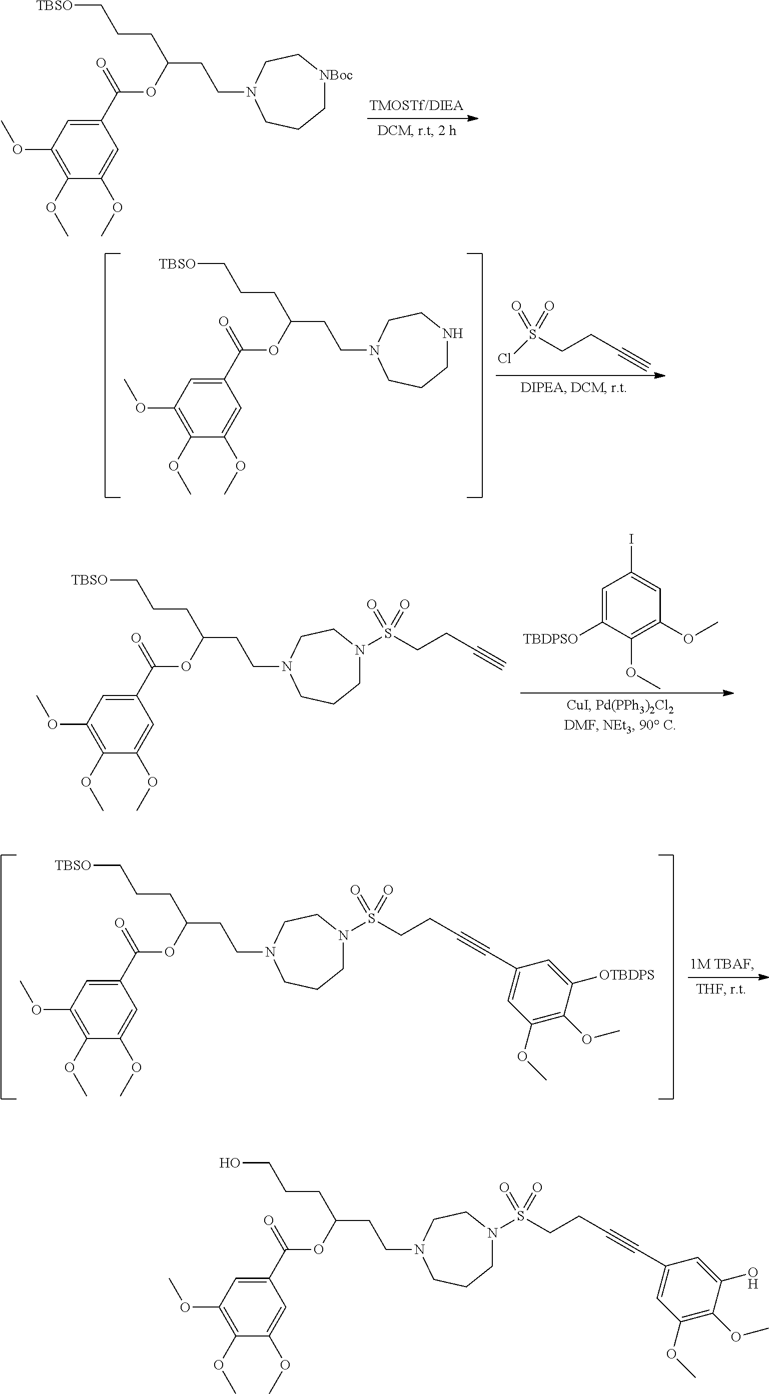

To a solution of intermediate compound 32 (486 mg, 0.778 mmol, 1.0 equiv) and DIPEA (0.54 mL, 3.11 mmol, 4.0 equiv) in DCM (7.5 mL), was added TMSOTf (0.42 mL, 2.33 mmol, 3.0 equiv) at 0° C. The resulting solution was stirred for 2 h at room temperature, and then quenched by the addition of water (10 mL). The organic phase was separated and washed with water (10 mL), followed by brine (10 mL). The organic layer was dried over anhydrous sodium sulfate and concentrated. The residue was dissolved in DCM (7.75 mL) and was added DIPEA (0.27 mL, 1.55 mmol, 2.0 equiv), followed by but-3-yne-1-sulfonyl chloride (0.103 mL, 0.932 mmol, 1.2 equiv). The reaction mixture was stirred at room temperature overnight. The reaction mixture was quenched with water (20 mL) and extracted with DCM (3×50 mL). The combined organic layers were washed with brine (1×50 mL), dried over anhydrous Na2SO4 and concentrated under reduced pressure. The residue was purified by silica gel column chromatography, eluting with EA/ethanol (0-30% gradient) to afford 1-(4-(but-3-yn-1-ylsulfonyl)-1,4-diazepan-1-yl)-6-((tert-butyldimethylsilyl)oxy) hexan-3-yl 3,4,5-trimethoxybenzoate (330 mg, 66% yield over 2 steps) as a yellow oil.

LCMS (ESI position ion) m/z: 641.9 (M+H)+ (calculated: 641.3) 1H NMR (400 MHz, CDCl3-d) δ 7.28 (s, 2H), 5.29-5.16 (m, 1H), 3.91 (s, 9H), 3.63 (t, J=6.4 Hz, 2H), 3.48-3.38 (m, 4H), 3.15 (t, J=7.5 Hz, 2H), 2.74-2.67 (m, 6H), 2.59 (t, J=7.3 Hz, 2H), 2.09-2.02 (m, 1H), 1.94-1.70 (m, 5H), 1.68-1.51 (m, 7H), 1.26 (t, J=7.1 Hz, 1H), 0.88 (s, 9H), 0.03 (s, 6H).

To a sealed microwave vial charged with 1-(4-(but-3-yn-1-ylsulfonyl)-1,4-diazepan-1-yl)-6-((tert-butyldimethylsilyl)oxy)hexan-3-yl 3,4,5-trimethoxybenzoate (226 mg, 0.353 mmol, 1.0 equiv), intermediate compound 34 (219 mg, 0.424 mmol, 1.2 equiv), bis(triphenylphosphine)palladium(II) dichloride (25 g, 0.035 mmol, 0.05 equiv) and copper(I) iodide (14 mg, 0.071 mmol, 0.1 equiv) under a nitrogen atmosphere was added triethylamine (1.1 mL) and DMF (1.1 mL) and the reaction mixture was stirred overnight at room temperature.

The reaction mixture was cooled to room temperature and was diluted with water (50 mL) and extracted with EtOAc (3×50 mL) The combined organic components were washed with brine (1×50 mL), dried over anhydrous MgSO₄ and concentrated under reduced pressure to afford a brown oil. The residue was dissolved in anhydrous THF (3.4 mL) and 1 M TBAF (1.52 mL 1.52 mmol, 4 equiv.) was added dropwise at 0° C. under a nitrogen atmosphere. The reaction mixture was stirred for 2 h at room temperature. The reaction was quenched by the addition of water (50 mL) at room temperature. The resulting mixture was extracted with EtOAc (3×50 mL). The combined organic layers were washed with brine (1×50 mL), dried over anhydrous MgSO₄ and concentrated under reduced pressure. The residue was purified by reverse flash chromatography (column, C18 silica gel; mobile phase, MeCN (0.1% Formic Acid) in water (0.1% Formic Acid), (0-60% gradient), UV 254 nm and 220 nm) to afford a yellow oil. To the residue was added a saturated solution of NaHCO₃ (50 mL) and extracted with CHCl3/MeOH (9:1, 3×20 mL). The combined organic layers were dried over anhydrous MgSO₄ and concentrated under reduced pressure to afford the intermediate compound 35 (90 mg, 37% yield over 2 steps) as a light yellow oil.

LCMS (ESI position ion) m/z: 679.5 (M+H)+ (calculated: 679.3)

1H NMR (400 MHz, CDCl3-d) δ 7.28 (s, 2H), 6.66 (d, J=1.8 Hz, 1H), 6.52 (d, J=1.8 Hz, 1H), 5.32-5.21 (m, 1H), 3.91 (s, 9H), 3.89 (s, 3H), 3.84 (s, 3H), 3.69 (t, J=6.2 Hz, 2H), 3.47 (q, J=5.5, 4.6 Hz, 4H), 3.23 (t, J=7.5 Hz, 2H), 2.88 (t, J=7.4 Hz, 2H), 2.76-2.67 (m, 4H), 2.63-2.58 (m, 2H), 1.96-1.76 (m, 4H), 1.58-1.55 (m, 6H).

### Synthesis of Final Compounds

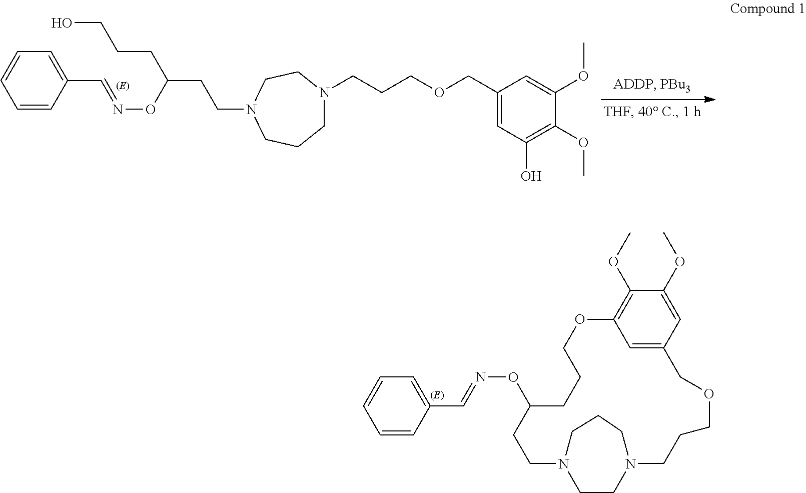 [SMILES: COc1cc(COCCCN2CCCN(CCC(CCCO)O/N=C/c3ccccc3)CC2)cc(O)c1OC ; COc1cc2cc(c1OC)OCCCC(O/N=C/c1ccccc1)CCN1CCCN(CCCOC2)CC1]

To a stirred mixture of ADDP (327 mg, 1.31 mmol, 1.5 equiv) in THF (10 mL) was added n-tributyl phosphine (0.326 mL, 1.31 mmol, 1.5 equiv) at room temperature under nitrogen atmosphere and the mixture was stirred for 30 mins. Intermediate compound 3 (473 mg, 0.870 mmol, 1.0 equiv) in THF (5.8 mL) was added and the resulting mixture was stirred for additional 1 h at 40° C., and then allowed to cool down to room temperature. The reaction was quenched with 50% brine (30 mL) at room temperature. The resulting mixture was extracted with EtOAc (3×50 mL). The combined organic layers were dried over anhydrous Na₂SO₄ and concentrated under reduced pressure. The residue was purified by reverse flash chromatography (column, C18 silica gel; mobile phase, MeCN (0.1% Formic Acid) in water (0.1% Formic Acid), (10-50% gradient). UV 254 nm and 220 nm), affording the compound 1 (190 mg, 41% yield) as a colourless solid.

LCMS (ESI position ion) m/z: 526.4 (M+H)+ (calculated: 526.3)

1H NMR (400 MHz, CDCl3-d) δ 8.06 (s, 1H), 7.56-7.48 (m, 2H), 7.44-7.32 (m, 4H), 6.69 (d, J=1.8 Hz, 1H), 6.46 (d, J=1.7 Hz, 1H), 4.56-4.17 (m, 3H), 4.24-4.08 (m, 2H), 3.86 (s, 3H), 3.82 (s, 3H), 3.67-3.51 (m, 2H), 3.42-3.35 (m, 2H), 3.18-2.92 (m, 4H), 2.90-2.54 (m, 6H), 2.20-1.75 (m, 10H)

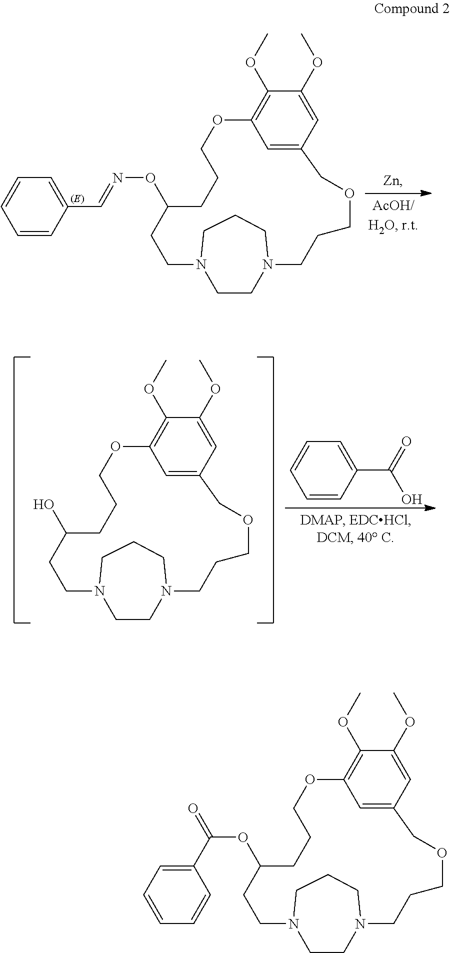

To a mixture of compound 1 (90 mg, 0.171 mmol, 1.0 equiv.) in acetic acid (1.80 mL) and water (1.80 mL) was added zinc dust (223 mg, 3.42 mmol, 20 equiv.) and the reaction mixture was stirred at room temperature for 3 h. The reaction mixture was quenched with 1 M aq. Na2CO3 (10 mL) and extracted with EtOAc (3×10 mL). The combined organic components were washed with brine (10 mL), dried over anhydrous Na₂SO₄ and concentrated under reduced pressure. The residue was dissolved in DCM (0.9 mL) and DMAP (41.9 mg, 0.342 mmol, 2.0 equiv), EDC·HCl (52.0 mg, 0.342 mmol, 2.0 equiv) and benzoic acid (32.0 mg, 0.257 mmol, 1.5 equiv) were added and the reaction mixture was stirred overnight at 40° C. The reaction was quenched with H2O (10 mL) and extracted with DCM (3×10 mL) The combined organic layers were washed with brine (10 mL), dried over anhydrous Na₂SO₄ and concentrated under reduced pressure. The residue was purified by reverse flash chromatography (column, C18 silica gel, mobile phase, MeCN (0.1% Formic Acid) in water (0.1% Formic Acid), (10-50% gradient), UV 254 nm and 220 nm), affording the compound 2 (6 mg, 7% yield) as a colourless solid.

LCMS (ESI position ion) m/z: 527.5 (M+H)+ (calculated: 527.3)

1H NMR (400 MHz, CDCl3-d) δ 8.07-7.99 (m, 2H), 7.63-7.54 (m, 1H), 7.50-7.42 (m, 2H), 6.72 (s, 1H), 6.46 (s, 1H), 5.62-5.51 (m, 1H), 4.53 (d, J=12.2 Hz, 1H), 4.34 (d, J=12.2 Hz, 1H), 4.19-4.11 (m, 2H), 3.86 (s, 3H), 3.81 (s, 3H), 3.71-3.50 (m, 2H), 3.33-3.27 (m, 2H), 3.12 (J=22.8, 12.6, 5.6 Hz, 4H), 2.98-2.66 (m, 3H), 2.65-2.49 (m, 4H), 2.13-1.77 (m, 10H)

### Compound 3, Compound 4 and Compound 5

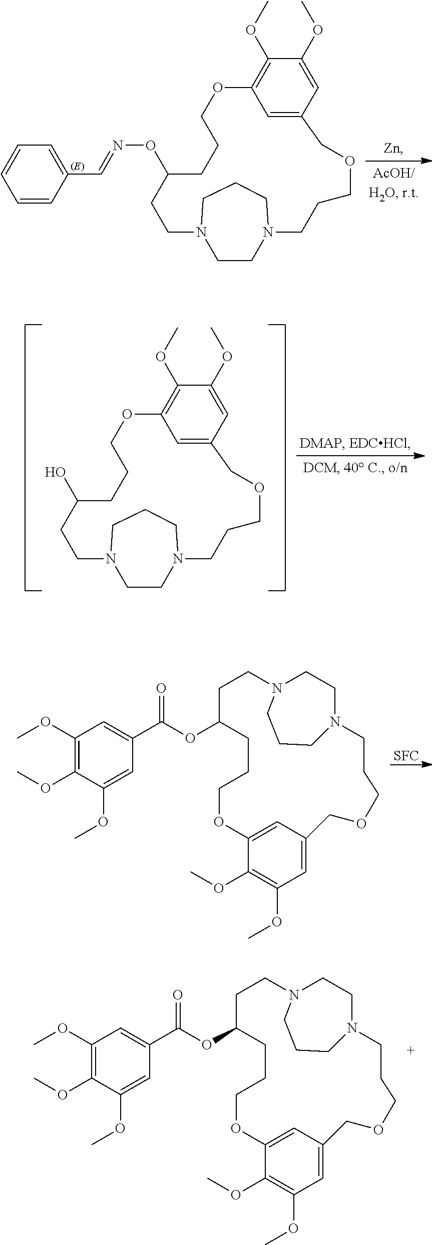

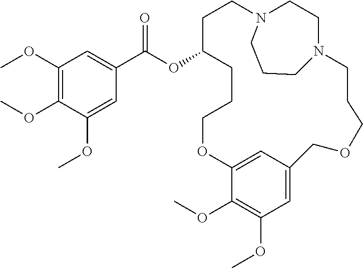 [SMILES: COc1cc(C(=O)O[C@H]2CCCOc3cc(cc(OC)c3OC)COCCCN3CCCN(CC2)CC3)cc(OC)c1OC]

The compound 3 was prepared from compound 1 (0.284 mmol, 1.00 eq) and 3,4,5-trimethoxybenzoic acid (52 mg, 0.426 mmol, 1.50 equiv) using the protocol described for compound 2 The residue was purified by reverse flash chromatography (C18; mobile phase, MeCN in water, 20% to 60% gradient in 18 min; detector, UV 220 nm. affording the compound 3 (60 mg, 34% yield) as a colourless oil.

LCMS (ESI position ion) m/z: 617.5 (M+H)+ (calculated: 617.3)

1H NMR (400 MHz, CDCl3-d) δ 7.30 (s, 2H), 6.74 (d, J=1.8 Hz, 1H), 6.49 (d, J=1.8 Hz, 1H), 5.71-5.47 (m, 1H), 4.58 (d, J=12.2 Hz, 1H), 4.34 (d, J=12.2 Hz, 1H), 4.21-4.11 (m, 2H), 3.95 (s, 6H), 3.94 (s, 3H), 3.89 (s, 3H), 3.84 (s, 3H), 3.78-3.56 (m, 2H), 3.45-3.25 (m, 2H), 3.23-3.04 (m, 3H), 3.02-2.72 (m, 2H), 2.72-2.52 (m, 6H), 2.33-1.83 (m, 8H), 1.80-1.66 (m, 1H).

The compound 3 (60 mg) was purified by CHIRAL-HPLC with the following conditions: Column: CHIRALCEL OD-H, 2*25 mm, 5 μm; Mobile Phase A: Hex (0.1% 2M NH3-MeOH)-HPLC, Mobile Phase B: EtOH-HPLC; Flow rate: 25 mL/min; Gradient: 30% B to 30% B in 12 min; Wave Length: 220/254 nm; RT1(min): 6; RT2(min): 9; Sample Solvent: EtOH-HPLC; Injection Volume: 1.5 mL; Number Of Runs: 4.

Compound 4 (22 mg, 37%):

Chiral HPLC (condition 1): retention time=3.8 min, ee=99%

LCMS (ESI position ion) m/z: 617.3 (M+H)+ (calculated: 617.3)

1H NMR (400 MHz, CDCl3-d) δ 7.32 (s, 2H), 6.81 (s, 1H), 6.59 (s, 1H), 5.57 (brs, 1H), 4.50 (s, 2H), 4.26-4.15 (m, 2H), 3.93-3.82 (m, 12H), 3.77 (s, 3H), 3.71-3.50 (m, 2H), 2.89-2.50 (m, 12H), 2.01-1.69 (m, 10H).

Compound 5 (20 mg, 33%):

Chiral HPLC (condition 1): retention time=2.9 min, ee=99%

LCMS (ESI position ion) m/z: 617.3 (M+H)+ (calculated: 617.3)

1H NMR (400 MHz, CDCl3-d) δ 7.32 (s, 2H), 6.81 (s, 1H), 6.59 (s, 1H), 5.57 (brs, 1H), 4.50 (s, 2H), 4.26-4.15 (m, 2H), 3.93-3.82 (m, 12H), 3.77 (s, 3H), 3.71-3.50 (m, 2H), 2.89-2.50 (m, 12H), 2.01-1.69 (m, 10H).

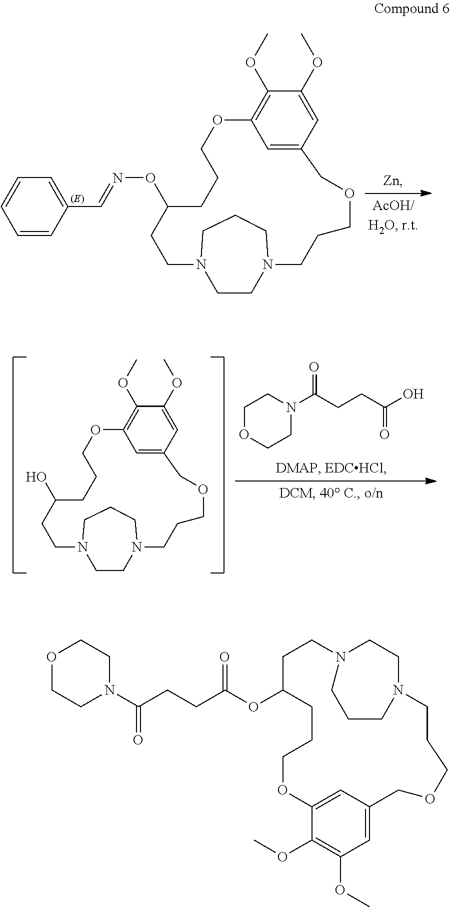

The compound 3 was prepared from compound 1 (0.071 mmol, 1.00 eq) and 4-morpholino-4-oxobutanoic acid (26.56 mg, 0.142 mmol, 2.00 equiv) using the protocol described for compound 2. The purification of the crude by reverse flash chromatography (Column: YMC-Actus Triart C18 ExRS, 30*150 mm, 5 μm; Mobile Phase A: Water (10 mmol/L NH4HCO3), Mobile Phase B: ACN; Flow rate: 35 mL/min; Gradient: 53% B to 82% B in 8 min, 82% B; Wave Length 210 nm; RT1(min): 7.9;) afforded the compound 6 (10 mg, 23%) as a light yellow oil.

LCMS (ESI position ion) m/z: 592.4 (M+H)+ (calculated: 592.4)

1H NMR (400 MHz, CD3OD-d4) δ 6.79 (s, 1H), 6.60 (s, 1H), 5.33 (brs, 1H), 4.48 (s, 2H), 4.21-4.06 (m, 2H), 3.85 (s, 3H), 3.78 (s, 3H), 3.71-3.58 (m, 10H), 2.92-2.70 (m, 14H), 1.98-1.51 (m, 1OH).

### General Procedure 1

To a stirred solution of intermediate compound 4 (1.00 equiv) and the selected acid (2.00 equiv) in DCM (3 mL) were added EDC·HCl (30.74 mg, 0.16 mmol, 2.00 equiv) and DMAP (19.59 mg, 0.16 mmol, 2.00 equiv) at rt. The resulting mixture was stirred for 2 h at rt. The reaction was quenched with 3 mL of a saturated solution of NH₄Cl. The aqueous layer was extracted with DCM (2×3 mL). The combined organic layers were washed with brine (1×3 mL), dried over anhydrous Na₂SO₄. The crude product was purified by Prep-HPLC with the following conditions Column: Xselect CSH F-Phenyl OBD Column 19*150 mm 5 μm, n; Mobile Phase A. Water (0.05% TFA), Mobile Phase B: ACN; Flow rate: 20 mL/min; Gradient: 10% B to 30% B in 8 min, 30% B; Wave Length: 220 nm nm)

The general procedure 1 was used to prepare the compound 6 to compound 16, compounds 33 to 34 and compound 38.

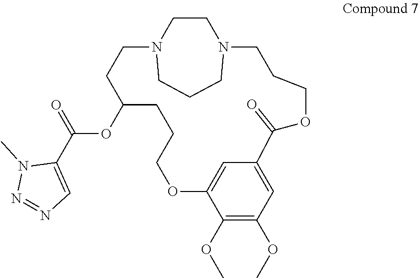 [SMILES: COc1cc2cc(c1OC)OCCCC(OC(=O)c1cnnn1C)CCN1CCCN(CCCOC2=O)CC1]

The compound 7 was prepared by the general procedure 1 using the intermediate compound 4 (35 mg, 0.08 mmol, 1.00 equiv) and 3-methyl-1,2,3-triazole-4-carboxylic acid (20.38 mg, 0.16 mmol, 2.00 equiv). After purification by Prep-HPLC (RT (min): 6.6) the compound 7 (24 mg, TFA salt, 54%) was isolated as an off-white solid.

LCMS (ESI position ion) m/z: 546.3 (M+H)+ (calculated: 546.3)

1H NMR (400 MHz, CD3OD-d4) δ 8.23 (s, 1H), 7.40 (s, 1H), 7.35 (s, 1H), 5.23 (br, 1H), 4.46-4.32 (m, 3H), 4.31 (s, 3H), 4.11 (br, 1H), 3.88 (s, 3H), 3.83 (s, 3H), 3.51-3.31 (m, 5H), 3.13-2.74 (m, 6H), 2.29-1.91 (m, 11H).

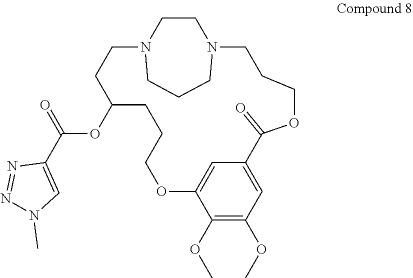 [SMILES: COc1cc2cc(c1OC)OCCCC(OC(=O)c1cn(C)nn1)CCN1CCCN(CCCOC2=O)CC1]

The compound 8 was prepared by the general procedure 1 using the intermediate compound 4 (35 mg, 0.08 mmol, 1.00 equiv) and 1-methyl-1,2,3-triazole-4-carboxylic acid (20.38 mg, 0.16 mmol, 2.00 equiv). After purification by Prep-HPLC (RT (min): 6.2) the compound 8 (17 mg, TFA salt, 39/o) was isolated as an off-white solid.

LCMS (ESI position ion) m/z: 546.3 (M+H)+ (calculated: 546.3)

1H NMR (400 MHz, CD3OD-d4) δ 8.44 (s, 1H), 7.32 (s, 2H), 5.46 (br, 1H), 4.50 (br, 1H), 4.48-4.16 (m, 3H), 4.12 (m, 3H), 3.90 (s, 3H), 3.87-3.72 (m, 4H), 3.66-3.32 (m, 7H), 3.20-3.02 (m, 4H), 2.34-2.02 (m, 10H).

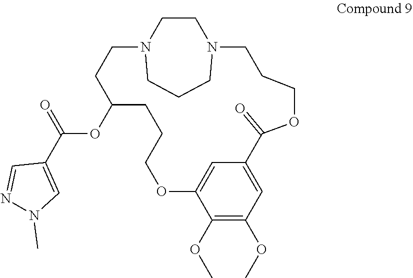 [SMILES: COc1cc2cc(c1OC)OCCCC(OC(=O)c1cnn(C)c1)CCN1CCCN(CCCOC2=O)CC1]

The compound 9 was prepared by the general procedure 1 using the intermediate compound 4 (35 mg, 0.08 mmol, 1.00 equiv) and 1-methylpyrazole-4-carboxylic acid (20.22 mg, 0.16 mmol, 2.00 equiv). After purification by Prep-HPLC (RT (min): 7.9) the compound 9 (9 mg, TFA salt, 20%) was isolated as an off-white solid.

LCMS (ESI position ion) m/z: 545.4 (M+H)+ (calculated: 545.3)

1H NMR (400 MHz, CD3OD-d4) δ 8.17 (s, 1H), 7.88 (s, 1H), 7.42 (s, 1H), 7.33 (s, 1H), 5.44 (br, 1H), 4.45-4.30 (m, 3H), 4.08 (br, 1H), 3.94 (s, 3H), 3.90 (s, 3H), 3.84 (s, 3H), 3.83-3.39 (m, 6H), 3.10-2.70 (m, 6H), 2.40-2.30 (m, 2H), 2.05-1.89 (m, 8H).

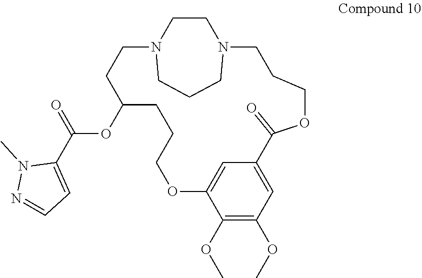 [SMILES: COc1cc2cc(c1OC)OCCCC(OC(=O)c1ccnn1C)CCN1CCCN(CCCOC2=O)CC1]

The compound 10 was prepared by the general procedure 1 using the intermediate compound 4 (35 mg, 0.08 mmol, 1.00 equiv) and 2-methylpyrazole-3-carboxylic acid (20.22 mg, 0.16 mmol, 2.00 equiv). After purification by Prep-HPLC (RT (min): 6.9) the compound 10 (28 mg, TFA salt, 64%) was isolated as an off-white solid.

LCMS (ESI position ion) m/z: 545.4 (M+H)+ (calculated: 545.3)

1H NMR (400 MHz, CD3OD-d4) δ 7.50 (s, 1H), 7.42 (s, 1H), 7.35 (s, 1H), 6.92 (s, 1H), 5.49 (br, 1H), 4.46-4.35 (m, 3H), 4.11-4.10 (m, 4H), 3.88 (s, 3H), 3.81 (s, 3H), 3.52-3.43 (m, 6H), 3.14-2.74 (m, 6H), 2.40-2.20 (m, 2H), 2.15-1.85 (m, 8H).

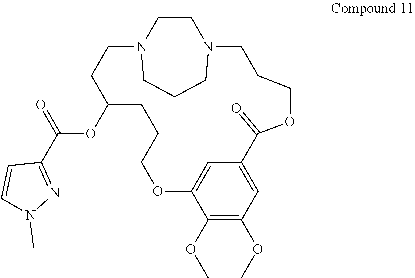 [SMILES: COc1cc2cc(c1OC)OCCCC(OC(=O)c1ccn(C)n1)CCN1CCCN(CCCOC2=O)CC1]

The compound 11 was prepared by the general procedure 1 using the intermediate compound 4 (35 mg, 0.08 mmol, 1.00 equiv) and 1-methylpyrazole-3-carboxylic acid (20.22 mg, 0.16 mmol, 2.00 equiv). After purification by Prep-HPLC (RT (min): 6.6) the compound 11 (24 mg, TFA salt, 55%) was isolated as an off-white solid.

LCMS (ESI position ion) m/z: 545.3 (M+H)+ (calculated: 545.3)

1H NMR (400 MHz, CD3OD-d4) δ 7.68 (s, 1H), 7.43 (s, 1H), 7.34 (s, 1H), 6.81 (s, 1H), 5.50 (br, 1H), 4.50-4.30 (m, 3H), 4.09-4.06 (m, 1H), 3.96 (s, 3H), 3.88 (s, 3H), 3.82 (s, 3H), 3.69-3.37 (m, 6H), 3.14-2.72 (m, 6H), 2.30-1.80 (m, 10H).

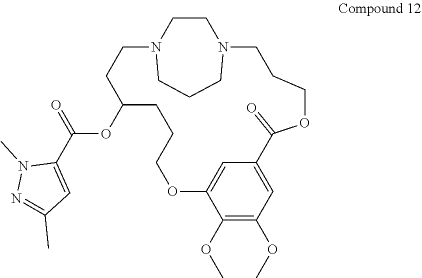 [SMILES: COc1cc2cc(c1OC)OCCCC(OC(=O)c1cc(C)nn1C)CCN1CCCN(CCCOC2=O)CC1]

The compound 12 was prepared by the general procedure 1 using the intermediate compound 4 (35 mg, 0.08 mmol, 1.00 equiv) and 2,5-dimnethylpyrazole-3-carboxylic acid (22.47 mg, 0.16 mmol, 2.00 equiv). After purification by Prep-HPLC (RT (min): 8.9) the compound 12 (26 mg, TFA salt, 58%) was isolated as an off-white solid.

LCMS (ESI position ion) m/z: 559.3 (M+H)+ (calculated: 559.3)

1H NMR (400 MHz, CD3OD-d4) δ 7.41 (s, 1H), 7.35 (s, 1H), 6.69 (s, 1H), 5.46 (br, 1H), 4.42-4.35 (m, 3H), 4.11 (br, 1H), 4.05 (s, 3H), 3.88 (s, 3H), 3.85 (s, 3H), 3.70-3.42 (m, 6H), 3.13-2.69 (m, 6H), 2.40-1.80 (m, 13H).

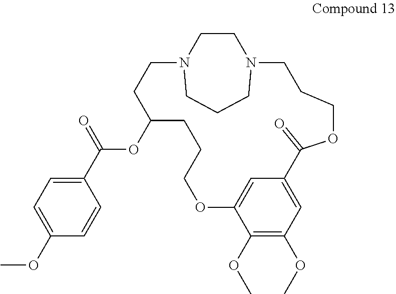 [SMILES: COc1ccc(C(=O)OC2CCCOc3cc(cc(OC)c3OC)C(=O)OCCCN3CCCN(CC2)CC3)cc1]

The compound 13 was prepared by the general procedure 1 using the intermediate compound 4 (35 mg, 0.08 mmol, 1.00 equiv) and p-anisic acid (24.40 mg, 0.16 mmol, 2.00 equiv). After purification by Prep-HPLC (RT (min): 7.1) the compound 13 (16 mg, TFA salt, 58%) was isolated as an off-white solid.

LCMS (ESI position ion) m/z: 571.4 (M+H)+ (calculated: 571.3)

1H NMR (400 MHz, CD3OD-d4) δ 7.99 (d, J=6.6 Hz, 2H), 7.43 (s, 1H), 7.34 (s, 1H), 7.00 (d, J=6.6 Hz, 2H), 5.48 (br, 1H), 4.50-4.35 (m, 3H), 4.08 (br, 1H), 3.90 (s, 3H), 3.88 (s, 3H), 3.83 (s, 3H), 3.69-3.40 (m, 6H), 3.13-2.72 (m, 6H), 2.38-2.27 (m, 2H), 2.01-1.80 (m, 8H).

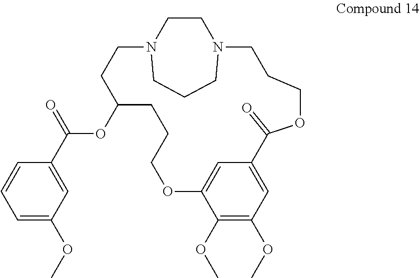 [SMILES: COc1cccc(C(=O)OC2CCCOc3cc(cc(OC)c3OC)C(=O)OCCCN3CCCN(CC2)CC3)c1]

The compound 14 was prepared by the general procedure 1 using the intermediate compound 4 (35 mg, 0.08 mmol, 1.00 equiv) and m-methoxybenzoic acid (24.40 mg, 0.16 mmol, 2.00 equiv). After purification by Prep-HPLC (RT (min): 7.1) the compound 14 (25 mg, TFA salt, 55%) was isolated as an off-white solid.

LCMS (ESI position ion) m/z: 571.4 (M+H)+ (calculated: 571.3)

1H NMR (400 MHz, CD3OD-d4) δ 7.64 (d, J=8.0 Hz, 1H), 7.55 (s, 1H), 7.48-7.40 (m, 2H), 7.37-7.18 (m, 2H), 5.52 (br, 1H), 4.43-4.39 (m, 3H), 4.09 (br, 1H), 3.90 (s, 3H), 3.86 (s, 3H), 3.83 (s, 3H), 3.56-3.35 (m, 6H), 3.13-2.71 (m, 6H), 2.40-2.25 (m, 2H), 2.03-1.89 (m, 8H).

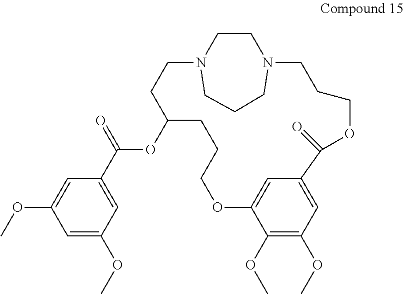 [SMILES: COc1cc(OC)cc(C(=O)OC2CCCOc3cc(cc(OC)c3OC)C(=O)OCCCN3CCCN(CC2)CC3)c1]

The compound 15 was prepared by the general procedure 1 using the intermediate compound 4 (35 mg, 0.08 mmol, 1.00 equiv) and 3,5-dimethoxybenzoic acid (29.21 mg, 0.16 mmol, 2.00 equiv). After purification by Prep-HPLC (RT (min): 7.9) the compound 15 (29 mg, TFA salt, 60%) was isolated as an off-white solid.

LCMS (ESI position ion) m/z: 601.4 (M+H)+ (calculated: 601.3)

1H NMR (400 MHz, CD3OD-d4) δ 7.44 (s, 1H), 7.34 (s, 1H), 7.13 (s, 2H), 6.73 (s, 1H), 5.51 (br, 1H), 4.44-4.39 (m, 3H), 4.09 (br, 1H), 3.87 (s, 3H), 3.81 (s, 9H), 3.57-3.41 (m, 6H), 3.13-2.65 (m, 6H), 2.40-2.25 (m, 2H), 2.05-1.97 (m, 8H).

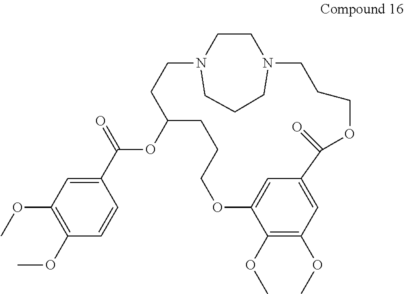 [SMILES: COc1ccc(C(=O)OC2CCCOc3cc(cc(OC)c3OC)C(=O)OCCCN3CCCN(CC2)CC3)cc1OC]

The compound 16 was prepared by the general procedure 1 using the intermediate compound 4 (35 mg, 0.08 mmol, 1.00 equiv) and veratric acid (29.21 mg, 0.16 mmol, 2.00 equiv) After purification by Prep-HPLC (RT (min) 6.7) the compound 16 (25 mg, TFA salt, 52%) was isolated as an off-white solid.

LCMS (ESI position ion) m/z: 601.4 (M+H)+ (calculated: 601.3)

1H NMR (400 MHz, CD3OD-d4) δ 7.71-7.69 (m, 1H), 7.53 (s, 1H), 7.44 (s, 1H), 7.33 (s, 1H), 7.03 (d, J=8.4 Hz, 1H), 5.49 (br, 1H), 4.42-4.39 (m, 3H), 4.89 (br, 1H), 3.89 (s, 3H), 3.87 (s, 3H), 3.85 (s, 3H), 3.81 (s, 3H), 3.53-3.42 (m, 6H), 3.08-2.72 (m, 6H), 2.35-2.27 (m, 2H), 2.03-1.87 (m, 8H).

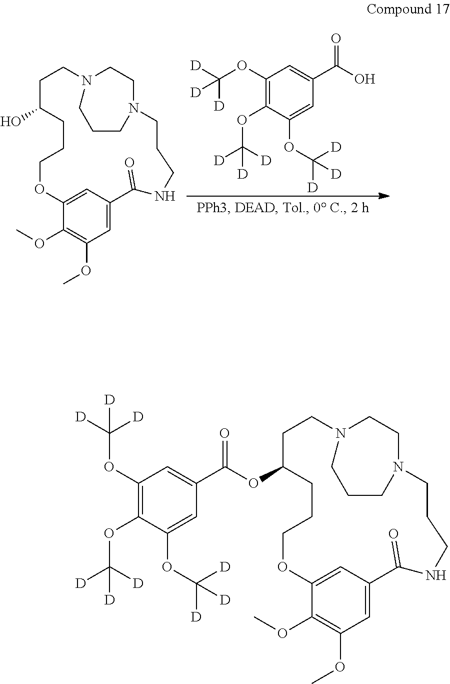 [SMILES: COc1cc2cc(c1OC)OCCC[C@H](O)CCN1CCCN(CCCNC2=O)CC1 ; [2H]C([2H])([2H])Oc1cc(C(=O)O)cc(OC([2H])([2H])[2H])c1OC([2H])([2H])[2H] ; [2H]C([2H])([2H])Oc1cc(C(=O)O[C@@H]2CCCOc3cc(cc(OC)c3OC)C(=O)NCCCN3CCCN(CC2)CC3)cc(OC([2H])([2H])[2H])c1OC([2H])([2H])[2H]]

A mixture of intermediate compound 6 (840 mg, 1.93 mmol, 1 eq), intermediate compound 7 (1.28 g, 5.79 mmol, 3 eq) and triphenylphosphine (2.53 g, 9.64 mmol, 5 eq) in toluene (20 mL) was added DEAD (1.68 g, 9.64 mmol, 1.75 mL, 5 eq) dropwise at 0° C. The mixture was stirred at 0° C. for 2 hr under nitrogen atmosphere. The reaction mixture was diluted with H₂O (100 mL) and extracted with DCM (3×60 mL). The organic layer was washed with brine, dried over anhydrous Na₂SO₄ and concentrated under reduced pressure. The residue was purified by column chromatography (SiO2. Petroleum ether/Ethyl acetate=0/1 to DCM/MeOH=10/1, (Rf=0.45)) followed by a purification purified by prep-HPLC (column: Phenomenex luna C18 150*40 mm*15 um; mobile phase: [water (FA)-ACN]; B %: 8%-38%, 10 min) and then by chiral SFC (Column. Chiralpak AD-3 50-4.6 mm I.D., 3 um. Mobile phase: Phase A for CO2, and Phase B for IPA+ACN (0.05% DEA). Gradient: 40% IPA+ACN (0.05% DEA) in CO2. Column temperature: 35° C. System back pressure: 100 bar) to afford the compound 17 (absolute stereochemistry) (260 mg, 20% yield) as white solid.

Chiral SFC: retention time=6.138 min, ee=99.3%

LCMS (ESI position ion) m/z: 639.2 (M+H)+ (calculated: 639.4)

1H NMR (400 MHz, CD3OD-d4) δ 7.31 (s, 2H), 7.20 (d, J=1.9 Hz, 1H), 7.13 (d, J=1.8 Hz, 1H), 5.50 (br s, 1H), 4.32 (br d, J=8.0 Hz, 1H), 4.19 (br d, J=5.9 Hz, 1H), 3.89 (s, 3H), 3.81 (s, 3H), 3.65-3.57 (m, 1H), 3.50-3.39 (m, 1H), 3.01-2.93 (m, 1H), 2.92-2.80 (m, 3H), 2.76 (br t, J=6.7 Hz, 2H), 2.73-2.58 (m, 4H), 2.55 (br t, J=6.4 Hz, 2H), 2.00-1.86 (m, 5H), 1.86-1.69 (m, 5H)

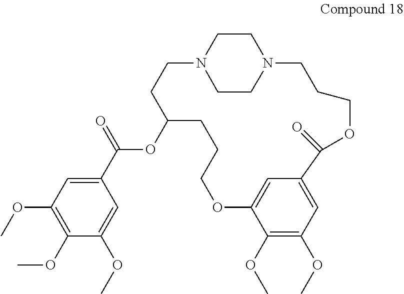 [SMILES: COc1cc(C(=O)OC2CCCOc3cc(cc(OC)c3OC)C(=O)OCCCN3CCN(CC2)CC3)cc(OC)c1OC]

The compound 18 was prepared from the intermediate compound 10 (40 mg, 0.076 mmol, 1.00 equiv) and 3,4,5-trimethoxybenzoic acid (70.52 mg, 0.355 mmol, 1.50 equiv) using the protocol described for compound 2. The purification of the crude by Prep-HPLC with the following conditions Sunfire Prep C18 OBD Column, 50*250 mm, 5 μm; Mobile Phase A: Water (0.05% TFA), Mobile Phase B: ACN; Flow rate: 90 mL/min; Gradient: 10% B to 45% B in 12 min, 45% B; Wave Length: 220 nm afforded the compound 18 (10 mg, TFA salt) as an off white solid.

LCMS (ESI position ion) m/z: 616.4 (M+H)+ (calculated: 616.3)

1H NMR (400 MHz, CD3OD-d4) δ 7.33 (s, 3H), 7.26 (s, 1H), 5.29-5.28 (m, 1H), 4.35-4.32 (m, 2H), 3.93 (s, 3H), 3.89 (s, 6H), 3.86 (s, 3H), 3.84 (s, 3H), 3.61-3.55 (m, 2H), 3.30-3.04 (m, 12H), 2.20-1.86 (m, 8H)

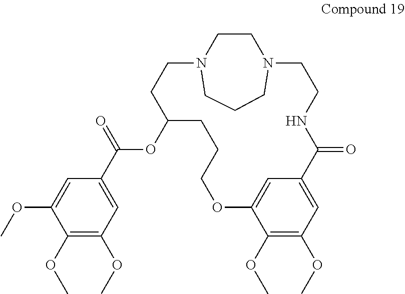 [SMILES: COc1cc(C(=O)OC2CCCOc3cc(cc(OC)c3OC)C(=O)NCCN3CCCN(CC2)CC3)cc(OC)c1OC]

The compound 19 was prepared from the intermediate compound 12 (20 mg, 0.047 mmol, 1.00 equiv) and 3,4,5-trimethoxybenzoic acid (15.10 mg, 0.071 mmol, 1.50 equiv) using the protocol described for compound 2. The purification of the crude by Prep-HPLC with the following conditions Column: SunFire Prep C18 OBD Column, 19*150 mm, 5 μm 10 nm; Mobile Phase A: Water (0.1% TFA), Mobile Phase B: ACN; Flow rate 20 mL/min; Gradient: 25% B to 45% B in 16 min, 40% B; Wave Length: 220 nm afforded the compound 19 (5.5 mg, TFA salt) as a yellow solid.

LCMS (ESI position ion) m/z: 616.3 (M+H)+ (calculated: 616.3)

1H NMR (400 MHz, CD3OD-d4) δ 7.60 (s, 1H), 7.41 (s, 2H), 7.27 (s, 1H), 5.35 (brs, 1H), 4.44-4.30 (m, 2H), 3.93-3.71 (m, 17H), 3.61-3.29 (m, 8H), 3.03-2.96 (m, 4H), 2.38-1.81 (m, 8H).

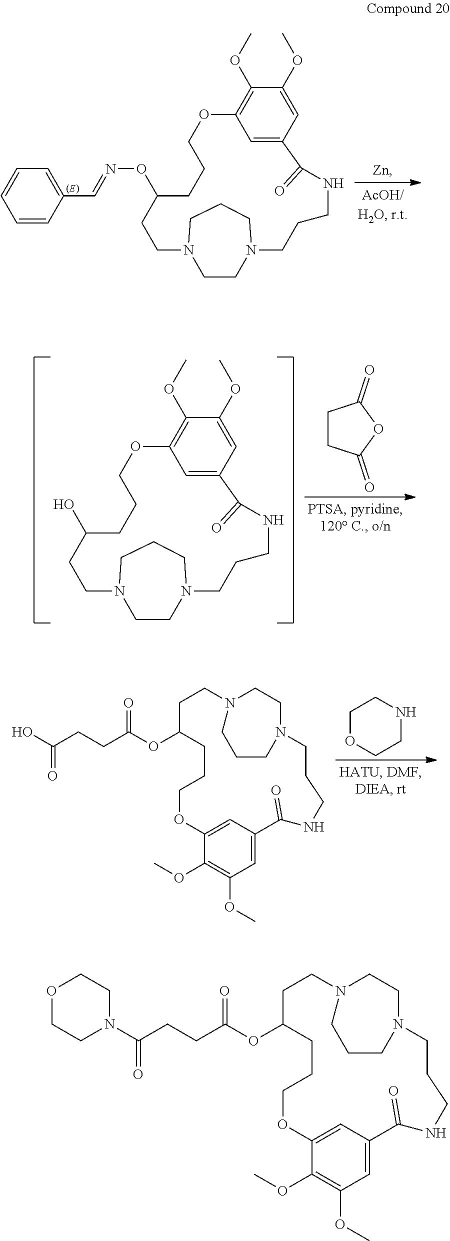

To a mixture of intermediate compound 13 (0.171 mmol, 1.0 equiv.) in acetic acid (1.80 mL) and water (1.80 mL) was added zinc dust (223 mg, 3.42 mmol, 20 equiv.) and the reaction mixture was stirred at room temperature for 3 h. The reaction mixture was quenched with 1 M aq. Na2CO3 (10 mL) and extracted with EtOAc (3×10 mL). The combined organic components were washed with brine (10 mL), dried over anhydrous Na₂SO₄ and concentrated under reduced pressure. The residue was dissolved in pyridine (1 mL) and added PTSA (0.50 equiv), dihydrofuran-2,5-dione (4.00 equiv) was added. The reaction mixture was stirred for 16 h at 120° C. The reaction solution was concentrated under vacuum to afford crude product, which was purified by Prep-HPLC to give the corresponding acid as a colorless oil.

To the acid (30 mg, 0.056 mmol, 1.00 equiv) in DMF (2 mL) were added HATU (31.94 mg, 0.084 mmol, 1.50 equiv) and DIEA (10.86 mg, 0.084 mmol, 1.50 equiv) and morpholine (1.50 equiv). The reaction mixture was stirred for 8 h at room temperature. The reaction was quenched by the addition of water (5 mL) at room temperature. The resulting mixture was extracted with EtOAc (3×5 mL). The combined organic layers were washed with brine (5 mL), dried over anhydrous Na₂SO₄. After filtration, the filtrate was concentrated under reduced pressure. The organic layer was concentrated under vacuum to afford crude product, which was then purified by Prep-HPLC to afford the compound 20.

LCMS (ESI position ion) m/z: 605.4 (M+H)+ (calculated: 605.4)

1H NMR (400 MHz, CD3OD-d4) δ 7.35 (s, 1H), 7.23 (s, 1H), 5.10 (s, 1H), 4.32-4.24 (m, 2H), 3.88 (s, 3H), 3.83 (s, 3H), 3.67-3.52 (m, 18H), 3.31-3.27 (m, 4H), 2.77-2.54 (m, 4H), 2.32 (s, 2H), 2.11-2.02 (m, 4H), 1.91-1.81 (m, 4H).

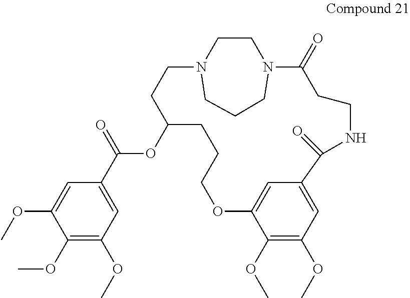 [SMILES: COc1cc(C(=O)OC2CCCOc3cc(cc(OC)c3OC)C(=O)NCCC(=O)N3CCCN(CC2)CC3)cc(OC)c1OC]

The compound 21 was prepared from the intermediate compound 15 (300 mg, 0.768 mmol, 1.00 equiv) and 3,4,5-trimethoxybenzoic acid (1.50 equiv) using the protocol described for compound 2. The residue was purified by reverse flash chromatography with the following conditions: Column: SunFire Prep C18 OBD Column, 19*150 mm, 5 μm; Mobile Phase A: Water (0.05% TFA), Mobile Phase B: ACN; Flow rate: 20 mL/min; Gradient: 20% B to 40% B in 7 min; Wave Length: 220 nm; RT1(min): 6.9, affording the compound 21 (10 mg, TFA salt) as an off white solid

LCMS (ESI position ion) m/z: 644.4 (M+H)+ (calculated: 644.3)

1H NMR (400 MHz, CD3OD-d4) δ 7.52-7.25 (m, 4H), 5.41-5.20 (m, 1H), 4.60-4.10 (m, 5H), 3.97-3.89 (m, 17H), 3.85-3.08 (m, 7H), 2.80-1.81 (m, 10H).

### General Procedure 2

To a stirred solution of intermediate compound 4 (55 mg, 0.13 mmol, 1.00 equiv) and selected hydroxyl aromatic (2.00 equiv) in THF (3 mL) were added DTAD (63.58 mg, 0.25 mmol, 2.00 equiv) and triphenylphosphine (66.09 mg, 0.25 mmol, 2.00 equiv) in portions at rt. The resulting mixture was stirred for 2 h at rt. The reaction was quenched with a saturated solution of NH₄Cl (2 mL). The resulting mixture was extracted with ethyl acetate (3×5 mL). The combined organic layers were washed with brine (1×3 mL), dried over anhydrous Na₂SO₄. After filtration, the filtrate was concentrated under reduced pressure. The crude product was purified by Prep HPLC as precised in the compounds description.

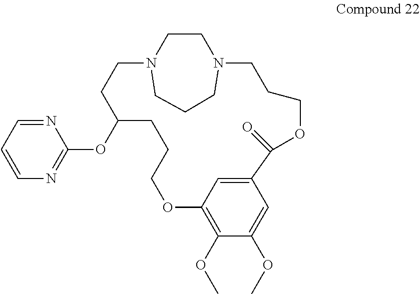 [SMILES: COc1cc2cc(c1OC)OCCCC(Oc1ncccn1)CCN1CCCN(CCCOC2=O)CC1]

The compound 22 was prepared by the general procedure 2 using 2-hydroxypyrimidine (24.21 mg, 0.25 mmol, 2.00 equiv) After purification by Prep-HPLC with the following conditions (Column: XSelect CSH Prep C18 OBD Column, 19*150 mm, 5 μm; Mobile Phase A: Water (0.05% HCl), Mobile Phase B: ACN; Flow rate: 20 mL/min; Gradient: 5% B to 20% B in 8 min, 20% B; Wave Length: 220 nm) the compound 22 (5.3 mg, TFA salt, 54%) was isolated as an off-white solid.

LCMS (ESI position ion) m/z: 515.3 (M+H)+ (calculated: 515.3)

1H NMR (400 MHz, CD3OD-d4) δ 8.61 (s, 2H), 7.42 (s, 1H), 7.37 (s, 1H), 7.14 (s, 1H), 5.41 (s, 1H), 4.49-4.00 (m, 8H), 3.90 (s, 3H), 3.83 (s, 3H), 3.81-3.48 (m, 8H), 2.50-2.30 (m, 6H), 2.23-2.00 (m, 4H)

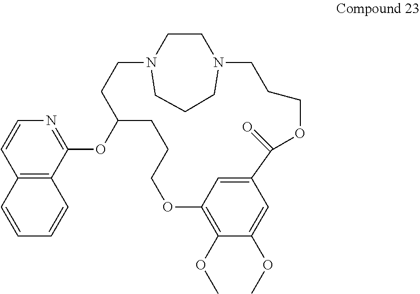 [SMILES: COc1cc2cc(c1OC)OCCCC(Oc1nccc3ccccc13)CCN1CCCN(CCCOC2=O)CC1]

The compound 23 was prepared by the general procedure 2 using 1-hydroxyisoquinoline (36.58 mg, 0.25 mmol, 2.00 equiv) Ater purification by Prep-HPLC with the following conditions (Column: XBridge Shield RP18 OBD Column, 19*150 mm, 5 μm; Mobile Phase A: Water (0.05% TFA), Mobile Phase B: ACN; Flow rate: 20 mL/min; Gradient: 14% B to 33% B in 10 miii, 33% B; Wave Length: 220 nm) the compound 23 (4.7 mg, TFA salt) was isolated as an yellow solid.

LCMS (ESI position ion) m/z: 564.4 (M+H)+ (calculated: 564.3)

1H NMR (400 MHz, CD3OD-d4) δ 8.29 (d, J=6.0 Hz, 1H), 7.89-7.88 (m, 1H), 7.80 (d, J=6.0 Hz, 1H), 7.75-7.60 (m, 2H), 7.44 (s, 1H), 7.34 (s, 1H), 7.30-7.28 (m, 1H), 5.78 (s, 1H), 4.47-4.41 (m, 3H), 4.13-4.11 (m, 1H), 3.89 (s, 3H), 3.76 (s, 3H), 3.40-2.75 (m, 11H), 2.29-1.99 (m, 11H)

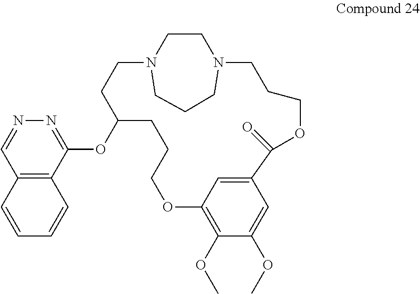 [SMILES: COc1cc2cc(c1OC)OCCCC(Oc1nncc3ccccc13)CCN1CCCN(CCCOC2=O)CC1]

The compound 24 was prepared by the general procedure 2 using phthalazinone (36.83 mg, 0.25 mmol, 2.00 equiv). After purification by Prep-HPLC with the following conditions (Column: Kinetex EVO C18 Column, 21.2*150, 5 um; Mobile Phase A: Water (0.05% HCl), Mobile Phase B: ACN; Flow rate: 20 mL/min; Gradient: 13% B to 28% B in 8 min, 28% B; Wave Length: 220 nm) the compound 24 (8.5 mg, TFA salt) was isolated as a light yellow solid.

LCMS (ESI position ion) m/z: 565.3 (M+H)+ (calculated: 565.3)

1H NMR (400 MHz, CD3OD-d4) δ 8.60 (br, 1H), 8.37 (s, 1H), 7.98-7.90 (m, 3H), 7.52 (s, 1H), 7.35 (s, 1H), 5.47-5.33 (m, 2H), 4.51-4.17 (m, 6H), 3.86-3.47 (m, 13H), 3.30-3.13 (m, 2H), 2.50-1.54 (m, 10H)

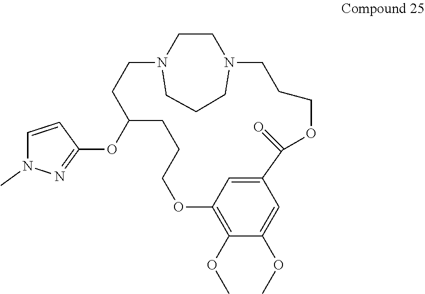 [SMILES: COc1cc2cc(c1OC)OCCCC(Oc1ccn(C)n1)CCN1CCCN(CCCOC2=O)CC1]

The compound 25 was prepared by the general procedure 2 using 1-methylpyrazol-3-ol (24.72 mg, 0.25 mmol, 2.00 equiv) After purification by Prep-HPLC with the following conditions (Column: Xcelect CSH F-pheny OBD Column, 19*250 mm, 5 μm; Mobile Phase A: Water (0.05% HCl), Mobile Phase B: ACN; Flow rate: 20 mL/min; Gradient: 5% B to 20% B in 8 min, 20% B; Wave Length: 220 nm) the compound 25 (5 mg, TFA salt) was isolated as An off-white solid.

LCMS (ESI position ion) m/z: 517.3 (M+H)+ (calculated: 517.3)

1H NMR (400 MHz, CD3OD-d4) δ 7.43-7.37 (m, 3H), 5.73 (br, 1H), 4.84-4.01 (m, 9H), 3.87 (s, 3H), 3.83 (s, 3H), 3.70-3.31 (m, 11H), 2.39-2.00 (m, 10H)

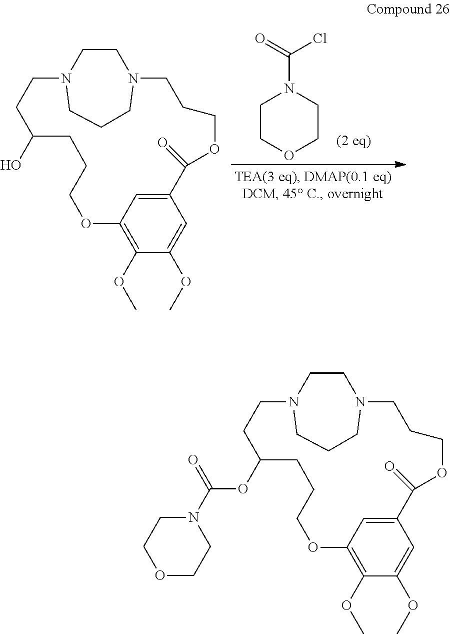

To a stirred solution of intermediate compound 4 (50 mg, 0.12 mmol, 1.00 equiv), TEA (57.95 mg, 0.58 mmol, 5.00 equiv) and DMAP (1.40 mg, 0.01 mmol, 0.10 equiv) in DCM (3 mL) were added morpholine-4-carbonyl chloride (51.39 mg, 0.35 mmol, 3.00 equiv) at rt. The resulting mixture was stirred for 12 h at 50° C. The mixture was allowed to cool down to rt. The reaction was quenched by the addition of a saturated solution of NH₄Cl (3 mL). The aqueous layer was extracted with DCM (2×3 mL). The combined organic layers were washed with brine (1×3 mL), dried over anhydrous Na₂SO₄. After filtration, the filtrate was concentrated under reduced pressure. The crude product was purified by Prep-HPLC with the following conditions (Column: Sunfire Prep C18 OBD Column, 50*250 mm, 5 μm; Mobile Phase A Water (0.05% TFA), Mobile Phase B: ACN; Flow rate: 90 mL/min; Gradient: 5% B to 5% B in 3 min, 5% B to 25% B in 12 min, 25% B; Wave Length: 220 nm; RT1(min): 12) to afford the compound 26 (4.5 mg, TFA salt) as a off-white solid.

LCMS (ESI position ion) m/z: 550.3 (M+H)+ (calculated: 550.3)

1H NMR (400 MHz, CD3OD-d4) δ 7.42-7.38 (m, 2H), 4.90 (br, 1H), 4.60-3.20 (m, 31H), 2.35-1.95 (m, 9H)

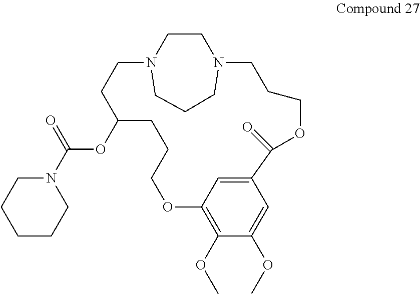 [SMILES: COc1cc2cc(c1OC)OCCCC(OC(=O)N1CCCCC1)CCN1CCCN(CCCOC2=O)CC1]

To a stirred solution of intermediate compound 4 (50 mg, 0.12 mmol, 1.00 equiv) and TEA (17.38 mg, 0.17 mmol, 1.50 equiv) in DCM (3 mL) was added 4-nitrophenyl carbonochloridate (25.39 mg, 0.13 mmol, 1.10 equiv) in portions at rt. The resulting mixture was stirred for 1 h at rt. To the above mixture was added piperidine (24.38 mg, 0.29 mmol, 2.50 equiv) dropwise at rt. The resulting mixture was stirred for additional 2h at rt. The reaction was quenched with a saturated solution of NH₄Cl (3 mL). The aqueous layer was extracted with DCM (2×3 mL). The combined organic layers were washed with brine (1×3 mL), dried over anhydrous Na₂SO₄. After filtration, the filtrate was concentrated under reduced pressure. The crude product was purified by Prep-HPLC with the following conditions (Column: Xselect CSH F-Phenyl OBD Column 19*150 mm 5 μm, n; Mobile Phase A. Water (0.05% TFA), Mobile Phase B: ACN; Flow rate: 20 mL/min; Gradient: 14% 13 to 22% B in 10 min, 22% 13; Wave Length: 220 nm; RT1(min): 8.9), to afford the compound 27 (22 mg, TFA salt) as a off-white solid

LCMS (ESI position ion) m/z: 548.4 (M+H)+ (calculated: 548.4)

1H NMR (400 MHz, DMSO-d6) δ 7.44 (s, 1H), 7.33 (s, 1H), 5.10 (br, 1H), 4.50-4.30 (m, 3H), 4.10 (br, 1H), 3.88 (s, 3H), 3.83 (s, 3H), 3.60-3.20 (m, 11H), 3.00-2.70 (m, 5H), 2.40-1.40 (m, 16H)

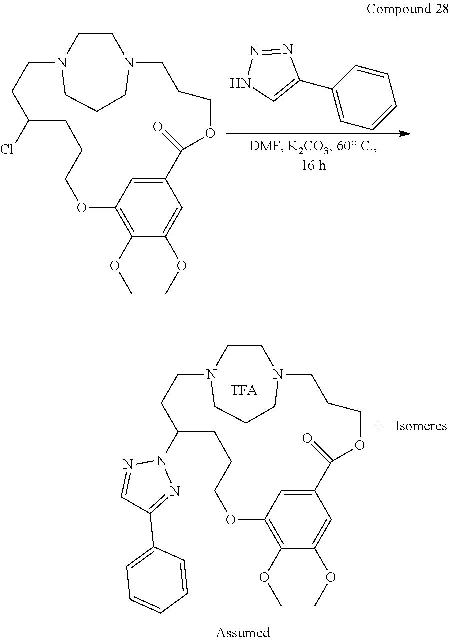

A mixture of intermediate compound 16 (70 mg, 0.15 mmol, 1.0 equiv), K2CO3 (42.5 mg, 0.31 mmol, 2.0 equiv) and 1H-1,2,3-triazole, 4-phenyl- (33.5 mg, 0.23 mmol, 1.5 equiv) in DMF (2 mL) was stirred for 16 h at 50° C. The reaction was then quenched with water (8 mL), and extracted with EtOAc (3×5 mL) The combined organic layers were washed with brine (8 mL), dried over anhydrous Na₂SO₄, and concentrated under reduced pressure. The crude product was purified by Prep-HPLC with the following conditions: Column: Sunfire Prep C18 OBD Column, 50×250 mm, 5 μm; Mobile Phase A: Water (0.05% TFA), Mobile Phase B: ACN; Flow rate: 65 mL/min; Gradient: 10% B to 45% B in 12 min, 45% B; Wave Length: 220 nm) to afford the compound 28 (7.1 mg, TFA salt) as an off-white solids.

LCMS (ESI position ion) m/z: 564.4 (M+H)+ (calculated: 564.4)

1H NMR (400 MHz, CD3OD-d4) δ 8.09 (s, 1H), 8.81-7.79 (m, 2H), 7.44-7.31 (m, 5H), 5.07 (br, 1H), 4.84-4.41 (m, 3H), 4.05 (br, 1H), 3.87 (s, 3H), 3.78 (s, 3H), 3.69-3.47 (m, 5H), 3.12-3.08 (m, 1H), 2.89-2.65 (m, 3H), 2.40-1.93 (m, 11H), 1.56 (br, 1H).

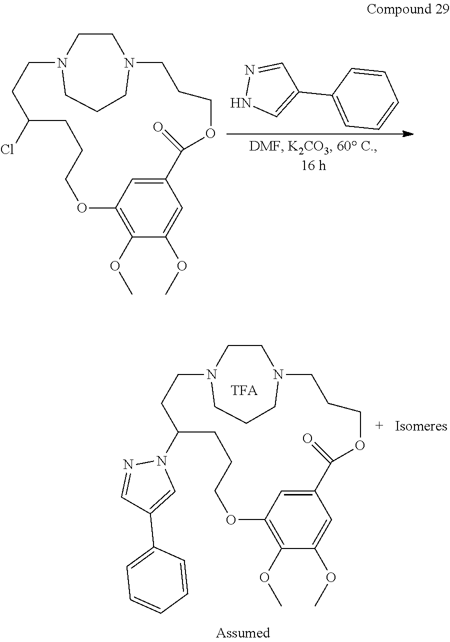

A solution of 1H-tetrazole, 5-phenyl- (28.5 mg, 0.198 mmol, 1.50 equiv) in DMF (5 mL) was treated with K2CO3 (36.5 mg, 0.264 mmol, 2.00 equiv) at 50° C., After stirring for 1 h, the intermediate compound 16 (60 mg, 0.132 mmol, 1.00 equiv) was added. The resulting mixture was stirred for 16 h at 60° C. The resulting mixture was filtered, the filtrate was purified directly with Pre-HPLC with the following conditions: Column: SunFire Prep C18 OBD Column, 19*150 mm, 5 μm 10 nm; Mobile Phase A: Water (0.05% TFA), Mobile Phase B: ACN; Flow rate: 18 mL/min; Gradient: 15% 13 to 40% B in 7 min, 40% B; Wave Length: 220 nm) to afford the compound 29 (5.5 mg, TFA salt) as off-white solid.

LCMS (ESI position ion) m/z: 563.4 (M+H)+ (calculated: 563.4)

1H NMR (400 MHz, DMSO-d6) δ 10.03 (br, 1H), 8.23 (s, 1H), 7.91 (s, 1H), 7.61-7.57 (m, 2H), 7.37-7.15 (m, 5H), 5.58-5.01 (m, 4H), 4.22-4.07 (m, 5H), 3.83 (s, 3H), 3.73 (s, 3H), 3.61-2.96 (m, 8H), 2.27-1.85 (m, 10H)

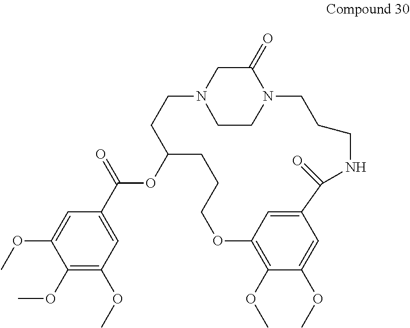 [SMILES: COc1cc(C(=O)OC2CCCOc3cc(cc(OC)c3OC)C(=O)NCCCN3CCN(CC2)CC3=O)cc(OC)c1OC]

The compound 30 was prepared from the intermediate compound 17 (30 mg, 0.06 mmol, 1.00 equiv) and 3,4,5-trimethoxybenzoic acid (52 mg, 0.426 mmol, 1.50 equiv) using the protocol described for compound 2. The residue was purified by reverse flash chromatography (Column: XSelect CSH Prep C18 OBD Column, 19*150 mm, 5 μm; Mobile Phase A: Water (0.05% TFA), Mobile Phase B. ACN; Flow rate: 20 mL/min; Gradient: 10% B to 65% B in 8 min, 65% B; Wave Length: 220 nm&254 nm). affording the compound 30 (4.6 mg, TFA salt) as an off-white solid.

LCMS (ESI position ion) m/z: 630.4 (M+H)+ (calculated: 630.4)

1H NMR (400 MHz, CDCl3-d) δ 7.33 (s, 3H), 7.18 (s, 1H), 5.37 (br, 1H), 4.85 (s, 2H), 4.40-4.24 (m, 2H), 3.91 (s, 3H), 3.87 (s, 6H), 3.83 (s, 3H), 3.81 (s, 3H), 3.72-3.29 (m, 8H), 3.14-2.91 (m, 2H), 2.20-1.80 (m, 8H)

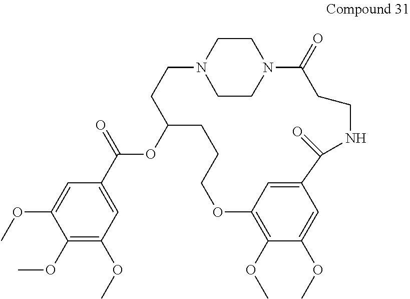 [SMILES: COc1cc(C(=O)OC2CCCOc3cc(cc(OC)c3OC)C(=O)NCCC(=O)N3CCN(CC2)CC3)cc(OC)c1OC]

The compound 31 was prepared from the intermediate compound 19 (50 mg, 0.09 mmol, 1.00 equiv) and 3,4,5-trimethoxybenzoic acid (48.72 mg, 0.24 mmol, 2.00 equiv) using the protocol described for compound 2. The residue was purified by reverse flash chromatography (Column: SunFire Prep C18 OBD Column, 19*150 mm, 5 μm; Mobile Phase A: Water (0.05% TFA), Mobile Phase B: ACN; Flow rate: 20 mL/min; Gradient: 25% B to 35% B in 7 min; Wave Length 220 nm; RT1(min): 6.9). affording the compound 31 (18.3 mg, TFA salt) as an off-white solid.

LCMS (ESI position ion) m/z: 630.3 (M+H)+ (calculated: 630.3)

1H NMR (400 MHz, CDCl3-d) δ 7.34-7.32 (m, 3H), 7.25 (s, 1H), 5.22 (br, 1H), 4.80-4.10 (m, 4H), 3.92 (s, 3H), 3.87 (s, 6H), 3.83 (s, 6H), 3.81-3.51 (m, 5H), 3.41-2.81 (m, 6H), 2.50-1.89 (m, 7H)

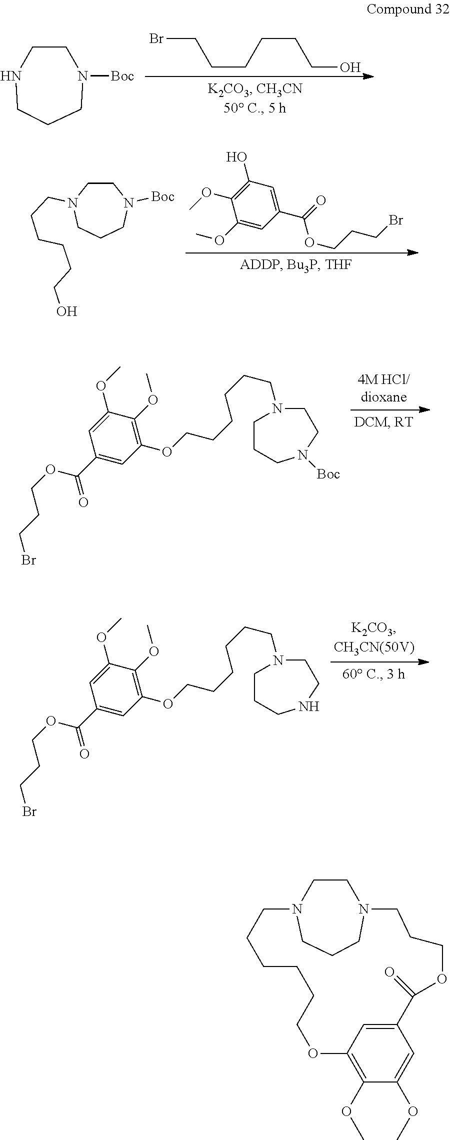

To a stirred solution of tert-butyl 1,4-diazepane-1-carboxylate (500 mg, 2.496 mmol, 1.00 equiv) and K2CO3 (690.05 mg, 4.99 mmol, 2.00 equiv) in MeCN (10 mL) was added 6-bromo-1-hexanol (452.05 mg, 2.496 mmol, 1.0 equiv) at 50° C. The resulting mixture was stirred for 5 h at 50° C. The resulting mixture was diluted with DCM (30 mL). The resulting mixture was filtered; the filtrate was concentrated under reduced pressure. The residue was purified by silica gel column chromatography, eluted with PE/ethyl acetate (5:1) to afford tert-butyl4-(6-hydroxyhexyl)-1,4-diazepane-1-carboxylate (450 mg, 60% yield) as a colorless oil.

LCMS (ESI position ion) m/z: 301.3 (M+H)+ (calculated: 301.3)

To a solution of ADDP (74.99 mg, 0.300 mmol, 1.50 equiv) and n-Bu₃P (60.61 mg, 0.300 mmol, 1.50 equiv) in dry THF (4.0 mL) under N2. After stirring for 15 min, tert-butyl4-(6-hydroxyhexyl)-1,4-diazepane-1-carboxylate (60 mg, 0.200 mmol, 1.00 equiv) and 3-bromopropyl 3-hydroxy-4,5-dimethoxybenzoate (63.74 mg, 0.200 mmol, 1.00 equiv) in THF (2 mL) was added. The mixture solution was stirred for 0.5 h at 40° C. The reaction was then quenched by the addition of H₂O (10 mL). The resulting solution was extracted with ethyl acetate (2×15 mL). The organic phase was dried by Na₂SO₄ and concentrated. The crude product was purified by Flash-Prep-HPLC with the following conditions: Column (C18-I, 20-40 μm), mobile phase (MeOH/H2O=30% to 100%:7 min, 100%:3 min); Detector (254 and 220 nm) to afford the tert-butyl 4-(6-(5-((3-bromopropoxy)carbonyl)-2,3-dimethoxyphenoxy)hexyl)-1,4-diazepane-1-carboxylate (78 mg, 65% yield) as an off-white solid.

LCMS (ESI position ion) m/z: 601.3 (M+H)+ (calculated: 601.3)

To a stirred solution of tert-butyl 4-(6-(5-((3-bromopropoxy)carbonyl)-2,3-dimethoxyphenoxy)hexyl)-1,4-diazepane-1-carboxylate (78 mg, 0.130 mmol, 1.00 equiv) in DCM (3 mL) was added HCl (gas) in 1,4-dioxane (3 mL, 4 M) at room temperature. After stirring for 3 h, the resulting mixture was concentrated under low temperature. The crude product was used in next step directly.

LCMS (ESI position ion) m/z: 501.2 (M+H)+ (calculated: 501.2)

A solution of 3-bromopropyl 3-((6-(1,4-diazepan-1-yl)hexyl)oxy)-4,5-dimethoxybenzoate (50 mg, 0.093 mmol, 1.00 equiv) and K₂CO₃ (28.26 mg, 0.205 mmol, 2.20 equiv) in CH₃CN (5 mL) was stirred for 3 h at 60° C. The resulting mixture was filtered; the filtrate was concentrated under reduced pressure. The crude product was purified by Prep-HPLC with the following conditions (Column: SunFire Prep C18 OBD Column, 19*150 mm, 5 μm; Mobile Phase A: Water (0.05% TFA), Mobile Phase B: ACN; Flow rate: 20 mL/min; Gradient: 15% B to 35% B in 7 min, 35% B; Wave Length: 220 nm; RT1(min): 6.9) to afford the compound 32 (33 mg, TFA salt) as an off-white solid.

LCMS (ESI position ion) m/z: 421.2 (M+H)+ (calculated: 421.2)

1H NMR (400 MHz, CDCl3-d) δ 7.34 (s, 2H), 4.38-4.37 (m, 2H), 4.18-4.14 (m, 2H), 3.88-3.73 (m, 10H), 353-3.39 (m, 6H), 3.25-3.24 (m, 2H), 2.31-2.28 (m, 4H), 1.94-1.89 (m, 4H), 1.87-1.78 (n, 4H)

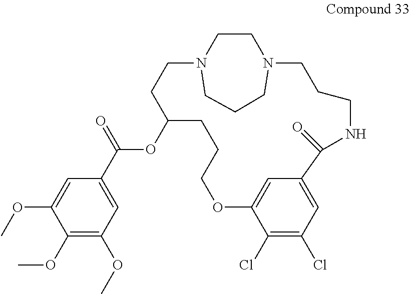 [SMILES: COc1cc(C(=O)OC2CCCOc3cc(cc(Cl)c3Cl)C(=O)NCCCN3CCCN(CC2)CC3)cc(OC)c1OC]

The compound 33 was prepared by the general procedure 1 using the intermediate compound 20 (40 mg, 0.09 mmol, 1.0 equiv) 3,4,5-trimethoxybenzoic acid (29 mg, 0.14 mmol, 1.5 equiv). After purification by Prep-HPLC (Column Sunfire Prep C18 OBD Column, 50×250 mm, 5 μm; Mobile Phase A: Water (0.05% TFA), Mobile Phase B: ACN; Flow rate: 90 mL/min; Gradient: 10% B to 45% B in 12 min, 45% B; Wave Length: 220 nm; RT1 (min): 12) the compound 33 (21 mg, TFA salt) was isolated as an white solid.

LCMS (ESI position ion) m/z: 638.3 (M+H)+ (calculated: 638.3)

1H NMR (400 MHz, CD3OD-d4) δ 7.64 (s, 1H), 7.59 (s, 1H), 7.24 (s, 2H), 5.25 (br, 1H), 4.38-4.31 (m, 2H), 3.75-3.72 (m, 9H), 3.70-3.37 (m, 8H), 3.29-3.04 (m, 6H), 2.18-1.87 (m, 10H)

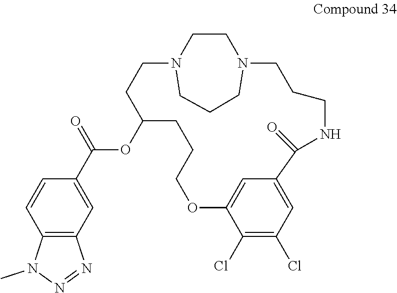 [SMILES: Cn1nnc2cc(C(=O)OC3CCCOc4cc(cc(Cl)c4Cl)C(=O)NCCCN4CCCN(CC3)CC4)ccc21]

The compound 34 was prepared by the general procedure 1 using the intermediate compound 20 (30 mg, 0.068 mmol, 1.00 equiv) 1-methyl-1H-benzo[d][1,2,3]triazole-5-carboxylic acid (25 mg, 0.14 mmol, 1.5 equiv). After purification by Prep-HPLC (Column: Sunfire Prep C18 OBD Column, 50×250 mm, 5 μm; Mobile Phase A: Water (0.05% TFA), Mobile Phase B. ACN; Flow rate 90 mL/min; Gradient: 10% B to 45% B in 12 min, 45% B; Wave Length: 220 nm; RT1 (min): 12) the compound 34 (TFA salt) was isolated as an white solid.

LCMS (ESI position ion) m/z: 603.2 (M+H)+ (calculated: 603.2)

1H NMR (400 MHz, CD3OD-d4) δ 8.72 (s, 1H), 8.22-8.20 (m, 1H), 7.84 (d, J=8.8 Hz, 1H), 7.71-7.68 (m, 2H), 5.43 (br, 1H), 4.47-4.43 (m, 2H), 4.36 (s, 3H), 3.63-3.62 (m, 2H), 3.48-3.12 (m, 12H), 2.22-2.15 (m, 4H), 2.08-1.97 (m, 6H)

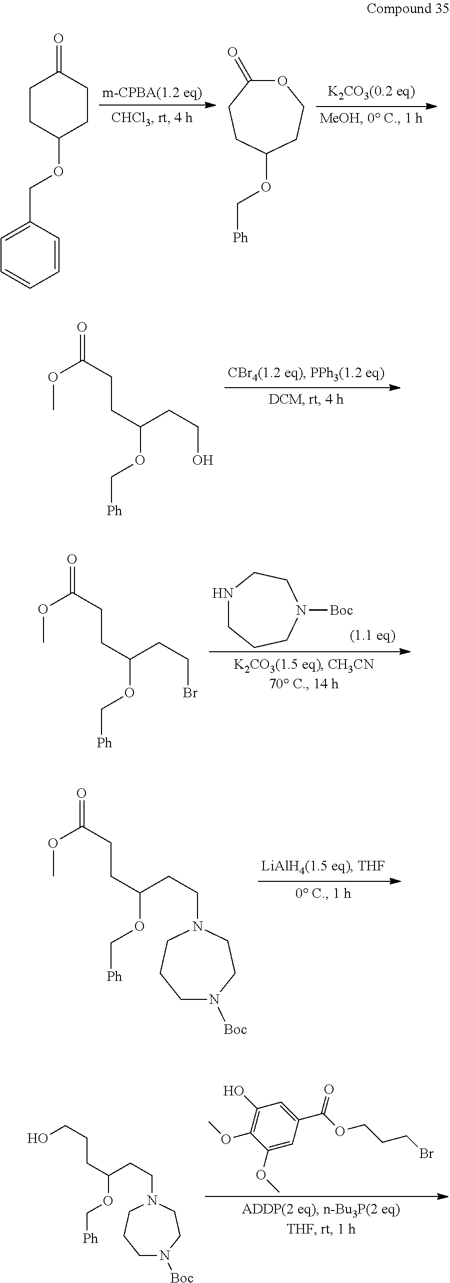

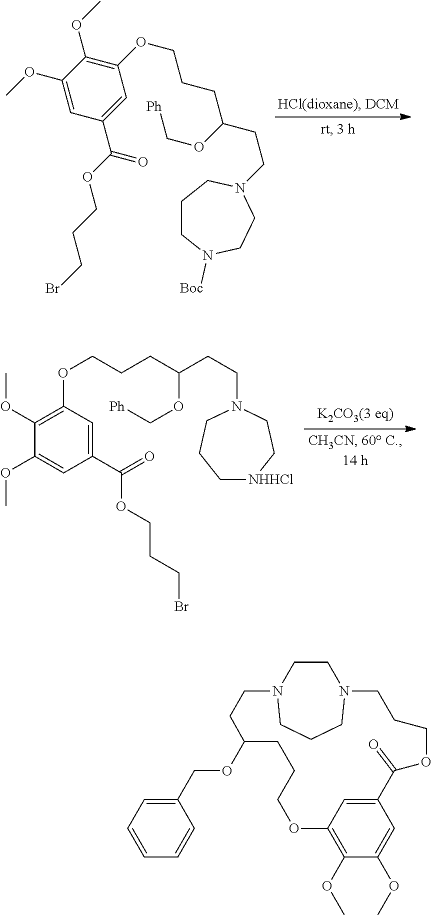

To a stirred solution of 4-(benzyloxy)cyclohexan-1-one (5 g, 24.48 mmol, 1.00 equiv) in CHCl₃ (50 mL) was added m-CPBA (5.07 g, 29.37 mmol, 1.20 equiv) in portions at room temperature under N2 atmosphere. The resulting mixture was stirred for 4h at room temperature under N2 atmosphere. The resulting mixture was filtered, the filter cake was washed with CHCl₃ (2×10 mL). The resulting mixture was washed with a saturated solution of Na₂SO₃ (2×50 mL) and brine (50 mL). The resulting solution was dried with Na₂SO₄. The resulting mixture was concentrated under reduced pressure. The residue was purified by silica gel column chromatography, eluted with THF/PE (1:50-1:20) to afford 5-(benzyloxy)oxepan-2-one (5.1 g, 94% yield) as a colorless oil.

LCMS (ESI position ion) m/z: 221.1 (M+H)+ (calculated: 221.1)

To a stirred solution of 5-(benzyloxy)oxepan-2-one (5.1 g, 23.15 mmol, 1.00 equiv) in MeOH (50 mL) was added K₂CO₃ (0.64 g, 4.631 mmol, 0.20 equiv) in portions at 0° C. under N2 atmosphere. The resulting mixture was stirred for 1 h at 0° C. under N2 atmosphere. The reaction was quenched with H₂O (100 mL). The mixture was acidified to pH 3-4 with concentrated solution of HCL. The resulting mixture was extracted with ethyl acetate (3×100 mL) The combined organic layers were washed with brine (1×100 mL), dried over anhydrous Na₂SO₄. After filtration, the filtrate was concentrated under reduced pressure. The residue was purified by silica gel column chromatography, eluted with THF/PE (1:20-1:4) to afford methyl 4-(benzyloxy)-6-hydroxyhexanoate (5.3 g, 82% yield) as a colorless oil.

LCMS (ESI position ion) m/z: 253.2 (M+H)+ (calculated: 253.1)

To a stirred solution of methyl 4-(benzyloxy)-6-hydroxyhexanoate (4.9 g, 19.42 mmol, 1.00 equiv) and PPh3 (6.11 g, 23.31 mmol, 1.20 equiv) in DCM (100 mL) was added CBr₄ (7.73 g, 23.31 mmol, 1.20 equiv) in portions at room temperature under N2 atmosphere. The resulting mixture was stirred for 4h at room temperature under N2 atmosphere. The reaction was quenched with 1-20 (100 mL) at rt. The organic layer was separated. The aqueous layer was extracted with DCM (2×100 mL). The combined organic layers were washed with brine (1×100 mL), dried over anhydrous Na₂SO₄. After filtration, the filtrate was concentrated under reduced pressure. The residue was purified by silica gel column chromatography, eluted with THF/PE (0:100-1:20) to afford methyl 4-(benzyloxy)-6-bromohexanoate (5.5 g, 90% yield) as a light-yellow oil.

LCMS (ESI position ion) m/z: 315.1 (M+H)+ (calculated: 315.0)

To a stirred solution of methyl 4-(benzyloxy)-6-bromohexanoate (5.5 g, 17.45 mmol, 1.00 equiv) and tert-butyl 1,4-diazepane-1-carboxylate (3.84 g, 19.19 mmol, 1.10 equiv) in CH₃CN (100 mL) was added K₂CO₃ (3.62 g, 26.17 mmol, 1.50 equiv) at room temperature under N2 atmosphere. The resulting mixture was stirred for 14h at 70° C. under N2 atmosphere. The mixture was allowed to cool down to rt. The resulting mixture was filtered, the filter cake was washed with ethyl acetate (2×30 mL). The filtrate was concentrated under reduced pressure. The residue was purified by silica gel column chromatography, eluted with THF/PE (1:20-1:3) to afford tert-butyl 4-[3-(benzyloxy)-6-methoxy-6-oxohexyl]-1,4-diazepane-1-carboxylate (7 g, 92% yield) as a yellow oil.

LCMS (ESI position ion) m/z: 435.3 (M+H)+ (calculated: 435.3)

To a stirred solution of tert-butyl 4-[3-(benzyloxy)-6-methoxy-6-oxohexyl]-1,4-diazepane-1-carboxylate (400 mg, 0.92 mmol, 1.00 equiv) in THF (20 mL) was added LiAlH4 (52.40 mg, 1.38 mmol, 1.50 equiv) in portions at 0° C. under N2 atmosphere. The resulting mixture was stirred for 1 h at 0° C. under N2 atmosphere. The reaction was quenched by the addition of H₂O (0.5 mL) at 0° C., followed by 0.5 mL 15% NaOH and 0.5 mL H₂O. To the above mixture was added 2 g Na₂SO₄. The resulting mixture was stirred for additional 30 minutes at rt. The resulting mixture was filtered, the filter cake was washed with ethyl acetate (2×20 mL). The filtrate was concentrated under reduced pressure. The residue was purified by silica gel column chromatography, eluted with THF/PE (1:2-3:1) to afford tert-butyl 4-[3-(benzyloxy)-6-hydroxyhexyl]-1,4-diazepane-1-carboxylate (330 mg, 88% yield) as a yellow oil.

LCMS (ESI position ion) m/z: 407.3 (M+H)+ (calculated: 407.3)

To a stirred solution of tert-butyl 4-[3-(benzyloxy)-6-hydroxyhexyl]-1,4-diazepane-1-carboxylate (210 mg, 0.517 mmol, 1.00 equiv) and 3-bromopropyl 3-hydroxy-4,5-dimethoxybenzoate (181.33 mg, 0.569 mmol, 1.10 equiv) in THF (10 mL) were added ADDP (258.61 mg, 1.034 mmol, 2.00 equiv) and n-Bu₃P (209.00 mg, 1.034 mmol, 2.00 equiv) at rt. The resulting mixture was stirred for 1 h at rt. The reaction was quenched with a saturated solution of NH₄Cl (5 mL). The resulting mixture was extracted with ethyl acetate (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with THF/PE (1:5-1:1) to afford tert-butyl 4-[3-(benzyloxy)-6-{5-[(3-bromopropoxy)carbonyl]-2,3-dimethoxyphenoxy}hexyl]-1,4-diazepane-1-carboxylate (250 mg, 68% yield) as a brown yellow oil.

LCMS (ESI position ion) m/z: 707.3 (M+H)+ (calculated: 707.3)

To a stirred mixture of tert-butyl 4-[3-(benzyloxy)-6-{5-[(3-bromopropoxy)carbonyl]-2,3-dimethoxyphenoxy}hexyl]-1,4-diazepane-1-carboxylate (250 mg) in DCM (10 mL) was added HCl(gas) in 1,4-dioxane (5 mL) at rt. The resulting mixture was stirred for 3 h at rt. The resulting mixture was concentrated under reduced pressure. This resulted in 3-bromopropyl 3-{[4-(benzyloxy)-6-(1,4-diazepan-1-yl)hexyl]oxy}-4,5-dimethoxybenzoate hydrochloride (150 mg, crude, HCl salt, used without purification) as a light-brown solid.

LCMS (ESI position ion) m/z: 607.3 (M+H)+ (calculated: 607.3)

To a stirred solution of 3-bromopropyl 3-{[4-(benzyloxy)-6-(1,4-diazepan-1-yl)hexyl]oxy}-4,5-dimethoxybenzoate hydrochloride (50 mg, 0.078 mmol, 1.00 equiv) in CH₃CN (3 mL) was added K₂CO₃ (21.46 mg, 0.156 mmol, 3.00 equiv) at rt. The resulting mixture was stirred for 14 h at 60° C. under N2 atmosphere. The mixture was allowed to cool down to rt. The resulting mixture was filtered, the filter cake was washed with ethyl acetate (2×3 mL). The filtrate was concentrated under reduced pressure. The crude product was purified by Prep-HPLC with the following conditions Column: Sunfire Prep C18 OBD Column, 50*250 mm, 5 μm; Mobile Phase A: Water (0.05% TFA), Mobile Phase B: ACN; Flow rate: 90 mL/min; Gradient: 10% B to 45% B in 12 min, 45% B; Wave Length 220 nm; RT1(min): 12; to afford the compound 35 (13 mg, TFA salt) as a light-yellow solid.

LCMS (ESI position ion) m/z: 527.4 (M+H)+ (calculated: 527.4)

1H NMR (400 MHz, CD3OD-d4) δ 7.30 (s, 1H), 7.29-7.20 (m, 6H), 4.60-4.30 (m, 4H), 4.25-4.15 (m, 2H), 3.90 (s, 3H), 3.86 (s, 3H), 3.75 (br, 1H), 3.31-2.90 (m, 12H), 2.10-1.70 (m, 10H).

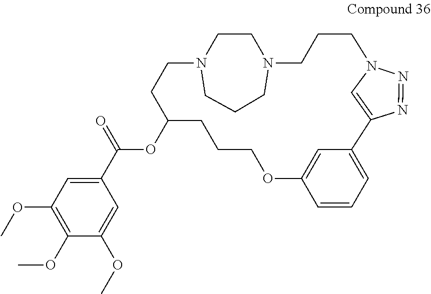 [SMILES: COc1cc(C(=O)OC2CCCOc3cccc(c3)-c3cn(nn3)CCCN3CCCN(CC2)CC3)cc(OC)c1OC]

The compound 36 was prepared from the intermediate compound 21 (17 mg, 0.171 mmol, 1.0 equiv.) and 3,4,5-trimethoxybenzoic acid (11 mg, 0.051 mmol, 1.5 equiv) using the protocol described for compound 2. The residue was purified reverse flash chromatography (column, C18 silica gel; mobile phase, MeCN (0.1% Formic Acid) in water (0.1% Formic Acid), (10-50% gradient), UV 254 nm and 220 nm affording the compound 36 (10 mg, 50% yield) as a colourless solid.

LCMS (ESI position ion) m/z: 594.5 (M+H)+ (calculated: 594.3)

1H NMR (400 MHz, CDCl3-d) δ 8.07 (s, 1H), 7.68 (s, 1H), 7.41-7.31 (m, 2H), 7.27 (s, 2H), 6.94 (d, J=7.6 Hz, 1H), 5.17-5.10 (m, 1H), 4.62-4.52 (m, 2H), 4.35-4.28 (m, 2H), 3.91 (s, 6H), 3.90 (s, 3H), 2.97-2.72 (m, 5H), 2.70-2.40 (m, 6H), 2.22-1.98 (m, 4H), 1.98-1.73 (m, 6H).

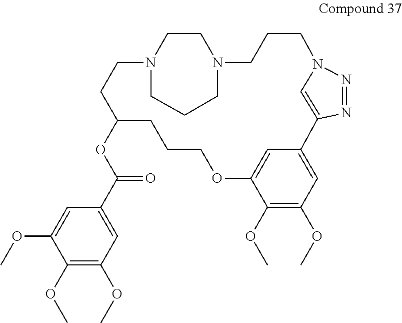 [SMILES: COc1cc(C(=O)OC2CCCOc3cc(cc(OC)c3OC)-c3cn(nn3)CCCN3CCCN(CC2)CC3)cc(OC)c1OC]

The compound 37 was prepared from the intermediate compound 23 (33 mg, 0.1059 mmol, 1.0 equiv.) and 3,4,5-trimethoxybenzoic acid (19 mg, 0.088 mmol, 1.5 equiv) using the protocol described for compound 2. The residue was purified reverse flash chromatography (column, C18 silica gel; mobile phase, MeCN (0.1% Formic Acid) in water (0.1% Formic Acid), (10-50% gradient), UV 254 nm and 220 nm) affording the compound 37 (10 mg, 26% yield) as a colourless solid.

LCMS (ESI position ion) m/z: 654.3 (M+H)+ (calculated: 654.3)

1H NMR (400 MHz, CDCl3-d) δ 7.20 (s, 2H), 7.14 (s, OH), 7.07 (d, J=1.8 Hz, 1H), 5.17-5.12 (m, 1H), 4.56-4.47 (m, 2H), 4.35-4.21 (m, 2H), 3.88 (s, 3H), 3.85-3.83 (m, 11H), 3.81 (s, 3H), 2.98-2.87 (m, 3H), 2.83-2.70 (m, 2H), 2.69-2.47 (m, 7H), 2.18-2.07 (m, 4H), 2.04-1.94 (m, 2H), 1.88-1.70 (m, 4H).

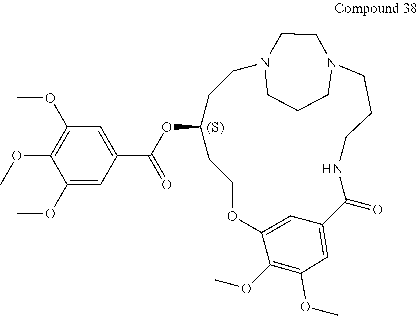

The compound 38 was prepared by the general procedure 1 using the intermediate compound 24 (30 mg, 0.071 mmol, 1.00 equiv) and 3,4,5-trimethoxybenzoic acid (22.65 mg, 0.106 mmol, 1.50 equiv). After purification by Prep-HPLC (Column Xselect CSH prep C18 OBD, 19*150 mm, 5 μm; Mobile Phase A: Water (0.05% TFA), Mobile Phase B: ACN; Flow rate: 20 mL/min; Gradient: 12% B to 42% B in 8 min; Wave Length 220 nm; RT1(min): 10) the compound 38 (17 mg, TFA salt) was isolated as a white solid.

LCMS (ESI position ion) m/z: 616.4 (M+H)+ (calculated: 616.3)

Chiral HPLC (condition 2): retention time=9.1 min, ee=99.9%

1H NMR (400 MHz, CD3OD-d4) δ 7.30 (s, 1H), 7.24 (s, 2H), 7.13 (s, 1H), 5.43-5.39 (m, 1H), 4.57-4.51 (m, 1H), 4.46-4.40 (m, 1H), 3.89 (s, 6H), 3.84-3.81 (m, 9H), 3.65-3.43 (m, 8H), 3.37-3.21 (m, 4H), 3.09-2.99 (m, 2H), 2.28-2.24 (m, 8H)

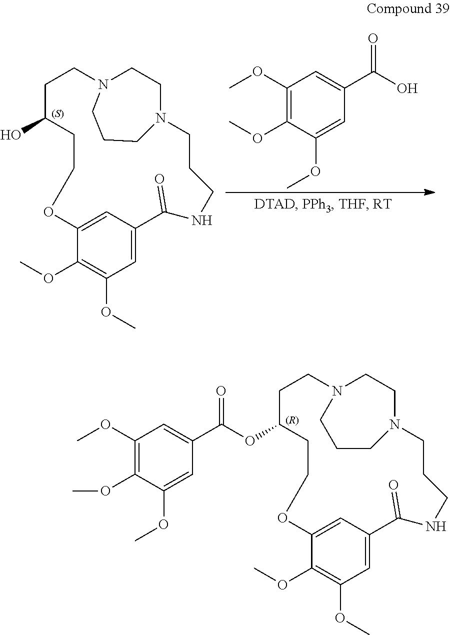

To a stirred solution of intermediate compound 24 (30 mg, 0.071 mmol, 1.00 equiv) and 3,4,5-trimethoxybenzoic acid (22.65 mg, 0.106 mmol, 1.50 equiv) in THF (5 mL) were added PPh₃ (37.33 mg, 0.142 mmol, 2.00 equiv) and (E)-N-[[(tert-butoxy)carbonyl]imino] (tert-butoxy)formamide (32.77 mg, 0.142 mmol, 2.00 equiv) at room temperature under nitrogen atmosphere. The resulting mixture was stirred for 3 h at room temperature under nitrogen atmosphere. The resulting mixture was concentrated under reduced pressure. The crude product was purified by Prep-HPLC with the following conditions: (Column: Xselect CSH prep C18 OBD, 19*150 mm, 5 μm; Mobile Phase A. Water (0.05% TFA), Mobile Phase B: ACN; Flow rate: 20 mL/min; Gradient: 12% B to 42% B in 8 min; Wave Length: 220 nm; RT1(min) 10) to afford the compound 39 (15 mg, TFA salt) as an off-white solid.

LCMS (ESI position ion) m/z: 616.4 (M+H)+ (calculated: 616.3)

Chiral HPLC (condition 2): retention time=6.47 min, ee=66.1%

1H NMR (400 MHz, CD3OD-d4) δ 7.25 (s, 1H), 7.20 (s, 2H), 7.08 (s, 1H), 5.41 (br, 1H), 4.55-4.50 (m, 1H), 4.41-4.35 (m, 1H), 3.87 (s, 6H), 3.82 (s, 3H), 3.78 (s, 6H), 3.56-3.42 (m, 8H), 3.23-3.07 (m, 4H), 2.90-2.85 (m, 2H), 2.19-1.99 (m, 8H)

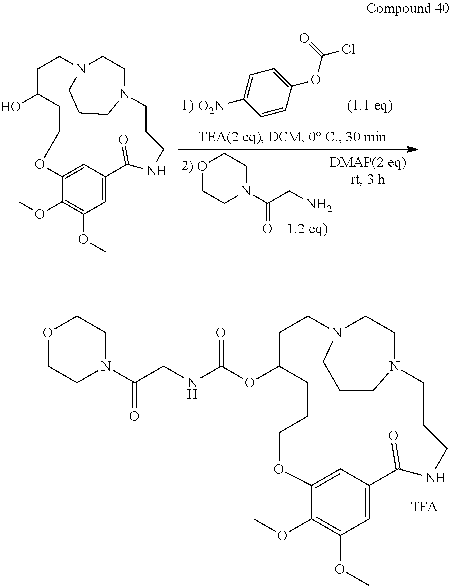 [SMILES: COc1cc2cc(c1OC)OCCC(O)CCN1CCCN(CCCNC2=O)CC1 ; NCC(=O)N1CCOCC1 ; O=C(Cl)Oc1ccc([N+](=O)[O-])cc1 ; COc1cc2cc(c1OC)OCCCC(OC(=O)NCC(=O)N1CCOCC1)CCN1CCCN(CCCNC2=O)CC1]

The 12-hydroxy-7⁴,7⁵-dimethoxy-8-oxa-5-aza-1(1,4)-diazepana-7(1,3)-benzenacyclotetradecaphan-6-one was synthetized from the intermediate compound 13 as described in the protocols for compound 20.

To a stirred solution of 12-hydroxy-7⁴,7⁵-dimethoxy-8-oxa-5-aza-1(1,4)-diazepana-7(1,3)-benzenacyclotetradecaphan-6-one (100 mg, 0.23 mmol, 1.00 equiv) and TEA (46.46 mg, 0.46 mmol, 2.00 equiv) in DCM (5 mL) was added 4-nitrophenyl carbonochloridate (50.90 mg, 0.25 mmol, 1.10 equiv) in portions at 0° C. The resulting mixture was stirred for 30 min at 0° C. To the above mixture was added DMAP (56.10 mg, 0.46 mmol, 2.00 equiv) and 2-amino-1-(morpholin-4-yl)ethanone (39.72 mg, 0.28 mmol, 1.20 equiv) at rt. The resulting mixture was stirred for additional 3h at rt. The reaction was quenched with a saturated solution of NH₄Cl (5 mL). The aqueous layer was extracted with DCM (3×5 mL). The combined organic layers were washed with brine (1×5 mL), dried over anhydrous Na₂SO₄. After filtration, the filtrate was concentrated under reduced pressure. The residue was purified by reverse flash chromatography with the following conditions. Column: Xselect CSH F-Phenyl OBD Column 19*150 mm 5 μm, n; Mobile Phase A: Water (0.05% TFA), Mobile Phase B: ACN; Flow rate: 20 mL/min; Gradient: 5% B to 22% B in 7 min, 22% B; Wave Length: 220 nm&254 nm; RT(min): 117) affording the compound 40 (38.8 mg, TFA salt) as a off-white solid.

LCMS (ESI position ion) m/z: 606.4 (M+H)+ (calculated: 606.3)

1H NMR (400 MHz, CD3OD-d4) δ 7.32 (s, 1H), 7.17 (s, 1H), 5.10-4.90 (m, 2H), 4.36-4.24 (m, 2H), 4.20-4.13 (m, 1H), 3.97-3.84 (m, 4H), 3.81 (s, 3H), 3.70-2.98 (m, 21H), 2.30-1.70 (m, 10H)

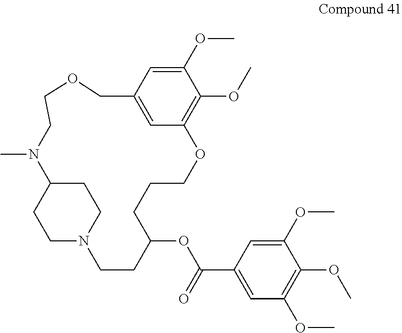

The compound 41 was prepared by the general procedure 1 using the intermediate compound 25 (150 mg, 0.36 mmol, 1.00 equiv) and 3,4,5-trimethoxybenzoic acid (110 mg, 0.53 mmol, 1.50 equiv). After purification by reverse flash chromatography (column Column: Sunfire Prep C18 OBD Column, 50*250 mm, 5 μm; Mobile Phase A: Water (20 mmol/L NH₄HCO3), Mobile Phase B: ACN; Flow rate: 80 mL/min; Gradient: 38% B to 85% B in 17 min, 85% B; Wave Length: 220 nm; RT1(min): 16.9) the compound 41 (6 mg) was isolated as a colorless solid.

LCMS (ESI position ion) m/z: 617.3 (M+H)+ (calculated: 617.3)

1H NMR (400 MHz, CD3OD-d4) δ 7.35 (s, 2H), 6.85 (s, 1H), 6.62 (s, 1H), 5.54-5.44 (m, 1H), 4.50 (br, 2H), 4.25-4.09 (m, 2H), 3.94-3.82 (m, 12H), 3.79 (s, 3H), 3.70-3.52 (m, 2H), 3.00-2.91 (m, 1H), 2.82-2.77 (m, 2H), 2.72-2.62 (m, 1H), 2.58-2.43 (m, 3H), 2.42 (s, 3H), 2.04-1.76 (m, 10H), 1.71-1.52 (m, 2H).

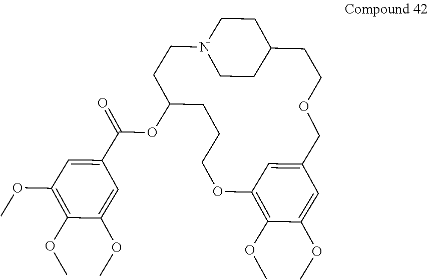 [SMILES: COc1cc(C(=O)OC2CCCOc3cc(cc(OC)c3OC)COCCC3CCN(CC3)CC2)cc(OC)c1OC]

The compound 42 was prepared by the general procedure 1 using the intermediate compound 26 (50 mg, 0.127 mmol, 1.00 equiv) and 3,4,5-trimethoxybenzoic acid (40.44 mg, 0.19 mmol, 1.50 equiv). After purification by Prep-HPLC with the following conditions (Welch Ultimate XB-C18, 50*250 cm, 10 μm; Mobile Phase A: 0.1% TFA, Mobile Phase B: ACN; Flow rate 90 mL/min; Gradient: 10%-45% B-12 mim hold 4 min) 10) the compound 42 (15 mg) was isolated as a white solid.

LCMS (ESI position ion) m/z: 588.4 (M+H)+ (calculated: 588.3)

1H NMR (400 MHz, CD3OD-d4) δ 7.36-7.32 (m, 2H), 6.90-6.23 (m, 2H), 5.25 (br, 1H), 4.43-4.27 (m, 4H), 3.87-3.69 (m, 17H), 3.37-3.21 (m, 6H), 2.21-1.47 (m, 13H).

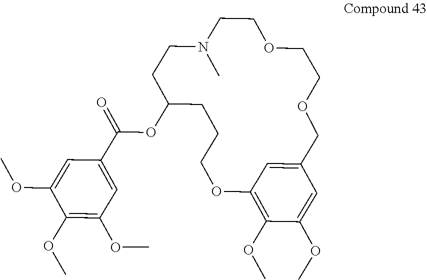 [SMILES: COc1cc(C(=O)OC2CCCOc3cc(cc(OC)c3OC)COCCOCCN(C)CC2)cc(OC)c1OC]

The compound 43 was prepared by the general procedure 1 using the intermediate compound 27 (130 mg, 0.34 mmol, 1.00 equiv) and 3,4,5-trimethoxybenzoic acid (215.81 mg, 1.02 mmol, 3.00 equiv). After purification by reverse flash chromatography with the following conditions: Column: Welch Ultimate AQ-C18, 50*250 mm*10 μm; Mobile Phase A: Water (20 mmol/L NH4HCO3), Mobile Phase B: ACN; Flow rate 80 mL/min; Gradient: 45% B to 90% B in 20 min, 90% B; Wave Length: 220 nm; RT1(min): 18.9) the compound 43 (75.2 mg, 38% yield) was isolated as a yellow oil.

LCMS (ESI position ion) m/z: 578.4 (M+H)+ (calculated: 578.3)

1H NMR (400 MHz, CD3OD-d4) δ 7.33 (s, 2H), 6.88 (s, 1H), 6.00 (s, 1H), 5.27-5.24 (m, 1H), 4.53 (s, 2H), 4.25-4.21 (m, 2H), 3.89 (s, 6H), 3.85 (s, 6H), 3.78 (s, 3H), 3.69-3.59 (m, 6H), 2.67-2.56 (m, 4H), 2.28 (s, 3H), 1.98-1.91 (m, 6H)

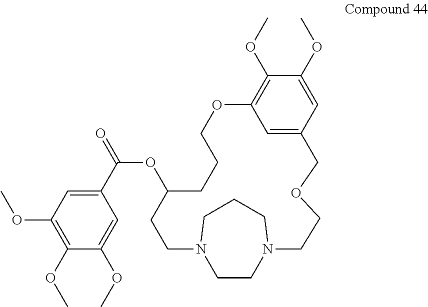 [SMILES: COc1cc(C(=O)OC2CCCOc3cc(cc(OC)c3OC)COCCN3CCCN(CC2)CC3)cc(OC)c1OC]

The compound 44 was prepared from the intermediate compound 30 (30 mg, 0.059 mmol, 1.0 equiv) and 3,4,5-trimethoxybenzoic acid (18 mg, 0.086 mmol, 1.5 equiv) using the protocol described for compound 2. The residue was purified by reverse flash chromatography (column, C18 silica gel; mobile phase, MeCN (0.1% Formic Acid) in water (0.1% Formic Acid), (10-50% gradient), UV 254 nm and 220 nm) affording the compound 44 (10 mg, 28% yield) as a white solid.

LCMS (ESI position ion) m/z: 603.4 (M+H)+ (calculated: 603.3)

1H NMR (400 MHz, CDCl3-d) δ 7.27 (s, 2H), 6.78 (s, 1H), 6.46 (s, 1H), 5.48 (t, J=6.3 Hz, 1H), 4.53 (d, J=11.7 Hz, 1H), 4.41 (d, J=11.5 Hz, 1H), 4.28-4.17 (m, 1H), 4.16-4.06 (m, 1H), 3.91 (d, J=2.4 Hz, 12H), 3.86 (s, 3H), 3.83 (s, 3H), 3.50-3.23 (m, 7H), 2.98-2.85 (m, 2H), 2.85-2.61 (m, 3H), 2.17-2.00 (m, 3H), 1.96-1.79 (m, 4H), 1.71-1.51 (m, 3H)

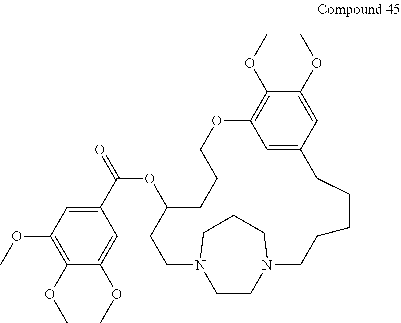 [SMILES: COc1cc(C(=O)OC2CCCOc3cc(cc(OC)c3OC)CCCCCN3CCCN(CC2)CC3)cc(OC)c1OC]

To a stirred mixture of ADDP (42 mg, 0.167 mmol, 1.5 equiv) in THF (1.0 mL) was added n-Bu₃P (42 μL, 0.167 mmol, 1.5 equiv) at room temperature under nitrogen atmosphere and the mixture was stirred for 30 mins. The intermediate compound 33 (70 mg, 0.111 mmol, 1.0 equiv) in THF (1.0 mL) was added and the resulting mixture was stirred for additional 1 h at 40° C., and then allowed to cool down to room temperature. The reaction was quenched with 50% brine (30 mL) at room temperature. The resulting mixture was extracted with EtOAc (3×50 mL). The combined organic layers were dried over anhydrous Na₂SO₄ and concentrated under reduced pressure. The residue was purified by reverse flash chromatography (column, C18 silica gel, mobile phase, MeCN (0.1% Formic Acid) in water (0.1% Formic Acid), (10-60% gradient), (UV 254 nm and 220 nm), affording the compound 45 (5 mg, 7% yield) as a colourless solid.

LCMS (ESI position ion) m/z: 615.6 (M+H)+ (calculated: 615.4)

1H NMR (400 MHz, CDCl3-d) δ 7.27 (s, 2H), 6.44 (d, J=1.8 Hz, 1H), 6.42 (d, J=1.7 Hz, 1H), 5.54-5.43 (m, 1H), 4.16-4.07 (m, 2H), 3.93 (s, 3H), 3.92-3.91 (m, 6H), 3.91-3.90 (m, 3H), 3.88-3.84 (m, 3H), 3.80 (s, 3H), 3.38-3.21 (m, 3H), 3.20-3.00 (m, 2H), 3.00-2.84 (m, 3H), 2.81-2.71 (m, 2H), 2.62-2.52 (m, 4H), 2.06-1.66 (m, 6H), 1.66-1.55 (m, 3H), 1.49-1.36 (m, 2H)

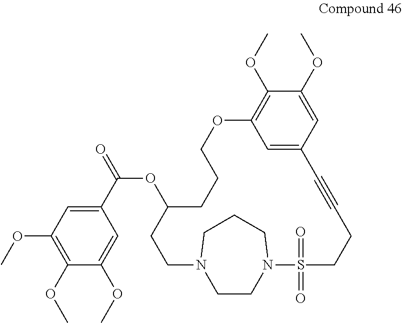 [SMILES: COc1cc(C(=O)OC2CCCOc3cc(cc(OC)c3OC)C#CCCS(=O)(=O)N3CCCN(CC2)CC3)cc(OC)c1OC]

The compound 46 was prepared from the intermediate compound 35 (90 mg, 0.133 mmol, 1.0 equiv) using the protocol described for compound 45. The residue was purified by reverse flash chromatography (column, C18 silica gel; mobile phase, MeCN (0.1% Formic Acid) in water (0.1% Formic Acid), (10-60% gradient), (UV 254 nm and 220 nm) affording the compound 46 (43 mg, 49% yield) as a white solid.

LCMS (ESI position ion) m/z: 661.7 (M+H)+ (calculated: 661.3)

1H NMR (400 MHz, CDCl3-d) δ 7.27 (s, 2H), 6.80 (d, J=1.7 Hz, 1H), 6.65 (d, J=1.7 Hz, 1H), 5.47-5.36 (m, 1H), 4.25-4.13 (m, 1H), 3.90 (s, 9H), 3.86-3.83 (m, 6H), 3.80-3.60 (m, 2H), 3.59-3.48 (m, 1H), 3.42-3.26 (m, 3H), 2.95-2.89 (m, 2H), 2.86-2.78 (m, 2H), 2.76-2.54 (m, 2H), 2.00-1.90 (m, 5H), 1.87-1.78 (m, 1H), 1.78-1.67 (m, 2H), 1.67-1.37 (m, 3H).

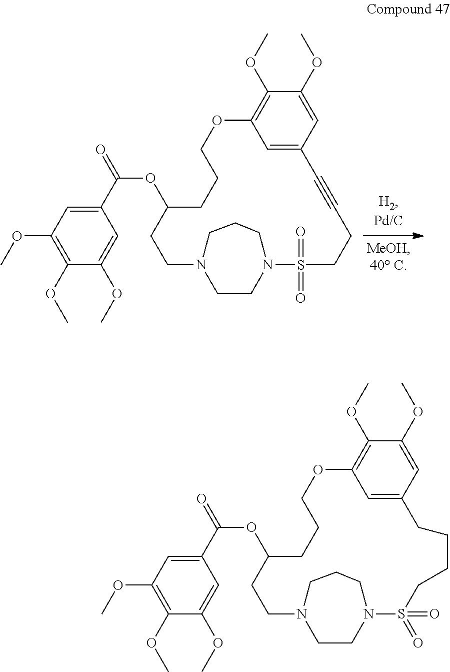 [SMILES: COc1cc(C(=O)OC2CCCOc3cc(cc(OC)c3OC)C#CCCS(=O)(=O)N3CCCN(CC2)CC3)cc(OC)c1OC ; COc1cc(C(=O)OC2CCCOc3cc(cc(OC)c3OC)CCCCS(=O)(=O)N3CCCN(CC2)CC3)cc(OC)c1OC]

A solution of compound 46 (26 mg, 0.039 mmol, 1.00 equiv) and 10% palladium on carbon (21 mg) in methanol (2.50 mL) under H2 atmosphere (Endeavor, 60 psi) was stirred at 40° C. for 4 h. The reaction mixture was filtered through celite and washed with methanol. The filtrate was evaporated in vacuo to afford a colourless oil. The residue was purified by reverse flash chromatography (column, C18 silica gel; mobile phase, MeCN (0.1% Formic Acid) in water (0.1% Formic Acid), (15-50% gradient), (UV 254 nm and 220 nm), affording the compound 47 (8 mg, 31% yield) as a colourless solid.

LCMS (ESI position ion) m/z: 665.6 (M+H)+ (calculated: 665.3)

1H NMR (400 MHz, CDCl3-d) δ 7.29 (s, 2H), 6.43 (d, J=1.8 Hz, 1H), 6.39 (d, J=1.7 Hz, 1H), 5.51 (s, 1H), 4.22-4.13 (m, 1H), 4.10-4.02 (m, 1H), 3.92 (d, J=4.5 Hz, 9H), 3.85 (s, 3H), 3.81 (s, 3H), 3.41-3.19 (m, 6H), 2.97 (t, J=7.4 Hz, 2H), 2.71-2.48 (m, 7H), 2.14-1.98 (m, 2H), 1.94-1.76 (m, 2H), 1.73-1.65 (m, 1H), 1.64-1.56 (m, 6H).

### II. Biology Examples

### Example II.1. Assay for ENT1 Activity

### Example II.1.a Binding Assay

### Purpose

The present assay aims at showing that the compounds of the present disclosure can bind to human ENT1. The principle of the assay is a competition between the compounds of this present disclosure and Sahenta-DY647, an ENT1 inhibitor that emits fluorescence (Ex=630 nm, Em=670 nm). By measuring the fluorescence at the end of the assay we could assess the binding potency of the compounds of the present disclosure.

### Method

JAR cells expressing ENT1 were bought from ATCC® (HTB-144TM). Cells were cultured in RPMI 1640 medium (LONZA®, #BE12-702F/U1) supplemented with 10% FBS (GIBCO®, #10270-106), 10 mM Hepes (LONZA®, #BE17-737E), 1 mM Sodium Pyruvate (LONZA®, #BE13-115E) and 2% Penicillin/Streptomycin (LONZA®, #DE17-603E) at 37° C. and 5% CO2. The assay was conducted on the following buffer: HBSS (LONZA®, #LO-527F) supplemented with 10 mM Hepes (LONZA®, #BE17-737E) and 0.1% BSA (Miltenyi®, #130-091-376) on the day of the assay.

JAR cells were resuspended in the described buffer. Compounds of the present disclosure and Sahenta-DY647 were diluted 200× in the described buffer.

A total of 50 000 cells were pre-incubated for 30 min at 4° C. with the compounds of the present disclosure before adding the corresponded IC90 of Sahenta-DY647 (100 nM) and incubate once more for 30 min at 4° C. The total volume of the reaction was 100 μL (50 μL of cells, 25 μL of the compounds of the present disclosure and 25 μL of Sahenta-DY647) in a 96 well plate, U-bottom (Greiner®, #650-180). The plates were washed 2× by centrifugation (4 min, 400 rcf at 4° C.) in the same buffer. Cells were re-suspended in 70 μL of the buffer and 50 μL was transferred to a Black 384 Optiplate (PerkinElmer®, #6007279). Fluorescence (Ex=630 nm, Em=670 nm) was acquired on a Spectramax i3x (Molecular Devices®).

### Results

Results obtained from this protocol are summarized in Table 5.

### Example II.1. b Functional Assay: Uridine Transport Inhibition Assay

### Purpose

The aim of this study was to determine the potency of equilibrative nucleoside transporter 1 (ENT1) inhibitors by measuring ENT1-mediated transport is the cellular uptake assay. The human ENT1 transporter can be stably expressed in Madin-Darby Canine Kidney II (MDCKII) cells via transduction. Uridine is efficiently transported by ENT1 and is used as probe in the assay as 3H-uridine. The interaction is detected as the modulation of the initial rate of 3H-uridine transport by human ENT1 into MDCKII-ENT1-LV cells stably expressing ENT1 uptake transporter.

### Results

Results obtained from this protocol are summarized in Table 6.

TABLE 3
Assay parameters

| | Incubation time | | Reference | Signal/ |
|---|---|---|---|---|
| | (min) and | Probe substrate | inhibitor | Noise |
| Transporter | temperature | (concentration) | (concentration) | ratio |
| ENT1 | 1 min at 25° C. | Uridine | Dilazep | >3 |
| | | (1 μM) | (2 μM) | |

### Example II.1.c Functional Assay: T Cell Proliferation Assay

### Purpose

The aim of this study was to determine the potency of equilibrative nucleoside transporter 1 (ENT1) inhibitors to rescue proliferation by stimulated primary human T cells incubated in the presence of 100 uM Adenosine triphosphate (ATP), in baseline conditions (condition A) or in the presence of various proteins known to bind small molecules (condition B)

Condition A: X-VIVO15

Condition B: X-VIVO15, 2% Human Serum Albumine (HSA) and 0.1% α-1-Acid Glycoprotein (AAG)

TABLE 4
Materials for Functional Assay

| Product | Source | Cat number |
|---|---|---|
| RPMI 1640 | LONZA | BE12-702F/U1 |
| FBS | GIBCO | 10270-106 |
| X-VIVO15 | Lonza | BE02-060Q |
| Human Serum Albumine (HSA) | Sigma-Aldrich | A1653 |
| α1-Acid Glycoprotein (AAG) | Sigma-Aldrich | G9885 |
| Dynabeads CD3/28 activation | Thermo Fisher | 11132D |
| beads | Scientific | |
| CFDA, SE | Life Technologies | C1157 |
| Dipyridamole | Tocris | 0691 |
| ATP | Sigma Aldrich | A6419-1G |

### Method

Cryopreserved purified human CD3+ T cells were thawed and washed twice with RPMI1640 medium, UltraGlutamine containing 10% hiFBS.

Cells were suspended in PBS containing 10% hiFBS. Cells were stained with CFSE by adding 2 μM solution in PBS, to get a final 1 μM CFSE solution. Cells were incubated while rotating for 5 minutes. Reaction was stopped by adding PBS with 10% FBS and cells were centrifuged for 5 minutes at 1500 rpm.

Cells were resuspended at 1.6×106 cells/mL, either in X-VIVO15 medium or in 4% Human Serum Albumin and 0.2% α-1-Acid Glycoprotein. 50 μL of cell suspension (8×104 T cells) was added to wells of sterile round-bottom 96-well plates. Cells were activated by adding 50 μL of anti-CD3 anti-CD28 coated microbeads, suspended either in XVIVO-15 medium or in 4% HSA and 0.2% α-1-Acid Glycoprotein, at a ratio of one microbead per two cells.

Serial dilutions of the ENT1 inhibitors were prepared in X-VIVO15 from 10 mM stock solutions in DMSO, and 50 μL was added to the wells.

ATP powder was diluted in X-VIVO15, and 50 μL of this compound was added to the wells to reach a final assay concentration of 100 μM. Final volume of 200 μL.

The experiments were also performed in 384 well plates—all volumes reduced by a factor of 4 (12.5 μL) with a final volume of 50 μL.

Experiments were performed in duplicate. The cells were placed in a 37° C. humidified tissue culture incubator with 5% CO2 for 72 hours for 96 well plates, 96 hours for 384 well plates After 72 or 96 hours, proliferation was measured determined by CFSE dilution via flow cytometry.

### Results

Results are detailed below in Table 6. Compounds of the present disclosure have good ENT1 inhibitory properties.

### Example II.2. Results of ENT1 Inhibition

### Results

The potency as been determined in the binding assay as is reported in Table 5. The compound of the present disclosure presents a similar potency as compared to dilazep against ENT1.

The IC50 has been binned following the ranges: IC50 below 0.001 μM: +++; IC50 between 0.001 and 0.02 μM: ++; IC50 between 0.02 and 0.5 μM: +, between 0.5 and 1.0 μM: −

TABLE 5

| Compounds | Binding Assay IC50 |
|---|---|
| Dilazep | ++ |
| Compound 1 | + |
| Compound 2 | ++ |
| Compound 3 | +++ |
| Compound 4 | ++ |
| Compound 5 | + |
| Compound 6 | ++ |
| Compound 7 | ++ |
| Compound 8 | ++ |
| Compound 9 | ++ |
| Compound 10 | ++ |
| Compound 11 | ++ |
| Compound 12 | ++ |
| Compound 13 | ++ |
| Compound 14 | ++ |
| Compound 15 | ++ |
| Compound 16 | ++ |
| Compound 17 | ++ |
| Compound 18 | + |
| Compound 19 | ++ |
| Compound 20 | + |
| Compound 21 | ++ |
| Compound 22 | + |
| Compound 23 | ++ |
| Compound 24 | ++ |
| Compound 25 | − |
| Compound 26 | ++ |
| Compound 27 | ++ |
| Compound 28 | ++ |
| Compound 29 | ++ |
| Compound 30 | ++ |
| Compound 31 | + |
| Compound 32 | − |
| Compound 33 | + |
| Compound 34 | − |
| Compound 35 | − |
| Compound 36 | + |
| Compound 37 | ++ |
| Compound 38 | − |
| Compound 39 | + |
| Compound 40 | + |
| Compound 41 | ++ |
| Compound 42 | ++ |
| Compound 43 | ++ |
| Compound 44 | ++ |
| Compound 45 | ++ |
| Compound 46 | + |
| Compound 47 | ++ |

### Discussion of Results in Table 6

The potency has been determined in two independent functional assays: (1) a transporter assay using cell lines, and (2) a proliferation assay including our primary target immune cells, T cells. Assay (2) also includes a Condition B representative for the challenging conditions in the tumor microenvironment (TME), containing elevated levels of proteins known to bind small molecules (which has a negative impact on potency). A summary of potencies identified in these assays is reported in Table 6. The compound of the present disclosure presents a maintained, or strongly improved potency as compared to dilazep in all functional assays. In particular, the compounds of the present disclosure present a significantly improved potency in the T Cell proliferation assay in baseline conditions (condition A) and in conditions mimicking the TME (condition B) as compared to dilazep. As the compound of the present disclosure has greatly improved potency compared to dilazep in a biologically relevant functional assay, this implies a significantly better safety window regarding off-targets.

The IC50 has been binned following the ranges. IC50 below 0.0001 μM: ++++; IC50 below 0.001 μM: +++; IC50 between 0.001 and 0.02 μM: ++; IC50 between 0.02 and 0.5 μM: +, above 0.5 μM: −

TABLE 6

| | | (2) T Cell |
|---|---|---|
| | T Cell proliferation | proliferation |
| | (Condition A) | (Condition B) |
| COMPOUNDS | IC50 | IC50 |
| Dilazep | ++ | + |
| Compound 2 | ++ | − |
| Compound 3 | ++++ | +++ |
| Compound 4 | ++++ | +++ |
| Compound 6 | +++ | − |
| Compound 9 | ++++ | ++ |
| Compound 10 | ++++ | + |
| Compound 11 | +++ | − |
| Compound 12 | +++ | ++ |
| Compound 15 | +++ | ++ |
| Compound 16 | ++++ | ++ |
| Compound 17 | ++++ | ++ |
| Compound 19 | +++ | + |
| Compound 20 | ++ | − |
| Compound 21 | ++++ | ++ |
| Compound 23 | ++ | + |
| Compound 24 | ++++ | + |
| Compound 28 | +++ | + |
| Compound 29 | +++ | + |
| Compound 30 | +++ | + |
| Compound 33 | ++ | − |
| Compound 37 | +++ | + |
| Compound 40 | + | + |
| Compound 41 | +++ | + |
| Compound 42 | +++ | + |
| Compound 43 | +++ | + |
| Compound 44 | ++++ | + |
| Compound 47 | +++ | ++ |

1. A compound represented by Formula (I): 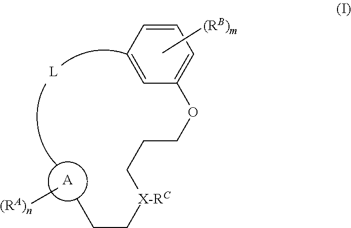
or a pharmaceutically acceptable salt thereof,
wherein
L is an optionally substituted C₃-C₇ alkylene chain, wherein one, two, or three methylene units is optionally and independently replaced with —O—, —N(R¹)—, —C(O)—, —C(O)O—, —C(O)N(R¹)—, —S(O)₂—, a 5-membered heteroaryl, —CH═CH—, or —C≡C—;
A is selected from the group consisting of —N(R^{A})— and 5-7 membered heterocyclyl;
X is —C(H)— or —N—;
each R^{A} is independently selected from the group consisting of halogen and optionally substituted C₁-C₆ alkyl;
each R^{B} is independently selected from the group consisting of optionally substituted C₁-C₆ alkoxy and halogen;
R^{C} is selected from the group consisting of hydrogen, optionally substituted benzyl, —OR², —OC(O)R², —C(O)R², —OC(O)OR², —N(R²)₂, and —OC(O)N(R²)₂;
each R¹ is hydrogen or optionally substituted C₁-C₃ alkyl;
each R² is independently selected from the group consisting of optionally substituted C₁-C₆ alkyl, —(CH₂)₀₋₃phenyl, —(CH₂)₀₋₃C(O)R³, 5-10 membered heteroaryl, 3-7 membered heterocyclyl, and —N═CH-phenyl, wherein each R² is optionally substituted with one, two, or three instances of R⁴;
or two instances of R² may be joined together with the atom on which they are attached to form a 5-10 membered heteroaryl or 3-7 membered heterocyclyl, optionally substituted with one, two, or three instances of R⁴;
each R³ is 5-10 membered heteroaryl or 3-7 membered heterocyclyl, wherein R³ is optionally substituted with one, two, or three instances of R⁴;
each R⁴ is selected from the group consisting of halogen, —OH, —NH₂, —CN, —NHR¹, optionally substituted C₁-C₆ alkyl, optionally substituted C₁-C₆ alkoxy, and —S(O)₂C₁-C₃ alkyl;
or two instances of R⁴ may be joined together with the atom on which they are attached to form a 5-10 membered heteroaryl or 3-7 membered heterocyclyl, optionally substituted with one, two, or three instances of oxo, halogen or C₁-C₃ alkyl;
n is 0, 1, 2, or 3; and
m is 0, 1, 2, or 3.

2. The compound of claim 1, wherein L is an optionally substituted C₃-C₇ alkylene chain, wherein a methylene unit is replaced with —C(O)N(R¹)—.

3. The compound of claim 1, wherein L is an optionally substituted C₃-C₇ alkylene chain, wherein a methylene unit is replaced with —C(O)O—.

4. The compound of claim 1, wherein L is an optionally substituted C₃-C₇ alkylene chain, wherein a methylene unit is replaced with —O—.

5. The compound of claim 1, wherein L is an optionally substituted C₃-C₇ alkylene chain, wherein a methylene unit is replaced with —S(O)₂—.

6. The compound of claim 1, wherein the compound is represented by Formula (I-a): 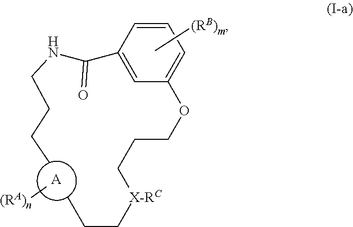
or a pharmaceutically acceptable salt thereof.

7. The compound of claim 1, wherein the compound is represented by Formula (I-b): 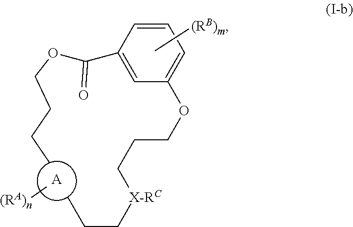
or a pharmaceutically acceptable salt thereof.

8. The compound of claim 1, wherein
R² is 5-6-membered heteroaryl, wherein R² is optionally substituted with one, two, or three instances of R⁴.

9. The compound of claim 1, wherein A is selected from the group consisting of

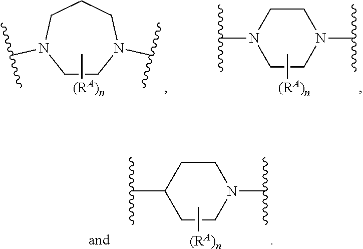

10. The compound of claim 9, wherein A is selected from the group consisting of

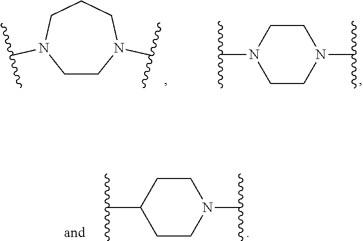 [SMILES: *N1CCCN(*)CC1 ; *N1CCN(*)CC1 ; *C1CCN(*)CC1]

11. The compound of claim 1, wherein the compound is represented by Formula (I-c): 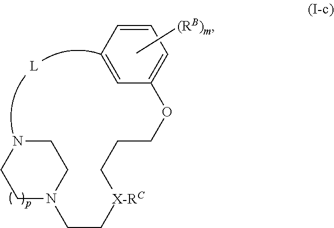
or a pharmaceutically acceptable salt thereof
wherein
p is 1 or 2.

12. The compound of claim 1, wherein R^{C} is —OC(O)R².

13. The compound of claim 12, wherein R² is 5-10-membered heteroaryl, wherein R² is optionally substituted with one, two, or three instances of R⁴.

14. The compound of claim 13, wherein R^{C} is selected from the group consisting of

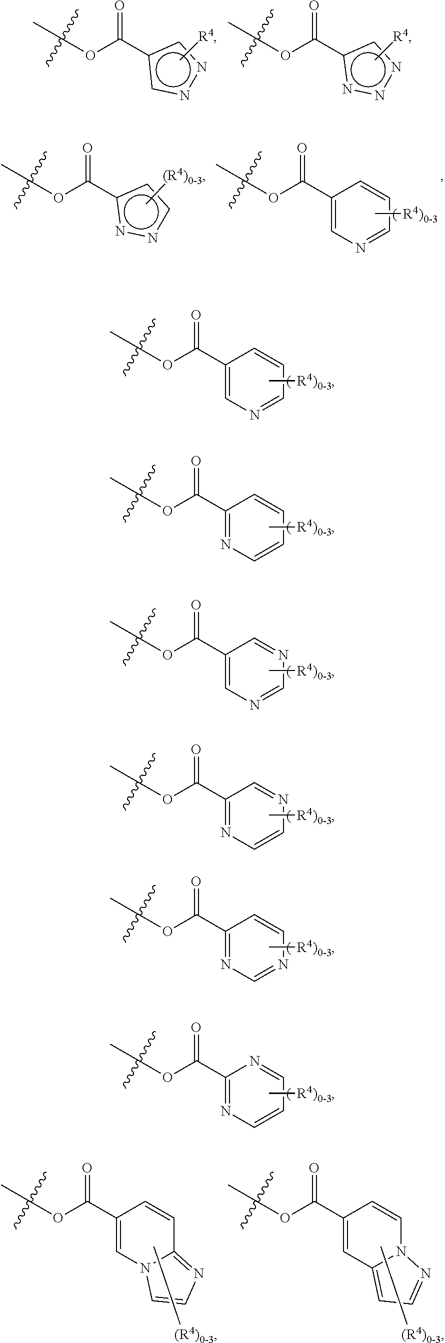

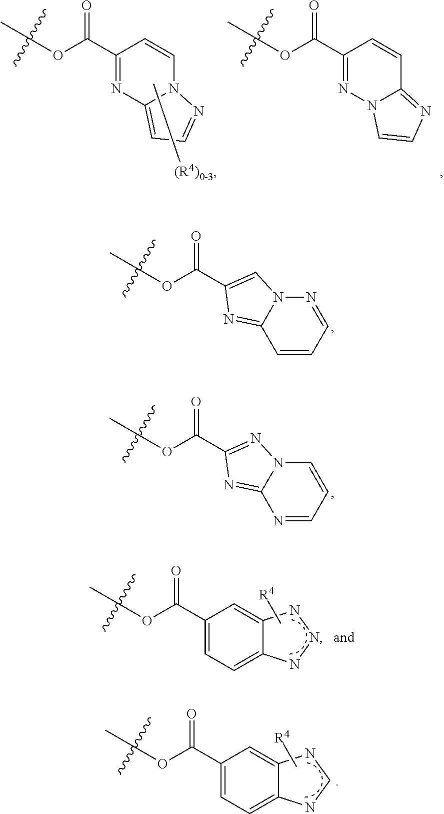

15. The compound of claim 12, wherein R² is 5-10-membered heterocyclyl, wherein R² is optionally substituted with one, two, or three instances of R⁴.

16. The compound of claim 15, wherein R^{C} is selected from the group consisting of

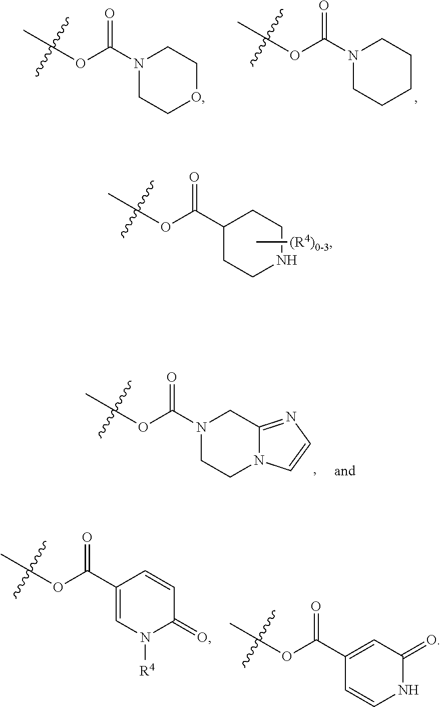

17. The compound of claim 12, wherein R² is —(CH₂)₀₋₃C(O)R³.

18. The compound of claim 17, wherein R^{C} is selected from the group consisting of

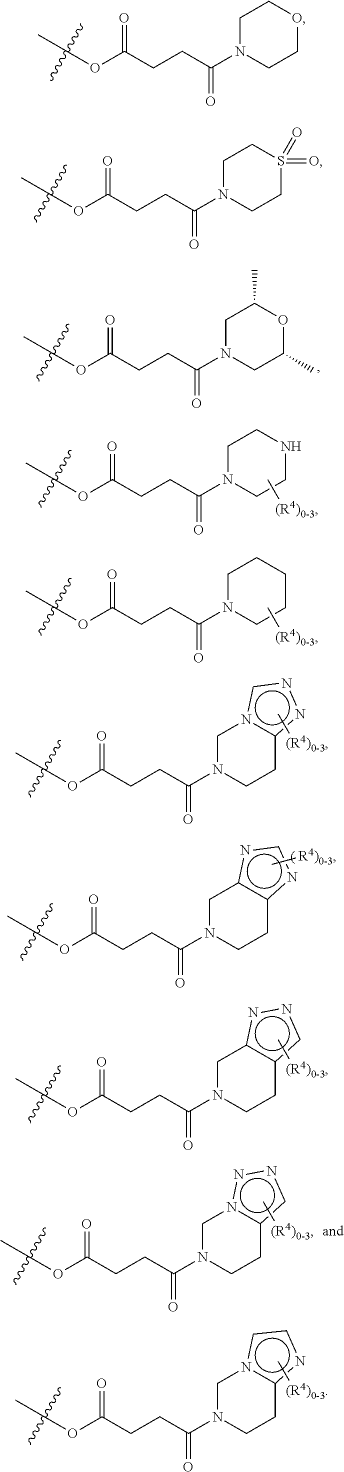

19. The compound of claim 1, wherein R^{C} is selected from the group consisting of

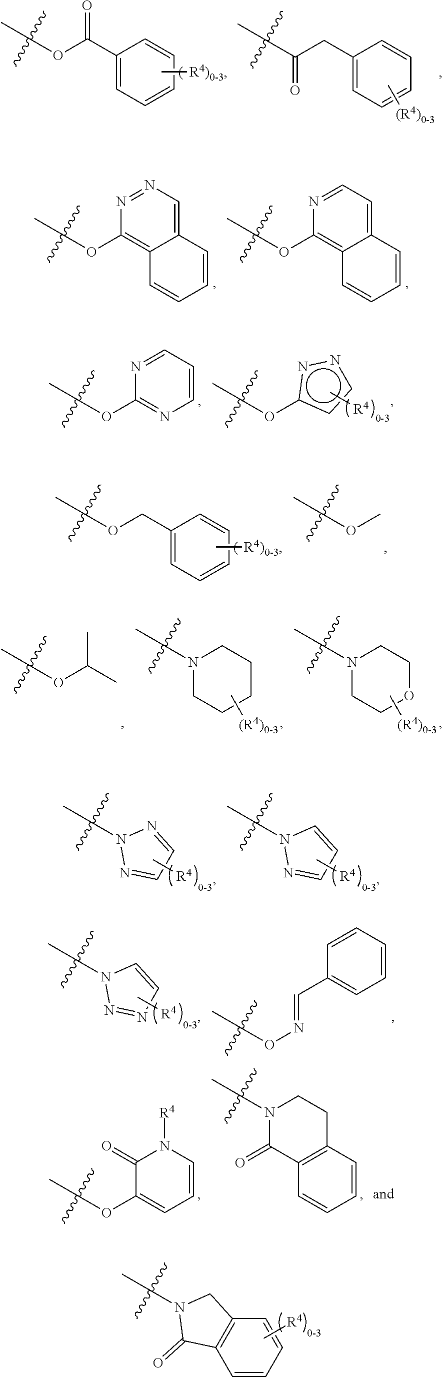

20. The compound of claim 1, wherein X is —C(H)—.

21. The compound of claim 1, wherein n is 0.

22. The compound of claim 1, wherein m is 0.

23. The compound of claim 1, wherein m is 1.

24. The compound of claim 1, wherein m is 2 or 3.

25. (canceled)

26. The compound of claim 1, wherein each R⁴ is selected from the group consisting of halogen, optionally substituted C₁-C₆ alkyl, optionally substituted C₁-C₆ alkoxy, and —S(O)₂C₁-C₃ alkyl.

27. (canceled)

28. A compound selected from the group consisting of

| Compound No. | Structure |
|---|---|
| 1 | 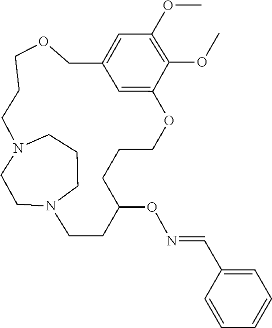 |
| 2 | 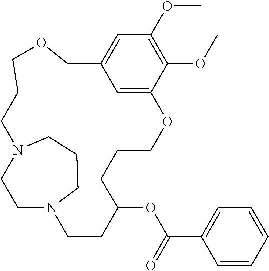 [SMILES: COc1cc2cc(c1OC)OCCCC(OC(=O)c1ccccc1)CCN1CCCN(CCCOC2)CC1] |
| 3 | 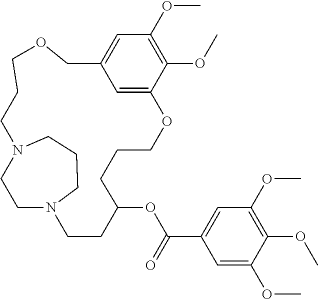 [SMILES: COc1cc(C(=O)OC2CCCOc3cc(cc(OC)c3OC)COCCCN3CCCN(CC2)CC3)cc(OC)c1OC] |
| 4 | 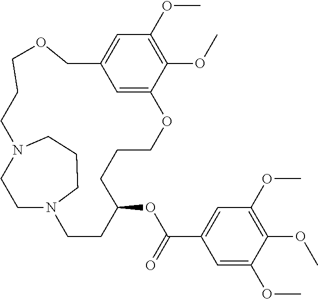 [SMILES: COc1cc(C(=O)O[C@@H]2CCCOc3cc(cc(OC)c3OC)COCCCN3CCCN(CC2)CC3)cc(OC)c1OC] |
| 5 | 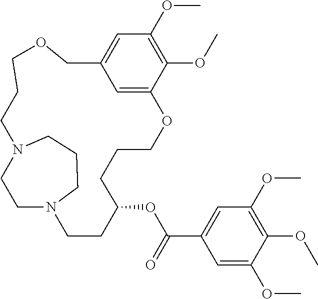 [SMILES: COc1cc(C(=O)O[C@H]2CCCOc3cc(cc(OC)c3OC)COCCCN3CCCN(CC2)CC3)cc(OC)c1OC] |
| 6 | 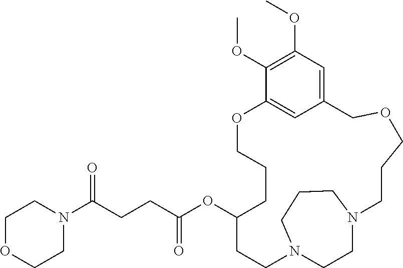 [SMILES: COc1cc2cc(c1OC)OCCCC(OC(=O)CCC(=O)N1CCOCC1)CCN1CCCN(CCCOC2)CC1] |
| 7 | 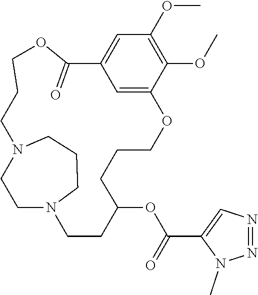 [SMILES: COc1cc2cc(c1OC)OCCCC(OC(=O)c1cnnn1C)CCN1CCCN(CCCOC2=O)CC1] |
| 8 | 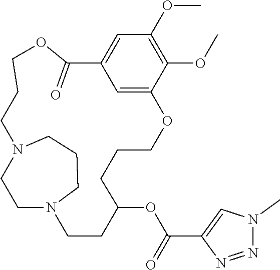 [SMILES: COc1cc2cc(c1OC)OCCCC(OC(=O)c1cn(C)nn1)CCN1CCCN(CCCOC2=O)CC1] |
| 9 | 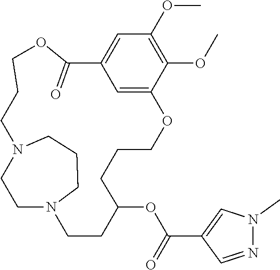 [SMILES: COc1cc2cc(c1OC)OCCCC(OC(=O)c1cnn(C)c1)CCN1CCCN(CCCOC2=O)CC1] |
| 10 | 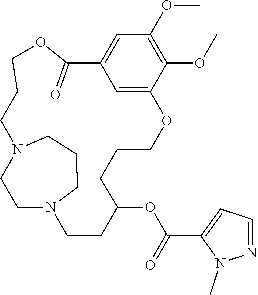 [SMILES: COc1cc2cc(c1OC)OCCCC(OC(=O)c1ccnn1C)CCN1CCCN(CCCOC2=O)CC1] |
| 11 | 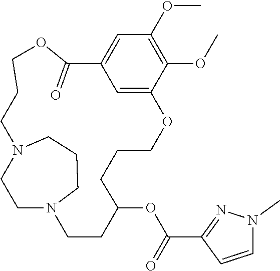 [SMILES: COc1cc2cc(c1OC)OCCCC(OC(=O)c1ccn(C)n1)CCN1CCCN(CCCOC2=O)CC1] |
| 12 | 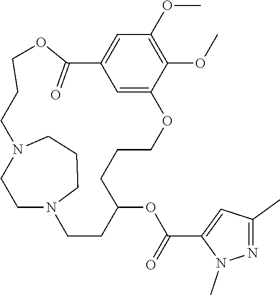 [SMILES: COc1cc2cc(c1OC)OCCCC(OC(=O)c1cc(C)nn1C)CCN1CCCN(CCCOC2=O)CC1] |
| 13 | 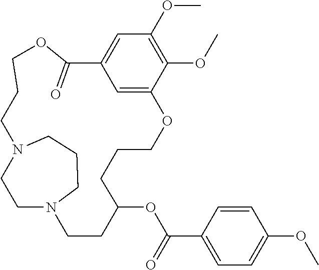 [SMILES: COc1ccc(C(=O)OC2CCCOc3cc(cc(OC)c3OC)C(=O)OCCCN3CCCN(CC2)CC3)cc1] |
| 14 | 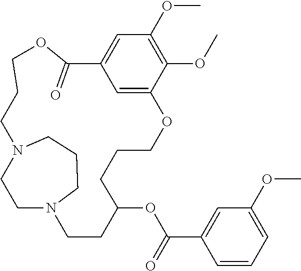 [SMILES: COc1cccc(C(=O)OC2CCCOc3cc(cc(OC)c3OC)C(=O)OCCCN3CCCN(CC2)CC3)c1] |
| 15 | 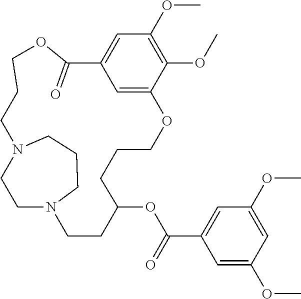 [SMILES: COc1cc(OC)cc(C(=O)OC2CCCOc3cc(cc(OC)c3OC)C(=O)OCCCN3CCCN(CC2)CC3)c1] |
| 16 | 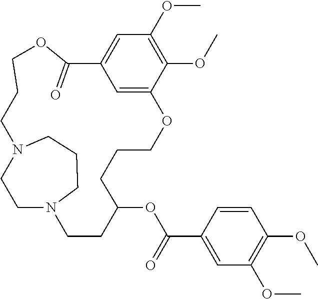 [SMILES: COc1ccc(C(=O)OC2CCCOc3cc(cc(OC)c3OC)C(=O)OCCCN3CCCN(CC2)CC3)cc1OC] |
| 17 | 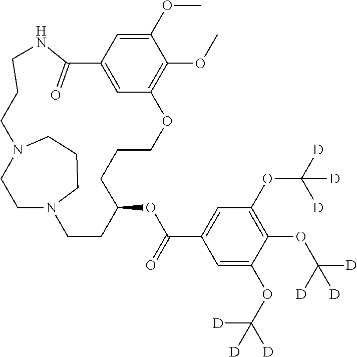 [SMILES: [2H]C([2H])([2H])Oc1cc(C(=O)O[C@@H]2CCCOc3cc(cc(OC)c3OC)C(=O)NCCCN3CCCN(CC2)CC3)cc(OC([2H])([2H])[2H])c1OC([2H])([2H])[2H]] |
| | (absolute stereochemistry) |
| 18 | 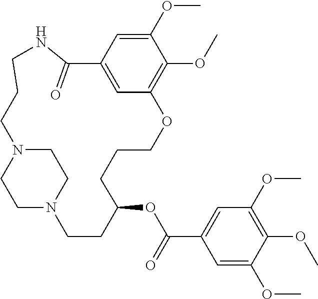 [SMILES: COc1cc(C(=O)O[C@@H]2CCCOc3cc(cc(OC)c3OC)C(=O)NCCCN3CCN(CC2)CC3)cc(OC)c1OC] |
| 19 | 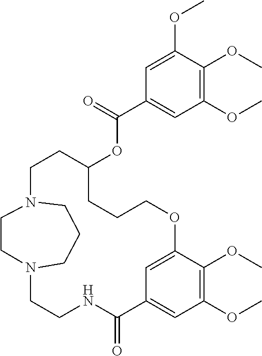 [SMILES: COc1cc(C(=O)OC2CCCOc3cc(cc(OC)c3OC)C(=O)NCCN3CCCN(CC2)CC3)cc(OC)c1OC] |
| 20 | 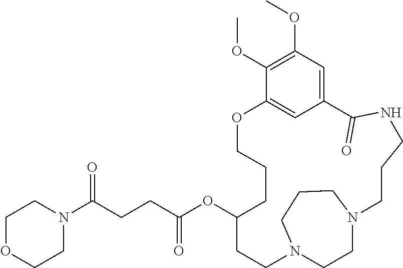 [SMILES: COc1cc2cc(c1OC)OCCCC(OC(=O)CCC(=O)N1CCOCC1)CCN1CCCN(CCCNC2=O)CC1] |
| 21 | 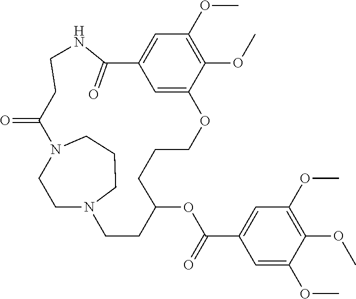 [SMILES: COc1cc(C(=O)OC2CCCOc3cc(cc(OC)c3OC)C(=O)NCCC(=O)N3CCCN(CC2)CC3)cc(OC)c1OC] |
| 22 | 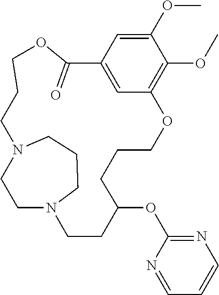 [SMILES: COc1cc2cc(c1OC)OCCCC(Oc1ncccn1)CCN1CCCN(CCCOC2=O)CC1] |
| 23 | 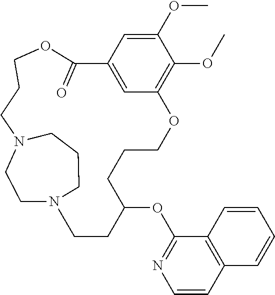 [SMILES: COc1cc2cc(c1OC)OCCCC(Oc1nccc3ccccc13)CCN1CCCN(CCCOC2=O)CC1] |
| 24 | 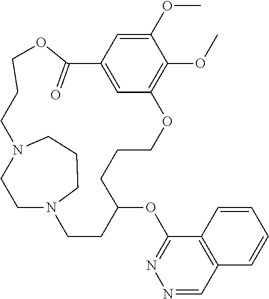 [SMILES: COc1cc2cc(c1OC)OCCCC(Oc1nncc3ccccc13)CCN1CCCN(CCCOC2=O)CC1] |
| 25 | 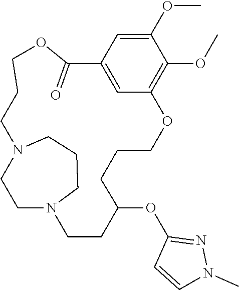 [SMILES: COc1cc2cc(c1OC)OCCCC(Oc1ccn(C)n1)CCN1CCCN(CCCOC2=O)CC1] |
| 26 | 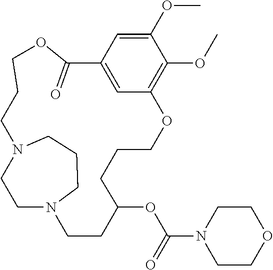 [SMILES: COc1cc2cc(c1OC)OCCCC(OC(=O)N1CCOCC1)CCN1CCCN(CCCOC2=O)CC1] |
| 27 | 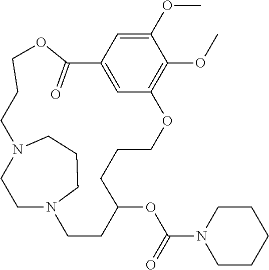 [SMILES: COc1cc2cc(c1OC)OCCCC(OC(=O)N1CCCCC1)CCN1CCCN(CCCOC2=O)CC1] |
| 28 | 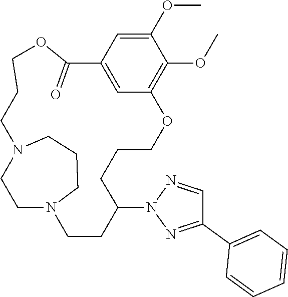 |
| 29 | 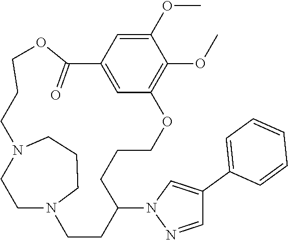 [SMILES: COc1cc2cc(c1OC)OCCCC(n1cc(-c3ccccc3)cn1)CCN1CCCN(CCCOC2=O)CC1] |
| 30 | 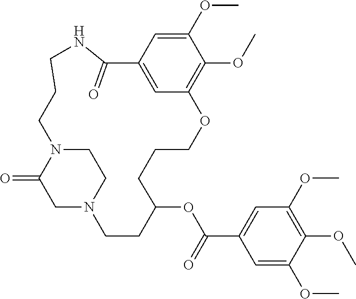 [SMILES: COc1cc(C(=O)OC2CCCOc3cc(cc(OC)c3OC)C(=O)NCCCN3CCN(CC2)CC3=O)cc(OC)c1OC] |
| 31 | 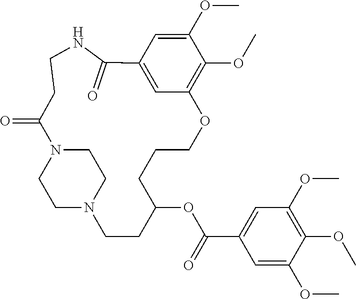 [SMILES: COc1cc(C(=O)OC2CCCOc3cc(cc(OC)c3OC)C(=O)NCCC(=O)N3CCN(CC2)CC3)cc(OC)c1OC] |
| 32 | 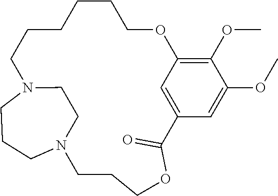 [SMILES: COc1cc2cc(c1OC)OCCCCCCN1CCCN(CCCOC2=O)CC1] |
| 33 | 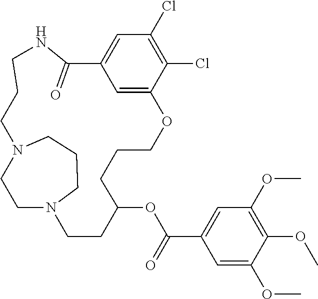 [SMILES: COc1cc(C(=O)OC2CCCOc3cc(cc(Cl)c3Cl)C(=O)NCCCN3CCCN(CC2)CC3)cc(OC)c1OC] |
| 34 | 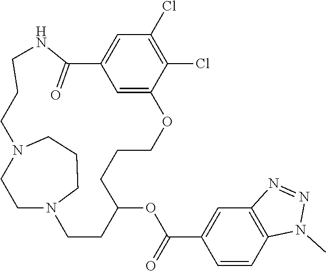 [SMILES: Cn1nnc2cc(C(=O)OC3CCCOc4cc(cc(Cl)c4Cl)C(=O)NCCCN4CCCN(CC3)CC4)ccc21] |
| 35 | 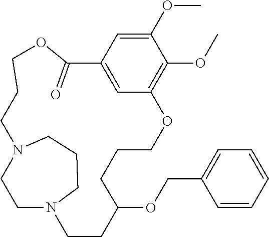 [SMILES: COc1cc2cc(c1OC)OCCCC(OCc1ccccc1)CCN1CCCN(CCCOC2=O)CC1] |
| 36 | 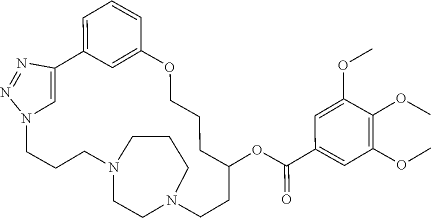 [SMILES: COc1cc(C(=O)OC2CCCOc3cccc(c3)-c3cn(nn3)CCCN3CCCN(CC2)CC3)cc(OC)c1OC] |
| 37 | 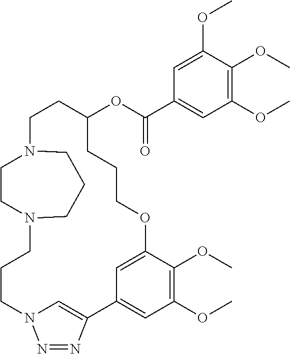 [SMILES: COc1cc(C(=O)OC2CCCOc3cc(cc(OC)c3OC)-c3cn(nn3)CCCN3CCCN(CC2)CC3)cc(OC)c1OC] |
| 38 | 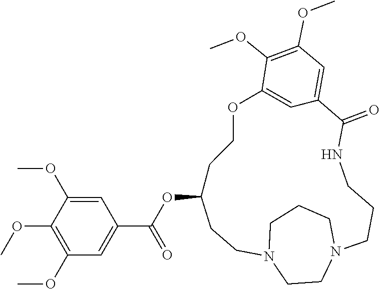 [SMILES: COc1cc(C(=O)O[C@H]2CCOc3cc(cc(OC)c3OC)C(=O)NCCCN3CCCN(CC2)CC3)cc(OC)c1OC] |
| 39 | 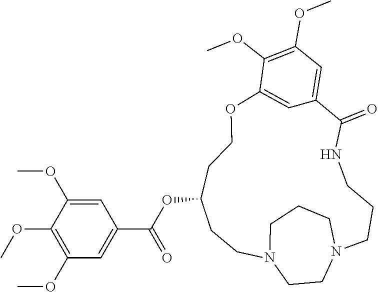 [SMILES: COc1cc(C(=O)O[C@@H]2CCOc3cc(cc(OC)c3OC)C(=O)NCCCN3CCCN(CC2)CC3)cc(OC)c1OC] |
| 40 | 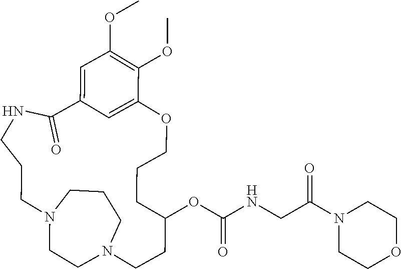 [SMILES: COc1cc2cc(c1OC)OCCCC(OC(=O)NCC(=O)N1CCOCC1)CCN1CCCN(CCCNC2=O)CC1] |
| 41 | 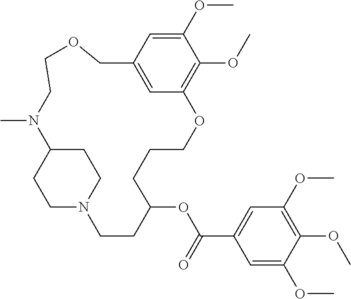 |
| 42 | 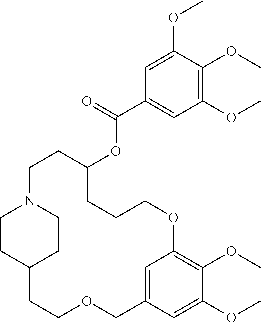 [SMILES: COc1cc(C(=O)OC2CCCOc3cc(cc(OC)c3OC)COCCC3CCN(CC3)CC2)cc(OC)c1OC] |
| 43 | 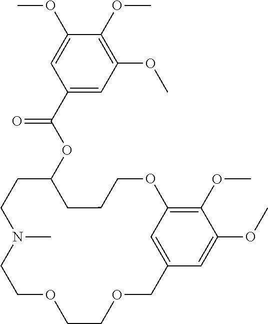 [SMILES: COc1cc(C(=O)OC2CCCOc3cc(cc(OC)c3OC)COCCOCCN(C)CC2)cc(OC)c1OC] |
| 44 | 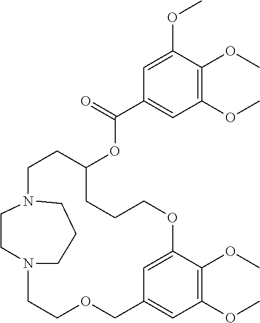 [SMILES: COc1cc(C(=O)OC2CCCOc3cc(cc(OC)c3OC)COCCN3CCCN(CC2)CC3)cc(OC)c1OC] |
| 45 | 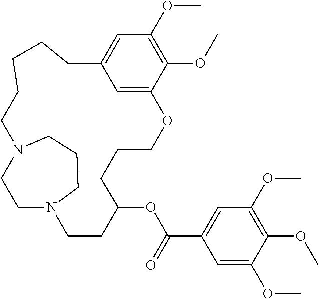 [SMILES: COc1cc(C(=O)OC2CCCOc3cc(cc(OC)c3OC)CCCCCN3CCCN(CC2)CC3)cc(OC)c1OC] |
| 46 | 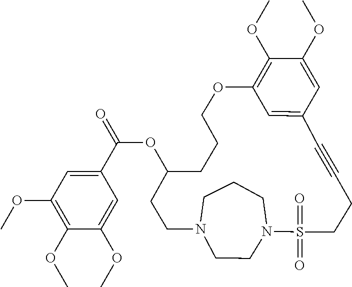 [SMILES: COc1cc(C(=O)OC2CCCOc3cc(cc(OC)c3OC)C#CCCS(=O)(=O)N3CCCN(CC2)CC3)cc(OC)c1OC] |
| 47 | 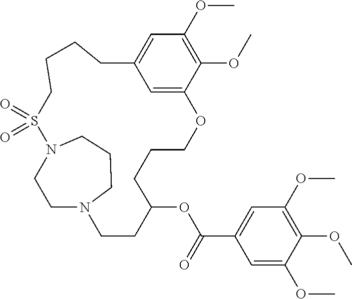 [SMILES: COc1cc(C(=O)OC2CCCOc3cc(cc(OC)c3OC)CCCCS(=O)(=O)N3CCCN(CC2)CC3)cc(OC)c1OC] |

or a pharmaceutically acceptable salt thereof.

29. A pharmaceutical composition comprising a compound according to claim 1 and a pharmaceutically acceptable excipient.

30. A method of inhibiting ENT1 in a patient need thereof, comprising: administering to said patient an effective amount of a compound according to claim 1.

31. A method of treating cancer in a patient need thereof, comprising: administering to said patient an effective amount of a compound according to claim 1.

32. A method of treating cancer in a patient need thereof, comprising: administering to said patient a combination of a compound according to claim 1 and an adenosine receptor antagonist.

33. The method according to claim 32, wherein the adenosine receptor antagonist is an A2A or A2B receptor antagonist.

34. The method according to claim 32, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(S)-5-amino-3-(2-(4-(2,4-difluoro-5-(2-(methylsulfinyl)(ethoxy)phenylpiperazin-1-yl)ethyl)-8-(furan-2-yl)thiazolo[5,4-e][1,2,4]triazolo[1,5-c]pyrimidin-2(3H)-one; and salts thereof.

35. (canceled)

36. (canceled)

37. A combination comprising a compound according to claim 1 and an adenosine receptor antagonist.

38. The combination according to claim 37, wherein the adenosine receptor antagonist is an A2A or A2B receptor antagonist.

39. The combination according to claim 37, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(S)-5-amino-3-(2-(4-(2,4-difluoro-5-(2-(methylsulfinyl)ethoxy)phenylpiperazin-1-yl)ethyl)-8-(furan-2-yl)thiazolo[5,4-e][1,2,4]triazolo[1,5-c]pyrimidin-2(3H)-one;
and salts thereof.

40. (canceled)
